SEQUENCE LISTING (2 sequences)

SEQ ID NO: 1 - DNA, 113604 bp, Human
  misc_feature 7175, 7204, 36973, 66372, 76921, 81512, 88727: n = G or C
tgtcagaaaaagatacaacatttaataacgatggaatgtaaatatcaaatatttttatttacaaacaaataaaagtttttatatagaactaaaatgatttctataacacctgttttcactgttcttcaatatttctcttttaacttttcaaagattttctttttaaaatttttttttgtagagatgggatctcactatgttaacccagtctggtctcgaactcctggcatgtgatcctccctccttggcctcccaaaatgctgggattacaggcaaaagccaccacgcccagccaagattttctttttttattgtctgtatttaagatatgcaacaagatgttctgacgtacatatgtggagtgattaccacagacaagcaaattaacataccgttaacatattacaccaaccattaacatgtccatgttcgtgtgtgttttgggggggggtatgtgtggtaagagcacctaaaacctactctcttggcagatttccagtatgcaatgtgtacaatgcttataaacatttttctttattaaaacaaaacaaaaaaccgccacatatatggaaagagctgtgctggatgcctaaggaggaagcttgatggttctaagcaaaggccaaaaagtggtctgatctatggaaaaactggaaagccggcaaaatttgctgtgagagccctttctctgctcccatctgtgctgatctgcttttttcctacaagagcccattggccttttatagttcctggggaaaatgaagcccccacggttgtgcctccttgagttccaggacttccccctctccttcccatccagttttaacccccacacacctgtggcctgcacgctgggggttcttttctgtttcttttgatcctcttcctctttgaaaatcatctttgtaaaacaaacctaatackgcaccatttccgtccagattcatgctccaggaagaaagggtgctggaggctacaggggggcctcacagcccatgtgatctctgtgttacattcattttccactaacagaaatcaaagaacataccctgccattcggcctgtgacaggggtctttgttaactttgggtcttgctaaagttcagtaatgtcagggcaaacagaacacaaggaagctgagacgttctctggctttccgttgcaaactaggcctgttgatgatggtgacaagcttctcaatcccagaagcatgaagccagcaaggctgggaaagcaccttgggggaaggctccatcagaagagaatcaactttatcaaaacttggtttgctctatcacagcagcggcatttcagaagcatcctaccaagttgcttgtttcattgataaactaaagaaaccctacatgtttggagagttcttggtgaggcctgttcattggaagtcgtcatgcttgtgtgtatcttagagaagaagaaaattccagtagttcytcagtcaaatggtgtaatccactccagaatcattgccatctcttctaatattctgaaccaggcacagagaaagtagaagctcagtgcatagctaaatgaaattaccagagattctcaatgcccccatttccagcttttcacaaaaccattgtgctcacattaatagcatcaaggaaagcttcctactctgtgagctcaattagaaacgtcatgtattgttaatggttttgaaaaggtgaaaactttcttttccagagtcttttycatcggaatgataatcttagtaccttgtaaatagatgaggtggttgatttcatcacagccagaatctagaattatcaccattcttttgggatacagtgagagcttttttccagccagacacagaatgggcaatacaggtaaggtccctgttgtcatggagctcaagttctgttagagacaggaaagaaaaaaacaatcaataaaacaggaaaacttcataattcaaagaagccacgcacagctgtgagactatgcacaggcaccattcacactgtagactaagtgacagctgcccccgggttaagccctgccttgcctgggcaacagtacatggcatccccacacaattcagataatacaaagtgctatcagggaaatgtgaagaaggaagaagctgtgccaagattgggggaaaagcattagaggcagagagagcagcgtatgcaaagatgctgaggcagtccgtataattgcaccatgaagaggtccctgtcctaatctctggaagctgtgaatatgttaccttacctggtaaaatggactttgcaggtgtgattattaagttaaggatcttgagatgggaagatgatcttggattatccagatgggacaaatgcaatcacaaggctccttataagaagcaggcagggggctcagggagtgggagaagatgtgatcacaaaagcagaggctgggccgggtgcggtggctcatgcctgtaatcccagcactttgggaggttgaggcggacagatcatttgaggtcacgaatttgagaccagcctggccaacatagtgaaaccccatctctactaaaaatatacaaaaattagcctggcatggtggcaggcatctgtagttccagctactagggaggctgaggcaggaggatcacttggacccaggaggcggaggttgccgtgagtcaagatcgtgccgctgcgctccagcctgggtgacagaatgagactctgaaaaaaaaaaagaagaaagagaaagaaggaaagaaagaaagaaagaaggaaggaaggaaagaaagaaggaaagaaagaaagaaagagaaaaagaaagaaaaaagaaagagaaagaaagaaaaagaaagaaaagcagaggctggagggatgccaggatgccagggagagggccaagagccaaagaatgtgggtggcctctggaagctgcaaaaggtaaagaaatggattcacactccctcctcaacccccagagcatccaaaagaaactggctctgctgccatcttgatgttaacccagtgaaatccactttggacttctgacccccagaactttaagataatattataaatttgtgttgtttaagtcactaagtttgtggtcatttgttataacagcaatgggaagctaatacagattcaaagacaaaaacaaaacaaaaagcagagaagcaggatcagactatgtgagaagatgagctcgcagaaaggaccagcggccagtttttccatggcctggccagctgcactaagcattccgggttttattttcagggtgaaaggaacccaatggagagtttcaagcaggggaattgcgggctgtggtttgtgtttccaaaagatcccactggtccctcttagaaatatgtgaatagggacagagtgcaaagccagaggcgtgagcggccagcagtccaggtgtgagctgagaagtagctgtgcggggacgtgtttggagagtcaaattattactataataataattattattatttagagatggtgtcttgctctgtggcccaggctggaatgcagcggcatgatcccggttcacggcaatctctgcctcccgggttcaagcgattctcctgcctcagccacccaagtagctgggattacaggcacctgccaccacacctggctaatttttgtatttttggtagaaacggggtttcaccatgttggccaggctggtctcgagctcctgacctcaggtgatccaccctcctcagcctcccaaagtgctgggactacaggtgggagccactgtgcccagcctggagcgtcaaatcaaaacaaaaaaaccaacagagcagtaagaaacatatttcaatttattaccttcggggctaggggaagagagacaagttctaagaggcttttcgtgcaaaaatggaaagaactaggtttaaggcaacagtgagaaatgacaatgaaaaaggcaagcttctgakaacagctgtctgtttggctggtgagacggattgtgactcttctttgcaattggccrttgtactagtttatggcacaaaaacccaggcacagttttcaaagaagttgagatggggtattaagggtctggtgggttcgatgtcacccagcacaaactcatacacccacgttcaacctgtgcagagkcttttctttaacaggatgcagagtcaacagtatttgctagtgaattgggggtgtggcagggtgaccaaaaaaaaaaaaaaaaaaaagaaatctaagttaattctttggttttttggcttacacaactagaaagaaagtggagtcactttactgtgataggataggagtaggtttggtaggagaattgagttatgtttggacatctgaggttgagatgcctattagacatctgagtgaaaatgtcaagtgagcatcttgacatttgattctgaaattcagagaagagkactggactggagatacacatttgcaagtcccctacaaatacatggattttaaagaaatcaactttattgtggtatagtttacataaaattatacacacccattttaagtgcatggttcaatgagttttactcaggtaaccacctacaacaaccaagatatagaacaattctatcaccctccaaaattgtctcttaatcctttgcagtcaatcttcccctcatctggtcatagaaaactactaatctgctttctgtcacgagggatggcttttgtctttctggaatttctagaaatggaatcaaacagtacaacttctttgtgtctgacttcttttgctcatataatctttgtgggattcatccctgttggtgcatgtatcatttatttgttcttttttattgttgagcaatatttcattgtgtgaataaaacacaatttgtttacccacttatctgttgagggatatttggcttatttccagtttttagctattttgaataaagctgctataaacattcatgagtttttgtgtggcatattttgaaaatttctctaggtaaatacctagaaggggcattgttgggaacttttgatctagttaccaaactgtttacaagtggctgtatcattttacattcctaaggcaatgtatgaaaatccacttagcccacatcctcaccaacacttggtgttgtcagtctttttaaatgtagccattctggccaggtatggcggctcatgcctgtaatcccagcactttggaaggctgaggtgggcagatcaccctgaggtcaggagtttgagatgagcctggccaacatggcaaaaccccgtctctactaaaaatacaaaaattagctgggtatggtggtgtgcactgtaattccagctactcttgttgaggcaggagaatcacttgaaccctggacagggaggttgcagtgagccgagatctcaccactgcacttcagtctgggggacagtgagagattccatctcaaaaaaaaaaaaaaagtagtcattctggtgagtgcgtagtggtatctcattgtaattacaagttatatttccaatgacgttgagcatttatgtgcttattgtatatattttttggtagtgtctgcataaacctactgtccattattattgagtcaattttttttcaaatgatgagttctttatgtattctagatgcaaatctgttgtatattctagatacaaattcatggtggttatttttttcagtatattacttgcctttttattttcttaattatgttcttcaaggagtaaaagactttaataatgatgaggcccaatatatcaatattttccttttatgatccatgtgtttcagtctttgtttcctccatctgcttttaagattttctcattgcttttgattttaagcattttatgcaaaggtgtgactttctttgaaataattctctttgagctttttagttcttgaactcatggcttgatttttttaaaaagcagttttggaaaaaatctcattcactatttcttcaaatattgyttcagcttcagccactttctcctctctttctgggactccaactgtacttctgttagtcctttttcctgtatctctcatactttcttgggtgtatcctattattttgttctgtgcttcactgtagatattttctgctgactagatttctaattcactaatcctctcttcagctttgcctatgctgctggtaaatccatccactaaattgatacatttaaaaaatagatttctgcggagattctatttattcttgggctgactctggaaatttatatttttctggmamattatatatttcatccaaaacttcaaattgtatttccagagtgactttttatttagttgattctatatttatttgttttctcttttgttcctttctgtttttgttctaaagtttcatttattctacatttttagtttattttttggttcttttgctttttgagtttaatgagttcctattattttccttttatttatttttattttttagagacagggtttcactctatcacccagactggagtgcagtggcaccataatagctcagtctaacctagaactcctgggctcaagcctcttcccacctcagcctcctgagtagctagggctacagctatgcaccaccattcccagataattttaaacaatttcttttttatagagatagagtctcagtgtctcactatgtcacccaagctgttctcaaactcctggcctcaagtgatcctcctgcctcagcctcccaaagcagtgggattacaggtataagccaccatgcccagcctcttttccttttagtgaattcatttactgtataaatttgcctctaaggttcacattgtctgcatcccagaagttttcaaatatatggcagttcctttgtaatttaatatcttattatttatgtgttttcctctttatccatatgttacgtgatgatatacttattttttggttttcagatatatcaaggtgtacagactatatttttgctgatttataattcaactgcattgtagtaatggaacccagtctacataaagctgatatttaaaaatttggttggacttcttttgtgacccagcacatggtgagtttttaaaaccgttttatggacacttataaagaatgcacatcatttgttggttaggtgtrggttctctccatctattttataaaggtaattaattctattactcaattctatatgtttrctttttaaaaaaatctatcantttctgaaacctaattcctaccattgtgnatgcctcaatttcttgtaaatctgcctgtttcactttgaatattttgagtccatatacaagcccatgattttactatcttcttcgcaggttgtttcttttatcaaattgtagtgacatttaatatcctatggatcacctgaaatttcagtttttcttagatrtctgcattttagccttagcttgatatgttttttcatcaccttattttcaactttttatttgtttttattttagatgtgcatctttattttgtattttattttttgagacagagtcttgctgtttcacccaggctggagtgtagcagcgtgatcttggctcactgcaacctctgcctcccaggttcaagtgattcttatgcctcagcctcccaagtagctgggattacaggcatatgccaccatgcctggctgatttttatatttttagtagagatgaggtttcgccatgttggccagggtggtctcgaactcttgggttcaagtgatccgcccgtctcgccctcccgaagtgctgggattacaggtgtgagccaccagccccagcctagatgtttatcttttaagaagcatatagctagattttattttgttattcagtttaataatacttcttttaataggtacttattttaaaggatattgtgattatagatatattcagatattatcttagtcattggggcagttatagtaaaaattataaactggatgacttataaataacaaaaagttattgctcatagtttggaggctggcaggtccaagaccaaggcactggcagatgcggtgtctggtgaggtctccctctttgattcatagaccgtgccttctagctgcgacctcacatggtggaaaggggaaggcaacctccctgtagcctcttttaaaagggcattaatcgcattcacggggtctccatcctcttggcctaaccacctcccaaaagccctaccttttagtaatatcacatggggagttagaatttcactatatgaattttggggggacacaaacatttatgccacagcagatatctttctaccaccttatttggtgatttctgggttttgtttgtttgtttaagacagagtctcgctctgtcggccaggctggagtgcagtggcaccatctcggctcaatgcaaccttcgcctccccggttcaagcgattctcctgcctcagcctcccaagtagctgggattacggacgtgtgccaccacgcctggctaatttttgtatttttagtagagactgggtttcaccattttggccaggctggtcccgaactgctgagttcaggtgatccacccgcctcggcctcccaaagttctgggattacaggcgtgagccaccatgtccggctggtgatttctgtttaaaagttttttcttaaagtgttttttcccacctagtttttcattgaatgggtaaaacattctacatttgcttttattaaaacaagaaatgaattttgctgcatttcaatttatagattttactatcctacctcgtgccaggttctgtgctaagtgctgtatatatctgtgatcacatttaacttttataacaagccaaatgagcaggaactcttatctctatcttacagacgaagaatccaaagaccagggacagtaagtaatttgctcacctggtttgccagcctccatgacacatcgccgtccagttctgcctttaattaccaaagcacaacacgctgctttgattcccctctcctcggcgccagaattcaagagtgaagttaaaccgcaagggctgagktagaagattggcctcagttccctgttcccaccagcaggtggcaccgtctcctagcggaattcttacttgaacgttttgcttccatttctgcagaggcatggtgaacacagttacaccaccaaagtgttcctcctggctgagtttgcctatcttgttcagtgaagacaacccatgaggacaaatggtgttaatgagaagcttttgcggagttacagagatcctcgtatttctttaaaatacacctaataacgttaactctgcaataatttgtagatcatgttaaatcttagctatcttcctcttgccacccagtgtgcttcaagccacatggttcagagcaccatttaatgtgaaactccaattttaaaacaaagtgaaccttccttttacaaaaccatgagacaagttacagagtaatgaccacccacatgaccttgaagtgattttgagtgagtgagtgtaacttccgtggctgccatttaaattggattcaaatccaaatggctccacctccatgtcatcagacctcttgtgccctgattcccttggctaagttcayagtaccttccacatcaggttgtggcaatgattacctgaggttaatacgataaaagcacatggtaagcactcctaaatgatagccaatataaagactcagttctcccaattccaagggtccccaccatgatagaaaaggatcttttggtaaatagagtatgtttagctcttgctaggtctttaaatactttgctgggggccaggcaccatggctcacacctgtaatcccaccgccttaggagactgaggctggaggatcctttgcggccaagagtttgagaccagcctgggcaacacagcaagaccctatttctacaaaaataaaaataaaaattaaccaggctttgtacacacttgtagtcccattacttgggaggctgaggcaggaggatccctcaagcccaagagttcaaagctgtagtgagctatgattgcgccactgcactccagcctgggtgacagagtaagactctgtttcaaaacaacaacaacaaacaaaaacctcaaaacctctttgttggacttaacttccagctcctccatgtagtaccttagtacccttgcagcccgtttctcttttacaagacaacaatgttgttataaactcatttggawgtggtcccgtggaggagtatttaccagaatctagcttatttagcgtcttcagaacacggcacttgcctggaattatactgaccccctcaacccataccaaccacccagagatggctgttcttggctcctctccctggggccctgtccttcccacatcgtcttcttcttctttcttcttctttcttcttccttcttcttccttcttcttccttcttcttccttcttcttccttcttcctcttcctcttcctcttcttctttcttctttcattgagacagagtctcactctgtcacccagcctggagtgccgtggtatgatttcagctcactgtaacttctgccttgtggattcaagtgattctcctgcctcagcctccagagtagcagggactacaggtgtgtgccaccacacctggctaatttttacatttttaagtagagacggggtttcaccatgttggcaaggctggtcttgaactcctgatctcaggtgatctgcccgcctcagcctcccaaagtgctgggattacaggcgtgagccaccccacccagcccttcccacgtattctggcagggaatgctgttgtcccccaagcctaccctaagaggaagacttcttctggggaaagatgttcactgtacccaggccctgccctggctggagctggcaggaagggtcccagagcaggaacttgtgccactctgcccaaagccagagtccctgaggcacacaccccatcaggcaccaaggtgaattccaactgccagttagtatttaactttccacatacgattagattaaacatgtgggttcataaaagcataggattgcagactgcagttgcaagggcttagatggttgtaaggtgaaggtgcccagcaggctgaggcttgtgtgcaacccagaagagagctcgctaacrccagcaagaaggttcagaacagcctggctttggaaaggaatttcatcctgcccacacactgcataggtaagtcttagcacacattctttattttttgaggaattaagtaacaaagttatctatgtgccttttccagaaaatgataaaaggaatgattttcctggtacatggcctggctcctcatccactcttccttctttccttcttgtgttttccttactcatttctttgttaattgccttagaatgaaaattttgagagtttttaaaatggaggattcatggtaaacgtaggtaatcatattgttttctcttcttgatataaaaatgaaagactttgctgccttttataggcccaggtgatgtgagcgatctaccatgtttcaagaaaagaaaactttggggctgggcgcggtggctcacgcctgtaatcccagcactttgagaggctgaggcaggcggatcacctgaggtcaggagatcgagaccagcctggccaacatagtgaaaccccatctctactaaaaatacaaaaaaaattagcctggcgtggtggctggcgcctgtagtcccagctactcaggaagctgaggcaggagaacggcatgaacccgggaggtggagcttgcagtgagccgagatcacgccactgcactccagcctgggcgacagagtgagactccatctcaaaaaaaaaaaaaagaaagaaaagaaaactctggactttggggtcaaatgagtgttactttcctaatagtgtcctgattgctgttgtcatgaataacacacattcatgacaggaatggctggaattaggggatcattctgtagcctggagacagggcacaactaatgacatgtgtaagctcaaatcatggtcttgatcttatgtcttgtacccagttgagccaactggtcacagcaatgaaaacagtgagttattggaatgtgtgacctctgctaggacagtcagtgctggacactggcttgggtgatgtgagttctagtccaggcactgtggccaacttgagaggcttgtgatcttggacaggtgacttaagccctctaggctatagttattccacctatcagagagcaaaccagcctaaatgatctccaggggcccagcctgtgctaggactcagcaagaagcattcactggaaatgtaggtcctcctaggttgatacacatgaattgcccatatttgaccatttctaacctatataaatggctatttcatataattccagagaacataaatggtagttgtcttagcattactaaagtaaatgcctattatgatattctacttaggggtaggataagtatgtataccaaatatggtttgtttcgatttgatttttgagacagggtctcactgtcactgctgagtgcagtggtgtgatcatggcttactgcagccttgacctcccaggttcaagctatcctcccacctcagcctcctgagtagctgggactataggagtgtgccatcacatccagctatttttttatgttttgtagaggtggtgtctcgctatgttgtccaggctgatctcgaactcctgggttcaagcgatcctcccacctcggcctcccgaagtgctgggattacaggtgtgaaccactgtgcctggcctccaaatatggttgatgtctatcagtcagttaaacagtaattctgggaataaaaaattgaaatcaacccacttataattggaatgtcttagcataatgtccttcaacgaagctgctttcacacactgtgatttgtttttttcctgtggtcatggagcaggcatgggccactcggccacatctcatgcatccgtattcaaaagccaaatcccttttggatcctgtttatttggcctggccacgggtgagcacttagacatttaatccctataggccctttcatccctgtgattaagtcttatcaaaaagcacctcctgaccggcttagcagtggggcctttgttcacattagaagggttgaacaaataatgggcagttggggctgcttagctctaaaaggctggtgaacgctgccatgcctgcacctggaaacaaacccaaatgactccagtggaattcagcactgaagtccctcatctcaaagaccttttgtggcagagactcttggatgggccttaggggtcccaggagtcccctgaaattgaatgtagagcttcctacgtgcataggtatactttcttggggaaaaattaagtcacatcattttattttacttttcgagggatctttaacaccgcccctcccccacccccaattcccacaccccttaagaataaattaagaatcactgttctggtagtttccagttgaattccacagaggaactgtcattcattcacaccttcattcaacagatttttagtaaagatttgctacgtacccatcgctgtgtagggtcccgggattcagagatgagtaaagcaatccctgccttccgggggctcaagctctcctgtcatcgggactcagttactgaatctcactaaacatcctgaaggtaggagtttatagagtggttttgaggatcacatgaataagcacacaatacatgggtaattcaaaaacgaaaacaaggccgggcacggtggctcacgcctgtaatcccagcactttgggaggccaaggtgggcggatcacgaggtcaggagatcgagaccatcctggctaacatggtgaaaccccgtctgtactaaaaatacaaaaaattagccgggcttcgtggcgggtgcctgtagacccagctactcaggaggctgaggcaggagaatggcgtaaacccgggaggcagagcttgcagtaagccgagatcgcgccactgcactccagcctgggcaaaagagactccatctcaaaaaaaaaaaaaaaaaaaaaaaaaaaggaaaaaaacaaaactaacatggtcatttgcagaaggggcagaaaaagggtctctgcctagacctggggaggtcagggaaagtactatggattggtaacaaccggctgggcttcctacaagagaaaaagactatactcacagagccagaccccatctcaaaaaaaaaaaaaaaaaaaagtctggcatggtggctcaaacctgtaatcccagcaccttgggaggctgaagcagggggatcacttgagcctaggagtttaacaacatagtgagacctcatcactacttttttattttaaaaaagagttaataaaaaataaaatgaaaataaaagggtaaaagagccagtggcaaagtcttgagtggattaaagccagctcagctaactttcacagcagactatatcattttaaaggggaaaaagcacatctctgttacattgcttaggaaatatgcttggtatataccctggggcaatcttatctatttgttaagtttccttccaacccactagcctgtgtggccaggagagggagacaaagatcttagagctctctaaataatagaacttaaaacatcagacagagaagagtatattatcttggtgatggtaattctcaatgaggaaaatcctggggagggatgttctgtgggagaatgcctgcaagtttatttgtttagtaggtttgattattcagctgattgaaattcctttcccagatggggagatctgattctcttttcatgaaggaaagaaaagtcacatgctaaagagacgggcatgtctttagaacggcagcaggcaaacccactgctgggatcctggggcttttactagtggctagtcacaggtttacctcctgcctgtgctccttctagctgtgttgaaacccacttgccccatctatgaaccgtgttcagctccattttctgagcccccttatctttttgtccatacctgttgcaactctttgcacgttgcattgtcattgatttggtctctcccattcaactgagcctctcacagagttcctgtcacctctgcagtttcatcgcctagcatagtacctggcactttaattcatgcatcaaatgtccattgagtgccttctatgtgttagacatctgctataccgagctagacaaagttggcagacatgacagccgagtggaaaagatgagcccctaaaccaataatcacacacacacacacacacacacacacacacaatatatatatatatatatatatatatatatatgtatgttatatatatgtatgttatatatatgtatgtatgtatgtattaaaaaatcttggccgggagcggtggctcacacctgtaatctcagcactttgggaggccgaggcaggtggatcacgaggtcacgagatcgagaccatcctggctaacatggcgaaaccccgtctctactaaaaatacaaaacattagccaggcgtagtggcggttgcctgtagtcccagctacttgggaggctgagacaggagaatcacttgaacctgggaggcagaggttgcagtgagccaagatcgcgccactgcactccagcctgggtgacagagcgagactccgtctcaaaaaaacaaaaaatttttgccttgcaatcgtttgccttgatgttatgtctaaagccccacaattctctaaaaacagagatgtataaaaaagcacacgtatataattctctgaaaacagaatataaatgagtcattgctccatttaactgacatttgttgagtgcttgttataaatatggcattattctagctggtgtgaggttaccaattttttttaaacaaaagtaatatgaatatatacacacacatttagtgactgcatatgtgatgtgtgcttttgaagaaaaaggagatgctgttggaggaaaatggtggtggtggtgggaagtgatttagagtagaaccagggaagtctcagaagtgacaactagctggaacctaaagaacgaggaggtagcaggtggaagagaaaggcaaaggcattctaggttgagagaataggatgtgataatgtcccgaggaaagagagcttactgacagggagggaagatgtcaggtgtgaccgaactgtagtgagcaaagggtaactgaggaggtggtcaggagcctgctcagccaatggggtaaatactgttaaggaattaggacttgattttaagaacaaccatcgcatcattttaaaagcaaacaaattgcactataatttccctcttcaaaaaggcacattggctgtgcacggtggctgacacatgtaatcccagcactttgggaggctgaggcgggtggatcacctgaagtcaggagttcgagaccagcctggccaatgtgttgaaaccccgtctctaccaaaaatacaaaagttagccaggcgtggtgacatgtgcctgtaatcccagctacttgggaggctgaggcatgagaattgcttgaactggggaggcggaggtttcagtgagcagagatcgtgccaccccactccagcctggacgacagagcaagattccgtcttaagaaaaaaaaaagggcacattgatggctattcaaggcagagaggggcacatataaccccaaagagatggctctggggagggttgtgttgtattacattgttggcattgtattatccaggtgagagatgctggaggctgggcggtgccagtggtgatgaaaaggagagatggatttgaaacataggaataatctctcagattgtttcttggcatcacttacctaaaatgcttctttcaaatatagatgtacacacccctccctttaggatacttgggacaatgtgccacttagacataggggatggaacaaattggagagtctgtcaatgccccctgcaatcttttctcttgatgttatctcataatgccccacaattctctaaaaacagagaacataaatgagtcattggtccattccactgacgtttgttgagtgcttgttatgaatgtggcattattctagctcttgtaaggttaccaattttttaaaaaacaaaagtaatgcaagactgctgatgaaaatttggaatatgagaaaagcataaagaagaaaatacatatctttaagcacaccacccactgttaacattctgatctatgtacttctaatattttctccattttcatatgtacacatacatttatttacatgcatatataaatatcaaagtgtatatatataattttctctgccatttaaatttttactgtgtaacaatcatggattgtaaaaaaagtgaataaaatgtacgcagtcagtttaaagactaacaaaatatgcattaaatcaccagccaggttaagaagaaatactattacttataccctggcatctccctcccacctttacatagccaaatccagaaaagatccgttttcctaaccttgttcgcctattttattatttaaattgcagcaggagggaagcatgtctactttatccaatttcacacagacgctggaagacgtcttccgaaggatttttattacttatatggacaattggcgccagaacacaacagctgagcaagaggccctccaagccaaagttgatgctgagaacttctactatgtcatcctgtacctcatggtgatgattggaatgttctctttcatcatcgtggccatcctggtgagcactgtgaaatccaagagacgggaacactccaatgacccctaccaccagtacattgtagaggactggcaggaaaagtacaagagccaaatcttgaatctagaagaatcgaaggccaccatccatgagaacattggtgcggctgggttcaaaatgtccccctgataagggagaaaggcaccaagctaacatctgacgtccagacatgaagagatgccagtgccacgaggcaaatccaaattgtctttgcttagaagaaagtgagttccttgctctctgttgagaattttcatggagattatgtggttggccaataaagatagatgacatttcaatctcagtgatttatgcttgcttgttggagcaatattttgtgctgaagacctcttttactttccgggcaagtgaatgtcattttaatcaatatcaatgatgaaaataaagccaaatttgaagtaaagtgtctgggcagtggctgtggggatagaaaggagagatttacaaatcattgaatcttctttctcatgaaacatcatttgtgtgtgacaaattcaatttataaataacccagatgtattatgtagaagctgaggctcaaaagctatcacttgcttaccagacggacataggagcatttatctgtaatattaattcatgagtgtggagtctgaagagatgaataaacaaaccataagattactttacatttattgttttcctggcctttaacctatttagaagtcttaagacagaacaaacatttttctttttctttttctttttcttttgagacatggtctctctctgtcacccagcctggagtgcagtggtgcaatctcagctcactgcagcctcaacctcccgggctcaagtgatcctcccacctcagcctccctagtagctgggactacaggcacgtgtgccaccacacccagctaacttttgtatttttttgtaaaaacagggtctcactatgttgcccaggctggtctcgaacctgaacaaacatttcaaaggacaaataatccataccagagaagtagagtatttaagaagtacccagtataacaaaacatattttaaaactaacatttaaagttttgcagaaaactaatcttaaaaagttctcattatttaagaaaaaaaaataaaaagttataatgtcgctttaaaaatgtattcttttaacttgatttagttttcctctatttataattagttgttagcatttatgtttaagaaactaaaggatacagaaagggtctaaattgctgatgccctctgaagacctagacaggaactacttaatatcttgcaccatgtggtgcaggatatcatagaatgtcagggctgatcattctactgttggcagagaccacttcacttacagatgagagaagggcagtccactgagaggagacaatttcattcactaattcggtcaggcaacattgacctacttggtccactggcctagaccccaagagtataaagatgagcaaggccgggcacagtggctcacacctgtaatcctagcactttgagaggctgaggtgggcagatcacctgaggtcaggagttcaagaccagcctggccaacatggtgaaactccatctctactaaaaatataaaaattaaccgcgtgtggtggcaggagcctgtaatcccagctactggggagactgaggcatgagaatcacttgaacccgggagggggagattgcagtgagccgagattgcatcattgcactccagcctgagtgacagatgctaaacatcatagtacaatgtgacaaggtcctaacagagatcaatgcaaaggggacacagccagccagcacaaggacaggagggcatgcctaatgcaggtcaaggtcttctctctcagagcagctgagagtagcaggtcaatggcagcagagagatgtggggcctcagcatcccatggcttcatgcctcctagtttaccctgttctcctccccatgccccagccaaggcacagcaacgatgggcaaggcctcaagcctcagggtgctaggacaaaatttagaaaaagaggctcttcttcagagaatgcttgtagaactcgttattccaatcacaaggtttgtctctttaaaattacagagtgagatatgtacaaggtatctacttcctaataacagatttgcaattatgccaactgaagcattcagtacagttagagaaaaccatccatattccaagagcagatgtaggaagagtggcttccctcctcagatcagaaacccagaaatgttgtcccacccagaaacatccatctcagagaggccagagcagccatcaggctttaaatcccagccctctgctctgcatccagacagaaatccgaggtttccatcaggtgacaaagaccctctccttaaccaaactgtcaagctcctctgagccctcttcttgactagagcccaaccatggccctataaaaactgcagactctcagcacacatgatttcgcccacctttgcacactaagagacataaacgctagcataggttctaagagctgaaagctaaagcgcctgcccgagaaaagtgaatgcggcctgaagaatttactaattgttccaaccaaaacctggtgacaggcagatagtcccctgatccctctcttaaggcagttactttagaaagtttgcaattataaatcctttctctctcccttgagatgtatatcttctaccattcagaactgtattgtctctctgaaatgcaaacattcaaactctccttgctggatgggtgccttgctctaacttactgctccccatcacagacagaagtttgtttctactctagataggagccaattaacaaacccagatcacactgaccaaccccttcccactttctatgcatttccacttcctggactctgctcaagccccatccccactcagttacctttgcacaaagggaagttgagctgggcctcttccctctggcaatagctaatgatttcagtcaatccttactgctttaactggctttctttacctttgacacaggtaaacacatggagagcaaaatcgaggtttttctggccgggtgcagtagctcatgcctgtaatcccagcactttgggaggccaaggtgggaggatcacttgagctcaggagtttgagaccagcctggccaacatgatgaaaccccatctctactaaaaatacaaaaattagctgggtgtggtggtgggtgcctgtaatcccagctacttgggaggctgaggcaggagaattgcttgaacctgggaggcagaggttgcagtgaaccgagattgcatcactgcactctagccttggcgactgagtgagactccatctcaaaaaaaaaaaaaaaaaaaatcgaggttttcctaattaagtacattttattatcatcactgaaagtacaggtggtaacatagagggttatcagccaacttcacttttggggaatgggagaaatgctgactctctccaagcatgttgggtgtctagtggttgaagccatttgccaagattgtcaccctaggatccactcccaccaaacctgggcttttcactttcaacccagcaactgaaaatgccagttcaaacaacttgctgtttttttctaccccacttgcttttagagttccttctgcctgttttattggctccatataacctgaataccacttatttcttaaagcatagctcagatgctattttaaaaggagcccaggatagtggctgatttgatagaatctatacccagattacccagggtcaagtcccagctctggtacctgcggcctttaaaaccacgaaaaaattactttaatctctgtgtctccatttcctcatttgtgaaatggttatcattatagcacgtcccttacagccttgttatgagacttaggcaatagccactagtgcttagaacaaagctatttttgtaactttctccaggaacacttcccttaacagaaccaacccctccacccctcagtttgttcttccctccacaccctctaacattctaacataaccacaaagagtcctgatgggatttggagttacactgcctgggttcgaatctcaattccgccactgccattcgcgcgttttctattgccagccaccttactctcccctggcctcagtttcctcatccttagaaggggagcagagcacatggtggtaactccctgcacattcctctctttccttgtattgggtgcccagtttgtgccctcacatggcgctagcactgagtgggctcaaactctgtacgcgtttactaagcatattgactgaagaaatctggaaacctagtaccgcggcaccatatcgttaccccaaaggaaaatgcatgcacgctgtcagagatgacgaacactgcgtctggaaacttcttaagggcacccacgtgtcctcagctgcagacagcagcgaggagacacccagggaattcgagacagcggaaggcggaagggtcccgcaacaacccaccctccagctcaggtgagttcagagtgagaacgcaccgccaggcttggacaaaggcacccggcctacaccccagcggctccccgccggggcctacgtggacttcagcctccagccacagggacaagagctgctggccagggctgcccgcctgggctcactgcgcctgcgcagtgagcagcgcgccccaggtcttctgcccgggcccactgcgcctgcgcacggagtagtgcactctcgtcggcggcaccggcccactgcgcctgagcacgtagcggtgcatttcgggacctgtagttttccccggcaggacggtagaagtcgtggtttgtgcgcggccaggcgctggagcctccgctgccgggagcagtaagtgtgtgacgtcggggtagaagggagtgacccaaattccaaaagctctttgggatgctgcgatgtcgcggccggccccgcgctcgggttttccctcctagacaaaagtctgccggctcccggtcgcgccgggtcggggatccggaaggtgaaggccgccaggccccacctgcggggcgcccctgctggacctggccgtcgggcgccgtcaacccgttgagcagcgtgttccggctggcacgtggcccgggcggggcccaggattggttcaagcctacggtgttggtccccggagagtctagggagacaagcaatcccctggaatggtgggggaagcgatgacagcccctggtcctcatccgcagctctgggggaagtcggggggtggggagggcgggtgttgctccctgagtgttgggggaagggtatggggagaggaccctgaactagcccccaggttacccaggaggagctgaggcccagagaggttcagcgactcgcccagggttgcacagcgagcacaggcaccgacgtcgccctccgaggcctgggcttccagcagggagagacccggacacctgtcatcgcttctcggtggatccctgaaatgttgagttgtggagtctgggcagctgagatcgggcagggctggtttcttgtaggcccaggcttccgtgtagagggccaagtgatgcccaaggttcacctggcagccccctctctggacctacccctccttatgattgggtgaagggttgggtgaaaagggtagaggccgggaatgagaacagcttcagaaagctcagacaaagggcgcagcatgattcgtggctggaaggagacagcaagcgatagactgatccttgaatttgttagtgtgccaagaagaaaaagtattaatagattgtggacgacacattatccatattgctttagttggtctaaccaaaataagcgaatagcttttttgtttctaagagaaacctgacaaaggaagacagggtatttttgcggtgaaggaaatagaaatatttggagttgtatctaagccacttgttacttttgtgttttaagctaagatcatggataggtccagggaaagttaaaaatttcccgcacttcttagattttatgcccctcaaaaacatccccaccttgttggcttttgcagttcaaccttcagataccagctccctcgtttctaatattgcattaggtgtacatatggcagcagagcaaatagcttactgatacttttagcttttttcttctcatttgcaaaagactctatgaaaatggctggcttgggcatgtaattgaagggaggtggggaaaagtggtaattcagaggcagtgggtgggaatgagcaaagcatgtcagtgtggttcaccctcttgactcccacctcaccacagcccttctgctacaattgcagattgactctaaatatgtttctttcctaaagagcttgaattttatcactccaggtatgaagttgaggcagctgccagtatattttggcagtcaggttctgtgatatcagagaggatggtgaattgtgaattccagagttgcagaattgttctttagattctgattttttaaatgacagcactttggtttggagggttaatgacttaccccaggtcatatgccacaccatgggtaacaccaagagtagagctcagatctccagttgtcctggtcctcaggctactgatctttattccctgccctgctatcttggaatgactgcattttgccctggatgtcgctagcctgtatccttcaggttggcatgtccagtcatggaaggagagagatttaacataacaacaatggctgacattcagtgctttctgtgtgctggccatggtgcttagtgtttacagtctaatctgcaagagaatttacagatgggcaagttgaggctgagagaggccaagtaacttgtccaaggtcacactgctagtgaacatagcacatgttttccagagggccagaccactggatgttttttcattcatttcctcctgtaggatcgttggacctatttctcgttcttgactttgggaatcaatattctacgtacatcaattcactgggtatgcagttttgcctctgaaaattttgagggaacagccagactcatctactgtatttgtatacaactcaattaaagcaggaattgtaaaaataaaatttgtaagatctttaacattttaatatacaacattagctaatcactaagattactagagatactcaaagtgaaaattgtagcaacaggttataacatgttaggagcatatttctttagggcagtcagaatctgctgcttcttaaagacaagtgggccatttacacatgaaggtaacaagcacattcagccaccatcattatagttaaacagatctatgatttaaattcctatcgctaccttatctgactttgaaaaagtcatggggaaaacttggctaccttgtgccaactgctagcttgttttcaagatattataatcttgaatagatggaggatgaactttttatacttagatagctttgtaattgaaagtttgtataaaaacatcttgcctgaagttcatcttatccccattctatctaaaggcctttgaaattttcagccacttttcttaattatgacggtaagtacatttcaagagaagtgtttccctgacttttgaatgcaaagctctctgcctgtgtcaggatggtgccgaggttaaagccctggagccagactggatgggttcgaatcccaggcccacctgcgagaccctcgatgtgttacttaaattttatttcctcctctctaaagtggaggcagtaagctgttttatggggtagttgtgagggctaaatgtcttaactcatctaagcactttagccacttttttccatctgacacaaaaaagttaagctatgattattgtgctataaagcattggatttcagaagaagtaggggcactaaacaccatctgcttgacacctttcttcacttacagatgggactgaagctctagagggaagtcacttaccagagggtgtaggttcttcatccagagctgaagtcttcttgggggtatgtgtcatattctaagagtagggacctacaaggccttggagcgaatcccaaggctcgggctgccagccctgccttctcatttccatatgccatggtgtggcatatgacctggggtaatatcctctgaaccaaagtgctgtcatttaaaaaatcagaatctaaagaacaattctgcaactctggaattcacaattcaccatcctctctgatatcacttccttccctaccttctactaggtctccctcaagctttagagaaaattytgcctctgaattattgcatctgacaatttttctgccctgtcacttattcccttgctcccagataatcttcgaaaaaccaagatgagtttaattaacactcagaggacttgacaaagacactcactcccaaaccagcttgcgtttaggtctggaggcaggtggagggacagattttagacttgggcccttaggttcacagatgaatgggatgggagcccatgtcccctcagaagcggcgctgtgctgctggcggctacagacagctggtgaggagagctttctgcttagcaagggccaggcccgtctgggccctcgcccagcccatccacttcccaccagtctctcaatcgccttgtcaggacacagcccacctctctgtggagctcactttctgttcacattccctctctccatcaaagagacatctttctaaggtggtctgccctaggaactccaaattgacctgcttctttcttcctgcccagatcagaccttccagctgcctctcatgtacttgtctgttgggggcttgtgttgatcaggagtgaattcacagtctaccatgaattggaaggtgagtatcgttttaaatatttatggcttggggttttttcttcctcctgattgtgaaaattcagaataacagttctacaccagtagcttgattaaaaagaaaaagtaggtgaaagcataatatttatgtttcattttaagttaaaacataaatgtacatttattcgctggacttctggaagaaggccaggccttttcttcgagtgtgcattcactaacttcaaatcttcctcacttttcttacaaaaactatacctttaaagctttacctgcaattttgcattgtgctttctttttttacacattttttttgtagagatagggctccactatgttgcccaggctggtcttgaactcctgggctcaagcagtcctcctgcctgagcctcccaaagtgtcgggattacaagaatgaaccactgtgctcaggccgttttggttcctttaaagtagatgcagtggactgaatgtttgtgtttccccaagttcatatgttgcaaccgtagtgcccagtgtgatgatatttggagttggagcttgtaagaggtaattaggtgatgaggctggagccctaatgtttggaatagtgagcttattaaaagggctccagggagctctcttaccctctttctgccatgttgggggtacaacaagaagccagccgtcagcagcctggaagaggactccgctagaacccccctgtactgcgccctgatcctcagctttcagcctttcgaactgtgacaagtacctctttaataagtcacccagtctgtggtcctttgttctaggagctgaattgactaagacagtggattaagatcttatgagcagtgcatacacaaaatctttccagtgtttcatactctttcctaatcttttacagttgacttgccaacagcattttttttccaacgcaaacttgagtctttcaaagtattcaacttagttttcataaaaacttttgctttacacagtcatatttcacaagcgtaatgtttaaataagttatggaacatagtatcaagtacaacttaaataaactgcttggcgagtaaacacacctgacccctgtgaaacattagattcagctggtgggagcagaagttcaagggcagccagagagtaggtcagcaatcaggttccaccgagggaaaggagaatgtcatcttaagtcccggaagtcaataaggtgaggtggaggttgtttaagagagcagccactaaaatatattatagtcactttgcaaagtctaatatcaagcaaaaatcatacattgtctcaccatctagaaatggctactattaacaatctcggtatattcatctttttctgtatatatgtgtggcgtgttttcatgcataggatcttattttacgtgttttttcaattattataagcattttcttcaaaacatccagtggttttctattgcaaggagaaactggaaaggtctggaagcaggatcagggagccaggaaggtagctttcccatcttccccagctgtgtggggtgaggggctcggcaggccctgcaggagggctgagggcccaggaacttgtgtaagttaaagatggcagagtgagtgagctgtgaagggtgggaagatatagaacagtttgtttagcatgccttgaggatcagagctcacagtggaaaggttgggaaggaaagaagaggcagtgagaggatggagaggggaaggagcggaaagcagtgtggggtgaggtttagggaggtcattcgcccactgcagtggctaagtcagtagaggagagaggcagtaactggtactaagggccagggttcaaagcattaaacctcatgcctcaaggtggtgtttctcactcctgagcactagttaaggcaaggatttcgagccccactcccagagtttctgattgggtagatctggctggggcctgagaatttgcacttcttataagttccaggcggtgctggtgcacactggtcgaaggcaatgcgtgggaagttttcctctttaattgtagagtgacaccaacccatgtgaccactcgggccagtcttgcttgtgacagttttttctgccatcaggaacaaagtctgcccaaaccttcccagcttctgcaccagggaagtggctaccagggagcagcttcgtgtttaaacacagccccatctcgtgtagtgttagaaaggaatggccgcaggccgggcgtggtggctcatgcctgtaatcccagcactttgggaggccgaggcaggcagatcactttgagctcaggagtttgagaccagcctggacaatgtggcgaaaccccgtctctacaaaaaaatacaaagattagctgggcatggagatgcgtacgtgtagtcccagctactcgggaagctgaggctggagaattgcttgagcctgggaagtggaggttgcagtgagccgagatcatgcccctgcactccagcctgggcgacagagtgagaccctgtctcaaaagaaaaaaaaaagaaagacagtcatggccctgattgcagagagctgcagaaggtggaaggttcagtagcccccagtgcgtctggtggccttccccctctggctcagtgggccatggccrgcagcgacagtcaacagtgctacctgtgcgttagcaacaagtatggcctcattatttaaaaacttagttattcccatttcacagatattggggttttgtttttaaaaattgatgtagatctaggccaggcatggtggctcacccctgtaatcctagcactttgggaggctgaggtgggcagatcacatgaacccaggagttcagcaccagcctgggcaacatagtgggaccccagctctacaaaaaatcagaaaaaattagctgggcgtggtgtcatgtgtctgtagtcccgtctactcgggaggctgaggtgggaggattgcttgagcctgggaggtcagggctgtgggaagccgtgatcatgccactgtactccagcctagttggagtctcaaaaaaatattcatatagatccagtccaccctrgcagcattcattttcctccctgaaggtctgtatgtttcaagagatgtaaggggtttgttaaaaggaaattggaggaaggggttcataccactgaaggttagtgcctaagagaggggcaggaagggggccctggagctcttcgctttaccctgtgaatgttcttgacctctgctgcccttgtgctgcgtccttctcagtccacacttctgcctcttgccgtgcgtctccactgcctgtaaaacaaagtgaacactgaagcctcccactagggtccattggctgatgcgtttccatttccatgggttttctaacttctggatgagagagtacattcctgcaattgctaaagctaagtttcctatctggattgtagacagctatgggcagtaacatgggctttgttatrttagtaatagggccccggccaggtgcagtggctcacacctgtaatcctagcactttgggaggccgaggtgggcggatcacgaggtcgggagttggagaccagccggccaacatggtgaaaccctgtctctactaaaaatacaaaaaattagctgggcatgatgccgcatgcctgtaatcccagctacttgggaggctgaggcaggagaattgcttgaacccaggaggtggaggttgcagtgagctgagatggtgccattgcactccagcctgggtgacagagcaagactctgtctcgagaaaaataataataataatagggccccataggtttattcagagagactgagaaagctggaagagattagcttttcccagtgtgagtcattgcctcaggtagcctggaaaatcctagcaaacaaaaagaagtttatacaacaacattcttttcatagctggtttgtatgcatggcgtcaaaccttcacctctaaaatgtgaaccttcaagaaaacaggatttcagggtttactggagggaggggattagcctaggtctgagggaagaagaacctggaaatgaagtcagtgtttaaagctccttatatttaccagagtaaaaagttgaaaagtttcacttttggaactttttagtcttttgtactgatgccagagtgatctttctgaagtgtatgtgttgtgcttattctttactgttaaaacgatatcatggttgaaaactattagctaattactgagtgttcttgtgttcttactgttttagtaaaattaaaacgatttaagtatttgcgtccctgcctcctcccatgattttcattgtatttctatcatatcatgctatatccttctgcaaatatccatacataaccagttaaatgatttcagaggtagcgagtctagttgcctctggaaaattcagtagccaagccatagtgtatttgcatattgtaaatgtgaagtggatgggtgtgaggaatgaatcatatatagtacaggacagcgtgatgctacagagttgggctttggagcatttggagctgggtcagccctgcctcgctgactgctggcctccccgcctctgcattttctcggctccaccgcagtcagggcgagccatctgctcatggaggtggctaagggcagaagggaaagccgcataaggcactttgcatgccgtgagttcccagtctacagcagctgatgctacagcttctaagcgtgaaatccacatctagttctgagtcataaagagtttygatacaatcataggaacattcatctacatacactgtgatttcatgaatttcagtttgttgaaaatcaggcatcagtggaagggagactggccccggggttagaatgtccttccaactggctcctcatgagggagctgtgggaccttcaacctctcaacctccaggagctcttctttccttgctcatataaccagggggttgagtaggtccccttgaaagttatttccagccccccggttctgtgagcatattgtacacactaactaggttcagatcaacttcggttagactattagagggaggtgcacatgtatcccccacagtggaatctcattggtatttcatataataagtgattgacagaaataaggatttcattgggataaaatcctacctggtcctctaaaataatgattgctcaaccagagcataccttttcactatttgggagggaatttttaatcacacaaaaagcacatacatatcattcaggtcatgctaaccatgtgtatggaactaatattgctttgaagtactatttgcaatatatagaatttcacacaaaaaacctactagtgcaaagagtgagtaattcaaatgcatagtgtttccctagactttattttagtgaatggggttgcaagagcagttagtaagtagccatgttttaatgttttgagttctgtcgtgttttattttaccagcaagtgccagtgctagtgagtctctagaaacaagagaaaatcccagagtactaaagctgcagtttccagaagcaaggtctgattcacccttactttgtagatgaaggggcaggagctcagaaagaaaggagctcattgcctgacccaggtcacacggtcagtcggtggtaaagtagagctctgacccaggcctccgggctccagctcctttcactggatctggctgctgcctcagaagcaagggcctgggtgatcagcaggttgcacggttgagctgtgagagaccagagtccccacgcctgtggatgaccggtggtcccctccatgaagccagccgcaagcaagcagcaaagagcagagctgtaacttgactgttggccccatggggatagagaccttttctgcttggtctccagtgtagctccagccccctatcctcgtgcctgttgcagaataggtgctaagtaaatatttgttgactgaaggatcataaaagaaacctcccatatcggtgatggaacatttagttagcatggcttctttcttcttgaaggttcttgagcaygtgcccctgctgctgtatatcttggcagcaaaaacattaattctctgcctgacatttgctggggtgaaaatgtatcaaagaaaaaggttggaggcaaaacaacaaaaactggaggctgaaaggaagaagcaatcagagaaaaaagataactgaaggtgagtccacagtacccaaccttgcaaatgggagctggccagtgggttggggtgaccaatcaatgaacaagagaggtctgagacctccctgtccgtcgggtctgaagggctgcgtgggggcatgtggcctcacctgttctctaaggtagaactgctccataaagggccaggtgtgcagatcctggtcctgggatgtgagtgctgctgagccaaggtgcacggagcattagttcatccttcttgaaacctgcggtggcaatggttcttgacaggtattgggttaagaattgaggactcagtgacagctgtggaccttcttcccagagaagcacacatactgaaaccatctgcatttgtgtgtggggagaagtttgcagatgtggggagtcacaggtcatttgaaattccaagattaagaaaccctgttacctttaagataaagtctgactccatggtatgacaaataaatcccttcccagtctggtcttaacccagacctcatttgccacatgtgcccactccccatccccctgcaggccattcctcagccaccatggcctttggccttgcttattaggttcttcccaaggacctcctcaccaggcctgagggtacactcatcgttgtcacccagtgcctatcacagtgcacactggtgggcttagaacacacttggggagttacaatacatcaaggcaccagaaagttcctgttaagcccctgttacagatttaacacatgacaattcacagttaccttgattccagtgtgttgatgcagtttcttcactttgcaggttgagttctgtactttaaaaatcaggggattcagccaggtgtggtgactcatgcctgtaatcccagtgctttgggaggcaaaggtgggaggatcacttgagccaatgagttcaagaccagcctgggcaacatagtgaaaccctgtctctacaaaaattaaaaaaaaaaaacaaaaaaaaccaaaaaaaaaaaccaaaaaaaaacttagctgcacacttactgggtgtgtagtcccaactactcaggagactgaggtggaggattgcttgaggcccagaagttcaaggctgcagtgaggcatgatcacaccactgcactccagcctgagtgacagagcgagaccctgtctaaaaaaaaaaaaggattcaaatatggtggcggtggggttctagatctgtggttcccaaacctagctaatgatcagaattgcccaggtgtttgtttaaatgaagattcccagcttcagcccagagagattccataatggctctggttaggtttgggtgttggtttttgttgttgtttttaagataggggatcctactctgtcatctaggctagagcggtgcagtggcacaatctcggctcactgcagcctcagcctcctgggctcaagcagtcctcccaactcagcctcccgagtaggtaggactacaggagcacgccaccacacccggctaggtttttaaaacaatttttagtagagaaggggtcttgctgtgttgcccaggctggtcttgtactcctgggctcaagcgatactttcgcctcagcctcctgaagtgctgaggttacaggtgtgagccactgtgcccagcctctgttcttttgaaaacccattagataatccttaggttgtcttctcagcaacaggtcattgtaggaaccactgggctaggtggcctcccaaacccagcccactctgagattccatgctgaattcccttgcagtgccgtcaggcttgttgaggctaaagacctactgatgaggaatagtagcaacacctactaagtggttattctgtgctggggactttgctaagcattcatgcacaactgtgttattcagccctgatgactctgtgaggcatgttcagattgaaagaatggctctctctacatggtgaagaacagagtcagaaattgatcccaggtcaaatgcattcgatcagcatggccaagcccaagctgtgctaccttcctgaatacaggcaagtgagctcgtagggatgcttcactctgttactcaccacttccggcagctgcccactcgctggtccccagtgaactgtaggcttttgctagatagaagaagttactttctttctttctttctttctttctttttttttttttttttaaggtgtttgattgactgaaatttaggagtaggcattacgatggggaggagagaaattaaaaagggtgagggaaggcagtttaaattaaaatgttgctgattgaatttatattcctgacaatcccattttgtgtgctaaactgatcaaaggaagaaaagatgagatggaagatcataaaggctttgttcctcccacaaacatcagcagagacctgcatttaagtcaggcctggatggcttagaagcaactcagggagttggtcttcctctctaggctggcagcttcttaaagactagagacttgcttcaaacaaaaagcgttttcaggccgggcgcggtggctcacgcctgtaatcccagcactttgggaggccgaggcgggtggatcacttgaggtcaggagttcaaggccagcctggccaacatggcgaaaccccatctctactaaaaatacaaaacttagctgggcgtggtggcacgtgccagtaatcccagttacttgggaggccgaggcacgagaatcacttgaacctgggaaacagaggttgcagtgagctgagattgtgccactgcactccaggctgggtgacagagtgagactgtttcaaaaaataaataaataaataaaaggggggtggggtgtttcagatggaagggaaactgatgctaaaaatacattggttaataaatagacttgagtgatagacttgagtggtgtccgcttgttaagtttaaatggctgagcatacgtctttatgctgagcagtaaacatcgggtatactcttatcaaacatttcctactcatccctttggtattcccttctagattctgccatgtaaatgtcagcttgagtggactccagctgagaagaaagagaagaaagacttaattattgaataatttgtcagaggataaactcccaacctagacctttcacttaaaatagtgtgaatttgtatatgtttttaaaagaaccagtactggccgggtatgctggcttttacctgaaatcccagcactttgggaggccgaggcgagtggatcgcctgagatcgggagtttgagaccagcctggccaacatggtaaaatcctgtctctactaaaaatataaaaattagccaggtgtagtggcgcgcgtctgtaatcccagctactcgggaggctgaggcaggagaattgcttgaatccgggaggtggaggttgcagtgagcctaggtcgtgccactgccctccagcctgggtgacagagcgactgcgtctccaaaaaaaaaaaggtaaaattaaaattaaaaaaaataataataaccagtattttgtttactaaaataaaatgcctttgtaaaaaaaggagtcgtggcctttggaataagtcaaattgtgtatctctttctctttctcttacacagcaccctctaccccgtgttgtaaagcggggggttttgtaaacttacacctcccccaccatctcaagctggggggtcccaggtgagaggcttccatagaggacaaggtggtgcagaaacatctgctgtgggagtggggtccccagcactgggtgcttcggccagctacccccgaccccaggccccctcataggctgccctcccataccctcctttctcgtcttttcctcctacaggtgctacacccctgtgagagtgttttggagtgttttcattgttagggtggagggaggctgtgtgtgtccaggaaaggtgactcctgtgttaaccatgagggtcctcgcagggaggaatcgttgggagccctagggtgtgttttgtcctctcctcacctgtttgctccttgggatttgctgatgagaaatgaagggtagggcaccctagtagccactggaaccaagggcagggaggatgggaagatgttttactcagcacctaacacacgcagatccctgtgacaagagctcatgctctcccacttcttcgcaagaccccagagtggatggggagtgaggtggcagcagctggcactggaagcagtgcggagtgtttggtctggttgctgatggctgcatgggaaacttgcaggagtgtgtgttagtaaacgtctccccgtcctggcccagtttgtgtcgaacatgcgttttccatgtggggagtcaggggagttccatcttaaattgcactgtgcttgctggatgctcttcagnacaatttaggaagcaggaaagaatttacaaagttctgaggacagacagaccctgctcctacaagctgcagtgctcaccatagtcaaagtggactttcacgtaagcccagacctcattctctctgaaggaggccgctccagcctttgccaggagccctggtgactttattctgcctaatcctgctgcggcctggggtcctgttagaacgtgaatggaagaccacagcagaggtgggatgcccttggatttctgccatccccacgctttcgtgwcatgctcagatggggcctagaactgaccctgggccgtggcctaccatcctccctttgtcagggcctccttgcaccctggcaggttaccccacccaccctggcccatgttcctgccccaggggcctggcctctctgctgcccaccctgcaggtgtagggtatcacctgctcctgccttgcctggcatcagacctgctaccttggcaccacttcctccctcatgcccacccgcctgtgtgcctcataagtccaaggcgggggatctgctgaccagtagacactcatgtgctaaacacaagcgcttttctaggctttgggatttaaagctacactttggaatttgtggaagatctggccatcttggaaaattaggtagaaggtgacataaggactggactaaaccactgatcatcccaacagtgcccgtggcttttctgttttttgtttttgcttttgtttttttagagatgaggacgtgctgtgtccctcaggttggagtgcaatggtacaatcatagcttattgcagccttgaactcctgggctcaagcgatcttcccacctcagcctcccgagtagctgggactaggccctgctaatttatttatcttttgtttagagacaagggtcacgctgtactgcccaggctgggtgtaggttttttctgaagagcatttgggagttttgtttttgcttggttacttttcctatgcacccttctaccactagggggagatgattaatcactaattgaagggattttgttcgttttttatgttttgggtttttttgtttgtttgtttgtttgtttcaataaagaaagagtttaattgcagtaaggcaggccgcgcaggagatggcgttcttattcaaatcggtctctctgaaggctcagaggttaggggttttcaaggcggagttcttgctatcattccactccttaggtacatgaagttggtagatgtgtagtttgatgttaaattattgggtggatgcatgcaccgctggttgtagagactggtaaagcccactagcaggaccccacctggaccaaagcaatccctcaacccgctggrccatgaccgagaacaaacacaaaggacctgaaatgcgttgtgaaggccagaagccgacatccacattctccacccacggagagccccagagtccctcatgcacatcctgcttgatctattacacacattcacacattcgcaacacatttgtttggttttcaagcttacaacatattagaaacagaaaggaagaaaggctgtcagcagcagaaatacctttgagcaagagggacggtctttgagaagcagacttgagaactcaccgtgtgctcttcatgcgccagacactgcggcagccacagcgtcccacatgggatgccacacgtgatgatgttatgttcatggtgatgacctcaggcgtgaagaagaggttcagccgtttcacacagtctgtttaacaagcacatacataacacagacatacgtgaggaatctcagaaaccaaataattcaaacaaagagtctgggattctttcaaaagcgttgcctctgcccaagctttcttcaaattctgtctatagggaaacgtagctgtcaatgtctcattcccgaagacttccagatgcctggatctttagagttctccacctcaccccgagttgattacacaaatgttcctggggctttgcttagtgccctgctctgtgccaggccccacaggcagagatggcagggacccaggcctgacgttggagagctcctgacccactgccggaaacacacgcaccatcacaccatgagggagctcccccatgcagatctcatctgtgtcagagtgaagccagaggatggacggtggagagtctagaaggagaagagaaaggaggatgagttctcatacatgagcaagcaggagaggccatttaaaatgcacactctggcctggtgcagtggctcatgcctgtaatcctagtggaggccgagacaggaggatcacctgaggtcaggagtttgagaccagcctggccaacatggtgaaaccctgtctctactaaaagaaaaccaaaaattagctgggcgtggtggtgcatgcctgtaatcccagctcctccggaggctgaggcaggagaattgcttgaacccgggaggtggaggttgcagtgagcagacatcgcaccactgcactccagcctgggtaaaagagtgagactctgtgtcaaaaaaaaaaaaaaaaccaacctaaaaaataaaaataaaaataaaatgcagactcctgggctggattccaggtccactgcatcagaacctgcaggagaaggaccaggaatttgtgatacgaacatccccaggcaattcttgaccctccttttgagacctacactgtagaagatgggcagagggagaggcagcaggagcccaacctgggaaggagccatcgggaaaggtgggaggaggggcaggagacagcgcacgcgaggcagcaaatccttcagcccttactccccaagagctcacagctgcctccacagagggtaacggtatcattatccccttttcgcagataaggaaactgaggcagagaggccctgcctaaggtcccccagctggtgggaggcagagacaggaaccagccctcatggtctcactgtgagactggacttttcacagctgtgcggtcgagtctgagccaagtaaagtaaagcgagtttttgtacttcgaagtctgggacataaaatcttcaagacgtcagcatcagtgacacgatggtgacagaggccagcattgcttgttgtatctttttccatcctgttcctatctacacttccattcttctgttcattctgcttgcatttcccacccagatggctttcacggacacacacacacacacacacaccgcacacacacacatcactcacagacgcaccctgtgcccaatgtcaaaagacaaaactgcaacacgtttagtcatagacctcattgtcttttattcttgattcatgaatggggcagcctcccttctataaaacagagcaagagctcccaccggacaattgcagaacagtgggctttgtaaggtggggacaaggaaacagaacaatagaaaagaagctgatgggttaacatcaggttacttcaggacctcctaatcacgctgactcaggtagaccagaagctcctgttttcaggaaaaactaatctgtttggggacatacctgcttccttattaaagttttgggttgattatatggctcttagcatgactgactccattttggtttggtttgatctggtctgttggggcctagtgcaggatctcagtccaaaacaatagcctcccataatttttgtttaatgctgggtcagcggtaggcttggtccacttgcgcttctgcctgggtgggccttcctcttttcctccttttctctctgtggtgaaatccccattcttctttctgtcctccacttcagtttcacctcttcctgcaaccctgcccacagctcttcagcgccaggccctggactcagctctcactacgcaaacctccaacatctcactgagtgggaggtggcccccacctccacaccagacctggaccttgagggtccaggcttgttttccttggtactcctggccctgacgcacccagccaggcacacagaaggtgctccagtatttgatgagtgaatgactggtagcaccagaggaaagggagcagggagtatggccaagaccattagctgccttcctcagtgttccttctccccttctccctagtaatagaaccctgacttttacctggccgtatggtcactcaaaataaaggactacatttcccaccttctctcgcaaccaagcctggccaatcaggtttaagtggaagtgtagtgtgggacttcctggaaggatctttaaaagggacaggatgggcccctcttcctccctttctcctttttggctgcctggaatactaatgcaatcgcttgttccgcagccactttggactaagaggtgagtttgagacctgaaccaaatctaggacaatggagtaaaaagataggatcttgggctctcagtgaccatggatccatcatttcacccctgaactgacaccttgacacttcttatttttggtggtggtggtggggtagcttctattttgttggcatctctgtggtctattcatagttcacctgttttagctgacacatggagccagttagagatgggcgcaagggcttcctgatatgaagacttggattctggtcctgaccctcctgttactactgttacgtacgtggctaggtgagtcggtcaccaccctagcctggcctttgtaaaatgagctcaatgatcactgccctccctactgtacagtgtttgtctgaactgaatgaggtcaagcatgtaaagatggtttgcatgtggcaggacaatcacaaatggaaggagtgtatactacctcggtgggcaactcagccaccagttggccaggcaggggctccatccaacccaattggattgagccaatggccaacgaaccccatttgctaatttacctcttaggtccttgtaggggcagcaccatttcctaatgcacccccaccttgaaagccaccttgatccgtgggagggagaggggctgtgggtattagtgatggggaaggccagagaggctgggatgttccatcagccaaccactcagaaggagaatagtgccaccaacatcaggagctcacttctagtggacatgtctagagatatgtggctcagccctgcgttgctgcgtgtggtcacccgctcttaactaaatacagtgctctaaatatggctgctcccaaagagactgtgggctgtcctcaccagccatcctgtcccacccccaccagaagaaacctcttcttattattaattccctaaccgattgcagatattgcagatggtcttaaaggaaatgccagagaacaaagtttcctctgatcaaagtatcttaagatgtgtccaccctcattagatgccagcgataaaaaggaacaaaaatttttctcaaagagtgaaaaggaaatgagatcagtgatggcaggtctaattgtggacatgtttgcttcgaccttaccatggaccactcactcagtgggtgccagacacaatgcagtggcttggcgcaggttatgtgattttttttttttttttgagatggagtcttgccctgtcacccagtctggagtgcagtggcatgatctcggctcactgcaacctccgcctcccaggttcaagcgattcttctgcctcagcctcctgagtagctgggattacaggcatgtgccaccatgccaggctaatttttgtatttttagtagagatgggtttttgccatgttggccaggctggtctcgaactcccaacctcaaatgatccgcccaccttggcctcctaaagtgttgggattacaggcatgagccactgctcctggccaggttatgtgattttaaagtcccactgtattttattaaggagttgattgagcctcaaggaagtaaagcaacttatccagaacatatagctacgaagctagaaactcaagattccaactaggtccatccaattccaaaagcccggtgaactttctgttactttctatgggaagttttcaggtttagtataaagcaacatttcttaattatgaatacaatcttatgtaaccaaattgcacattaaagggtattccttttgatccttctttcctcttttctaataatttttacaaggcctcaaaggaatagaactctaaatctgttttattaaattgctttagttttcaaacaaaaatagctcctgttggtttatctttagctgataacctaaaaacattccttttttatcgctgtaaaataatgtgtctttaaaggatattctgtctcctctttaattttatgttgaaataaaattttttggtgtccaggggagttggccattttagcggctggtgtctcatctgaggtcagaggtgagtcttgcgacatagaaatgatgggggtgaaaaaggaaaagctcagcttcaataactaagctaagcacggtggtgaggagcatgggcttcagaatctcttggccccagtgccttgccagtgcctcagtttcctcatctgtaaaacaactgacaggattaagagaattaaattataatagctatgcgttagtggcatcatgaagaatggctctaagcatgaattctgggttgaatctctctgggcctcagttttccacatttatttatttatttatttatttatttatttattttgagagggagtcttgctctgtcacccaggctggagtgcagtggcacaatctcggctcactgcaacctccacctccaggattccagtgattctgctgcctcagccccctgagtagctgagattacaagtgcccgccatcacacctgtctaattttcgtattttagtagagatggggtttcaccatgttggttgggctggtctcgaaccccttacctcaaatgatccgcccacctcggccccccaaagttctgggattacaggagtgagccacggtgcccagcacgtgtagctgttggtgcctattattccagaaccgcagaggcatggaataaacgggatgtagggcaaaacccactcatctcccaaaactcaggccacccacacgtgggcctgcacaatgacagcacactgaagtgaccaaggaagtttgagtaccatggtttgctctcaaggaacttaaatcctaaaacgtacatgaaatcctgagggaaaaagacctcacaaatatactacacaataggagttatttcaccaggttaattgccaatgagaaatgcaatgtttctattggcagagcaagacttcctttagcacctgtggctggctgaatagtggccaccaaagatatcaaggtcctattccctggaacctatgaatgtcgctttagatggaaaaagttctgcctatgtgaccaagttaaggattttgagatgtgaagatggttttagattactcaggtgggccctgaatgcaatctcaggtgtccttgtaagagggaggcagagggagatttgacacaggatgagaaagtggtgtggccacaagcccaggaatgccggtggccaccagaagctggaagaggcaagaaacaggtcctccctggagcctctggagagagtgctgccctgccaacagcttgcctttggctgtcttctctgtcctgtcctgtcctatcctttttcttttctttctctctctttttttttgtttttttggagatggagtcttgctctgttgcccgggcaggaatgtgatggtgtgatctcggttcactacaacctctgcctcccaggttcaagtgattctccttcctcagccacccaagaggtggaattacaggcgcccaccaccatgcccagcaaatttttgtatttttagtagagatggagtttcaccatgttggccaggctggtctccaactcctgacctcaagtgctcctccgacctcaggctcccaaagtgctgggattacaggcatgagccactgagccaccgtgcctggcctctttcccttcccgtcccccttcctctttcccttccctttccctcccctgcccccttccccccgccccctccctctcccctgccccctccccctccctctcccctctcctccctcccctctttccttcctccctcccttccctccctcccttccttccttctctctttctttcttctttctttctctctctctctcccctccctctcctccttagcccccctcctttctcccttctctctctttctttttgagacagggtctcactctatcacccaggctggcatgcagtggtgcgatcagagctcactgtagcctcaagctcctaggctcaagcggtcctcctgcctcagcctcccgagtagctgcaaccacagccactacatctggcttaatttctgttgttttaagccaccaagcctgtggtaatttgtttcaacagccacaacaaattaatatagcactgttgactgtcaagtgaggcctgcaacagtggaaactttatagttctgagtggtcagctgtttgaggtgtgattaatctccaggaaaaatgttaccaggattccctttgtaaccatgacaaaatgctgagaagtggtggacacttagttgctgaaaagcactgaacgttcgctttcatctgacaaagtctttctgaataatacagggaggttcgggagggaaagaaggcaagcaaacgatgggatgctttctgcacgtggctgtcaggaaattctgggtagaaattctttctttctccttttcttttctttttgagacagagtctgactctgttacccaggttggagcgcagtgatacaatctctgctcaatacaacctccgcctcccagggctcaagtgattctcccacctcagcctccctagcagctgggaacataggtgtgcgccaccatgcccagcgaattttggtattttttgtagagacgaggtttcaccatgttgctggcctcgaacttctgagctcaagcaatcctttcaccttggccttccaaattgctcgtacaggcataagccactgtgcccagtccagaaatgttcataaattgttttttttcatagaccttattttttagagccattttttagattcatgcagaattgagtgaattgagtgggtgccccaccacccaccaccgctaccaacatcccatgtaagtgtacaaaattttttttttcttttacaggcagggtttcactttgtcacccaggctggagtgtagtggacaaccatagctcactgtagtctcaaacttctggctcaagtgatcctcctccctcagcctcccaagtagctaggactatagatgtatgccactcagcccagctatttttaaatatttttgtagagatgaggtcttgctgtgtttcccaggctggtcttgaactcgtgacctcaagcagtcctcctgcctcggcctcccaaagtgctgggattacaggtgtgagccattgtgcccagccataaattattctttttgcactcctcggaatgattacctctgaaatgactgtctgaatgtacataaccaacagtgaattttatgatatgtttaataggggtagtctttttctataaaaatgaaaaagacaaccacagattcttacaacagacatcggcagcagccccaaacctaaatagggaggtgttgtcccagctgcaagtggctcagctacagggtctcgagatgaaactccacggcacagtctaatgacctgctggaattcctggagttggcggggagattggctggataagtggtctctgcagacccccatgcgtgccacccctttgcccaccatagcccacttgaacccaccactgtcataactttccaggtcctaactgggaccacctactccccttggagggtccggatggagtttcccctctgcggctgcctctcactcatcctccaccactttgccgacaaggaaggaaggacaatcgggaggagggagtcttgcttggcgaccatctggacaatatctcgcccatggcaagctggatccctgtggattactttggtgggtggaaacttggcacactatttaatttgggctttttgggggatgactgcacactggttctctttttttctcagacctaaatttcgaccactcaactttcagcctgctcacccctgcttattgccatccacctttgggctgggttagacctaatcctccccttgacaaagccctcaaagtagccttcacaagataagagtcgagacccctaacttaccaacaaattttaatgaaacttctagtaaactcaaagcgctttgccaacatccttaggagcaacccagttctcgcagcaccccatgtgaggtcgctgttgaagtatctttggctgcagtggtgtggaattcatctcaacagagttgacggtaaagaccagacatgtagcagcctcactgcttgttggaggaaccacccttaagaaaatttcccaagatagtagttttcccatctccgtactttgctagttaatgttctccacttgcttgcgtttatcctttcaattcctttgctaattaaattgctgaaaagtatacattggtaagaatagccactgtcaatggactgcaaacaaaactcgtttctgttattgatgataaaccatttcagagaagaccaggctgagtctaaagggttcagataattacaaggggaaggcagagagagtagttcatggccacaggacttggccacctctgggcaacacataatgcttgctaagctaatgactgtagggatatagaactggccctcagcctctctgtcttcctttggcctctgctcttcctgctgccttctctctttgaccacgatgacacactttccataggccttcctcaccacatcacagaactcagagaagaggctgtcttcttgtttgatacacagctcgtctcttaggaacacccgatatttccaaggcacccatccttgactaccygcagcatgcacaatacaccaagttggggtttgtatgaaatatccatcactaatatcttccccagttacgagactttctgggatattggtttccccaaaatatacggtcttaaatccatcatattgcagtgttctcagggttttcagatattggaggcattgcttcctcttgatgccatcaaattgacttctgataatgacatttcttaaaagagggcttgcaaagcgaggcatggtggcttctcaagtgtttttaaaagcttgcacatctgcatctggggagagatggcaagtgggggatgatgacctcataaagggctttcttacagtggtgccagtcccacacatacacttcagatttcaagcatcatggaataaatactcagaacaactccctgctgacccataaaatatgggaattgcagatgctacccataaaaatgctactttgacatcacttctgcaataggtctgagacagaggttggcaatcttttaatagtaatgagcctacccatttggcttaagatggggagaaatgtttacattcattcatccatttcaaaaatatgtactgatcacctgctgtacaccaggcattgtgccaggcaccaggggtatagtcatgacacacccagggagtcctggacttacgtgttgtgtgtctgtgtatgcatagaatttgtgccagttgccttcccatttgcacaactaagagtgattttttccaggtggcagaaggaaggtatggcaaattgcaaaagaaagtcagtctgcacacctagcttccactggmgcttgcaggtctttttttttttttttttttttttttttttctgagagggagtctctctctgttgcccaggctggagtgcagtggcacgatctcggctcactgcaacttccgcctcccgggttcaagtgattctcctgcctcagcctcccgagtagctgggactacaggcaccttccaccatggccggctaattatttgtatttttagtagagatggggtttcaccgtgttagtcaggatggtctcgatctcctgaccttgtgatccgcccgcctcggcctcccaaagtgctgggattacagatgtgagccactgcgcccacccggagcttgcagttattgaactaattcaatacctcatcttgaaagcacttttaattttatatactcaggcaaaatgacagtttgcttcaaactctaaccatctcttcctttgtattttcttgcctctttaatcagagctaaagacatttcataaaatgggcatgaaggattccttcaaatgaagacgtggacaaaatgattggtcaggtcctttgctctactgttgaatggaggaggattttttttttttttccctcacacaggggttttcttggagctcaagtttggatgaccccagacagtaagataatctcatcatggtaaagttaatatgaaatatgtggtctccaaacagcctctcccagaggccaggatcagcaggtttgagtggataattggcttgtggtcattttctcataggatttttcttttagtagtggaaactgtttttcaaatcaaatttggatgccaactatgtggaacagaagtgtggctgctctggtggaagtggcaatggtagtcctagagtctccctgtcagccacaccctttgtctccccctacccaagggaccctgtggcctggaaccgcagtgtgaaatgctatatagtgcaatgaagtcaattcgaagacaagagttctttgcctttctcatctaatttttagttatggatatgagaygcttgttcagaagtatggaaaagtatatataatatgttatcttttagatgtgggtgtaaatatgcttatgtatgcaatatgcttatattttaacgcataaacaacatgaataaagcaaacactctagacttctccaaatgtatcttgttttacagttttcattttggaaaatgtcaacatttttacattaaaaaatattactcagccataaaaaagaatgaaatcacgtctcttgcagcaacatggacagaactggaggccattattctaagtgaaataattcagaaacagaaagtcagatgccacatgttctcacctttaagtgggagctaaataatgtgtacacatgggtacagaatgtaaaataatggacttcgaaagggaggctgagatgggaggaccatttgaggccaggagtttgagacaagcctggccaacatggtgaaactgcttctctactaaaatgcaaacaaattagccagacatggtggctgacacctgtaatctcagcactttgggaggccaaagtgggtggatcacttgaggtcaggagttcaagaccagcctggccaacatagtgaaaccccatctcaactgaaaatacaaaaaaattaactgggcatagtggtgcgtgcctgtaatcccagctacttgggaggctgaggcacgagaatcatgagccgagattgcaccactgcactccagcctggacaacagagcaagactccgtctaaaaaaagaaaaaaaaagaggataggattagggtgagggatgagaaattatttaatgagtacgatgtacactactacactcaaagcccagacatcaccactgagcaatcaatccatttgacaaaactgcacacctgcacttgtaccccttaaatttatayacaaacaaaaacaaaggcaaatcaaaaataaaaataaaacaaaatgatctctaaacaatacaaacagtaactgatgaacctagctgcttatcatgtcrgttccaaaatcacacagagttgaatttctttcaaatgaccctacaacacagtattttgatcatatattctccagtagagtataagctaaggacaaagaaaaacacatgaaatcttaaatggtactcggtagttttattgttaataatgatgctggtattattattttgaaactcatgtccatttctcagctgccayttgatattaattttttttttttttgaggtggaatttcactatcactcaggctggagtgcagtggtgtgttctcagctcactgcaacctccacctcctgggttcaagcgattcttctgctttagactcccaagtagctgggactacaggcacgtgccaccacacctggctaatttttgtatttttagaagagacagggtttcgccatgttggccaggctggtctcgagctcctgacttcaggtgatctgcctgcctcagccttccaaagtgctgggattacaggcgcgagccactgcgcccagccagtgatttttaacctttatcaatctgatagacaaaaagatcatttcattgttttaacttctttaattatgagtaaatctggcaatatattatgaaaacaatgacaagaagaacttgatataaaatgcacaattcagacgtccttgaagatcattttagaggcagcatgaagtggggggtggcactggtgatgggggctgggtgtggagaaaaccagccaaaggaggactcatgggatmctggagcttttgactggggttcacttggakcccatactcaggctggttctgacagcagcacctgccaggcctcagcttaggggcacaatgtgggacacatggactgggggtgtggtcccagagcctaagaggcaccaacagagaggtctccgcagagactcatctgtgccccccacccaccaccccgggacaggccaagccagcgtctggccaggaactgcttttgcacaaggagccagaagtagtttgccccgataaatgggggcctggactcacgcaaaccattgcaccatggatggccagagaaactcagagaaccttcctgtgcttgttaacatactctctcacgtcctctgcagcctctgccaagccaagcagccagctcctaggactcccctccctccacctgaggctccttgtcctcccttcctcaggagtctccagcctcccgggacttcccctccccgctgcccactccagcagaggctgccaactgcctgggagagagaagtgggcttcctggggccacctccccaactttggagtgtttggaaggtgatggagcgaccactaggaggcagtgtggacaggtctctgtaggactgctcaggcagacacctttgcagggacctcccaggtcgggagccctccacacttttcccatgggagcttctccctccaccccgagtcactactatctgctttcctagaaggcacttctttacttctaattcttctccactgcccaggtaactgatattctcaagtgggacattgtaatttgtttaattcatttaaattgatttcatataattgggagataaagattgttcagttgcaagacaaagtcttaacttgaactctcaggacacgggtgggtccctaaactcaatacgtgagtgttgctgccgggctgttgggccatcttccacccgccatagatcacwttcttcatcaaagaagaaggaatatttagaaactggtagtacaaaaaaacaaacaaacaacaacaacaacaacaaaaaaccaaaacaacaaaaaacaccaaatcaccaaaaacaaacaaacaaaaaacaaataaaaacccaaagcagttgctcctataaatagatgtgtgtatacatgtggctggtatgaatcttatccacaaattcagttttgtgggaaacatcacatttatttatttaaatcaagtcatatgggacttgggcatggttggaggttcttaccccaccccacttcccaaggccagtgcacaggcagggccttgaggtcacccttagccgatgcttgggtctaggtgctcagacccaagcccctgtggtcccatcatgtgggcactggcatctttgctgaggctgagaatttcaaagccaggatccagcccatttaggtaaacccaaagtcactctcccaggtggcccagtcatcttcttgagaacaagagccatgagcctcagttccctgcctcaagagcctctgttcaaccctaggcttgtagacaactctgccccttcttctctcccttcagtgtcacggtcccctgtcccatccctctctgggacaggtaccacaacctccccaccatacacagggaaagggtcagccctcaggtttttggcctggcatcttgaatctcctcccaggcaacaaaccacagagggcctggcattctcctgtgaaaagcagggcggaaaggaaacacagagaacaaacccacagacaacaaacccacagaaaacaaacccacagaaaacaaacccgcagagaacaaacccacagacaacaaacccacatcaacaaacccacaacaacaaatctacaacaacaaacccacagagagcaagcccacagggaaccagccaaattatgtctgctgtgcatctcggcagacgatgctgccaccgtctgtgtatgagcatgtgtgtgtcagactttcccatcgtctccaaacttgttttcagaataatgcttccagtgaaatgagtcggccacatgaggtcacaaagcccctactctgttcagcacctggggtaagtaataatattttggagcacttagtgtggggagtagccctgacccctttacatgtcatgtcttagttcattcttgttgccatccttggaattgaggccaacatcatctgcccatttgccagacaagctgctcaggaggagagggccacagccccttatctcctcgccaaacaagagaagatccccagttgctttttttttctgtggaagagattcttttaaaaacatttttttcatggagaagaaaatctgaaaaaaaagaatgaaaccgaaccaatagtcccatagacagttagttgttgttgttgttgttttgtttgtttgtttgtttttgatgaatacagaaattgacccttctggtcttaaagcttgaaaattaaatttgttttatctgagttgcttcctcaggaaaggagcccaagtcctctccaaaagtatcagagaactgaaactcaccagatcatcttgtctagacaatgagacgtcaggccctccattcatcatgactgcttccttacccctcccgagttcctgttacatttcttccctgctatataaacccctaattttagtgggtccagaagatggatttgagactgagctccatctcctgggcagcagcacccaattaaagccttcttccctggcaatactgattgtctcaatgattgccttccttccttcctttctttttgagatagagtctcactctgtcacccaagctggagcacagtcgctctatcttggctcactgcaacctctgcctcccaggttcaagcacttctcctgcctcagcctcctgagtagctgggattataggtacccgctactacagctggctaatttttgtatttttttttttttatagagatggggtttcactatgttggccaggctggtctcaaactcctgacctcaggtgatccacctgcctcggcctcccaaagttctgggatgagaggtgtgagccatcacgcccagctgagtatgtgtgtgtgtgtgtatgcttatggggatgtgcaaatgtgtgtgtgaatgtgtgcacgtgtccttgtgaattgtgaatacccaggacttgagcacactcagttcctgatgcacttcctgttttctcagcagctgagctcaggcctggaactgagtgacagcacacccgggcacctgtctccctgggcacccctcccacgcctgcttcccacggcattcccagctcccaccactgggaaggagctggaatcatgagtcgggataatcaccraattctcttcgaccttcctcagctcctggtttgttaaggcaaacccccatctctggcttctcctggaacctcacctgggaaagaaagaggcagccccggagctggaagctgcttcagggctcacccggaacagcagactcaacctggacccatccaggcatctcctgggagtttcacccaattgcttctgcctggcaccagctcagaggttctgacagaattggcctggggtgagacctggcatccatgggatttttacaagcttccaggtgattctacagggaagccaaggtgagaacccctgtcctagaaccaggtctgatcaggggccggtggggaactgtgggtggagaacattagtgcttccagagcctcagggttggttttgaaaggaacgtaacacatttttttttctcacaaagacatatagagagagactttttaaaatagacatatatatagagagatgttttaataaagagaggtttgggtttatataagtaaaaaagatgatagaaaatagagaatgagattaggcttccccttgctctcaaaaaatggtttgagagtcttggaactggttccactctatgatagccatgagtactgttcgcccaacttttggttctcagccttccaggccccggtgggatgagacttccctgcccccacggttgagaggagccatgggcttattctagccaatgaatggtggatggacgtgactcgtgtctcttccaggctggagcatttaattgtccaggtgagatactcagggactcgtccctccagagctgagaatggccatgtttccagagggtctgcaggagcaacctgagtctcagagcacagccaccagcagacctgctgcacgcatgtggcgggggaaagaaagaaagccagctgtctgcagccactgagagtttggggtggttgtttctcatgacaaaaccagctcaccctgacttatacaaagtctttgagttatatagatggagaatgaggctcttgggtccctctattctcacaaagcaatagcctagctaaatccatctaactagggagcagaaaaggggatgtgctggcttgcacaccctagacagttgttcaagaagtcaggacaccaggcctggagtgatacttcagccatccttctaggtgagggtcttgaggccacacagacagaagtggcagagatgggacacacattcgtcttctcactcacagtctggcactgagctgtgggctgctgggacaccatgcccccatacgagtagccttcccactccttaccttgaaggaaaagtgttttttggacaaatacctgatggaaacattacatgggccttggaatctgttagatctagcttcctgaaactcttgctagctgtgtgacaatatacaagtttcttaacctctctgagcctcagtgctctaattacactcccctcatagagtttctaagagcatcctggggctggcacgtgtcaacgcacccagtatttgatagagtttgttaaacgttggttatcctctctccctatcgcacctcaaatggtaagggctgcctgccagcttccatatccccagcagtgccctgagttgttcagatgttcatccatctcccacagaactcaggttcctttggaggaagccacatcaagtcctgctccaagcttaagccagtcagcacattccatgctctgccccattgccagggttcaggagtatgctcgtgatctaagcacccccactcccagatacagctcaagattcttgcttggacttctgggcactcaggctcctccgagagggaatcaaacttacttctctccatgtcctttcctctaggagatgcgtctttcataaaactctcatcaacactgttcagacatgtcccaccccagcaggggacagcctgggctcaagctgggatccctactttatttattttctgctaattaaacttcctaatatactccacactaagtgtgcttgcaaggcagggggtgtgggataagcggccctgcctggctgggagagggggcagctccctgctgtactatgtattaataaakagacacatgcatggcagggcttgtctgggccttggtggcagcttaggacagaaggcacgtgacagtcaggggttcaaacaacccagggagaacactgcttcagggaagacagctcagcatcttcctggcaaagataatgacattgataatactctccaaagaatttcaggattttgagcaatcagaaaagcaacacagaaattcatgtcatcaaaacgatatggctctattggacacttaagacatttattggaggctcaacaacataatcctgctggttggttttacttcattgattttccgttgtgtctgattacattgctaatgctgatggtggatgagctacggctcttttcctgcctgtcctgaggtttatccaccaatgtttcagttctgttttaagatattgtcctaagcccccagcatcgcatgcatgctgtttttttgttttgttttgttttgttttttacaaagagttcatagcccgtggaagactctcctccatcacacacttaggttccctccacaccaggcctggaaggagtctagcttctggggactgtacatatgctgtggaccatgcagaacctggagaggcggtgaccccttctagaagtgatctgcctgaatccttccctctggaggaggcatttattaaatgccaggttcctgaaaggctctgagatgggcactccctctcctgagtcgtcccttccattactgctttcctatttctggccagggttccctggcccctcctccctgctcccatgggaccccagttcatccccatctttgctcaattgccctgcactgtagtaatccattggcactcttgtcttctccaggagaaatagttggaggagaagtttatagggtttcctgggccagggctggtctacagtcactggacagcaggaaacgacccttcggggcctaggagggccaaggctggtgggcaggtacagggggagccagcactgctgtccaccactgtgcagcctggaggctgtttcccatgaccctgctgatgggacccaaggcaccccaggccacccactcccctgcccccagcagggtgtcagctccccggcttccctgcatgcctgcctgacatggacagtgcaccttcgggccacacttgccctgctagcgagcctccagtgaactgggaattccacagagygtagaggactcgccccagcactgtgctgagaggcttcaccaaactgtagcctggcttccacctgcactaagctgcatccccagaggcgaccccagccctggctgagtcttggctcaagactttgcaatgcagccaaatcacaaaatgcacctcgtccagcccaccccgctaaaccattttcagtagttctcccctcaccgttctggaactttccatttccacgtggcccccacgttctgttttcatttctccttcagtccctttttgttccctttctgttctctctttgaagacctcagtcaccgttttctgagttggggttgagcttggtcggtactggaatctctttccgctgctgcaggagtctgaaggaatcagtcttgccgcctgtaacacatgtccagcgctgctttttctctgatgagtgtcttagtcagtttgggctgttaaaacaaatggcttaggcaacacacgtttctgtctcacagttctggaagctggaagtctgagatcaaggtgttggcagattcggtacccggtgaggacctgcttcctggttcgcgggtagaacacttcttgctgtgtcctcacaaggtgcagagagagaggggggtctggtgtctcttcctgtaagggcactgatcccatcatgggggccttacaatcgcgacctcatctaaacctcccgaaacctcatctaaatctcacctccatactatcacattggggattaaggctttaacatgtggatttcaggggacaaaggacaaaagcattcagtccatggcacatgctcagtgcctcgactcttgcaagtgccacaccacagcctctctggggctgtgtcctggaggcgtgtgccatgggccctgtgtgccatgggcaaggcgcacagcatcctcccggccaccccaccagcgagtgagctcctggcaccctggctctctctggtcacccatctaccagtcttggtgctcctgtgcactagaggaccagctgcctggggactgtgggccaactgtggccccggccacccacgaacttcccctccggccagtggctgcaatcacatcttctctaaagacgtctgaagcccagccttggggagccgagttggtccttccctgggtactgagccctagggaacccttgagagttctctttgtatctttgtagtttcttcctcaccacttaatcatttccttacaggaaacttcctgtgttcaagtgactgtatggtttctgcctccagcttcatttgtgggtgccataagagaggcaagcatggagcactaggcgcaggggatgggcaactggcaagcggggagatgcatgcagcgcacttagtgcctggtacataccaagtccttttagtctggatttcattatttttaaatgggtattgctatttttaaaagaatagttacaaatatttattgtgtgttttgaaataagtgggtcaagatcaataagatattgttgatcaattgatcaataagatatcttttattcttaaaaatcatattcttctggttcagtggggaagagactgccgacctgtatttacagcattatgtgataagtgttctccttttcaggtatgtattagtctgttctcatgctgtcaataaagacatacctgagactgggtaatttataaaggaaagatgcttaattgactcacaattcctcatggctgaggaggcctcaggaaacttacaatcatggcagaaaaggaagcaagcatatccttcttcgcatgatggcaggaaggagaaatacagagcaaagtggggaaagccccttataaaaccatcagatcttgtgagaacgcaatcactatcaagagaacagcatggaggtaattacccaccggctccctcccatgacgcatggggattatgggaactatagttcaagatgagatttgagtggggacacaggcaaaccatatcaaggtgactcctgcaagcacctacctccacccctccttcatccttgccctcattctacaatgatttggtgaaatctggtccctgcctcagttttacagcctccccatgactctggttacttcctgattagcttaaacgaaacctaactaggttgccctaggaaagcatttctgttcctgacaccccccatctgcctgctgcttccgttccacctgtatgtgtctgggcacatccctgcatccctttgctggcttctagcctactcacttcaagcatttatcccatgagtttcataaaatcgtagaagaaaagggcttgaggcagtggtggggaaatgataggaaagtcatttctggatgcattctgccatcctgcagatccctaaaccacctctccctctccattccctccctccagagaacagcttctccttgtctcctgtggaatagttccgcccacattcatgggcccttcctgtaccaaaactgtacaggtctctcttgcttaccaaacacttggcaaacaaatgtgccgtccttggaaaaattctgttgaataaaattttctctctttgatccatccaaatgttttacaaagtgctacagaagccatggaggaacaagcaattctgccttagggatcaaggtttcacacagggggtgatatctgagcaacagtgcttttttggtttgtttgttttgttttgagatggagtctcgatctgttgcccaggctggagtgtggtggcacaatctcggctcactgcaacctccgcctcccaggtttaagtgattctcctgcttcagcctcctgagtagttgggattacaggtgcccgccaccatacccagctaatttttgtatttttagtagagacggggtttcaccatgttggccaggctggtctcgaactcctgacctcaagtgatctgcccacctcggcctcccaaagtgctaggattataggcatgagccacagtgcccagccaacagtgcttttaattggcattttcttcaaagactttgatgtcctataggagggggcctatgactcagcctcagccaatcagagcgctccattccctgggtcacctgcacacctgctcttccctgatccactgcagtgccctcaccctgagatctgaaacttgagcagaggcactaaaaggcagacatgggagctgagctgtcttttgggagaatcctagtgagaaggttctccaactggggccgccaagtaagggcctcatggcagactaacccctctccttcctaaggctgggaggagctgctgtccttttgattctgtgagctacctcagttaccttcctcaaaatcacacacacgcgcacacacacacacacacacacacacacacacacacacatttgcatgcgctaggtagagctgttttccataattgccaacagaagactaactgtatttgaagaatgagctggcattcttctgctccggtagaagtcaaggcaatcagttatgagaatcagagcccacctgtgactccagaaagaggtgcataaataccaagaatttagtctctaaagtctttctttaagtccttttttaaaaaatgtgatgagtacatcacccaggaaaatcaaattgtaatgcaaccgagtcgatgcaagttttatttaggagatgggttacaatcacctggggaggctctagttaccttgatttggtctggtacaaaccctagcaccatcatccacagatccccagaggaagtcattcctggatgacttcctcatcgattttaaataatttccatttcagaggaaggcctttatctgacctgatcccctaaatattgggggaaacctacatagggacaaagacagcaggtgtctgcaatgttgagaatcagtgtgttctgtcactgtctctatcagggctggtggcacatgcaaatctctttcccactctccagttgaacactaacgccatggtgcccacaccttccttattagtccatgtacatggggtttgtcaagacagtggttcatggctctgaccctgagcatgtcagatttcaggggctttatgcaaaatatccataccagttggggtcatttcccatcagtattgctcacaatggagcctacaaacccctagttcccatccaacacatctccaaggcagactctcagaccagctcccagaaatgaggtgagtttagatcaggcagcagagaggtggcctaggaaggagtccttggagctcatgcacctgtgtctgggcaccaacaggaagatggtggcttttgctctttgggagatatctttggagccagtctctgaccacatgtccaacaggacaggcatccttggggtttccatggcagtctactgacagtcaggggtgaggattaaatggtacagagtctcactgagtgctctttgagaggtcaagcaatgagaagtcctgcaaatgattattgagctgaagtaagaagtgtaccgaatctgtttttcccctataaatataaaagcctataaatataaaaatcttggtgaaaaaaaatgatcccagcctcccacacagcacatcacacatcttctcttttcaaatttgactccaaggcccacttccttcgggaaatcatttatccagtggtatcatttaggatatttttggttgtgaggaacaaaagcctagctccaaaagacttaataaaaggatctcattggttcacaaactgaaaaactccagtggtaaatgaaggctctgggtacagttggtacaggctctggtctctgtaatttcctagttcttctcccttctagatgctgggtttttgccttcaagttggctttcttcatggtggcaaaatggatccagcaattctgtcagaggttttcgaagcagagtgactccatcttgattaaaggctgtgtaaaatgaggatgagacttgctggactgcattccaggagggtaggcattcttagtcacagggtgagacaggaggccagcaggattgatatcacaagacacaggtcacaaagaccctgctgataaaacaagatgcaataaagaagccagccaaaacccaccaaaaccaagatggtgattaatgtgacctctggtcttcctcactgctcattatatggtaattgtaatgcattagtgtggtaaaagacacttctactaactccatgacagcttacaaatgccatggcaatgtccagaagttaccctatatggtctaaaaggagaacctatatagtctaaaagaactgagggttctgagaaatccctgaccctttcctggaaaatttatgaataatccactccttgtttagcatacaatcaagaaataaccatagtgtactcagtcaagcagtccctgctgctgctctgcctatggagtagccattcttttgttctttactttcttaataaacttgctttcattttactttatggacttgccctcaattctttcttgtgcaagattcaagaaccctcccttggggtctggatcaggatcccattcccgtaacaatttcaggctcagatcggcttttaacaccatccagagcaagagaaagcttttttgttccagaattccccattaaagttctcctggtcactcttattgggttgtttcgcttagggtcaggtgtccatcctggtcccaagcaatgaggccaggagatgggatgcaacgactggatcaatctaggcctcttattcccactttttaaaacacttattattattattttttaaaaattatttttcattcagctttttcatttgaaacttattccaattcttgaactgggggtagtttcaactttcctagagctgtatgggtcctcaaatgaaaattcggggcagctggattagagaagggggaaatgcatgctgcaggggcaaccaacaaggggagattgtgccaattcactcttcctatcctcagattcacctaagttctgaccattcagccccatttgaatgcattctgtattcctatgactgtggattacatttttgtctacctttgtgtcttctgttttgtcctgccctataagcatctcaaacatatgcataaagcctatataaactttataaataaactaacacttctgttttcaacctgtaggatgatgacaatgatgatgacgacaatgatgatggtaatgatgtggaaaatgtgaaaagagaaagaaatacttggaaatatatctcaccctccataaacaaagctcggggtttaattctgacctgtatgagttcatggggtgaactgcagaccgctgtctgtggacaggaaaacgatatttcatctctagccccagggacatctccaaaagctgagctagatgaactttatataaattggtacaaaatataattttctctttgcctgctgaaagccatttctagaaattctgttaatcagaatctccctaagttaatcagtcatctagacagatcttatttcttttttagacaaagaaaagtatataagtaacaggtattggtaaaccacttgagtgaagcatatgatatctaatgtaaggaaatctaaaagtgtccacaggcaaaatctcatggattcaattgatagcacaggtcatcaactgacatgcagacggaattctcttgtggaacaagacaatacagccattgcttagagactaattgtcaaggaattagtcatttcctgtttcagaatagcatcatcaccaccaccattaatgccaacatcaaccaccaccacctacgccaccaccgttagcatcataaccaccaccaataacatcaccaacagcaacactgccatcaacataaaccatcaccaccaccaaaaccattagcatcacctagaaccaccagtcaccaccatcaccacttaccacaacaaggcttatatttacatacttattttacttttcgaaatacattcacatgcatggtttcattagatcttatctacttggtaaggttggcagatctgacatcattagcctcattttatctgtatggaaactaagttctagagaagcgaagtgatgtgtgaaaggacaccagagtgattgataatcaaatccagactagagtttggttcttctgactccaaaattaatacatttttcttaaaagaaaaaaattttttttgagacagggtctcactctgtcacccaagcttgagtgcagtggcatgatcacagcttactgcagcctcgacttcccaagctcaagcaatcctcccacctcagcctctcaagtacctgggaccataggcacatgcctggctaatgtgttttaaacattttttggctgggcacggtggctcatgcttgtaatcccagcactttggtaggccaaggcaggcggaccacaaggtcaggatatcgagaccagactggccaaaatggtgaaacctcatctctactaaaaatacaaaaaaattagccaggcgtggtggcacatgcctgtagtcccagctactcaggaggctgaggtaggagaattgcttgaacccaggaggcagaggttgcagtgagctgagattgtgacattgcactccagcctgggcgacaagagcaaactccgtctcaaaacaaaacaaaacaaaacaaaacaaaacaaaacaaaacaaaacaaaacttttttttttttttttttgtagaaacggggtctccctaggttgcccaggctggactcaatcttctgggctcaagtgatcctactgcctcagggtctctaaatgctgggattcaggcatgagccaccacacccagctccaatgctttttttgtcgtacctaattctttcaatgaaaatgaagaatttccaacttctgatattaacaactttggtcctatattcaagctagagtctttcaaataaaatagacttttaaaaccatctgtctccaaaccctaaatgtctcaggtgagcaactaagctgctcagtttatgtgactccccagaagttgaattttaacccagaactgactccaagttcattcttctttccacgacaaggagtcacctccttgtatgcccccaggagtctcccggattcctccgagaacagtggaatagtgctcctccccagagcacaggttttgccagtgaagattgaatttggctagaaaccgctgccctgctctctcttctcgaagcacctggaagtctgagaaggaactgggtggctggctctggtcacaaactagcagmcagaagcaccccttgtcagtgatgcacccccagtccccctcaagggctccaagtaaacccaaagctgctcccctccaagaagtctggggccaccctagggaaggcctcctggccttgactctcagggggtctctggggttgcggtttggggcccgctgcttccgccctttgcccccaggtgggcctggcagggctgcagcacagctctgttgctgatagacagggtggagcacttggcgaccttgccctgcagccctgtcattttgagttcagaggtcanatttgagtaataaacatcttctaaggacttgtcattctttctgaggatgttgctggccagccggaagacgaaaatcaccgcgtagatgccratgatggtgagtatataccaggcagcgctggttccgtctggcaccttcagagccctggtggagttggtgtcattcctcccctctgtgtggtcacccagcaggagccccaggagggtgctggcctgggtctggttggaggcttcatggggagtccacttggcccctgagaaacagagaggtccggatgmgatccagcgtcctgggctgagggctgcctggccacaccaaggagaatggagccctcatatccgtgaaaacgtgtcgctgctcaaagaggccttctctgaggcatgagcaggagtgtaacaacaggtatgtcaatatatttttaaaaatcaaaagagtccaaaacactattttgttgttgttttgttttgttttttgttttgtttgttttagagagacagagtctctgtcacccaggctggagtgcagtggcatgatcataacttactacagcctcaacctcctgggctcaattgatcctcctgcctcagcctcacaaatagacatgcagcaccatgccgggctaatttttttcttttttctctctctttttttttttgtagagatagggtcttgccatgttgaccaggctggttttgaattcctggtctcaagagctcctctcaccttagcctcccaagccctgggattacaggcaggagccactgtgcccagaaaaacactaagttcttgaataggagacacaacatcataaagatgtcagttatccctcaaataatttatacaacaaacataattgcaataaaaacagcaataggatttctttgtgaaatcaataaactattcatttagaaaaatcaactgttggccgggcatggtgtctcatgcctgtaatcccagcactttgggaggctaaggtgggaagattgcttgagcccaggaggttgagaccagcctggccaacatgacaagaccctgtctctacaagaaataaaaaaactagccaggtgtggtgtgcaagcctatggtcctaactactcaggaggctgaggccggaggatcacttgagcccaggaggttgaggctgcagtgagctgtgttcacaccactgcattccagcatgggaccctatttaaaaaaaacaaaaaaagaaagaaagaaaaagaaaaagaaaaatcaactgtcaagactaattagaaaaaaaaatctgaataaaaagaatgactaatgaattagcctagccacaaattttaawtcagccagctataaaaactaatttacatttttttcaatgaatgaaagctttatatgcacaaagcccagctgggacttgctgggctttgcagagtgtgtgggctgggggttcttcagaaccaggtacaactctccctataaaactacaacagtgctgggcatggtggctcacacctgtaatcccagcactttgggaggctgaggcaggtggatcacctgaggtcaggagttcgagaccagccctgccaaaatggagaaaccccgtctttactaaaaatacaaaaattaaccaggcgtggtggcacacacctgtagttccagctactagggaggctgaggcaggagaatcgcttgagtccaggaggtggaggttgcagtgagccaagtgatgcctgtagttccagcaagacagagcaagactctatcttaaaaagtaaaaaaataaaaaataaaactacaacagctaaaatagtgtgatgcctgtagttccagctactagggaggccgaggcaggagaatcgcttgagtccaggaggtggaggttgcagtgagccaagatcgggccactgcactccagcctgggtgacagagcaagactctgtcttaaaaaataaaaaaaataaaaaataaaactacaacagctaaaatagtgtggtgctgaaaacacaggcaagcagaccaatgaaacagagtaaaaacagcatcaatagttagcaattagaatttgatagctagctaataaaggagcatttctgatcggtgggaaaagatgaattattcaatatgtagcattgggggaaatagcattagatccacatctctccaccatatgaccagataaatcggtccagattaaaaaaaaaacagcccagataaatcaaatattttaacataaaaagtgaaataatttatagtactagagtacagcatggcagattttttctttatcatctcagagtggaatattcttttaagcataacaaaaattcagaagaaacaagaaatagaaatcaaattcaactacataaaaaaaattaagctatttcataccataaaaccaacaggcagatgacaaagtgcaatttatatcactgattttctaaatagccttcggttctgtaagaaaaagtttaaaactgcagtagaaaaatgtgcaaaagatatggacaaatagttcacagggaaaaaatgaacattcaacataagaagagcttctcaatatcactcatataagaaaaatgcaaattaagataataactagataccattttgttacctattggacttgcaaattcatgatgtttcagaataaactaacaaaaaaatggcttttttttgttcttttgtccagcttagaagaaaggtgtctaaattgggagcaaaggtggcaatgacgtggacttgacaccaaaaaaaaatttttttaaagaaaagaaacaagtgcctctgcatttcaggggtttaggattggcatttttaaaatgtcaacaaataaatgttcatatccacacttgacattttttccaaggagaattttaattgtataattgctggtaaattcatgcagccaacatggagggcacacggacaagatctatgagcattacaagtgcacttacctttgacccagcaattctatctctaggaatctatcctaaagatgctccagaacatctagagacaacatatgctgaaggttagtcattgcagtcctccttgtgatgacgaatgcctgggaacagcctgaatagcaccaactgagggatggtgaaatacattttggaacctccatgcagtggagtactacacagtcataaaaagcaatgagttttttatggtactgaatgttaataagtgaaaaaataagctaatggtgacatgctctgcaatgccacttgtaaagaagggggaagttatatgttatttgcttgtactttttttatgtatagaacatctctggaagaatgaataagaaattagtatctgcaattgcctctggggaagaaacctgggggaagaagatatattttttactgtttgcccttttgtacacttagtaccgtgtatacttatttttgaaaagcaagagtgtaccagttggtacttttctggtctccctggtgaggtgcccctgggtaaagccgttgtatgcccttgtaagaccagaagattaagatctcaattgctgttcaattcaaaactgttttctctgcttggagagctggtggagaaaatgaaacaatgaaaaccagagctgtagagtgcaatcctgtgagacatttcccagtggggccttactggctcaaacccccatttcttgctctaatgtgaacacagatgtatttaaaaacacatcataggatcaatcttgcagcctgctgtgyagaacaaaggtgctccaaaatgcttcccatttgatcgttgtttgttgctaattcattttgcgaacgcaagactcagagaggccagtattttttattatagttagttgccagaatgtgtgaatgagcttattacttttagatgaaggaagaaactatttaaaaattacttttcaaactacatgtgacaaagcccaggacaaatgaacagatttaattacataaaattagtcactcgcaagaaacaacaccacaagcataaatttacaccattgtttggtagaatggtttgagacattaaagtaaggaaggtgaaaaattcccgtaattattgcaacaaacaaacagacagcaaatcaacccaacaagaacacaatatccttatattagggcaagagaacttattgaaactcagaacacatgtataaactcatagaactttctagaaattgtcatagaatgatgcaacacattcaaatacaaataaaatatccccaactaagagctacacacagaacattaaattatttaaaaaccagtccattttctacacgaaagaaactcactatattaattactgcaatacattacattttacctttcttacaaaggtaaaagtaagttaggttgtatcttaatggacaaacatatcctgtagaagagagaaactttttcctctgtgctattttgtacttgtaatttaatgacgtgaaatatgtaaaatctcaacctgcccatccttgcattgtagctgagtactcacattccatggggtggtcttgtccttgactcttggaggggcaagttcaagcggctaccatgcacagaaggggaagatgatgaaaggagaactccgtctcctagggaagaatcagtcctactgcagttgagctgcactgagtttccagagtggggagtaatatgatcttccaacaatcttagggcagcaccaaacagaaacttagtaagtggatgactttgctttcatgcaattaatcagaggatccgatttgctgtgtcttctgttgcatcagaacagaaagcacttcccagctttgacttgttaagaagttctcaatcaaaacaaatttttaaaacgtgctggtattaaggaatctccatctctcaggtcccatcatgaactgaggtggccagaagctccccctgaggctggctctccgcttagagcttggatggctattgaattcccctgtgttctgcacctgttgcaggtgtggcagatggccaggtgtggcagagatctgtcatcatagggccaggaaactccatggtcaagagtcaccagcttcctctggacagtctcccagatgaggaaacccagacaggaagggagtgacaccccaagggtgacacacctgaggggacttgggctttccctgaggggtcagtgggcagtggactcctgtgccaggtggtgagaaatggctcttctctttcccagagtcacagaccccattggagttgaggtaggcttaattggaaagtgttagagtaagtgtctgcgggtaaagtttccccaggagcagggagggaaaagttggaagactggcaagttaaatcatccagccattgtttccagttccatttcttcctaatcctcactctaggactctaacttgccacgtttgtgatggttgctggtttttaagatacaatttgatgaaatttccatcaatggggtactgggtaagtaagttataaaataagccatatgatccagcaattctactcctgggtatcttcccaggagaaataaaaatgtaagtttacacaaaaacttgaacacacatgttcaaagcagcattatctgtaatagcaaaaaatggaaacaacccaaatatccaacaactgactaatgaataaataaaatgtggtttatccatacaatggaatgttattcagcaataaacaggaatgaagtactgatatatgccataacacggatgaaacttgcaaacattgtgctaaataaaagaagtcagtcacaaaggactacatattgtaggatttcatttatatgaaatgccaagaataggcaaatctacaaagatagaaaatagattagtggttcactagcgggagggattgggggtgataactaagggtatatagcatttttggaggggtaataaaacttctaaaattgtggtgctcactgtacacaatctgtgaatatacaaaaaaattgaatgcatactttaaatggatgaattttatggtatatgaattatatttcaataaaactgttaaaaattataatatacaagctgggtgcagtggctcacacctgtaatcccagcactttgggaggccgaggtgggtggatcccctgaggttgggagttcgagaccagcctgaccaacatggagaaaccctgtctctactaaaagtacaaaaaattagccgggcatagtggagcatgcctgtaatcccagttacttgggaggctgaggcaggagaattgcttgaacccaggaggcggaggttgcagtgagcagaggttgtgccattgcactccagcctgggcaataagagtgaaactccatctcaaaaaaaaaattataatatacatatacaatggagtattacacagctgtgaaaaagaacgaggaagctatttatgtactgatgtataaagctctctaaggtgtgctgttatgaaaaaggtaaagaagagagcatgttaacatgtatccaaaaattgagaggaagcatatatatatatatctgattttgccactgtaagcatttaaaacaccagtggaatatccaagaaattaagaagaggggttacctattggaggagagaaccaggtagatatatggcaggtgtgggagggagagctctcactaaatatttttatgctttaaatatttttaaccgtatgtgtattacctattcaataataaatgcacccatttgttagatatctttgttgaagattcatttggctcctgctgtctcttgctatgggatggaccatggcatcccccctctgccacacagacaagggatttggacactgccagtgggacgtgggaggggagagcacctgacccgtgataataaggggctcgtggcagtgataagggctgggagtcagggctctggccccagccacatccttgctgcatgaccctgggccagccccctcatctttgtgagcctcagtttcctcatctgtgaggtgaaggtggtgaaggaggtgaaggatgagcaggatcttatgtccttggtcctgagaaggcaggagagaagcctggggctctgtgtgggaagagccgctctctggggaggtatctgaatagatgagggagagcacaccgggcagccartgtgccagaggtggaggctttggagagtgtttcatttgtgaagtcaacagatttaacattcagatcaggaggacgttggcatgagatgtggggaatcataagctccaaaacaatcgtgagacagaaggaaagatggccttttgttgagcagccattctcctccacggagagtcctgtctagtctgcctgttgaaggggcactgatgttagggaatagatctgtgtcaaatgcttcccacctcccagaatcctgtgaggcaggagtattatccccatttaaagagaggacactcaggctcagggaagtgactggcccaatgtcccatagctcataggtgccagaggtgggtcatccacaccaaagtcattctccttccataccctgaatgtcaccttcacgctggacccaggatcctgtgtggtgaactgtctcgatcacttccctaaaggttaaatcataaactcttactgccaaggcatatccacgaccttaaactctccctgttgggcaaaaacaatctctgatgttaaaaggcaggatagtggatacttttcagggaagggtaaatgacaagggcatgaggggaactctgggtgccggtcatattctgttttacaggtttgttcaatttgagacacttcatagagctgtagccttgtgcacaggcacttttttgcatgcatcgtctgcttcaatataaacctcttcctgttgtcttgtttttgtttttgtttttgttttctcttgttttcttgtcctgctctgtcacccaggctggagctcagtggtgtgatctcagctcactgcagcccctgcctcccaggttcaagcgattcttctgctcggcctcctgagtagctgggattacagaggtgtgctaccacacctggcttccctgttgtttctttaatgtagaaagccctgatagatggtgggaaaacaaagtttaaggtattcatagaaaaatacaaatactatttttaaggattctatatctggccacatggtgccatctcacgaagagtgtcmccgtcccttgagggggagtggtcgggatcatggtcagtgtggggccctgcagctgcctgcttccctatgctgtgtggatgacgcccgcctccggtcattcccctgtgcttacataacagtgaaatrgaacaacctgtatcagcacgagggccaagaattttcttctgacttgtggatacctccttccttaggcctctgatcagtctggacaaatattgccctgaacgcaaccaagcaaagccactcacctggtaaatatttgtatgagctacagttctggaagaacaaattccaatatcctgcagtccccttgacatcaaagacccaactctcccagagggcaatggcttttttgtccactgagaagccagtcagcttcraagaaaggtgtctaaattgggagcaaaggtggcaatgatgtggacttgactccaaaagaaattttaaaaagaaaagaagtgcctttgcatttcagggggtcagtattggcatttttaaaatgtcaacaaataaatgttcatatccacacttgacattctttccaaggagaattttctagaggagacagacctcatcggtcagctctgatgccctgcagtgcaaaaagacattaaaaatgacggtaaaggacccctgcagagaacaactgagtctcttccttgccctgcgtctccagataaaggatgccctgcatccatcccctcctggctaagagcacagactccagaggctttttcctctcctggaggttaaagaggcatcacatatgtttaaaatctttaatttatatgtcacctttgtccttccttttaacttcatttttctcttatccagcatttagggactcatctttagggaggttcaaaggaaagctcatggcctttagaactggaagaaccatgttccagttgggacttgatcatttactaattgtgggattacagccaagtcacttcatccctctgctgtaaaaaaaaaaaaaaaaacaaaaaaaaacatatgatgacatttgtggaatggctccccaagccaaagagggcaaatattgtcacagctcatttcttctctcagttaattacttgcgtcctcggctgcctggctggcaggacaacctatattcgcctccctcttaaagcctcctgggttggccaggactccaagcggctttgtccagaatgagtagggtggttggcctggcctcctcagccaatcagagaggactagcatctgaacactcctctgtgctattgcttctagctgccacatggggacgctgttgaaacaccggcctggtgcagttggccatatgatgcttcagggtcttctgagacttcaagaatgtgctcacagggaaggtattagctctaaacacttgcctctgctagtttacatcacagaacagacagacaagactgttttgctccctcagctctctccttttcctagcttcagtcctggggagctcagaagctacagtttgttttttgttttttgtttttgtttttttcttgagggagtcttgctctgttgcccaatctggagttcagtggtgtgatcttggttcactgcaacctccgtctcccaggttcaagcaattctcctgcctcagcctcccgagtagctgggactacaggtgcctgccaccatgccaatctaatttctgcatttttagtagagtcaggatttcaccatgttggccaggctggtcttgaattcctgacctctggtgatcacccacctcagcctcccaaagttctgagattataggcgtaagccaccgcacccggccagaagccacagtttacaaatctgggggatttggggcatgggaacagaaacagaagagtcccaatgaaaggaagataccagctgagctgcccactctcccagctgcagttctcctgcccacagcaggccctagctgggacagggaggagccccagccttaaatcaaattcagaattttgtttatgacataagactgcacatcttaattactgaattaagactatattttccaacctatcatgactataggtgcagggcaagatcaaactccagtgtatgtggggcccgcagaagagatttaaagaaacagtgggggcagaaataaagctgtgtggttatcagatcccatgagtcttgtctgtaaggatgatggttacagtcgggatgctccagagtgcaaagccacatctcaaccagagttagtaacaagggagagtttactggttcatgtgaggaagagagaggaaggggagggctagccaaggggctggatgcaggaaggagggtccccagggttctctgtccccctcctgtcttccatctctgcctctctcagcaggttggcctaatttcctccgactgcagagaagcacacaagctgtggcacctggtgctcagactcacactgcaacacttccaccagtagatggcagagaggtactttcctgcctgttcagccangaaaatcccaggggatggctctgactagcctaagtcaggaacctgctgtgggcaatcactgtagcattaagatgggggccagtgatggagccggtctgcagcacatgctcagcaaaagacaaaacccgcctgttttagatcactccggctgcatcacagagtgtggattgaacaggcacagaactggaggcagagaaacaagttaggcagctgcaggcataatccaggcaggagatgacagtatttgaaagaaggagtgggagcaagtctggagagaagtcgatggatccaagagatttttagaaggtagaatgtgcagaacttaattagttggtgcagtgggttgaatggtgtctccctaaaagatatgttcacctggaacctcagcatgtgaccttatttggaataagggctcttgcagaagtaagtaaggtgagaatcttgaggtgagatcgtcctggattacagtggaccttgtatccaatggcaaatgtccttataagagacagaaaaggaaaagaaagagacacagggaagaagatgtgaagatggaggcagggattggagtgatgcagcctcaagccgcagaatgcctggagccaccagaggttgggagaggcaagaaaaggtcctcccctagagccttcacagggagtagcgtcctgccaacgctttgattttgagctggtctccagaactaagagagaatagatatctgtttttctaatccaccaagtttgtggttattttgatgcagggcaggcaagcccccaaattgggttgtagcctgagagggttcttgggttcattcaggaaggaattcaagggcaagctggtggtattagacagcaacttctgttgaagcagcagtggacagcagcagcagaggtcctgctctttgcagagcagggctaccccataggcagtgtgcccagagtagcagctcgaaggcagttctgtagtcctatttacacccacttttaattatatgcaaattaaggggcagattatgcagaaaattttagaaaaagagtgctaatttccaggttgtcgggttgttgccatggaaaggggccgcaacttccggtgaactccatagtatgtggcacacactggtgggcgtgtcccatggaaaggtgcttccgccctgtacctgttttagctagtccttaatatggtccagtatccgcgccctgcctttggagtcaagttcaacttcctacctcaattgatgatagcagtttctgaaaactaacacatgtagatataaatataagtccttaagtctatcattattatgcatatcctataggggagtcatcgcgaatgaaactgaacttattgtggttcattcattcagatatttatttaaaaatatttattaaagcttactgtctgccagtccgatactgcactaggtaagtgctggggttacaaacagaacaagatagacagattagttgcccgcatggaacttatatctagtgggaagagaagcaaaaaaaaagtaagcaagcaataaacagtaaaaaaaaaaatactgggatttgagccataaaaaaagaaataagatgcagaaatcagcaataaggaggttggggagaagatccttctttagaaagaattgccagagaaggtggttgggataggcagaaaaaatagtaatattcctcttttatcttcacctatattagatgatcaatagatatttcctgagaaatgaaggactgagtatattataagaaggtatgattaaaaacaatcaccagaatgaatggctaacaagcacatgaaaagatgctcagaatcattagtaatgaaagaaacacaaattaaaccacaatgagataccacttcacacataaaaaggaattaacacttgctggtgaggatgtggggaaatgtcatatttccccacagcagccatagtacactgctggtgggaatataatatgatgcatctgctatggaagagaatatagtggctcttcaaaacgttaatcctagaaagcctgggcatggtggctcccgcctataattccagcacttcgagaggccaaggtgagaggactgtttgagcccaggagtttgagagcagccttggtaacatagcaagaccctgtctctataaaaatcaaataaaaaataaatagaggaaaagcacattaatcatagaactgccatatccaccacttccactccttggtatataccccaaagaactgaaaacagctattcaaagaaatacttgcacatgagtgttcagattattaacggaaaccaaaaggtggaaataacccacatgtctaccaatggatgaatcaataaacaacacatggtctatccatacagtagaatattgttgagccataaaaaggagtgaagtgctggtacattgccagaacatcaaagacccttgaaaacattatgctaagtgaaataagccagatgcgaaaggacatgaattatatgatttcattgatataaaatgtccagaaaaggtaaaaaatatccattgagaccaaaagcagattgtggttgccccggactaaagaaagagtaattacttaattttcctgggggtttcctcttggcatgatgttctgtatacaggacatacaaaaagcctttattttttattcttagcaaatacttaattagtactcaccatgagctgggcatgttctaagtcactttccaattactaacaaatcacttaattatattgacacaaaaagaatgggcataatgcataaagcaaatacgaacataaaaaagaaatctccctattaatatcatttatgttgaattcaatgcagggagcatttaaataagataaagggagatacttcataatccacactggtcagctaacatcatgactatctatgcagaagataaaccagcatcaaaactcataaagaaaaatttatagagagtaagaaaaaaatgaagaaacagtttagaggtaggtaatttgaatttactgttcggtgcataaaagaacaaataggccagacgtggtggctcaggcctgtggtcccagcacttcgggaggccgaggcaggcagatctcgaggtcaggagttcgcgatcagcctgaccaacatggtgaaacctgtctctactaaaaatacaaaaaattagctgagtgtggtggcgtgcactgtaatcccagctactcaggaggctgaggcaggagaatcgcttgaacctgggaggcaggctgggcgcagtgactcacgtccgtaatcccagcactttgggaggccgaggcgggtggatcatgaggtcaggagatcgagaccatcctggctaacacggtgaaacctcgtttctactaaaaaaatacaaaaaaattaaccaggcatggtggtgggcacctgtagtcccagctactcgggaggctgaggcaggagaatggcgtgaacccgggaggaagagcttgcagtgagccgagattgcgccactgaactccagcctgggtgacagagcaagactctgtctcaaaaaaaaaaaaaaaaaaaaagaaagaaaatacaggccacacagatggggagatgataattgcaagttatatatttgataaaggactttcattcagaatatatgaaatagtcttacaatttaataaaagaggacaaacaacccagtaaaatgtaggaaaaatatttgaacagatgtttcaccaaggaaaaaatacaaatggctaatcagcacatgaaaagatgctcaacatcatttagtcattaaggaaatacgaactaaaaccaccataatatatcactacacacctgccagaatggctataattttaaaaaaatggacaatactgagtgctggtaaggatgtggaaaaacagaaactctcataccttgccagtggcaatgttaaatgatacagctattctggaaaacagtttggcattttcttaaaaatttaaacttattatatgacccaacaattccactcctaggtatctacccaagaaaaataaaaatacatgtccacacaaggggacttgtgcataatgttcatatcagccctatttgtaataacaccaaattggaaggaatccaaatgtccattaactatgaatggaaaaccaacattcttacaaataattcaacaataaaccttcatgaaccttagaaacattattctgagtgaaagaaaccagacacagaagaccacaaggtgtaggactgtatttatttgacatttctagagaaagcaaaactgtagagacagcagatcagtgactgccaggggctagagacggaggcaagggttgatacaagcaggcaggaggttgctttctgggctgatggaaatgttcttatgctggattgtggtaatggttcacaactgtataaattaacaaaaaattatcagactatacccttacaatggtatgtacatttcatccaagtaacgctgctttaaaatttgaaattaagcacctaatgatattaagaaatgaataacaaaataaacccaaagaaagcaggggggaaaaaaagcaattggaaaagatgagagcaaaaataatgaaaaaaaaaacatctataatacatctagcggttggttncttgaagaaaaagaaagaaagaaatgaaaaaatcattaactatcctaataaagaaacaaaggagaaagaacaaatatacaaaataagaattgtgaatgaaataattgtagacacagaggatatcaaatgagtgactcctcaatccctctgcaaatagattcaaaatcttgaccaaatggatgattttctaggaaaatataaattaccaaaactgaccaccaaagagattttaaaaatcagaaaatatcgtttatcacagagatggtaaaaaccttgataaaaagtcatttacccagagaagcatctggttccaacagctttgcaagtgcatcctattaaaactttattgattggcaaacgctaattttttttaatttttatttttaattatactttaagttctagggtacatgtgtacaacgtgcagttttgttacatatgtatacgtgtgccatgttggtgtactgcacccattaactcgtcatttacattaggtatatctcctaatgctatcccttccccctcccctctccccacgacaggccccagtgtgtgatgttccccactctgtgttcaagtgttctcattgttcaattcccacctatgagtgagaacatgcggtgtttggtcttctgtcctttcaatagtttgctcagaatgatggtttccagctgcatccatatccctacaaaggacatgaactcatccttttttatggctgcttagtattccacggtgtatatgtgccacattttcttaatccagtctatcattgctggacatttgggttggttccaagtctttgctattgttaatagtgccgcaataaacatacatgtgcatgtgtctttgtaacagcatgatttataatcctttgggtatataccctgtaatgggacggctgggtcaaatggtatttctagttctagatccttgaggaattgccacactgtcttccacaatggttgaactactttacagtcccaccaacagtgtaaaagtgttcctatttctccacatcctctccaacatctgttgtttcctgacttttaatgatcgcccttctaactggtgtgaaatggtatctcattgtggttttgatttgcatttctctgatggccattgatgatgagcgttttttcatgtgtctgttggctgcaaaaatgtcttcttttgaaaagtgtctgttcatatcctttgcccactttttgatggggttgtttgatttttttcttgtaaatttgtttaagttctttgtagattctggatattagccctttgtcaggtgggtagattgcaaaaattttcacccattctgtaggttgcctgttcactctgatggtagtttcttttgctgtgcagaagctctttagtttaattagatcccatttgtcaattttggcttttgctgccattgcttttggtgttttagacgtgaagtccttgcccatgcctatgtcctgaatggtattgcctaggttttcttctaggttttaggtcggacatttaagtctttaatccgtcttgaattaatttttgtataaggtgtaaagaagggatccaatttcagctttttacatatggctagccagttttcccaacaccatttattaaatagggaatcctttccccatttcttgtttttgtcaggtttgtcaaagatcaggtggttgtagatgtgtggtattacttccaagggctctgttctgttccattggttctgttctgtctctgttttcgtaccagtaccatgctgttttggttactgtagccttgtagtatagtttgaagtcaggtagcatgatgcctccagctttgttcttttggcttagaattgtcttggcaatgcgggctcttttttggttccatatggacgttaaagtagttttttccaattctgtgaagaaagtcattggtagcttgatggggatgccactgaatctataaattaccttgggcagtatggccattggcaaacactaatgtttttaaactgttctagagagcatggagaaaggagaaaaccttccaaattattcctgtgaagcttgcatgtcaatgattccataacaataactatagaatcaaataaccacaataaaagaaaaacacagaccaactccacttatggatatagatgtaaatattctaaatacaatattagctgatagatctaacactgcattaaaagatttgtggaaggagttgttcaatattaggaaatccactctgtgattatctcaagttagcaattagatgtatattcaatgctgaaataacagaagcaccccagtttagtcagaaataagacccaattacccattatcaccaccaccatttagtattgcactggggaattaccaattcagttagacaagagtggggaagaggtacaaaaactagaaagaaggtggcaaaaacaatcattgactgtatgattggaaaaaataagagaatcaattgcaaaaccattagaaagagcaggataattcaggaagctcagggggcacaaaataaatgtttttacaaaacaatatccaagaatctatattaacaacaatatctttgagatataattgaatagaagattccatttacaataggaaaccccaaagatagaacacccaagagttgcacaaaatttacacaaagaaaatctaaacaacagagggacaaaacggaagatttgactacatgcaagtatatttcctagtcttgggtagaaagactcatctgcataaagatgacaatccttcctgaattaatctataaatttagtataattccaatggaaatttcccttgttttgttgttgttgtgctgtttttgttttgttttccagactacactgaatgccaaatattccatttagtgattttcttcttcccttttcctttctaatgacatattttgtgcttttcagacctgcctttctttctctcggcaccaatgaataaagttccagctttaaggcttgaaaaatcacagcaaagttgcagcaaaattaaaaggaaaaaaatgttctttttttttcctgcagctgcagagagtggcagatagcatcctgcgtgataaacgcctattcttggctaggcgcagtggctcacgtctgtaatctcagcaacttgggaggccaaggcaggcaggtcacctgaggtcaggagttcgaggccagcctggccaacaaggtgaaaccccgtctctactaaaaatacaaaaattagttgggtggtggcgcacacctgtaatcccacctacttgggaggctgaggcaggagaattgcttgaacctgggacgtggaggttgcagtgagctgagatagtgccactgcactccagcctgggtgaaaagagtgagactctatctcaaaacaaacaaacaaacaaacacctatccttgcctatgtcattttaacaaaggaggaagtaaatcccctggatttcagaggctgatgctctgcccaagaaaagcaaccctaacttccccaaaggctaaaattcagactgattggctctggcagagatatttaaattgatacctctgtttcctcaaaggtataagcctttgcgaactttctttggtttctctcttctctcacaggaggcaggggataaacaaatatgttagatttcttatttaaacaaagagcttgagggttttgcctcatcgaaattaacagagacaagttgatgctaatatttttatggaaaatcgaatatgcaaaaatagccaaggaaattccagggaaaaagtaatgaaagaaaatatcaccaaaagatgttaaaacattttggaaagccacagaaattaaaagtgtttgatcctagcatataaacaagcagacaaggggctgggcatggtgactcatgcctgtaatcccagcactttgtgaggccgaggctggtggatcacccgaggtcaggagttcgagaccagcctggccaacatggtgaaacctcgtctgtactaaaaatacaaaaattagccaggcatggtggcacgcacctgtagtcccagctacttggcaggccgaggcaggagaattgctggaccctgggaagcagaggttgcagtaagccgagattgcaccactgcactccatcctgggcgacagagcaagactctatctcaaaattaaaataaacaaacaaacaaataaataaataaacaggcagatagatcagtggaacagaataaaatccagaaatagactgaaaacattcaggaaaacagtataaaataaaggggacatttcaaatcaatggagaaaagattagttatctcagaaatgaatgggacgattgagtagactgggaaagagtaaaactggagctctacacacaccaaaatacattccagatggggctaagattttatatatctatatatgtttaaataaagccatgaaagaactagagcaaacatgagagatttatttttataatcccagacggtggcaatctttccaagtgtggcacaaaagtcagaaatcattaaaaaaagactgataaatccaactacacaaagttagacatttctttatggcaaaaaatgctatcaaaaagtcaagagatcaatgataatgggggaaacatttgtaacacatacaataagctgtccaattttttaatagtcaaagactttaacattaagaaacagaccagctggctgggcatggtggctcgaggctgggggatcacttgaggtcaggagttcaagatcagtctggccaacatggcaaaaccccgtctctaccaaaaatacaaaaattagctgggcatggtggggcatggtggtgcatgccagtaatcccagctactcaggaggttcttctggcttctcagcttgcagacagcctattgtgggaccttatgattgtgtgagttaatacttaataaactcctgtttatattatgtgtgtgtgtgtgtatatatatgtgtgtgtgtgtgtgtgtgtatacacacatatactggaatatatgtatatacatatatacatatatacacatacatatatatacacatatacatatatacacatatatatacacatatacatatatacatatatacacgtatacatatatacatatatacacatatacataaatacatatatacacatatatatacatatactatatatatacatatacatatattcattccattagttctgtccctctagagaaccctgatgaatacagtgggctacacacctattggaatggccaaaacccagaacactgacaacaccaaatgctggtaaggatgtggcgttttttatccgcattcattgctgatggtaatgcaaaatagtgcagccagtttggaacacagtttggcagctctttacaaaacggcgtgtactcttaccatacgatccagaaactgtattcctaggtatctacccaaaggagttgaaaacttgtaaccacacaaaaacttgcacacagatgctcatagcaagctttatttattattgcccaaacttggaagcaaacaagatgtccatcagtaggtgaatggataaataaactgtggtgtatccacacagtagaatattattcagtgctaaaaagaaatgagctatcaagacatgaaaagacatggaggaaactgaaatgcatatgactgagtgaaagaagcccttatgaaaagctacatactgtatgactctaactatgtgacattctgaaaaaggcaaaactatggtgaaaacatcagtggttgccagcagttgagacgggtggggggaagataaccaggtagagcatagaggactttaagggcagcgaaaatgctctgtatattactacgatggtggatacatgtcattatacagcaggtccttggatgacactatctcattcaacatcattttgctataaagttgatgagaaaaaaaagtcaattcctagccaggccactgtctctgtggagggtgtgcgttctccccatgtctgtgtgggtttcctctgggtcctccagtttcctcccacatcccaaagctatgcacggtaggtgaactggcatgtctacatggtcccagtgtgagcgagtgtggaagtgggtgagtgtgccctatgatggaagaggaccctgtccagggttggtgtctgccttgaccctgtgcctctgggatgggctctgccatccacagctctgaagtggaataagccagtcaataatattctcgcttgttttttgttgttgttgttgtttgtttgtttttgtgacagagtctcactctgttgcccaggctaaagtgcagtggcactaactcggctcactgcaacctccacctccagggttcaagtgattcctgtgtctcagcctactgagtagctgggactacaggcatgcgccaccatgcccagctaatttttgtatttttagtagaatcaggattttgccacgttggccaggctggtcttgaactcttgacctcaggtgatctgcctgcctcagcctcccaaagtgctgggattacaggcgtgagccaccgtgctcagctttcacttgtttgtattaatctttcctaaatgtatgtatggctcacatttatttcaatgtttagtattagaagtgtttgaggtctttgtaagtttggtgatgttttgtgaccagaaacaggccataggaacttaactcttgtttatattaattagcttatggtaaaattggataaatgttttataagagacatgaaagggcatacagacacacaggagagaaggccacgtgaagatggaggtggaggagacagtgatgcagccacaagccaagggatgcaagcggccacctgcagttgagagaggcaggaaggatcctcagaaggcatggagcctacgaggaagcctggccctgctggtaccttaattttggacttccagcctccagaaccatgagagattacatttctgttgtttgaagccactgatttttgtggtcattggttatggcagccacaggaaataagataatcacccacttaattttcmtagaaaagctgtgttttgaaagtcctcttgaagcctgggttcctctctctgcatctcccagttttccctcaaagcttgtggattctccattcctcacattaactcaggcctttcattgccaagtgaccycgagtcctgccttcgcgggtgctgggggagccttcctgacccactggaagtggacctgcccatctccttgctgtgaaactncatgagggggcttgtgtctgaggattgtctggcgtgaggggagagacaccacgtggggacagaggagtggatgagcaggccggggcatgacggggccgtgacagggacctggccttccattctgtggaagcctgagacaagcagcaacttctctcattcctcctctctatgacaagacaggaactgggacactcaccttactaccctaattcgctgagcctcggaagaaaagcagcttagatttttaatcccatccaagatggaggccctcctgctcctgctgccttgttctcaccccctttcgtgatgtgcgaggccatcggaaggtgtggaatttctccactgattcctctcattgtccctttctccctactcctggggaggctgcaatggtgacctcatccaccttcagaggcaggtgctggaggaggaaaggatgtgggagttcaagccggctgcagaggcccaagagcccagatggtgtccttccagcaaactggagaggcactcctcctaccaggcagccactgccccactccagggcccctggctcagctagggaagtggggctgggtttcaccccctgctcatcccctaaggcccagtgctggactcagtgcagcacctgcccagccatctctagcagcggcataaagcataaaatcaaggccaatgttacgtgctgccttgacatgtggtaaaatgtgaagggcctcaagtggcctaaatgcaagctcctgtcccacctctgctcccataaatagggtctcccagctgggcaacccttctyatcccagggaccaggtaccacccctgtttgttgccaagtagcaggcttcagttccctgccagtctgcggaattatttaacaacctcatgaagaaaccaggggccactccaccctctgtattagcctgttctcaggcagctaataaagatacccaagactgggtaatttataaagaaaagaagtttaattgactcacagttccacatggcttgggaggcctcagaaaacctacaatcatggtagaaggggaggcaaacatgtcctccttcacgtggcagcaggaaggagaagtgctgagcaaaagggggaaaagtcccttataaatccatcagatctcatgagaattcactcactgtcatgagaacagcatggaggtaacccccacatgattcaatcacctcccactgggtccctcccacgacatgtggggattatgggaattacaattcaagatgagatgtggaaggggtcacggccaaaccatatcactcttgttactaccaaacctgctgtccaacaaccctgctgttcactctgctcttgagcaccacctcatgtggccctgcatagcctgcagtggcccttcccctgggctacgagtatatgtgactagaaaattgccgtgggtctcacctatccagtgttgggtgttgtgtgtccagccctagagtgggactccttccctcacgaatggggtgaatagaaggtgataaaaagatctgagtctagggatacctaggaggtggaatctcttctccatgcatagcatgagtgatcacaggcctgaaaccaaaagggacttaggtctgggggagagattattttccaggtgctgaatattcctgggataggggagggagctaaacaggttcctgcccaaaggaagtgagaagggggtcctagcaacttctcagggatttagagctgtgactccagggcctttgttcagaggagctaccttgcaaggaacttctagaagaatgcttctctttctcagcatccatcctcccatttcatagtcgtgcccacgatgggccccgtctccctgaacttgatggctgaatagaagtgtagcctcccaggggcatctaaaggcactcagagccccttacccagccccagcaggcacctgcctggctgcccggtcctcagggttccctgtgcattgagcaatatcctcaaagtgaccaccagggggcagcagcacccagactgccttccactgcacctgcagatcaacaaattccagtattttgggggaatatctgtgataacttggctactgctttactgacctcaggtaaatagacagaccaatgtgcttgaggagccaattgctttaaatctcctgactcattttttgtattaagayttgttttatttatgcaattattctgtttactcaaagactttaccagaagctgggtgcagtggctcatgcctgtaaccccagcactttgggatgccaaggtgagaggatcgttggagcccagacattggagaccagcctgggcaacatagtgagaccccatctctacaaaaaatttaaaaattagctgggcgccactcatggtggctcaggcctgtcatcccagaactttgggaggccaaggcaggtggatcacctgaggacaggagttcgagaccagcctggtcagcatggtgaaaccccgtttctactaaaaatacaaaaattagctgggtgtggcggtgggcacctgtaatcccagctactcgggaggctgagacagcagaattggttgaatctgggaggcagaggttgcagcgagccgagattacaccactgcactccagcctgggcaacagagtgaaactcagccctccatcccgaccccagaaaaaattacctgggcatggtggtttgagtctatagtcccagctactcaggaggccaaggtgggaggatagcttgagtctgggagggtgggagtctggcttgagtctgggagggcgaggctgcagtgagctatgattgcaccactgtactccagcctgggtgagagagccagaccctatctcaaaaaaaaaaaaaaaaagtaccagcccctatctacccattcatagctttatgtccatttcttttgtcttcaagcactggtakcctttacttatctctcctcacctgatctagtgtttacatctcatttgcgcccatagagaagtcatacactgatgtggattttagatagggcacgctctcaagacagccacatgtattattctgtgctcacacagcctggcctggagatgcaaagattatggaatccagaatctaaatgagaggatcagattaatgggatgttctcacagtgtcaggtgaggacagcctgatgcagcctttcatcatgaggctgggacctctgggtcccttggccccaggaccacactcgaggacatgcctgttcctgccaacatggctgggcagagttcctcttttctttccttttcttttcttttctcttctcttctcttcttttcttttttctttcttttcctcttttccttccttccttccttcctttcttctttcttctttctttcttttctttctttttctttttccttctttctatttttttttgaaatggagtcttgctctgttgcccaggctggagtgcagtggcacactcttggctcactgcaacctccacctcccgggttcaagcgattctcccacttcaggctcccaagtggctgggattacaggcacccaccaccacacccagctaatttttgtacttttagtagaaatggggtttcgccatgttggccaggctggtctcaaactcctgacctcaggtgatccacccgcctaggcctcccaaagtgttgggattacaggcgtgagccaccacaccctagccctgagtctgtttatgcttctgtcaggtgtggcatgggcctgcctgggagctattctttttctgtaaagcacaggcagttaatcagtggtctctgggaagaatccagctcagggttatatttcgtttgacccactcaagttttaaaaagtaaattagttgccaatgtgcaaacattagaagagttcacagcttctccaacaatacctagaagttcatccgatggtgcccgcattccctgctctgtctagatggtgcccacattccctgctctgtctagatggtgccgacataccctgatctgtccagacagtgcctacattccctgctccatctggatggtgcccacattccctgctctgtccagacggtgcccacattccctgctctgtctggacggtgcccacattccctgatctgtccggacagtgcccacattccctcctccgtccggaaggtgcccacattccctgctgtgtctggacggtgcccacatttcctgctccgtccagacagtgcccacattccctgctgtgtctagatggtgcccacattccctgctccgtccagacagtgcccacattccctgctctgtctagatggtgcccacattccctgctccgtccggacggtgcccacattccctgctccatccggacggtgcccacattccctgctccgtccggacggtgcccacattccctgctccgtccggacggtgcccacattccctgctctgtccagatggtgcccacattccctgctccatctggacggtgcccacattccctcctctgtctagacagtgcccacattccctgctccgtccggacggtgcccacattccctgctctgtctggacggtgctcacattccctgctctgtctagacagtgcccacattccctgctccatccagacggtgcccatactccctgctctgtctagatggtgtccacattccctgctccgtctagactgtgcccatattcgctgctggctgcaaatgcgaggagttgacagcagcctcccctttacaaggcaggaggtgccactgttcgccattgtctccacctagggcttcacttgctttctatctgcagacatcagagggacccacatctctctgttctgacacgctgtgtgttgatggcagagtttaattatccacatgcaatcttactttccttattcccaagtccgtggggctgcctcatcaaagcattgtaagaactgataaccatcttctagaagtatmatagtgatattaagaacacacatcacagatcatagtaaatggctttaattttttarcgaaatctcactactgcaaatgcattgttgtcctagctaatgaatgcayagagtattgcctgcaaaayaataattgagattctatttttaagaagcttagaacagtacatggtgcatagcaaagactctgtgtatgtgaagccagattttaaaatatggtaacaagtgtctgaaaatatgtggctcaatttgtctcccggttacttttccctctccccctttaaaatgtagaggaaggagaagaagagataagaggtttgtgagtgaagacaagggccctttaaggcctgggaagactaacgccatagggatctccctctgccttaaaaggcacaggaatcttagtggggaaaaagaagtggtgataaatagccagtccgtgtgcctggaatatcaaagtcagtgcgtgccagggatcacactgcgggtcacgtgcactctgggtctctctctgcaaacctgccctgcctcagtctgggaatatgcaactgcctaagaagggtctggcttacacaggggccatgagacgtggcaggcatagctgggctgctactggtcatgaatcctggacacggcaggcaaggtgtggtgtccatatgcattattcgggtggggcaaagatcacagctctcactagactttcagaggactttgtaacccaaagaaccactcatctcaaggactgtggtaactcaggggctgagccatgccagtgtttattatgtgaaacaaggactggaacctcacaagaccaagtctgtccatttgaggatggcccaagatgcacacgggctgcttttatcttatgcgcaggttttaaaaaaatatgtttcatttaaatattccatactcttcaggaatgcccaggcagctgagctttcaggatgtcgcattgcagaggactccaatgctacatatggcagctggagaccctttcaaggcaggtggcagaacggaggccctctctatctgctggggcagccctccgggtgccccgctggaaggcagagcagctccatctctgggtgggtgagaggtgctgcatgggctcactatagtatcccaatactgtatggcagtaggctgccagagtatcctaagctgggtggcttcaacaacaggtactgactcacagttctggaggccaaaagtttgaattcaagcagggctgtgcttcctctgaaacctgtgggagaggagccttcctggcttcttcccgacttcttgggatggggatgcgcatccatcctcggccttccttggtttgtggctgtgtcactgcaccctctgcctctgtcacggcatggtgtcctccctatgcatctgtgtctgaatttccctcttccgataaggactccagtcatattgggtgagggcccaccccaatgacctcatctcaactagatcatctgcaaagactctatttcccaattaggtcacattgaaggtacctgtctttttgggggatacaattcatctcacaaaaccggcccatcacctcaaaaggacctgccaccccagtgctatgtgtccctctctgcccagagccactccttcccctggctctcggggagtgggggcacctttccctgctcccacagtgaccgagcaccttccccttggtatgcattctgaagggggcatttttttctcctccatctcagccctgtacaaagcaagttctttctagattgaggtgtgtatgtgtgtctatgtatatgagtgtatgtgcctgtgtgttttcagggagatgtgtgcaggatgggtgcaagggaggagtggaaggcggaagggcaggaggaggatagagccacaagagtgagcacagaagtgacaagggcagaatcagtgtgtgcttgtgacaagtatggaaatgtcatgcctttaggttcagtcctataaggtaggtgtatcagtaagggcattgattctgcgaccttaacagagatcrgaataacagtggcttaaggaagagtggagtggatttctctctcctgtaaatctggcctggtgggtggtaaggaggatccacgtcgcccaggcccagatgtgtgtggctctttgagtgccccgttctttcccagtctcataactgctctccacctcctccacatccagccactgggaagcaggacaaagttagttaagggcacgttcttttctttccaaggattacttggacattacagtcttcacttccatgcctactggccagggcttagtcacacaaccttgctagctgcaagggagtctgggaaatgcagctgctattctcagaggccatgtcctcagggattctgctaaatttagcaaggcagggacagatatgggggaaccactgacagtctatcacaaaagaacgtgattttagagaaacagtgaaacagtgtcattaatccacccctcaccccttacaacagccaaaaagaaatccagtggtatccatcacaataaagagtatgagaggaatgtgattagaaaatcaggttgccaggcaggatgtgtccagttttagccagtggtggggttcatggggaaggcttcgtctcaggaaggtgtgtgttgggttgttctatggccagatggttttcaacgacatagcacgacctgtagctctccaggacccgggcctggacataggccttgtccttctcttgccaggcatcggactcgatgtagacgttgaatgggtcgttcgagtgctccagcttcttggagcggatgtagctcagcatgatgcccagggtgaagaagccgaagaatcccagtaccatgaggacgtagagggcctccagcttgccgtcactgctgcggggggacctgcgggccaggcccgacatgttgccaccctgctgaactgtctcctgccacagcttggtcagaaagggcgtcaccgctgtggtgttagacaggatcatcctgggcattaaggttccactgctgcagctcaaacttcccaggcacacctcttaaaggaaaaatgcaaccccaaatcaaaaagtacgtattggccaaaacccacacgtacgcacacacacgtatacaattttaaaatctcaggtgagaggggtgagctgacactccacaggccatggcatgtgccatcttgggtctgtgcaatgccttctcttgatagatgaatggatacattgattcccttctatttccatccaccactccaatctccacccctataggtgaccatcagaatgagcttctttaatatgcattgctgcatattgtggcgtatggggttgcatgtgtgcattttggtttacctaaatggtatcaggtcatccaactcattcagtctccttctttttcctcatgactgtgcttttgtggctcacttgcggtgctgggtgtgtgcattcccttgcttccaatgtttgtatacaacccatggtgaacacccacttcttttctctgccttctcccccagagatggacactgctgtggctgctgactccataaacaagggggagacaaatatccccatccttgacctcctatggacctaaaaaaaatcacgcatctcatacaactagttcctggcrgcttatgcaagactagtcagactggttgcccttggaagcaacgtgcaattggtggtctgtttcaccacagagcatctctctatgaacagttacattcatctgaatgaaaaatctatgctgtcacgtggtggacttcagaatgtctagggagatttcacagagagctcccctcgaagaggccagtgttgtagcttgtgctatgtttttccctccctgcccacacacaggcacacacacgtacacaatcttaaaacctcaggtgagaggggtgagctcacatgctccctagtccatggtatgtgccgtcttgggtctacacaatgccctctcttgattgagcaatggtacatggattgccttttatttccattcactactctctggctatgcagaaagtgacattttccctatcgtttaatcttgatatcactgtccctgtatactcagagtgggcctgggaattggaaaaattgtctccaagtagctgtaagattctgtcaggggtttggtttgctgtggaaaccccatctaggtgaccttgagatcattggtaagctgaaaaaaaacaggtcttgtttttatttatttatttatttatttatttaggtttgagcaaatgccagcctctacccccagttcctgctgggaaacaaaagctccgaggccaagttgttgatgtcacattccaaactcaagccagagggggccactgggagcttatcacacgtaagtgctcccactcagttctttctttttctgttttattgagacagggtctcactcttgtcactcaggctggagtacagtggcacaatcttggctcactgcagcctcaacctcctggactcaggtgatcctcctaccctaccctccagagtagatgggactataggtatgcaccaccatgactggctaattttcgtattttttgcagaggtgaggttgccctatgttgccgaggctggtcttgaactcctgggcttacaggatccgcccacctcggccccccaaattgctgggattacaggcatgagccaccctgcttgtcccctgctcacttcttaggagcttaaagtagctgagtagaacatggcctggagtagaacatggcctcggggggactgttgtaactacaggtgaaggatgtatttgggaagacagtttatggccagaatcrctatggaaagacaaattccaacacttgccgggacggcgctgtctttcccagccaggatggggactgtgacattgcacatcatcttgtgtaggacaaataacctcagaaacctagctcctctccagcttagacccagagctatttcttcattgaattggtttaattgtaaaacataccctgaacccagcaccagctgaagacatctggcacctttccgaggcccctcttcctctacccatctctgaactctggctgtgtctcagagttctgttacctgctctcttctcttcctactctcctctctcccagggtgactgcacctgctccaggctcacctgcatggccatgacccagggctctctttaagctccagagccatgcgtccagtgacctgctaaggagatggttccctttggccatccccaggctccttaaagttaacactccaccctgtcctgtcagagactggcccctgcctcattgagctgagtggcaacaccactcactccaaaatctgcatcactctcactgatgactgcaatctcatcatggcagttcccatcttgaaggccttccttggctcctccctgccttcagggtgaaactcctggtcttctgcatggatacaggccctaaatttgagagtctatgcatccctctccaattccactgttgccactgtgcccagaccctatgctccatggtccctgctggccccggagcctctacacattgtgcgtctccagctcagactgcgccttcttcttggcccatgaaacttctcagcaatgcctactcatgcttaaaattcagcccagctctcacctccttcccgaagcctgctctgatacayggggctggatcagcactgtgcacaccatgacccctgctaacctcatcatggtcaggatctccaggccccttatcccatccctgccctgccatccagcctggtgctgggcacgcaaccacacaggagctgcccatgaatgtttatcgaatagatgccaccagaacttaataccttttgaccagtggggcttgactctttataacctgcttactccaatgaacagatgccaatgagctgtctccgaagctctaactgactcccttttccagaagggcagtcatctcccaccctgaaccacagtctcagaaggcaggagtgaggagcagaaagagctcagattttgggattccactgccgccacaggtttggattctagctttgctacttcctggccacatgatcctggacagtttccttagaattgttcagtcaagttttttttttttttctttccaaagtagcgagaaacaccactgacatttgcgggctgttgaatcactgagcaggtgtgtagagtggctgacagcatgtggcacatggcaggtgcacactcagtggtcctgggtaggagtttattggtttttctacctcattaagaaattgctgcccaaggatttggggctttgggggtttaggcttggctttcctgtggctgaccatggcagctgtcttctctacgttgtggagagatcagacatgaatgagaatcaaagattgtttgtggcctttcctggtttctaggcttttgagtctgtgcagagatctgtcaggggttaagctgcctgggctcaagagattcaggtccttgttcttgtacaaaactagcatttagccccattctaaccatcggggtaggcaggaattgtttggtaacagatccaaactcaacgctcaaccatttctttttaaatgacccgaaaccacttatgaatgcataaaaccctgccccagaaaacagacagacctggacctgatactatgatgtaatttccaaaaacccagaatgatcacaattggcaaataattctgccaccaatcactgttagagagtctttccaacttcatgaccatgtgaaggtagaattatggcaggcgacatttgaagatccacaagttaattggtttaaaactgataaatccatacagcaaattaagagttacatctgcaattaattcataatagtgagttcactgagaaggcttgttacttagatccagatggactttcttatgtccaaagaagcaaccaaaaacatctgctttgaaaacctcccaagcccaaaccatcctcagccttgttctttagaatgctttagaatgaccttgttaaaatgcagattgctcctgtaatcccagcactttgggaggccaaagcaggtggatcacttgatgtcaggagtttgacaccagcctggccaacatactgaaaccccgtctctactaaaaatacaaaaataagacaggcgtggtggcgggcacctgtattcccagctatttgggaggctgaggcaggagaatcacttgaacccaggaggtggaggttgcagtgagccaagattgtgccattgcactcctgcctgggtgacacagcgagactctgtctcaaaaaaaaatgcagattgctgggctctattttcagagtttctgatttggtagaactggagcgggcctgggaatctgcattcctaacacattcccacgtggtgctaatactgctggtctggaggccctgcttggtgatctattggaatcaccgggggagcttttagaaaataatggttcctggatctcacccctagagattttaatgtctttggtctgggttcctgcctgactcagagactttttagaaacctcccaaatgatcctaatttgtagccaagattgagaaccactgggctgtggtgtgggaccctaggaaaatgaccaatggccttttgtgctgcagggtacctggaagaattttgcaaaaatatagaaatatgatctcactgactgtttttcaaatcttgtttgttttttacattttcttttttggccttgtttgcctctgatacagtctgaaaagaaattgcagaaagaaactctccagtcttcagtgtaacctcagctgtccccagtctcacacacgctggtgccttcaattacaattctcctgtcagagcttaagtccagctaattaactgcctttcaaatgacaaccctatatttttaaagaatttttttaaaacttcacatgtaatttattgcattgcttttgctaaatgtcctccacacccccaatgcctgctaggctgggtcgccatggtatttttgtgtaacgagtctcaaaatgagtttggcaatgtctccgtaatagtcagcatggtgtaaatgacagtctggatctgcatgtcatttgggattttatatcagattctctaggttcatttctatgatacgtgatgccaaagcacccacatgccccgtggctgcactttcagacagttggactcaaacagagtgggagagcaactgatccaacaatctgaattttcagaaaacggggctccttagagatgagatggcttgccaaaagtaatctctcctatcagaagtacatatcctcagcaaactaacgcaggagcagaaaaccaaacaccgcatattctcacttataagtgggagctgaacagtgagaacacatggacacagggaggggaacaacacacactggggcttgtcggggaaaggtgggtggggaagagcattagggaaaagagctaatgcacgctgggcttaacacctagatgatgggttgacaggtgccgcaaaccatcatggcacatatttatgtctgtaacaaacctgcacatcctgcacatgttccctggaacttaaaaaaaaaaaaaaagaaacaaaaacaaccaaccaaaaatatatctaaaatgtcatctgttagcaattgactcacatattattagtatagaaaagagcaattcccaggaccttgtacagaggaagcaggctcaaaacagctgaggaataggccacttttatcagatagcattggatccatgcacatgggggttggcttcttacctaaatatgccatcagaaatcatccttgttcctgtcccctcagcttttgtagcttgcacagtgagtaaagggatggtggaggcagaaatggtaggagccagagatgttcaaaatccatctgatgcttggcctgtgctgaacgttctcaaactgtggccttgtcaggcccagaaagtggggtggattcctggccgtttctgcttctgcctgggtgtgagatgtgaatgctgcccctactgaagggtagtgcaattttttttttttcttaaaagcttagacccagagctgctaatctactggaaatcactcaggacacagggctctgggcagctgcgctgagcgagacacctgcaaatggagaccaacggggcctccagcaccctggagttccgtaaggcccccagctgaacccaggggagaagagggcagtgggtggcgcctcgctgctctgggcacacaccacctcttctgacttctcccacgtgctccggctgtgtcgcctatcagcactgataacagcctggaagctttcagaacagaagctttcccagcatgggaaactctcattctttcttttttttaattttccaaagcctttatttctaagaccaactgtggcctacccgtgcataaactgggcaggctgtagacagcaggcttgccaagtaaatactacagccttcctcccaatattgagccctgtcccattgatcctgcaggggagatgtgtagggcatttgttcacggggaggccacaagtttgggcctctcatcactgtgacctcacacccctgttgagtgtgtcgtaaacagaggagccgttcttcaagccccctgccctgagtgcacccctctcattcttttgttattattagcaaattcccccagttcctgatcattctttcttaagcctttagtgactgggtaaggttcttgtggctgactccaagctttcttctaaaaggaatagattctaggggtgagtagaggagacaggaatgtcggagtcagagctcaggaatctgggttccagccccaggtcaatcctagataaactagagggctccttaacttacttccctaggttgactctgggtttttttatcacgctcgacaggactccttatgcatttcttcgaaagagcatccagtcttaacatcatcatttggcctcatttggtttaagaagcagaattagggtaaaagcaatgatggataagcctcattttggtgaatatgatcttattgagacaagaattctgagtcaattgtccttggaaccaactgtctattgttttcatctttatcacaacactatgtccaaattttggaaacatgtttccctcctagcagaaaagaagccacccagggccaccagatgcttcccgacacttggctggctttgtctggtcttctatccttcccctatccccagagtcagtgggtactaaggtggccagggtcgctcaaggaatcagaatgcaaccgtccagaggcccagaatcagactgtcctctctgattaggaaagtgtttcctgccacctcccagggggatgtggggtgtggcttgagtctggtgcttttaccaggagcttcccagacctctctatgggtgatggagagagagtctggggtgtgaaggatggaaatataaacactgaatactcagagagtcaggccagcgagctggtgaggagactccatcaaactcaatatgaaaacatgggtgggcgtttggggatggataatgaatgacagctgaagtcacacatcaggagggaaggaaggactctcatcttcagcaaatgacataaatctggggagcctcagtttcctcacctgaacagtgagaatgatggaatctaccctgagtatatatcccaggtctttgtgcagcatggaatcagcccccagccttcctagctcttgctttattttattttattttagagaccaagcctcgctctgtcactcaggctggagtgcagtggcgcgatcttgacttactacaacctcctccgcctcctgggttcaagtgattctcctgcctcagcctcctaagtagttgggtttacaggtgcatgccactatgcctggttaatttttgtatttttagtagagacggggtttcaccatgttgcccaggctcaacatgcctgggctgaagcgatccacctgccttggcctcccaaagtgctaggattacaggcgtgagccaccgtgcctggcctcttgttttatgtttgctgttctcgctgtaggtgaggcattcccaccatattcccttgttaaaatcctactggcccttcaaatgccagctcatatccacactgtgtctgaccctgatccagaatcctctccccttctttgaccccttgtatatatctttcctacagtgctggtcatattctaacttctaatcatttgtagacttatctgggcattttctcccaggaggtcccaaaggacagagaccatcttgctcacctttgtccccactccagcacccagaatgtttcaataaggttttcctgaattaagtgggaagcaaagtattagattcaataccactacaatgggaaagtgagtgaataattgataaagtatactatgctctggaaatagattattgagtaatcagaggaaaacctcttgcaatgtagtatgtttttatatattaaattttatattatttatttatatttatattattaacatatttttgttattcattttgtttattaagatttactctatatatatatgtgtgtgtgtgtgcgcgtgtgtgtgtgtgtaacccaccttatttctaaaaaaaaattttggcagtcagtgactattaatagtagagcacaaactcttaacctttttttcctcccgctttcccagagtccttatcaccttctaatataataaagttttcttaactgtgatgttgattgttcaacgtttgtctttcccaccaaaatgtaagctattgcgggcagggatctttgcatgggttgttcttcattgcatcccaggccactagaacagtgtgtggcatatggcaggagtgcaataaacattcagcgaatgcaggcaggcatgcgtgaacgcatgaataataagtgttcctttcaactaccagaaagagcgaaaccacacaggagctgtctcaccctcagagatttaagacgacagcaatgagtccctctgatgtcttctaagtgagctttattttcaaaacaaatcattttccaataacctatcaaaagcaggtcctacctgaatatccttggctatttcctctggatacagataatgccttcctccaatggatcctcggctcacctgatgattgtgtgaaactggccagagaaagcagagggatttttcctggtgattggaaatcagagtcacggctgaatttaagcaactgtgtgaactcagcaaagtccccaccacctggaccatacatgacagctatggttattgacaaggtccttccttaatggagctggaacctccttcttaatctaaatgtggactcaaatgaactgtcaattcacatagagcaatgtgacaaatccggggggcaaagcattatgcaatagattgggcacatgcgcacgtctctgctacttagtcaactttcttctaaagtttcccacttttcccattaccacaatcaagatcagatatcagtaatattctccgcgtcccatgccctttcctcagggagatgccaaggcccaagagctggtccccccaaaaagctgaaggtctttgaaaaaaagtggggactcaggtcccgtgagtggttttgattttcttttcttttgaaagttgcatgtaagtgttttccaaatactatacaagaatgtctataacttaataatggaggaatgttttcgttgttctgtgttggtgagatgctttcctatgcgtttgttgtataagtaagtgagaatggcagaaggaaaggaggggagaggctgatcatcctatcccgtctcccactctgggggtctcggcgcttccagcctgaagcgcgcccgctgcgcgtccggagacgcaggttccaggagccccccggggttgcccgactaggccacttgcgccccggaagagggccgcggaggtgggagaccctaacttacccgggtctgagatgccgagagagccgggtgtggagctgagtgcgcgcctgccgagcgctgaggccacagacagccccgccccggggcggcaccttctaagggcctgagcgctgcacagggatgggggcgggcggggcctcccagagccgccaggcgtcccgccccactccgcccacacgcacggcccagcccagggttccccgggaccaccccagaccagccccggcccccccgggtcctccacactctgcaccccagaccaacaccaacgcgcgtagggaagcgttttagatcctgtcggagaggctcaaggccggcagaaggtttgcattaggatcgagaaagccgaccaacggacagatctacctaccttcctgcgggagtttgaggttgccaggggggaagccactgcagccaggagaaaggccgtctgggaaccaccccaccctcggacgtgcgggccttcaaatatctctgaacataatcctccaaagaccgctcaacctccgctcccgacgactctttctagcctcgtcccgcaccccagctgtggccacacacctattaggcaaacatttatgaagcacccacttactgggtgtgcagccctgagctgggggtgcagctgtggaccagacaggagggggcccggagcggcagacagtcgctggaggcacccgagccttggcgagcacaccctaacgtccttggggcctttcagcccgagccgtccttgtccagagagcaaaccatgcaatgatgcaggtgacttcccagcaaatttcatagcgttgctcaccagcttggcaggcaagaggagaaggggcagtccccaaaagacaatcccatgaaccttctagggaatgacgtccaggctccaggctcctgctctgcaggcgggtcgcagaggcaggttcctgacctaggactagaagacattctctagggtcactgcctccatggtcttccttggcaggtcacttcttcctgggcttcgacctcggtgttctcatggggacgagggtgattggaggccctccaaggggtgcaccgatgtgtcctgtgcaccaggcagaaccagcattgccctacagtgtgggtgcaaaatgaacccacatggccacgttggaaagtcctgaaatgttcatagcctatgacatgaaattgcactgtgtgaaatctatttattctttttttttttttcttttttttctgagacggagtctcaccctgtcgccccggctggagtgcaatggcacgatctcggctcactgcaacctccacctccctggttcaagcgattctcctgcctcagcctcccgagttgctgggattacaggcaccggccaccataccctgctgattttttgttatttttagtagagacggagtttcgccatgttggccaggctggtctcgaactcctgacttcaggtgatccacccgcctcggcctcccaaagtgctgggattacaggtgtgagtcagcgtgcccagactgaaatctatttattctatggaaaggatcagagctgtagaaaaatccttatgcatgtaaaagttctttgtgtttttacttgcaataactagaatctaaacatccaaaaatagaaaatataggtaattaaattgtagtacatatgatattattctacatagtagaatattatgtggagtccttaaaatgtttacaaataatttataacaacatggggccgggcacagtggcttacacctgtaattccagcactttgggaggccaaggtgggtgggtcacctgaggttaggagttcaagaccagcctggccaacatggtgaaacctgtctctactaaaaacacaaaaatttagctgggcgtggtggtgggcacctgtaatcccagctacttgggagtctgaggcaagagattcacttgaacccaggaggcggaggttgcagtgagccaaggtcacgccactgcactccagcctgggcgacaagagtgaagctctgactcaaaacaaacaaacaaacaaaaacccaacaggggagatctttatattatcatgttacgtgaaaaatacaaaaacagaaaacaaaacaaaaaccccacaaaactcaaggccttaaattgtaaatatgagatgccaggcattattcccaaaatctataaggaagcacaccaaccaccatgttaacattgtctattagtggtaggcctattggagatttattatcttatttatgctacttcatgttttccttccttttttgttttgcaacaactctgtattagtctgttctcacactgttataaagaactgcctgaaactgggtaatttataaaggaaaaagtcttaattgattcacagttcagcatggctgaggcctcaggaacttacaatcatggccgaaggggaagcaaacatgtccttctttacatggcagcaggagagagaagtgcagagcaaagtggaaggaaaagccccgtataaaaccatcagatctcgtgagaactcactcattatcatgaaaacagcatggaagaaccgcctccatgatccaatcacttcccacaaagtccctcctgcaacatgtggggattacaatttggattacaattcaagatgagatttgggtggggacacagccaaaccatatcaaactctatgtactttaatatttaaggaaaattacataaactttatctgaaaattccctggattcttctcctcaaggtcatgctgtacatatgcaggatcctcctgcctacatctccaaatggataatggattgaagcaaaatgtttgtcccaccaaacattgatgtgtaacccttttagtaacaattcatagaccaggtaaacatgtgtggagcagcccagatcagctggagggagcattattctcatcagagtgtggaacaatgcctacccatctgtcagctggttatggtgaagacagtataaatggaagcactgtcagaaactaaaggcttatgtaagtgatgctgttcttatgtctcctcttcctctctatcccacatatgatgaattattttattatgatgatgctgtagttgttattttctatttgaaatgaaaagacatgattacagatgagaaagagtgtattttgttatccttgatgcacttgaaatggtttcctcttttttttttcctgttttcttcttcttccttctctcttcactctttcattcttgcctctctccattttataggtatgattgatcttgaaataatgatgataacagaatgatagcccatatagtccttgggtcacttctgtctctatttctctttcttccctcctctttttttcttggtccttttgctagatgggttgcgagcatggctccctgcccctctcagtgggtttatcttcattttccatgagctcccacctcactgcatgtgacatcaaagccaaagctccagccacttctcatcctttcttaggaaaatctcaagtcttaacttgaaagttgaatgtcctgcttttgttcctaactctgctcacaggatagtggaagaagaaaggctcagcctgttaccagaaagaacagatctgcagtgtaacccatctaggatgaagggtcatgtggacagttcttcactcttccctctggctgcccttcagttggtgtccagatgcccctgtgttagcctgggggcattccttgccctttggcccctcttttgttgcctttttttttcttagacaaggtctaactccgctgcccaggctggagtgcagtggtgcgaccattgctcattgcaacctcgaactcctggctcaagacattctccccgctcagcctcgtgagtagctggaactacagttgtacaccaccatgccttggctaatttaaaaagtctttttgtagggatgggagtctcaggctggtcttgaacttctggcctccagtgatcctcctgccttggcctcccaaagttttgggattacaggcgtgagccattgtgccctgccctttgttgctttttcactcctttaccctgtggtttctactatcctcagggacaactctcattgccctggggcttctggatcttcagcaagacacacccctaaaggcaaacaatctttctttagcagcagcagagcacaaacggagtgttgctgtatgctaaaagcatactatttcccccccatgagaaagtctaaggggtccaggggtcctgaagtccctatcctgcccccgccagtgacaggtgatggggaacagaatgctgaaagagaccccacccaaccatctggtgtgtcaacacccgcccctgccggtgctgtccacaggttgatccccatcccacatgggtggcttaacacattcatgtcttctgagagccatttgttgctccccagtctcttctgtcctccttctttgaccaacatttccctgtctgatttgcttggtcacaggtagcctgtgtcagtttcctcccaggactctctttgcctgggaataagtgctatacatgagaagcatccctcccaagcgctgtgtgtggcctcctggcattctcctaagacttcctagaggattgctctggttccaaaaagaaaccagccacaacctgtttggagccctgcaggatgttccagctcaccgacttcattccccactacttgcccctctaacagagcttctagaccagcagccaaggtgcctccattcactgtacatgactcacctttcccccttctagacacttcttcacgggtctagcctctgtttccagctttacacagcacaggctgatgcctgccactgaacgggatccaggactttttcaaatctcagatccccagcaaaggtatgacaatgggcaccctgaccagctctgagcctctaagcatagggtctatttgtttaattttgaattcacttttaagatttaaaaattgagagctctcatcttcaacaaaattcaggggatctagaaacatcatgcccacttgtggcagagtagccaactggcagaaactgatgctgatgctgtctgtttcctacacaaaatgcaaaccagagtttaccacaggccccgctactccacattctcctccacaaggacactcagcaggctcctgtgctgaacggcttgcctggttctgtaggtgtagggtttgttactctgttccaggacctattttccgcaaagccccttacctggctgtttcttcccttggcatacgtgagaactcctttcctcactgaattgctttgaccttgctaattagttgtgttgcaagtgctttttggctgtgtcagacatgtatgttctgtttatcattttagagcaatggttattaatttttttcagattcttcttgggaattagagaaaaacttatggctctcctccaccatcctgcccccacctaccccaatgcacacatgtgcaacatttgttctacaattcaagagctctctagacccctgatatgctaggcaattcgtggctgccacattaggaacccctgatttaggggaaggactgttttctccattagacaaactcaactgtgtagcatcaatttcatgggtctactgtgtactgtgcccaaaaaatgccactgaatgctgcctgactggtggatagcaagactgtccattaatgtggtcatttaggttgcctctgcccaggtcttgaggtcattggcactaattcacaacaccctcaagtcacccagggaagatgagacacagttggctgtagacccacagtttgggcattacagctgcccttgaagttgacaaataaccacaaccttcaaattgttatgaaaagagcacaaatccaattaagaaagcttttccaaaagaaataacagtgttcctaccccctctgtcactctccaccccctttttgtcccagaataatgttgtgctgataggaacatggataaattaattacagtctggaatgttattcatgggtaggaaagaacactaaatctactcgcacaatgtttgatatttaaagataaacattgcctttatgttttttttttaaacctcagtcagcctagtttacgaagacataggtataatccttttaaatgctgtggattttttaatcgcaaaggtaacaatatgctgggtgttttacccagccagagaaccaggagatgcaggaatgagattagcatctctttagttccttgcatatttgatattattttggtgtacctccaattcctgataacatagaagaactcttgtggttgaagtccctgaaatggaaggatattggtaaccctgaatttaaaacaagcacaggcagcctttgtgggaatgtgtgtgaaggtcaccttctagaaacaggactgtccatagccattgccatggtttctgtgtcatttcaaccagaaccttaggcctggaagtctggatggatgtgggttggcatggtcctctatgggcattaaatgaataaatggatatagcagagggagtatccagcatgactcaaagaaggatgagaggaaacatattcaaataaaatctttagaaaagcaaatttcaaaaaaaaatgcttaagtataaaatattttgatgacaaccatgattttcaaattgaattcttattctaagtaatggtctaatctgaacttagacctctttccttaatttttttctcaataagcctttggtgtctagtcagttcaattcagtatttactgagtctctatacagacagggtataaggcattaatcaaatgtatgtccaaaattgcccaccatgcagggcagagctaaaatgcctaacacccctcctctcaccaacacatcccccacccacatctccaaagacttcctggcagaggtgatctctgcctgctgggacagatgtataggctccaacagcagcagggtggcccctctgaccaccaccttgggacccacattgctcttagaactattcctcttttttcatccttgaagcccccagcaaagctcagcctgaatcaactttttctaggaatctgacaagtttccaggmtgatttcctgacccagtcagatcctcttccatctttctttgggtgttcaattttctac

SEQ ID NO: 2 - DNA, 113604 bp, Human
tgtcagaaaaagatacaacatttaataacgatggaatgtaaatatcaaatatttttatttacaaacaaataaaagtttttatatagaactaaaatgatttctataacacctgttttcactgttcttcaatatttctcttttaacttttcaaagattttctttttaaaatttttttttgtagagatgggatctcactatgttaacccagtctggtctcgaactcctggcatgtgatcctccctccttggcctcccaaaatgctgggattacaggcaaaagccaccacgcccagccaagattttctttttttattgtctgtatttaagatatgcaacaagatgttctgacgtacatatgtggagtgattaccacagacaagcaaattaacataccgttaacatattacaccaaccattaacatgtccatgttcgtgtgtgttttgggggggggtatgtgtggtaagagcacctaaaacctactctcttggcagatttccagtatgcaatgtgtacaatgcttataaacatttttctttattaaaacaaaacaaaaaaccgccacatatatggaaagagctgtgctggatgcctaaggaggaagcttgatggttctaagcaaaggccaaaaagtggtctgatctatggaaaaactggaaagccggcaaaatttgctgtgagagccctttctctgctcccatctgtgctgatctgcttttttcctacaagagcccattggccttttatagttcctggggaaaatgaagcccccacggttgtgcctccttgagttccaggacttccccctctccttcccatccagttttaacccccacacacctgtggcctgcacgctgggggttcttttctgtttcttttgatcctcttcctctttgaaaatcatctttgtaaaacaaacctaatacggcaccatttccgtccagattcatgctccaggaagaaagggtgctggaggctacaggggggcctcacagcccatgtgatctctgtgttacattcattttccactaacagaaatcaaagaacataccctgccattcggcctgtgacaggggtctttgttaactttgggtcttgctaaagttcagtaatgtcagggcaaacagaacacaaggaagctgagacgttctctggctttccgttgcaaactaggcctgttgatgatggtgacaagcttctcaatcccagaagcatgaagccagcaaggctgggaaagcaccttgggggaaggctccatcagaagagaatcaactttatcaaaacttggtttgctctatcacagcagcggcatttcagaagcatcctaccaagttgcttgtttcattgataaactaaagaaaccctacatgtttggagagttcttggtgaggcctgttcattggaagtcgtcatgcttgtgtgtatcttagagaagaagaaaattccagtagttcctcagtcaaatggtgtaatccactccagaatcattgccatctcttctaatattctgaaccaggcacagagaaagtagaagctcagtgcatagctaaatgaaattaccagagattctcaatgcccccatttccagcttttcacaaaaccattgtgctcacattaatagcatcaaggaaagcttcctactctgtgagctcaattagaaacgtcatgtattgttaatggttttgaaaaggtgaaaactttcttttccagagtctttttcatcggaatgataatcttagtaccttgtaaatagatgaggtggttgatttcatcacagccagaatctagaattatcaccattcttttgggatacagtgagagcttttttccagccagacacagaatgggcaatacaggtaaggtccctgttgtcatggagctcaagttctgttagagacaggaaagaaaaaaacaatcaataaaacaggaaaacttcataattcaaagaagccacgcacagctgtgagactatgcacaggcaccattcacactgtagactaagtgacagctgcccccgggttaagccctgccttgcctgggcaacagtacatggcatccccacacaattcagataatacaaagtgctatcagggaaatgtgaagaaggaagaagctgtgccaagattgggggaaaagcattagaggcagagagagcagcgtatgcaaagatgctgaggcagtccgtataattgcaccatgaagaggtccctgtcctaatctctggaagctgtgaatatgttaccttacctggtaaaatggactttgcaggtgtgattattaagttaaggatcttgagatgggaagatgatcttggattatccagatgggacaaatgcaatcacaaggctccttataagaagcaggcagggggctcagggagtgggagaagatgtgatcacaaaagcagaggctgggccgggtgcggtggctcatgcctgtaatcccagcactttgggaggttgaggcggacagatcatttgaggtcacgaatttgagaccagcctggccaacatagtgaaaccccatctctactaaaaatatacaaaaattagcctggcatggtggcaggcatctgtagttccagctactagggaggctgaggcaggaggatcacttggacccaggaggcggaggttgccgtgagtcaagatcgtgccgctgcgctccagcctgggtgacagaatgagactctgaaaaaaaaaaagaagaaagagaaagaaggaaagaaagaaagaaagaaggaaggaaggaaagaaagaaggaaagaaagaaagaaagagaaaaagaaagaaaaaagaaagagaaagaaagaaaaagaaagaaaagcagaggctggagggatgccaggatgccagggagagggccaagagccaaagaatgtgggtggcctctggaagctgcaaaaggtaaagaaatggattcacactccctcctcaacccccagagcatccaaaagaaactggctctgctgccatcttgatgttaacccagtgaaatccactttggacttctgacccccagaactttaagataatattataaatttgtgttgtttaagtcactaagtttgtggtcatttgttataacagcaatgggaagctaatacagattcaaagacaaaaacaaaacaaaaagcagagaagcaggatcagactatgtgagaagatgagctcgcagaaaggaccagcggccagtttttccatggcctggccagctgcactaagcattccgggttttattttcagggtgaaaggaacccaatggagagtttcaagcaggggaattgcgggctgtggtttgtgtttccaaaagatcccactggtccctcttagaaatatgtgaatagggacagagtgcaaagccagaggcgtgagcggccagcagtccaggtgtgagctgagaagtagctgtgcggggacgtgtttggagagtcaaattattactataataataattattattatttagagatggtgtcttgctctgtggcccaggctggaatgcagcggcatgatcccggttcacggcaatctctgcctcccgggttcaagcgattctcctgcctcagccacccaagtagctgggattacaggcacctgccaccacacctggctaatttttgtatttttggtagaaacggggtttcaccatgttggccaggctggtctcgagctcctgacctcaggtgatccaccctcctcagcctcccaaagtgctgggactacaggtgggagccactgtgcccagcctggagcgtcaaatcaaaacaaaaaaaccaacagagcagtaagaaacatatttcaatttattaccttcggggctaggggaagagagacaagttctaagaggcttttcgtgcaaaaatggaaagaactaggtttaaggcaacagtgagaaatgacaatgaaaaaggcaagcttctgataacagctgtctgtttggctggtgagacggattgtgactcttctttgcaattggccattgtactagtttatggcacaaaaacccaggcacagttttcaaagaagttgagatggggtattaagggtctggtgggttcgatgtcacccagcacaaactcatacacccacgttcaacctgtgcagagtcttttctttaacaggatgcagagtcaacagtatttgctagtgaattgggggtgtggcagggtgaccaaaaaaaaaaaaaaaaaaaagaaatctaagttaattctttggttttttggcttacacaactagaaagaaagtggagtcactttactgtgataggataggagtaggtttggtaggagaattgagttatgtttggacatctgaggttgagatgcctattagacatctgagtgaaaatgtcaagtgagcatcttgacatttgattctgaaattcagagaagaggactggactggagatacacatttgcaagtcccctacaaatacatggattttaaagaaatcaactttattgtggtatagtttacataaaattatacacacccattttaagtgcatggttcaatgagttttactcaggtaaccacctacaacaaccaagatatagaacaattctatcaccctccaaaattgtctcttaatcctttgcagtcaatcttcccctcatctggtcatagaaaactactaatctgctttctgtcacgagggatggcttttgtctttctggaatttctagaaatggaatcaaacagtacaacttctttgtgtctgacttcttttgctcatataatctttgtgggattcatccctgttggtgcatgtatcatttatttgttcttttttattgttgagcaatatttcattgtgtgaataaaacacaatttgtttacccacttatctgttgagggatatttggcttatttccagtttttagctattttgaataaagctgctataaacattcatgagtttttgtgtggcatattttgaaaatttctctaggtaaatacctagaaggggcattgttgggaacttttgatctagttaccaaactgtttacaagtggctgtatcattttacattcctaaggcaatgtatgaaaatccacttagcccacatcctcaccaacacttggtgttgtcagtctttttaaatgtagccattctggccaggtatggcggctcatgcctgtaatcccagcactttggaaggctgaggtgggcagatcaccctgaggtcaggagtttgagatgagcctggccaacatggcaaaaccccgtctctactaaaaatacaaaaattagctgggtatggtggtgtgcactgtaattccagctactcttgttgaggcaggagaatcacttgaaccctggacagggaggttgcagtgagccgagatctcaccactgcacttcagtctgggggacagtgagagattccatctcaaaaaaaaaaaaaaagtagtcattctggtgagtgcgtagtggtatctcattgtaattacaagttatatttccaatgacgttgagcatttatgtgcttattgtatatattttttggtagtgtctgcataaacctactgtccattattattgagtcaattttttttcaaatgatgagttctttatgtattctagatgcaaatctgttgtatattctagatacaaattcatggtggttatttttttcagtatattacttgcctttttattttcttaattatgttcttcaaggagtaaaagactttaataatgatgaggcccaatatatcaatattttccttttatgatccatgtgtttcagtctttgtttcctccatctgcttttaagattttctcattgcttttgattttaagcattttatgcaaaggtgtgactttctttgaaataattctctttgagctttttagttcttgaactcatggcttgatttttttaaaaagcagttttggaaaaaatctcattcactatttcttcaaatattgcttcagcttcagccactttctcctctctttctgggactccaactgtacttctgttagtcctttttcctgtatctctcatactttcttgggtgtatcctattattttgttctgtgcttcactgtagatattttctgctgactagatttctaattcactaatcctctcttcagctttgcctatgctgctggtaaatccatccactaaattgatacatttaaaaaatagatttctgcggagattctatttattcttgggctgactctggaaatttatatttttctggcaaattatatatttcatccaaaacttcaaattgtatttccagagtgactttttatttagttgattctatatttatttgttttctcttttgttcctttctgtttttgttctaaagtttcatttattctacatttttagtttattttttggttcttttgctttttgagtttaatgagttcctattattttccttttatttatttttattttttagagacagggtttcactctatcacccagactggagtgcagtggcaccataatagctcagtctaacctagaactcctgggctcaagcctcttcccacctcagcctcctgagtagctagggctacagctatgcaccaccattcccagataattttaaacaatttcttttttatagagatagagtctcagtgtctcactatgtcacccaagctgttctcaaactcctggcctcaagtgatcctcctgcctcagcctcccaaagcagtgggattacaggtataagccaccatgcccagcctcttttccttttagtgaattcatttactgtataaatttgcctctaaggttcacattgtctgcatcccagaagttttcaaatatatggcagttcctttgtaatttaatatcttattatttatgtgttttcctctttatccatatgttacgtgatgatatacttattttttggttttcagatatatcaaggtgtacagactatatttttgctgatttataattcaactgcattgtagtaatggaacccagtctacataaagctgatatttaaaaatttggttggacttcttttgtgacccagcacatggtgagtttttaaaaccgttttatggacacttataaagaatgcacatcatttgttggttaggtgtgggttctctccatctattttataaaggtaattaattctattactcaattctatatgtttactttttaaaaaaatctatcagtttctgaaacctaattcctaccattgtgcatgcctcaatttcttgtaaatctgcctgtttcactttgaatattttgagtccatatacaagcccatgattttactatcttcttcgcaggttgtttcttttatcaaattgtagtgacatttaatatcctatggatcacctgaaatttcagtttttcttagatgtctgcattttagccttagcttgatatgttttttcatcaccttattttcaactttttatttgtttttattttagatgtgcatctttattttgtattttattttttgagacagagtcttgctgtttcacccaggctggagtgtagcagcgtgatcttggctcactgcaacctctgcctcccaggttcaagtgattcttatgcctcagcctcccaagtagctgggattacaggcatatgccaccatgcctggctgatttttatatttttagtagagatgaggtttcgccatgttggccagggtggtctcgaactcttgggttcaagtgatccgcccgtctcgccctcccgaagtgctgggattacaggtgtgagccaccagccccagcctagatgtttatcttttaagaagcatatagctagattttattttgttattcagtttaataatacttcttttaataggtacttattttaaaggatattgtgattatagatatattcagatattatcttagtcattggggcagttatagtaaaaattataaactggatgacttataaataacaaaaagttattgctcatagtttggaggctggcaggtccaagaccaaggcactggcagatgcggtgtctggtgaggtctccctctttgattcatagaccgtgccttctagctgcgacctcacatggtggaaaggggaaggcaacctccctgtagcctcttttaaaagggcattaatcgcattcacggggtctccatcctcttggcctaaccacctcccaaaagccctaccttttagtaatatcacatggggagttagaatttcactatatgaattttggggggacacaaacatttatgccacagcagatatctttctaccaccttatttggtgatttctgggttttgtttgtttgtttaagacagagtctcgctctgtcggccaggctggagtgcagtggcaccatctcggctcaatgcaaccttcgcctccccggttcaagcgattctcctgcctcagcctcccaagtagctgggattacggacgtgtgccaccacgcctggctaatttttgtatttttagtagagactgggtttcaccattttggccaggctggtcccgaactgctgagttcaggtgatccacccgcctcggcctcccaaagttctgggattacaggcgtgagccaccatgtccggctggtgatttctgtttaaaagttttttcttaaagtgttttttcccacctagtttttcattgaatgggtaaaacattctacatttgcttttattaaaacaagaaatgaattttgctgcatttcaatttatagattttactatcctacctcgtgccaggttctgtgctaagtgctgtatatatctgtgatcacatttaacttttataacaagccaaatgagcaggaactcttatctctatcttacagacgaagaatccaaagaccagggacagtaagtaatttgctcacctggtttgccagcctccatgacacatcgccgtccagttctgcctttaattaccaaagcacaacacgctgctttgattcccctctcctcggcgccagaattcaagagtgaagttaaaccgcaagggctgagttagaagattggcctcagttccctgttcccaccagcaggtggcaccgtctcctagcggaattcttacttgaacgttttgcttccatttctgcagaggcatggtgaacacagttacaccaccaaagtgttcctcctggctgagtttgcctatcttgttcagtgaagacaacccatgaggacaaatggtgttaatgagaagcttttgcggagttacagagatcctcgtatttctttaaaatacacctaataacgttaactctgcaataatttgtagatcatgttaaatcttagctatcttcctcttgccacccagtgtgcttcaagccacatggttcagagcaccatttaatgtgaaactccaattttaaaacaaagtgaaccttccttttacaaaaccatgagacaagttacagagtaatgaccacccacatgaccttgaagtgattttgagtgagtgagtgtaacttccgtggctgccatttaaattggattcaaatccaaatggctccacctccatgtcatcagacctcttgtgccctgattcccttggctaagttcacagtaccttccacatcaggttgtggcaatgattacctgaggttaatacgataaaagcacatggtaagcactcctaaatgatagccaatataaagactcagttctcccaattccaagggtccccaccatgatagaaaaggatcttttggtaaatagagtatgtttagctcttgctaggtctttaaatactttgctgggggccaggcaccatggctcacacctgtaatcccaccgccttaggagactgaggctggaggatcctttgcggccaagagtttgagaccagcctgggcaacacagcaagaccctatttctacaaaaataaaaataaaaattaaccaggctttgtacacacttgtagtcccattacttgggaggctgaggcaggaggatccctcaagcccaagagttcaaagctgtagtgagctatgattgcgccactgcactccagcctgggtgacagagtaagactctgtttcaaaacaacaacaacaaacaaaaacctcaaaacctctttgttggacttaacttccagctcctccatgtagtaccttagtacccttgcagcccgtttctcttttacaagacaacaatgttgttataaactcatttggatgtggtcccgtggaggagtatttaccagaatctagcttatttagcgtcttcagaacacggcacttgcctggaattatactgaccccctcaacccataccaaccacccagagatggctgttcttggctcctctccctggggccctgtccttcccacatcgtcttcttcttctttcttcttctttcttcttccttcttcttccttcttcttccttcttcttccttcttcttccttcttcctcttcctcttcctcttcttctttcttctttcattgagacagagtctcactctgtcacccagcctggagtgccgtggtatgatttcagctcactgtaacttctgccttgtggattcaagtgattctcctgcctcagcctccagagtagcagggactacaggtgtgtgccaccacacctggctaatttttacatttttaagtagagacggggtttcaccatgttggcaaggctggtcttgaactcctgatctcaggtgatctgcccgcctcagcctcccaaagtgctgggattacaggcgtgagccaccccacccagcccttcccacgtattctggcagggaatgctgttgtcccccaagcctaccctaagaggaagacttcttctggggaaagatgttcactgtacccaggccctgccctggctggagctggcaggaagggtcccagagcaggaacttgtgccactctgcccaaagccagagtccctgaggcacacaccccatcaggcaccaaggtgaattccaactgccagttagtatttaactttccacatacgattagattaaacatgtgggttcataaaagcataggattgcagactgcagttgcaagggcttagatggttgtaaggtgaaggtgcccagcaggctgaggcttgtgtgcaacccagaagagagctcgctaacgccagcaagaaggttcagaacagcctggctttggaaaggaatttcatcctgcccacacactgcataggtaagtcttagcacacattctttattttttgaggaattaagtaacaaagttatctatgtgccttttccagaaaatgataaaaggaatgattttcctggtacatggcctggctcctcatccactcttccttctttccttcttgtgttttccttactcatttctttgttaattgccttagaatgaaaattttgagagtttttaaaatggaggattcatggtaaacgtaggtaatcatattgttttctcttcttgatataaaaatgaaagactttgctgccttttataggcccaggtgatgtgagcgatctaccatgtttcaagaaaagaaaactttggggctgggcgcggtggctcacgcctgtaatcccagcactttgagaggctgaggcaggcggatcacctgaggtcaggagatcgagaccagcctggccaacatagtgaaaccccatctctactaaaaatacaaaaaaaattagcctggcgtggtggctggcgcctgtagtcccagctactcaggaagctgaggcaggagaacggcatgaacccgggaggtggagcttgcagtgagccgagatcacgccactgcactccagcctgggcgacagagtgagactccatctcaaaaaaaaaaaaaagaaagaaaagaaaactctggactttggggtcaaatgagtgttactttcctaatagtgtcctgattgctgttgtcatgaataacacacattcatgacaggaatggctggaattaggggatcattctgtagcctggagacagggcacaactaatgacatgtgtaagctcaaatcatggtcttgatcttatgtcttgtacccagttgagccaactggtcacagcaatgaaaacagtgagttattggaatgtgtgacctctgctaggacagtcagtgctggacactggcttgggtgatgtgagttctagtccaggcactgtggccaacttgagaggcttgtgatcttggacaggtgacttaagccctctaggctatagttattccacctatcagagagcaaaccagcctaaatgatctccaggggcccagcctgtgctaggactcagcaagaagcattcactggaaatgtaggtcctcctaggttgatacacatgaattgcccatatttgaccatttctaacctatataaatggctatttcatataattccagagaacataaatggtagttgtcttagcattactaaagtaaatgcctattatgatattctacttaggggtaggataagtatgtataccaaatatggtttgtttcgatttgatttttgagacagggtctcactgtcactgctgagtgcagtggtgtgatcatggcttactgcagccttgacctcccaggttcaagctatcctcccacctcagcctcctgagtagctgggactataggagtgtgccatcacatccagctatttttttatgttttgtagaggtggtgtctcgctatgttgtccaggctgatctcgaactcctgggttcaagcgatcctcccacctcggcctcccgaagtgctgggattacaggtgtgaaccactgtgcctggcctccaaatatggttgatgtctatcagtcagttaaacagtaattctgggaataaaaaattgaaatcaacccacttataattggaatgtcttagcataatgtccttcaacgaagctgctttcacacactgtgatttgtttttttcctgtggtcatggagcaggcatgggccactcggccacatctcatgcatccgtattcaaaagccaaatcccttttggatcctgtttatttggcctggccacgggtgagcacttagacatttaatccctataggccctttcatccctgtgattaagtcttatcaaaaagcacctcctgaccggcttagcagtggggcctttgttcacattagaagggttgaacaaataatgggcagttggggctgcttagctctaaaaggctggtgaacgctgccatgcctgcacctggaaacaaacccaaatgactccagtggaattcagcactgaagtccctcatctcaaagaccttttgtggcagagactcttggatgggccttaggggtcccaggagtcccctgaaattgaatgtagagcttcctacgtgcataggtatactttcttggggaaaaattaagtcacatcattttattttacttttcgagggatctttaacaccgcccctcccccacccccaattcccacaccccttaagaataaattaagaatcactgttctggtagtttccagttgaattccacagaggaactgtcattcattcacaccttcattcaacagatttttagtaaagatttgctacgtacccatcgctgtgtagggtcccgggattcagagatgagtaaagcaatccctgccttccgggggctcaagctctcctgtcatcgggactcagttactgaatctcactaaacatcctgaaggtaggagtttatagagtggttttgaggatcacatgaataagcacacaatacatgggtaattcaaaaacgaaaacaaggccgggcacggtggctcacgcctgtaatcccagcactttgggaggccaaggtgggcggatcacgaggtcaggagatcgagaccatcctggctaacatggtgaaaccccgtctgtactaaaaatacaaaaaattagccgggcttcgtggcgggtgcctgtagacccagctactcaggaggctgaggcaggagaatggcgtaaacccgggaggcagagcttgcagtaagccgagatcgcgccactgcactccagcctgggcaaaagagactccatctcaaaaaaaaaaaaaaaaaaaaaaaaaaaggaaaaaaacaaaactaacatggtcatttgcagaaggggcagaaaaagggtctctgcctagacctggggaggtcagggaaagtactatggattggtaacaaccggctgggcttcctacaagagaaaaagactatactcacagagccagaccccatctcaaaaaaaaaaaaaaaaaaaagtctggcatggtggctcaaacctgtaatcccagcaccttgggaggctgaagcagggggatcacttgagcctaggagtttaacaacatagtgagacctcatcactacttttttattttaaaaaagagttaataaaaaataaaatgaaaataaaagggtaaaagagccagtggcaaagtcttgagtggattaaagccagctcagctaactttcacagcagactatatcattttaaaggggaaaaagcacatctctgttacattgcttaggaaatatgcttggtatataccctggggcaatcttatctatttgttaagtttccttccaacccactagcctgtgtggccaggagagggagacaaagatcttagagctctctaaataatagaacttaaaacatcagacagagaagagtatattatcttggtgatggtaattctcaatgaggaaaatcctggggagggatgttctgtgggagaatgcctgcaagtttatttgtttagtaggtttgattattcagctgattgaaattcctttcccagatggggagatctgattctcttttcatgaaggaaagaaaagtcacatgctaaagagacgggcatgtctttagaacggcagcaggcaaacccactgctgggatcctggggcttttactagtggctagtcacaggtttacctcctgcctgtgctccttctagctgtgttgaaacccacttgccccatctatgaaccgtgttcagctccattttctgagcccccttatctttttgtccatacctgttgcaactctttgcacgttgcattgtcattgatttggtctctcccattcaactgagcctctcacagagttcctgtcacctctgcagtttcatcgcctagcatagtacctggcactttaattcatgcatcaaatgtccattgagtgccttctatgtgttagacatctgctataccgagctagacaaagttggcagacatgacagccgagtggaaaagatgagcccctaaaccaataatcacacacacacacacacacacacacacacacaatatatatatatatatatatatatatatatatgtatgttatatatatgtatgttatatatatgtatgtatgtatgtattaaaaaatcttggccgggagcggtggctcacacctgtaatctcagcactttgggaggccgaggcaggtggatcacgaggtcacgagatcgagaccatcctggctaacatggcgaaaccccgtctctactaaaaatacaaaacattagccaggcgtagtggcggttgcctgtagtcccagctacttgggaggctgagacaggagaatcacttgaacctgggaggcagaggttgcagtgagccaagatcgcgccactgcactccagcctgggtgacagagcgagactccgtctcaaaaaaacaaaaaatttttgccttgcaatcgtttgccttgatgttatgtctaaagccccacaattctctaaaaacagagatgtataaaaaagcacacgtatataattctctgaaaacagaatataaatgagtcattgctccatttaactgacatttgttgagtgcttgttataaatatggcattattctagctggtgtgaggttaccaattttttttaaacaaaagtaatatgaatatatacacacacatttagtgactgcatatgtgatgtgtgcttttgaagaaaaaggagatgctgttggaggaaaatggtggtggtggtgggaagtgatttagagtagaaccagggaagtctcagaagtgacaactagctggaacctaaagaacgaggaggtagcaggtggaagagaaaggcaaaggcattctaggttgagagaataggatgtgataatgtcccgaggaaagagagcttactgacagggagggaagatgtcaggtgtgaccgaactgtagtgagcaaagggtaactgaggaggtggtcaggagcctgctcagccaatggggtaaatactgttaaggaattaggacttgattttaagaacaaccatcgcatcattttaaaagcaaacaaattgcactataatttccctcttcaaaaaggcacattggctgtgcacggtggctgacacatgtaatcccagcactttgggaggctgaggcgggtggatcacctgaagtcaggagttcgagaccagcctggccaatgtgttgaaaccccgtctctaccaaaaatacaaaagttagccaggcgtggtgacatgtgcctgtaatcccagctacttgggaggctgaggcatgagaattgcttgaactggggaggcggaggtttcagtgagcagagatcgtgccaccccactccagcctggacgacagagcaagattccgtcttaagaaaaaaaaaagggcacattgatggctattcaaggcagagaggggcacatataaccccaaagagatggctctggggagggttgtgttgtattacattgttggcattgtattatccaggtgagagatgctggaggctgggcggtgccagtggtgatgaaaaggagagatggatttgaaacataggaataatctctcagattgtttcttggcatcacttacctaaaatgcttctttcaaatatagatgtacacacccctccctttaggatacttgggacaatgtgccacttagacataggggatggaacaaattggagagtctgtcaatgccccctgcaatcttttctcttgatgttatctcataatgccccacaattctctaaaaacagagaacataaatgagtcattggtccattccactgacgtttgttgagtgcttgttatgaatgtggcattattctagctcttgtaaggttaccaattttttaaaaaacaaaagtaatgcaagactgctgatgaaaatttggaatatgagaaaagcataaagaagaaaatacatatctttaagcacaccacccactgttaacattctgatctatgtacttctaatattttctccattttcatatgtacacatacatttatttacatgcatatataaatatcaaagtgtatatatataattttctctgccatttaaatttttactgtgtaacaatcatggattgtaaaaaaagtgaataaaatgtacgcagtcagtttaaagactaacaaaatatgcattaaatcaccagccaggttaagaagaaatactattacttataccctggcatctccctcccacctttacatagccaaatccagaaaagatccgttttcctaaccttgttcgcctattttattatttaaattgcagcaggagggaagcatgtctactttatccaatttcacacagacgctggaagacgtcttccgaaggatttttattacttatatggacaattggcgccagaacacaacagctgagcaagaggccctccaagccaaagttgatgctgagaacttctactatgtcatcctgtacctcatggtgatgattggaatgttctctttcatcatcgtggccatcctggtgagcactgtgaaatccaagagacgggaacactccaatgacccctaccaccagtacattgtagaggactggcaggaaaagtacaagagccaaatcttgaatctagaagaatcgaaggccaccatccatgagaacattggtgcggctgggttcaaaatgtccccctgataagggagaaaggcaccaagctaacatctgacgtccagacatgaagagatgccagtgccacgaggcaaatccaaattgtctttgcttagaagaaagtgagttccttgctctctgttgagaattttcatggagattatgtggttggccaataaagatagatgacatttcaatctcagtgatttatgcttgcttgttggagcaatattttgtgctgaagacctcttttactttccgggcaagtgaatgtcattttaatcaatatcaatgatgaaaataaagccaaatttgaagtaaagtgtctgggcagtggctgtggggatagaaaggagagatttacaaatcattgaatcttctttctcatgaaacatcatttgtgtgtgacaaattcaatttataaataacccagatgtattatgtagaagctgaggctcaaaagctatcacttgcttaccagacggacataggagcatttatctgtaatattaattcatgagtgtggagtctgaagagatgaataaacaaaccataagattactttacatttattgttttcctggcctttaacctatttagaagtcttaagacagaacaaacatttttctttttctttttctttttcttttgagacatggtctctctctgtcacccagcctggagtgcagtggtgcaatctcagctcactgcagcctcaacctcccgggctcaagtgatcctcccacctcagcctccctagtagctgggactacaggcacgtgtgccaccacacccagctaacttttgtatttttttgtaaaaacagggtctcactatgttgcccaggctggtctcgaacctgaacaaacatttcaaaggacaaataatccataccagagaagtagagtatttaagaagtacccagtataacaaaacatattttaaaactaacatttaaagttttgcagaaaactaatcttaaaaagttctcattatttaagaaaaaaaaataaaaagttataatgtcgctttaaaaatgtattcttttaacttgatttagttttcctctatttataattagttgttagcatttatgtttaagaaactaaaggatacagaaagggtctaaattgctgatgccctctgaagacctagacaggaactacttaatatcttgcaccatgtggtgcaggatatcatagaatgtcagggctgatcattctactgttggcagagaccacttcacttacagatgagagaagggcagtccactgagaggagacaatttcattcactaattcggtcaggcaacattgacctacttggtccactggcctagaccccaagagtataaagatgagcaaggccgggcacagtggctcacacctgtaatcctagcactttgagaggctgaggtgggcagatcacctgaggtcaggagttcaagaccagcctggccaacatggtgaaactccatctctactaaaaatataaaaattaaccgcgtgtggtggcaggagcctgtaatcccagctactggggagactgaggcatgagaatcacttgaacccgggagggggagattgcagtgagccgagattgcatcattgcactccagcctgagtgacagatgctaaacatcatagtacaatgtgacaaggtcctaacagagatcaatgcaaaggggacacagccagccagcacaaggacaggagggcatgcctaatgcaggtcaaggtcttctctctcagagcagctgagagtagcaggtcaatggcagcagagagatgtggggcctcagcatcccatggcttcatgcctcctagtttaccctgttctcctccccatgccccagccaaggcacagcaacgatgggcaaggcctcaagcctcagggtgctaggacaaaatttagaaaaagaggctcttcttcagagaatgcttgtagaactcgttattccaatcacaaggtttgtctctttaaaattacagagtgagatatgtacaaggtatctacttcctaataacagatttgcaattatgccaactgaagcattcagtacagttagagaaaaccatccatattccaagagcagatgtaggaagagtggcttccctcctcagatcagaaacccagaaatgttgtcccacccagaaacatccatctcagagaggccagagcagccatcaggctttaaatcccagccctctgctctgcatccagacagaaatccgaggtttccatcaggtgacaaagaccctctccttaaccaaactgtcaagctcctctgagccctcttcttgactagagcccaaccatggccctataaaaactgcagactctcagcacacatgatttcgcccacctttgcacactaagagacataaacgctagcataggttctaagagctgaaagctaaagcgcctgcccgagaaaagtgaatgcggcctgaagaatttactaattgttccaaccaaaacctggtgacaggcagatagtcccctgatccctctcttaaggcagttactttagaaagtttgcaattataaatcctttctctctcccttgagatgtatatcttctaccattcagaactgtattgtctctctgaaatgcaaacattcaaactctccttgctggatgggtgccttgctctaacttactgctccccatcacagacagaagtttgtttctactctagataggagccaattaacaaacccagatcacactgaccaaccccttcccactttctatgcatttccacttcctggactctgctcaagccccatccccactcagttacctttgcacaaagggaagttgagctgggcctcttccctctggcaatagctaatgatttcagtcaatccttactgctttaactggctttctttacctttgacacaggtaaacacatggagagcaaaatcgaggtttttctggccgggtgcagtagctcatgcctgtaatcccagcactttgggaggccaaggtgggaggatcacttgagctcaggagtttgagaccagcctggccaacatgatgaaaccccatctctactaaaaatacaaaaattagctgggtgtggtggtgggtgcctgtaatcccagctacttgggaggctgaggcaggagaattgcttgaacctgggaggcagaggttgcagtgaaccgagattgcatcactgcactctagccttggcgactgagtgagactccatctcaaaaaaaaaaaaaaaaaaaatcgaggttttcctaattaagtacattttattatcatcactgaaagtacaggtggtaacatagagggttatcagccaacttcacttttggggaatgggagaaatgctgactctctccaagcatgttgggtgtctagtggttgaagccatttgccaagattgtcaccctaggatccactcccaccaaacctgggcttttcactttcaacccagcaactgaaaatgccagttcaaacaacttgctgtttttttctaccccacttgcttttagagttccttctgcctgttttattggctccatataacctgaataccacttatttcttaaagcatagctcagatgctattttaaaaggagcccaggatagtggctgatttgatagaatctatacccagattacccagggtcaagtcccagctctggtacctgcggcctttaaaaccacgaaaaaattactttaatctctgtgtctccatttcctcatttgtgaaatggttatcattatagcacgtcccttacagccttgttatgagacttaggcaatagccactagtgcttagaacaaagctatttttgtaactttctccaggaacacttcccttaacagaaccaacccctccacccctcagtttgttcttccctccacaccctctaacattctaacataaccacaaagagtcctgatgggatttggagttacactgcctgggttcgaatctcaattccgccactgccattcgcgcgttttctattgccagccaccttactctcccctggcctcagtttcctcatccttagaaggggagcagagcacatggtggtaactccctgcacattcctctctttccttgtattgggtgcccagtttgtgccctcacatggcgctagcactgagtgggctcaaactctgtacgcgtttactaagcatattgactgaagaaatctggaaacctagtaccgcggcaccatatcgttaccccaaaggaaaatgcatgcacgctgtcagagatgacgaacactgcgtctggaaacttcttaagggcacccacgtgtcctcagctgcagacagcagcgaggagacacccagggaattcgagacagcggaaggcggaagggtcccgcaacaacccaccctccagctcaggtgagttcagagtgagaacgcaccgccaggcttggacaaaggcacccggcctacaccccagcggctccccgccggggcctacgtggacttcagcctccagccacagggacaagagctgctggccagggctgcccgcctgggctcactgcgcctgcgcagtgagcagcgcgccccaggtcttctgcccgggcccactgcgcctgcgcacggagtagtgcactctcgtcggcggcaccggcccactgcgcctgagcacgtagcggtgcatttcgggacctgtagttttccccggcaggacggtagaagtcgtggtttgtgcgcggccaggcgctggagcctccgctgccgggagcagtaagtgtgtgacgtcggggtagaagggagtgacccaaattccaaaagctctttgggatgctgcgatgtcgcggccggccccgcgctcgggttttccctcctagacaaaagtctgccggctcccggtcgcgccgggtcggggatccggaaggtgaaggccgccaggccccacctgcggggcgcccctgctggacctggccgtcgggcgccgtcaacccgttgagcagcgtgttccggctggcacgtggcccgggcggggcccaggattggttcaagcctacggtgttggtccccggagagtctagggagacaagcaatcccctggaatggtgggggaagcgatgacagcccctggtcctcatccgcagctctgggggaagtcggggggtggggagggcgggtgttgctccctgagtgttgggggaagggtatggggagaggaccctgaactagcccccaggttacccaggaggagctgaggcccagagaggttcagcgactcgcccagggttgcacagcgagcacaggcaccgacgtcgccctccgaggcctgggcttccagcagggagagacccggacacctgtcatcgcttctcggtggatccctgaaatgttgagttgtggagtctgggcagctgagatcgggcagggctggtttcttgtaggcccaggcttccgtgtagagggccaagtgatgcccaaggttcacctggcagccccctctctggacctacccctccttatgattgggtgaagggttgggtgaaaagggtagaggccgggaatgagaacagcttcagaaagctcagacaaagggcgcagcatgattcgtggctggaaggagacagcaagcgatagactgatccttgaatttgttagtgtgccaagaagaaaaagtattaatagattgtggacgacacattatccatattgctttagttggtctaaccaaaataagcgaatagcttttttgtttctaagagaaacctgacaaaggaagacagggtatttttgcggtgaaggaaatagaaatatttggagttgtatctaagccacttgttacttttgtgttttaagctaagatcatggataggtccagggaaagttaaaaatttcccgcacttcttagattttatgcccctcaaaaacatccccaccttgttggcttttgcagttcaaccttcagataccagctccctcgtttctaatattgcattaggtgtacatatggcagcagagcaaatagcttactgatacttttagcttttttcttctcatttgcaaaagactctatgaaaatggctggcttgggcatgtaattgaagggaggtggggaaaagtggtaattcagaggcagtgggtgggaatgagcaaagcatgtcagtgtggttcaccctcttgactcccacctcaccacagcccttctgctacaattgcagattgactctaaatatgtttctttcctaaagagcttgaattttatcactccaggtatgaagttgaggcagctgccagtatattttggcagtcaggttctgtgatatcagagaggatggtgaattgtgaattccagagttgcagaattgttctttagattctgattttttaaatgacagcactttggtttggagggttaatgacttaccccaggtcatatgccacaccatgggtaacaccaagagtagagctcagatctccagttgtcctggtcctcaggctactgatctttattccctgccctgctatcttggaatgactgcattttgccctggatgtcgctagcctgtatccttcaggttggcatgtccagtcatggaaggagagagatttaacataacaacaatggctgacattcagtgctttctgtgtgctggccatggtgcttagtgtttacagtctaatctgcaagagaatttacagatgggcaagttgaggctgagagaggccaagtaacttgtccaaggtcacactgctagtgaacatagcacatgttttccagagggccagaccactggatgttttttcattcatttcctcctgtaggatcgttggacctatttctcgttcttgactttgggaatcaatattctacgtacatcaattcactgggtatgcagttttgcctctgaaaattttgagggaacagccagactcatctactgtatttgtatacaactcaattaaagcaggaattgtaaaaataaaatttgtaagatctttaacattttaatatacaacattagctaatcactaagattactagagatactcaaagtgaaaattgtagcaacaggttataacatgttaggagcatatttctttagggcagtcagaatctgctgcttcttaaagacaagtgggccatttacacatgaaggtaacaagcacattcagccaccatcattatagttaaacagatctatgatttaaattcctatcgctaccttatctgactttgaaaaagtcatggggaaaacttggctaccttgtgccaactgctagcttgttttcaagatattataatcttgaatagatggaggatgaactttttatacttagatagctttgtaattgaaagtttgtataaaaacatcttgcctgaagttcatcttatccccattctatctaaaggcctttgaaattttcagccacttttcttaattatgacggtaagtacatttcaagagaagtgtttccctgacttttgaatgcaaagctctctgcctgtgtcaggatggtgccgaggttaaagccctggagccagactggatgggttcgaatcccaggcccacctgcgagaccctcgatgtgttacttaaattttatttcctcctctctaaagtggaggcagtaagctgttttatggggtagttgtgagggctaaatgtcttaactcatctaagcactttagccacttttttccatctgacacaaaaaagttaagctatgattattgtgctataaagcattggatttcagaagaagtaggggcactaaacaccatctgcttgacacctttcttcacttacagatgggactgaagctctagagggaagtcacttaccagagggtgtaggttcttcatccagagctgaagtcttcttgggggtatgtgtcatattctaagagtagggacctacaaggccttggagcgaatcccaaggctcgggctgccagccctgccttctcatttccatatgccatggtgtggcatatgacctggggtaatatcctctgaaccaaagtgctgtcatttaaaaaatcagaatctaaagaacaattctgcaactctggaattcacaattcaccatcctctctgatatcacttccttccctaccttctactaggtctccctcaagctttagagaaaattctgcctctgaattattgcatctgacaatttttctgccctgtcacttattcccttgctcccagataatcttcgaaaaaccaagatgagtttaattaacactcagaggacttgacaaagacactcactcccaaaccagcttgcgtttaggtctggaggcaggtggagggacagattttagacttgggcccttaggttcacagatgaatgggatgggagcccatgtcccctcagaagcggcgctgtgctgctggcggctacagacagctggtgaggagagctttctgcttagcaagggccaggcccgtctgggccctcgcccagcccatccacttcccaccagtctctcaatcgccttgtcaggacacagcccacctctctgtggagctcactttctgttcacattccctctctccatcaaagagacatctttctaaggtggtctgccctaggaactccaaattgacctgcttctttcttcctgcccagatcagaccttccagctgcctctcatgtacttgtctgttgggggcttgtgttgatcaggagtgaattcacagtctaccatgaattggaaggtgagtatcgttttaaatatttatggcttggggttttttcttcctcctgattgtgaaaattcagaataacagttctacaccagtagcttgattaaaaagaaaaagtaggtgaaagcataatatttatgtttcattttaagttaaaacataaatgtacatttattcgctggacttctggaagaaggccaggccttttcttcgagtgtgcattcactaacttcaaatcttcctcacttttcttacaaaaactatacctttaaagctttacctgcaattttgcattgtgctttctttttttacacattttttttgtagagatagggctccactatgttgcccaggctggtcttgaactcctgggctcaagcagtcctcctgcctgagcctcccaaagtgtcgggattacaagaatgaaccactgtgctcaggccgttttggttcctttaaagtagatgcagtggactgaatgtttgtgtttccccaagttcatatgttgcaaccgtagtgcccagtgtgatgatatttggagttggagcttgtaagaggtaattaggtgatgaggctggagccctaatgtttggaatagtgagcttattaaaagggctccagggagctctcttaccctctttctgccatgttgggggtacaacaagaagccagccgtcagcagcctggaagaggactccgctagaacccccctgtactgcgccctgatcctcagctttcagcctttcgaactgtgacaagtacctctttaataagtcacccagtctgtggtcctttgttctaggagctgaattgactaagacagtggattaagatcttatgagcagtgcatacacaaaatctttccagtgtttcatactctttcctaatcttttacagttgacttgccaacagcattttttttccaacgcaaacttgagtctttcaaagtattcaacttagttttcataaaaacttttgctttacacagtcatatttcacaagcgtaatgtttaaataagttatggaacatagtatcaagtacaacttaaataaactgcttggcgagtaaacacacctgacccctgtgaaacattagattcagctggtgggagcagaagttcaagggcagccagagagtaggtcagcaatcaggttccaccgagggaaaggagaatgtcatcttaagtcccggaagtcaataaggtgaggtggaggttgtttaagagagcagccactaaaatatattatagtcactttgcaaagtctaatatcaagcaaaaatcatacattgtctcaccatctagaaatggctactattaacaatctcggtatattcatctttttctgtatatatgtgtggcgtgttttcatgcataggatcttattttacgtgttttttcaattattataagcattttcttcaaaacatccagtggttttctattgcaaggagaaactggaaaggtctggaagcaggatcagggagccaggaaggtagctttcccatcttccccagctgtgtggggtgaggggctcggcaggccctgcaggagggctgagggcccaggaacttgtgtaagttaaagatggcagagtgagtgagctgtgaagggtgggaagatatagaacagtttgtttagcatgccttgaggatcagagctcacagtggaaaggttgggaaggaaagaagaggcagtgagaggatggagaggggaaggagcggaaagcagtgtggggtgaggtttagggaggtcattcgcccactgcagtggctaagtcagtagaggagagaggcagtaactggtactaagggccagggttcaaagcattaaacctcatgcctcaaggtggtgtttctcactcctgagcactagttaaggcaaggatttcgagccccactcccagagtttctgattgggtagatctggctggggcctgagaatttgcacttcttataagttccaggcggtgctggtgcacactggtcgaaggcaatgcgtgggaagttttcctctttaattgtagagtgacaccaacccatgtgaccactcgggccagtcttgcttgtgacagttttttctgccatcaggaacaaagtctgcccaaaccttcccagcttctgcaccagggaagtggctaccagggagcagcttcgtgtttaaacacagccccatctcgtgtagtgttagaaaggaatggccgcaggccgggcgtggtggctcatgcctgtaatcccagcactttgggaggccgaggcaggcagatcactttgagctcaggagtttgagaccagcctggacaatgtggcgaaaccccgtctctacaaaaaaatacaaagattagctgggcatggagatgcgtacgtgtagtcccagctactcgggaagctgaggctggagaattgcttgagcctgggaagtggaggttgcagtgagccgagatcatgcccctgcactccagcctgggcgacagagtgagaccctgtctcaaaagaaaaaaaaaagaaagacagtcatggccctgattgcagagagctgcagaaggtggaaggttcagtagcccccagtgcgtctggtggccttccccctctggctcagtgggccatggccggcagcgacagtcaacagtgctacctgtgcgttagcaacaagtatggcctcattatttaaaaacttagttattcccatttcacagatattggggttttgtttttaaaaattgatgtagatctaggccaggcatggtggctcacccctgtaatcctagcactttgggaggctgaggtgggcagatcacatgaacccaggagttcagcaccagcctgggcaacatagtgggaccccagctctacaaaaaatcagaaaaaattagctgggcgtggtgtcatgtgtctgtagtcccgtctactcgggaggctgaggtgggaggattgcttgagcctgggaggtcagggctgtgggaagccgtgatcatgccactgtactccagcctagttggagtctcaaaaaaatattcatatagatccagtccaccctggcagcattcattttcctccctgaaggtctgtatgtttcaagagatgtaaggggtttgttaaaaggaaattggaggaaggggttcataccactgaaggttagtgcctaagagaggggcaggaagggggccctggagctcttcgctttaccctgtgaatgttcttgacctctgctgcccttgtgctgcgtccttctcagtccacacttctgcctcttgccgtgcgtctccactgcctgtaaaacaaagtgaacactgaagcctcccactagggtccattggctgatgcgtttccatttccatgggttttctaacttctggatgagagagtacattcctgcaattgctaaagctaagtttcctatctggattgtagacagctatgggcagtaacatgggctttgttatattagtaatagggccccggccaggtgcagtggctcacacctgtaatcctagcactttgggaggccgaggtgggcggatcacgaggtcgggagttggagaccagccggccaacatggtgaaaccctgtctctactaaaaatacaaaaaattagctgggcatgatgccgcatgcctgtaatcccagctacttgggaggctgaggcaggagaattgcttgaacccaggaggtggaggttgcagtgagctgagatggtgccattgcactccagcctgggtgacagagcaagactctgtctcgagaaaaataataataataatagggccccataggtttattcagagagactgagaaagctggaagagattagcttttcccagtgtgagtcattgcctcaggtagcctggaaaatcctagcaaacaaaaagaagtttatacaacaacattcttttcatagctggtttgtatgcatggcgtcaaaccttcacctctaaaatgtgaaccttcaagaaaacaggatttcagggtttactggagggaggggattagcctaggtctgagggaagaagaacctggaaatgaagtcagtgtttaaagctccttatatttaccagagtaaaaagttgaaaagtttcacttttggaactttttagtcttttgtactgatgccagagtgatctttctgaagtgtatgtgttgtgcttattctttactgttaaaacgatatcatggttgaaaactattagctaattactgagtgttcttgtgttcttactgttttagtaaaattaaaacgatttaagtatttgcgtccctgcctcctcccatgattttcattgtatttctatcatatcatgctatatccttctgcaaatatccatacataaccagttaaatgatttcagaggtagcgagtctagttgcctctggaaaattcagtagccaagccatagtgtatttgcatattgtaaatgtgaagtggatgggtgtgaggaatgaatcatatatagtacaggacagcgtgatgctacagagttgggctttggagcatttggagctgggtcagccctgcctcgctgactgctggcctccccgcctctgcattttctcggctccaccgcagtcagggcgagccatctgctcatggaggtggctaagggcagaagggaaagccgcataaggcactttgcatgccgtgagttcccagtctacagcagctgatgctacagcttctaagcgtgaaatccacatctagttctgagtcataaagagtttcgatacaatcataggaacattcatctacatacactgtgatttcatgaatttcagtttgttgaaaatcaggcatcagtggaagggagactggccccggggttagaatgtccttccaactggctcctcatgagggagctgtgggaccttcaacctctcaacctccaggagctcttctttccttgctcatataaccagggggttgagtaggtccccttgaaagttatttccagccccccggttctgtgagcatattgtacacactaactaggttcagatcaacttcggttagactattagagggaggtgcacatgtatcccccacagtggaatctcattggtatttcatataataagtgattgacagaaataaggatttcattgggataaaatcctacctggtcctctaaaataatgattgctcaaccagagcataccttttcactatttgggagggaatttttaatcacacaaaaagcacatacatatcattcaggtcatgctaaccatgtgtatggaactaatattgctttgaagtactatttgcaatatatagaatttcacacaaaaaacctactagtgcaaagagtgagtaattcaaatgcatagtgtttccctagactttattttagtgaatggggttgcaagagcagttagtaagtagccatgttttaatgttttgagttctgtcgtgttttattttaccagcaagtgccagtgctagtgagtctctagaaacaagagaaaatcccagagtactaaagctgcagtttccagaagcaaggtctgattcacccttactttgtagatgaaggggcaggagctcagaaagaaaggagctcattgcctgacccaggtcacacggtcagtcggtggtaaagtagagctctgacccaggcctccgggctccagctcctttcactggatctggctgctgcctcagaagcaagggcctgggtgatcagcaggttgcacggttgagctgtgagagaccagagtccccacgcctgtggatgaccggtggtcccctccatgaagccagccgcaagcaagcagcaaagagcagagctgtaacttgactgttggccccatggggatagagaccttttctgcttggtctccagtgtagctccagccccctatcctcgtgcctgttgcagaataggtgctaagtaaatatttgttgactgaaggatcataaaagaaacctcccatatcggtgatggaacatttagttagcatggcttctttcttcttgaaggttcttgagcacgtgcccctgctgctgtatatcttggcagcaaaaacattaattctctgcctgacatttgctggggtgaaaatgtatcaaagaaaaaggttggaggcaaaacaacaaaaactggaggctgaaaggaagaagcaatcagagaaaaaagataactgaaggtgagtccacagtacccaaccttgcaaatgggagctggccagtgggttggggtgaccaatcaatgaacaagagaggtctgagacctccctgtccgtcgggtctgaagggctgcgtgggggcatgtggcctcacctgttctctaaggtagaactgctccataaagggccaggtgtgcagatcctggtcctgggatgtgagtgctgctgagccaaggtgcacggagcattagttcatccttcttgaaacctgcggtggcaatggttcttgacaggtattgggttaagaattgaggactcagtgacagctgtggaccttcttcccagagaagcacacatactgaaaccatctgcatttgtgtgtggggagaagtttgcagatgtggggagtcacaggtcatttgaaattccaagattaagaaaccctgttacctttaagataaagtctgactccatggtatgacaaataaatcccttcccagtctggtcttaacccagacctcatttgccacatgtgcccactccccatccccctgcaggccattcctcagccaccatggcctttggccttgcttattaggttcttcccaaggacctcctcaccaggcctgagggtacactcatcgttgtcacccagtgcctatcacagtgcacactggtgggcttagaacacacttggggagttacaatacatcaaggcaccagaaagttcctgttaagcccctgttacagatttaacacatgacaattcacagttaccttgattccagtgtgttgatgcagtttcttcactttgcaggttgagttctgtactttaaaaatcaggggattcagccaggtgtggtgactcatgcctgtaatcccagtgctttgggaggcaaaggtgggaggatcacttgagccaatgagttcaagaccagcctgggcaacatagtgaaaccctgtctctacaaaaattaaaaaaaaaaaacaaaaaaaaccaaaaaaaaaaaccaaaaaaaaacttagctgcacacttactgggtgtgtagtcccaactactcaggagactgaggtggaggattgcttgaggcccagaagttcaaggctgcagtgaggcatgatcacaccactgcactccagcctgagtgacagagcgagaccctgtctaaaaaaaaaaaaggattcaaatatggtggcggtggggttctagatctgtggttcccaaacctagctaatgatcagaattgcccaggtgtttgtttaaatgaagattcccagcttcagcccagagagattccataatggctctggttaggtttgggtgttggtttttgttgttgtttttaagataggggatcctactctgtcatctaggctagagcggtgcagtggcacaatctcggctcactgcagcctcagcctcctgggctcaagcagtcctcccaactcagcctcccgagtaggtaggactacaggagcacgccaccacacccggctaggtttttaaaacaatttttagtagagaaggggtcttgctgtgttgcccaggctggtcttgtactcctgggctcaagcgatactttcgcctcagcctcctgaagtgctgaggttacaggtgtgagccactgtgcccagcctctgttcttttgaaaacccattagataatccttaggttgtcttctcagcaacaggtcattgtaggaaccactgggctaggtggcctcccaaacccagcccactctgagattccatgctgaattcccttgcagtgccgtcaggcttgttgaggctaaagacctactgatgaggaatagtagcaacacctactaagtggttattctgtgctggggactttgctaagcattcatgcacaactgtgttattcagccctgatgactctgtgaggcatgttcagattgaaagaatggctctctctacatggtgaagaacagagtcagaaattgatcccaggtcaaatgcattcgatcagcatggccaagcccaagctgtgctaccttcctgaatacaggcaagtgagctcgtagggatgcttcactctgttactcaccacttccggcagctgcccactcgctggtccccagtgaactgtaggcttttgctagatagaagaagttactttctttctttctttctttctttctttttttttttttttttaaggtgtttgattgactgaaatttaggagtaggcattacgatggggaggagagaaattaaaaagggtgagggaaggcagtttaaattaaaatgttgctgattgaatttatattcctgacaatcccattttgtgtgctaaactgatcaaaggaagaaaagatgagatggaagatcataaaggctttgttcctcccacaaacatcagcagagacctgcatttaagtcaggcctggatggcttagaagcaactcagggagttggtcttcctctctaggctggcagcttcttaaagactagagacttgcttcaaacaaaaagcgttttcaggccgggcgcggtggctcacgcctgtaatcccagcactttgggaggccgaggcgggtggatcacttgaggtcaggagttcaaggccagcctggccaacatggcgaaaccccatctctactaaaaatacaaaacttagctgggcgtggtggcacgtgccagtaatcccagttacttgggaggccgaggcacgagaatcacttgaacctgggaaacagaggttgcagtgagctgagattgtgccactgcactccaggctgggtgacagagtgagactgtttcaaaaaataaataaataaataaaaggggggtggggtgtttcagatggaagggaaactgatgctaaaaatacattggttaataaatagacttgagtgatagacttgagtggtgtccgcttgttaagtttaaatggctgagcatacgtctttatgctgagcagtaaacatcgggtatactcttatcaaacatttcctactcatccctttggtattcccttctagattctgccatgtaaatgtcagcttgagtggactccagctgagaagaaagagaagaaagacttaattattgaataatttgtcagaggataaactcccaacctagacctttcacttaaaatagtgtgaatttgtatatgtttttaaaagaaccagtactggccgggtatgctggcttttacctgaaatcccagcactttgggaggccgaggcgagtggatcgcctgagatcgggagtttgagaccagcctggccaacatggtaaaatcctgtctctactaaaaatataaaaattagccaggtgtagtggcgcgcgtctgtaatcccagctactcgggaggctgaggcaggagaattgcttgaatccgggaggtggaggttgcagtgagcctaggtcgtgccactgccctccagcctgggtgacagagcgactgcgtctccaaaaaaaaaaaggtaaaattaaaattaaaaaaaataataataaccagtattttgtttactaaaataaaatgcctttgtaaaaaaaggagtcgtggcctttggaataagtcaaattgtgtatctctttctctttctcttacacagcaccctctaccccgtgttgtaaagcggggggttttgtaaacttacacctcccccaccatctcaagctggggggtcccaggtgagaggcttccatagaggacaaggtggtgcagaaacatctgctgtgggagtggggtccccagcactgggtgcttcggccagctacccccgaccccaggccccctcataggctgccctcccataccctcctttctcgtcttttcctcctacaggtgctacacccctgtgagagtgttttggagtgttttcattgttagggtggagggaggctgtgtgtgtccaggaaaggtgactcctgtgttaaccatgagggtcctcgcagggaggaatcgttgggagccctagggtgtgttttgtcctctcctcacctgtttgctccttgggatttgctgatgagaaatgaagggtagggcaccctagtagccactggaaccaagggcagggaggatgggaagatgttttactcagcacctaacacacgcagatccctgtgacaagagctcatgctctcccacttcttcgcaagaccccagagtggatggggagtgaggtggcagcagctggcactggaagcagtgcggagtgtttggtctggttgctgatggctgcatgggaaacttgcaggagtgtgtgttagtaaacgtctccccgtcctggcccagtttgtgtcgaacatgcgttttccatgtggggagtcaggggagttccatcttaaattgcactgtgcttgctggatgctcttcaggacaatttaggaagcaggaaagaatttacaaagttctgaggacagacagaccctgctcctacaagctgcagtgctcaccatagtcaaagtggactttcacgtaagcccagacctcattctctctgaaggaggccgctccagcctttgccaggagccctggtgactttattctgcctaatcctgctgcggcctggggtcctgttagaacgtgaatggaagaccacagcagaggtgggatgcccttggatttctgccatccccacgctttcgtgacatgctcagatggggcctagaactgaccctgggccgtggcctaccatcctccctttgtcagggcctccttgcaccctggcaggttaccccacccaccctggcccatgttcctgccccaggggcctggcctctctgctgcccaccctgcaggtgtagggtatcacctgctcctgccttgcctggcatcagacctgctaccttggcaccacttcctccctcatgcccacccgcctgtgtgcctcataagtccaaggcgggggatctgctgaccagtagacactcatgtgctaaacacaagcgcttttctaggctttgggatttaaagctacactttggaatttgtggaagatctggccatcttggaaaattaggtagaaggtgacataaggactggactaaaccactgatcatcccaacagtgcccgtggcttttctgttttttgtttttgcttttgtttttttagagatgaggacgtgctgtgtccctcaggttggagtgcaatggtacaatcatagcttattgcagccttgaactcctgggctcaagcgatcttcccacctcagcctcccgagtagctgggactaggccctgctaatttatttatcttttgtttagagacaagggtcacgctgtactgcccaggctgggtgtaggttttttctgaagagcatttgggagttttgtttttgcttggttacttttcctatgcacccttctaccactagggggagatgattaatcactaattgaagggattttgttcgttttttatgttttgggtttttttgtttgtttgtttgtttgtttcaataaagaaagagtttaattgcagtaaggcaggccgcgcaggagatggcgttcttattcaaatcggtctctctgaaggctcagaggttaggggttttcaaggcggagttcttgctatcattccactccttaggtacatgaagttggtagatgtgtagtttgatgttaaattattgggtggatgcatgcaccgctggttgtagagactggtaaagcccactagcaggaccccacctggaccaaagcaatccctcaacccgctggaccatgaccgagaacaaacacaaaggacctgaaatgcgttgtgaaggccagaagccgacatccacattctccacccacggagagccccagagtccctcatgcacatcctgcttgatctattacacacattcacacattcgcaacacatttgtttggttttcaagcttacaacatattagaaacagaaaggaagaaaggctgtcagcagcagaaatacctttgagcaagagggacggtctttgagaagcagacttgagaactcaccgtgtgctcttcatgcgccagacactgcggcagccacagcgtcccacatgggatgccacacgtgatgatgttatgttcatggtgatgacctcaggcgtgaagaagaggttcagccgtttcacacagtctgtttaacaagcacatacataacacagacatacgtgaggaatctcagaaaccaaataattcaaacaaagagtctgggattctttcaaaagcgttgcctctgcccaagctttcttcaaattctgtctatagggaaacgtagctgtcaatgtctcattcccgaagacttccagatgcctggatctttagagttctccacctcaccccgagttgattacacaaatgttcctggggctttgcttagtgccctgctctgtgccaggccccacaggcagagatggcagggacccaggcctgacgttggagagctcctgacccactgccggaaacacacgcaccatcacaccatgagggagctcccccatgcagatctcatctgtgtcagagtgaagccagaggatggacggtggagagtctagaaggagaagagaaaggaggatgagttctcatacatgagcaagcaggagaggccatttaaaatgcacactctggcctggtgcagtggctcatgcctgtaatcctagtggaggccgagacaggaggatcacctgaggtcaggagtttgagaccagcctggccaacatggtgaaaccctgtctctactaaaagaaaaccaaaaattagctgggcgtggtggtgcatgcctgtaatcccagctcctccggaggctgaggcaggagaattgcttgaacccgggaggtggaggttgcagtgagcagacatcgcaccactgcactccagcctgggtaaaagagtgagactctgtgtcaaaaaaaaaaaaaaaaccaacctaaaaaataaaaataaaaataaaatgcagactcctgggctggattccaggtccactgcatcagaacctgcaggagaaggaccaggaatttgtgatacgaacatccccaggcaattcttgaccctccttttgagacctacactgtagaagatgggcagagggagaggcagcaggagcccaacctgggaaggagccatcgggaaaggtgggaggaggggcaggagacagcgcacgcgaggcagcaaatccttcagcccttactccccaagagctcacagctgcctccacagagggtaacggtatcattatccccttttcgcagataaggaaactgaggcagagaggccctgcctaaggtcccccagctggtgggaggcagagacaggaaccagccctcatggtctcactgtgagactggacttttcacagctgtgcggtcgagtctgagccaagtaaagtaaagcgagtttttgtacttcgaagtctgggacataaaatcttcaagacgtcagcatcagtgacacgatggtgacagaggccagcattgcttgttgtatctttttccatcctgttcctatctacacttccattcttctgttcattctgcttgcatttcccacccagatggctttcacggacacacacacacacacacacaccgcacacacacacatcactcacagacgcaccctgtgcccaatgtcaaaagacaaaactgcaacacgtttagtcatagacctcattgtcttttattcttgattcatgaatggggcagcctcccttctataaaacagagcaagagctcccaccggacaattgcagaacagtgggctttgtaaggtggggacaaggaaacagaacaatagaaaagaagctgatgggttaacatcaggttacttcaggacctcctaatcacgctgactcaggtagaccagaagctcctgttttcaggaaaaactaatctgtttggggacatacctgcttccttattaaagttttgggttgattatatggctcttagcatgactgactccattttggtttggtttgatctggtctgttggggcctagtgcaggatctcagtccaaaacaatagcctcccataatttttgtttaatgctgggtcagcggtaggcttggtccacttgcgcttctgcctgggtgggccttcctcttttcctccttttctctctgtggtgaaatccccattcttctttctgtcctccacttcagtttcacctcttcctgcaaccctgcccacagctcttcagcgccaggccctggactcagctctcactacgcaaacctccaacatctcactgagtgggaggtggcccccacctccacaccagacctggaccttgagggtccaggcttgttttccttggtactcctggccctgacgcacccagccaggcacacagaaggtgctccagtatttgatgagtgaatgactggtagcaccagaggaaagggagcagggagtatggccaagaccattagctgccttcctcagtgttccttctccccttctccctagtaatagaaccctgacttttacctggccgtatggtcactcaaaataaaggactacatttcccaccttctctcgcaaccaagcctggccaatcaggtttaagtggaagtgtagtgtgggacttcctggaaggatctttaaaagggacaggatgggcccctcttcctccctttctcctttttggctgcctggaatactaatgcaatcgcttgttccgcagccactttggactaagaggtgagtttgagacctgaaccaaatctaggacaatggagtaaaaagataggatcttgggctctcagtgaccatggatccatcatttcacccctgaactgacaccttgacacttcttatttttggtggtggtggtggggtagcttctattttgttggcatctctgtggtctattcatagttcacctgttttagctgacacatggagccagttagagatgggcgcaagggcttcctgatatgaagacttggattctggtcctgaccctcctgttactactgttacgtacgtggctaggtgagtcggtcaccaccctagcctggcctttgtaaaatgagctcaatgatcactgccctccctactgtacagtgtttgtctgaactgaatgaggtcaagcatgtaaagatggtttgcatgtggcaggacaatcacaaatggaaggagtgtatactacctcggtgggcaactcagccaccagttggccaggcaggggctccatccaacccaattggattgagccaatggccaacgaaccccatttgctaatttacctcttaggtccttgtaggggcagcaccatttcctaatgcacccccaccttgaaagccaccttgatccgtgggagggagaggggctgtgggtattagtgatggggaaggccagagaggctgggatgttccatcagccaaccactcagaaggagaatagtgccaccaacatcaggagctcacttctagtggacatgtctagagatatgtggctcagccctgcgttgctgcgtgtggtcacccgctcttaactaaatacagtgctctaaatatggctgctcccaaagagactgtgggctgtcctcaccagccatcctgtcccacccccaccagaagaaacctcttcttattattaattccctaaccgattgcagatattgcagatggtcttaaaggaaatgccagagaacaaagtttcctctgatcaaagtatcttaagatgtgtccaccctcattagatgccagcgataaaaaggaacaaaaatttttctcaaagagtgaaaaggaaatgagatcagtgatggcaggtctaattgtggacatgtttgcttcgaccttaccatggaccactcactcagtgggtgccagacacaatgcagtggcttggcgcaggttatgtgattttttttttttttttgagatggagtcttgccctgtcacccagtctggagtgcagtggcatgatctcggctcactgcaacctccgcctcccaggttcaagcgattcttctgcctcagcctcctgagtagctgggattacaggcatgtgccaccatgccaggctaatttttgtatttttagtagagatgggtttttgccatgttggccaggctggtctcgaactcccaacctcaaatgatccgcccaccttggcctcctaaagtgttgggattacaggcatgagccactgctcctggccaggttatgtgattttaaagtcccactgtattttattaaggagttgattgagcctcaaggaagtaaagcaacttatccagaacatatagctacgaagctagaaactcaagattccaactaggtccatccaattccaaaagcccggtgaactttctgttactttctatgggaagttttcaggtttagtataaagcaacatttcttaattatgaatacaatcttatgtaaccaaattgcacattaaagggtattccttttgatccttctttcctcttttctaataatttttacaaggcctcaaaggaatagaactctaaatctgttttattaaattgctttagttttcaaacaaaaatagctcctgttggtttatctttagctgataacctaaaaacattccttttttatcgctgtaaaataatgtgtctttaaaggatattctgtctcctctttaattttatgttgaaataaaattttttggtgtccaggggagttggccattttagcggctggtgtctcatctgaggtcagaggtgagtcttgcgacatagaaatgatgggggtgaaaaaggaaaagctcagcttcaataactaagctaagcacggtggtgaggagcatgggcttcagaatctcttggccccagtgccttgccagtgcctcagtttcctcatctgtaaaacaactgacaggattaagagaattaaattataatagctatgcgttagtggcatcatgaagaatggctctaagcatgaattctgggttgaatctctctgggcctcagttttccacatttatttatttatttatttatttatttatttattttgagagggagtcttgctctgtcacccaggctggagtgcagtggcacaatctcggctcactgcaacctccacctccaggattccagtgattctgctgcctcagccccctgagtagctgagattacaagtgcccgccatcacacctgtctaattttcgtattttagtagagatggggtttcaccatgttggttgggctggtctcgaaccccttacctcaaatgatccgcccacctcggccccccaaagttctgggattacaggagtgagccacggtgcccagcacgtgtagctgttggtgcctattattccagaaccgcagaggcatggaataaacgggatgtagggcaaaacccactcatctcccaaaactcaggccacccacacgtgggcctgcacaatgacagcacactgaagtgaccaaggaagtttgagtaccatggtttgctctcaaggaacttaaatcctaaaacgtacatgaaatcctgagggaaaaagacctcacaaatatactacacaataggagttatttcaccaggttaattgccaatgagaaatgcaatgtttctattggcagagcaagacttcctttagcacctgtggctggctgaatagtggccaccaaagatatcaaggtcctattccctggaacctatgaatgtcgctttagatggaaaaagttctgcctatgtgaccaagttaaggattttgagatgtgaagatggttttagattactcaggtgggccctgaatgcaatctcaggtgtccttgtaagagggaggcagagggagatttgacacaggatgagaaagtggtgtggccacaagcccaggaatgccggtggccaccagaagctggaagaggcaagaaacaggtcctccctggagcctctggagagagtgctgccctgccaacagcttgcctttggctgtcttctctgtcctgtcctgtcctatcctttttcttttctttctctctctttttttttgtttttttggagatggagtcttgctctgttgcccgggcaggaatgtgatggtgtgatctcggttcactacaacctctgcctcccaggttcaagtgattctccttcctcagccacccaagaggtggaattacaggcgcccaccaccatgcccagcaaatttttgtatttttagtagagatggagtttcaccatgttggccaggctggtctccaactcctgacctcaagtgctcctccgacctcaggctcccaaagtgctgggattacaggcatgagccactgagccaccgtgcctggcctctttcccttcccgtcccccttcctctttcccttccctttccctcccctgcccccttccccccgccccctccctctcccctgccccctccccctccctctcccctctcctccctcccctctttccttcctccctcccttccctccctcccttccttccttctctctttctttcttctttctttctctctctctctcccctccctctcctccttagcccccctcctttctcccttctctctctttctttttgagacagggtctcactctatcacccaggctggcatgcagtggtgcgatcagagctcactgtagcctcaagctcctaggctcaagcggtcctcctgcctcagcctcccgagtagctgcaaccacagccactacatctggcttaatttctgttgttttaagccaccaagcctgtggtaatttgtttcaacagccacaacaaattaatatagcactgttgactgtcaagtgaggcctgcaacagtggaaactttatagttctgagtggtcagctgtttgaggtgtgattaatctccaggaaaaatgttaccaggattccctttgtaaccatgacaaaatgctgagaagtggtggacacttagttgctgaaaagcactgaacgttcgctttcatctgacaaagtctttctgaataatacagggaggttcgggagggaaagaaggcaagcaaacgatgggatgctttctgcacgtggctgtcaggaaattctgggtagaaattctttctttctccttttcttttctttttgagacagagtctgactctgttacccaggttggagcgcagtgatacaatctctgctcaatacaacctccgcctcccagggctcaagtgattctcccacctcagcctccctagcagctgggaacataggtgtgcgccaccatgcccagcgaattttggtattttttgtagagacgaggtttcaccatgttgctggcctcgaacttctgagctcaagcaatcctttcaccttggccttccaaattgctcgtacaggcataagccactgtgcccagtccagaaatgttcataaattgttttttttcatagaccttattttttagagccattttttagattcatgcagaattgagtgaattgagtgggtgccccaccacccaccaccgctaccaacatcccatgtaagtgtacaaaattttttttttcttttacaggcagggtttcactttgtcacccaggctggagtgtagtggacaaccatagctcactgtagtctcaaacttctggctcaagtgatcctcctccctcagcctcccaagtagctaggactatagatgtatgccactcagcccagctatttttaaatatttttgtagagatgaggtcttgctgtgtttcccaggctggtcttgaactcgtgacctcaagcagtcctcctgcctcggcctcccaaagtgctgggattacaggtgtgagccattgtgcccagccataaattattctttttgcactcctcggaatgattacctctgaaatgactgtctgaatgtacataaccaacagtgaattttatgatatgtttaataggggtagtctttttctataaaaatgaaaaagacaaccacagattcttacaacagacatcggcagcagccccaaacctaaatagggaggtgttgtcccagctgcaagtggctcagctacagggtctcgagatgaaactccacggcacagtctaatgacctgctggaattcctggagttggcggggagattggctggataagtggtctctgcagacccccatgcgtgccacccctttgcccaccatagcccacttgaacccaccactgtcataactttccaggtcctaactgggaccacctactccccttggagggtccggatggagtttcccctctgcggctgcctctcactcatcctccaccactttgccgacaaggaaggaaggacaatcgggaggagggagtcttgcttggcgaccatctggacaatatctcgcccatggcaagctggatccctgtggattactttggtgggtggaaacttggcacactatttaatttgggctttttgggggatgactgcacactggttctctttttttctcagacctaaatttcgaccactcaactttcagcctgctcacccctgcttattgccatccacctttgggctgggttagacctaatcctccccttgacaaagccctcaaagtagccttcacaagataagagtcgagacccctaacttaccaacaaattttaatgaaacttctagtaaactcaaagcgctttgccaacatccttaggagcaacccagttctcgcagcaccccatgtgaggtcgctgttgaagtatctttggctgcagtggtgtggaattcatctcaacagagttgacggtaaagaccagacatgtagcagcctcactgcttgttggaggaaccacccttaagaaaatttcccaagatagtagttttcccatctccgtactttgctagttaatgttctccacttgcttgcgtttatcctttcaattcctttgctaattaaattgctgaaaagtatacattggtaagaatagccactgtcaatggactgcaaacaaaactcgtttctgttattgatgataaaccatttcagagaagaccaggctgagtctaaagggttcagataattacaaggggaaggcagagagagtagttcatggccacaggacttggccacctctgggcaacacataatgcttgctaagctaatgactgtagggatatagaactggccctcagcctctctgtcttcctttggcctctgctcttcctgctgccttctctctttgaccacgatgacacactttccataggccttcctcaccacatcacagaactcagagaagaggctgtcttcttgtttgatacacagctcgtctcttaggaacacccgatatttccaaggcacccatccttgactacccgcagcatgcacaatacaccaagttggggtttgtatgaaatatccatcactaatatcttccccagttacgagactttctgggatattggtttccccaaaatatacggtcttaaatccatcatattgcagtgttctcagggttttcagatattggaggcattgcttcctcttgatgccatcaaattgacttctgataatgacatttcttaaaagagggcttgcaaagcgaggcatggtggcttctcaagtgtttttaaaagcttgcacatctgcatctggggagagatggcaagtgggggatgatgacctcataaagggctttcttacagtggtgccagtcccacacatacacttcagatttcaagcatcatggaataaatactcagaacaactccctgctgacccataaaatatgggaattgcagatgctacccataaaaatgctactttgacatcacttctgcaataggtctgagacagaggttggcaatcttttaatagtaatgagcctacccatttggcttaagatggggagaaatgtttacattcattcatccatttcaaaaatatgtactgatcacctgctgtacaccaggcattgtgccaggcaccaggggtatagtcatgacacacccagggagtcctggacttacgtgttgtgtgtctgtgtatgcatagaatttgtgccagttgccttcccatttgcacaactaagagtgattttttccaggtggcagaaggaaggtatggcaaattgcaaaagaaagtcagtctgcacacctagcttccactggcgcttgcaggtctttttttttttttttttttttttttttttctgagagggagtctctctctgttgcccaggctggagtgcagtggcacgatctcggctcactgcaacttccgcctcccgggttcaagtgattctcctgcctcagcctcccgagtagctgggactacaggcaccttccaccatggccggctaattatttgtatttttagtagagatggggtttcaccgtgttagtcaggatggtctcgatctcctgaccttgtgatccgcccgcctcggcctcccaaagtgctgggattacagatgtgagccactgcgcccacccggagcttgcagttattgaactaattcaatacctcatcttgaaagcacttttaattttatatactcaggcaaaatgacagtttgcttcaaactctaaccatctcttcctttgtattttcttgcctctttaatcagagctaaagacatttcataaaatgggcatgaaggattccttcaaatgaagacgtggacaaaatgattggtcaggtcctttgctctactgttgaatggaggaggattttttttttttttccctcacacaggggttttcttggagctcaagtttggatgaccccagacagtaagataatctcatcatggtaaagttaatatgaaatatgtggtctccaaacagcctctcccagaggccaggatcagcaggtttgagtggataattggcttgtggtcattttctcataggatttttcttttagtagtggaaactgtttttcaaatcaaatttggatgccaactatgtggaacagaagtgtggctgctctggtggaagtggcaatggtagtcctagagtctccctgtcagccacaccctttgtctccccctacccaagggaccctgtggcctggaaccgcagtgtgaaatgctatatagtgcaatgaagtcaattcgaagacaagagttctttgcctttctcatctaatttttagttatggatatgagacgcttgttcagaagtatggaaaagtatatataatatgttatcttttagatgtgggtgtaaatatgcttatgtatgcaatatgcttatattttaacgcataaacaacatgaataaagcaaacactctagacttctccaaatgtatcttgttttacagttttcattttggaaaatgtcaacatttttacattaaaaaatattactcagccataaaaaagaatgaaatcacgtctcttgcagcaacatggacagaactggaggccattattctaagtgaaataattcagaaacagaaagtcagatgccacatgttctcacctttaagtgggagctaaataatgtgtacacatgggtacagaatgtaaaataatggacttcgaaagggaggctgagatgggaggaccatttgaggccaggagtttgagacaagcctggccaacatggtgaaactgcttctctactaaaatgcaaacaaattagccagacatggtggctgacacctgtaatctcagcactttgggaggccaaagtgggtggatcacttgaggtcaggagttcaagaccagcctggccaacatagtgaaaccccatctcaactgaaaatacaaaaaaattaactgggcatagtggtgcgtgcctgtaatcccagctacttgggaggctgaggcacgagaatcatgagccgagattgcaccactgcactccagcctggacaacagagcaagactccgtctaaaaaaagaaaaaaaaagaggataggattagggtgagggatgagaaattatttaatgagtacgatgtacactactacactcaaagcccagacatcaccactgagcaatcaatccatttgacaaaactgcacacctgcacttgtaccccttaaatttatatacaaacaaaaacaaaggcaaatcaaaaataaaaataaaacaaaatgatctctaaacaatacaaacagtaactgatgaacctagctgcttatcatgtcagttccaaaatcacacagagttgaatttctttcaaatgaccctacaacacagtattttgatcatatattctccagtagagtataagctaaggacaaagaaaaacacatgaaatcttaaatggtactcggtagttttattgttaataatgatgctggtattattattttgaaactcatgtccatttctcagctgccatttgatattaattttttttttttttgaggtggaatttcactatcactcaggctggagtgcagtggtgtgttctcagctcactgcaacctccacctcctgggttcaagcgattcttctgctttagactcccaagtagctgggactacaggcacgtgccaccacacctggctaatttttgtatttttagaagagacagggtttcgccatgttggccaggctggtctcgagctcctgacttcaggtgatctgcctgcctcagccttccaaagtgctgggattacaggcgcgagccactgcgcccagccagtgatttttaacctttatcaatctgatagacaaaaagatcatttcattgttttaacttctttaattatgagtaaatctggcaatatattatgaaaacaatgacaagaagaacttgatataaaatgcacaattcagacgtccttgaagatcattttagaggcagcatgaagtggggggtggcactggtgatgggggctgggtgtggagaaaaccagccaaaggaggactcatgggatcctggagcttttgactggggttcacttggagcccatactcaggctggttctgacagcagcacctgccaggcctcagcttaggggcacaatgtgggacacatggactgggggtgtggtcccagagcctaagaggcaccaacagagaggtctccgcagagactcatctgtgccccccacccaccaccccgggacaggccaagccagcgtctggccaggaactgcttttgcacaaggagccagaagtagtttgccccgataaatgggggcctggactcacgcaaaccattgcaccatggatggccagagaaactcagagaaccttcctgtgcttgttaacatactctctcacgtcctctgcagcctctgccaagccaagcagccagctcctaggactcccctccctccacctgaggctccttgtcctcccttcctcaggagtctccagcctcccgggacttcccctccccgctgcccactccagcagaggctgccaactgcctgggagagagaagtgggcttcctggggccacctccccaactttggagtgtttggaaggtgatggagcgaccactaggaggcagtgtggacaggtctctgtaggactgctcaggcagacacctttgcagggacctcccaggtcgggagccctccacacttttcccatgggagcttctccctccaccccgagtcactactatctgctttcctagaaggcacttctttacttctaattcttctccactgcccaggtaactgatattctcaagtgggacattgtaatttgtttaattcatttaaattgatttcatataattgggagataaagattgttcagttgcaagacaaagtcttaacttgaactctcaggacacgggtgggtccctaaactcaatacgtgagtgttgctgccgggctgttgggccatcttccacccgccatagatcactttcttcatcaaagaagaaggaatatttagaaactggtagtacaaaaaaacaaacaaacaacaacaacaacaacaaaaaaccaaaacaacaaaaaacaccaaatcaccaaaaacaaacaaacaaaaaacaaataaaaacccaaagcagttgctcctataaatagatgtgtgtatacatgtggctggtatgaatcttatccacaaattcagttttgtgggaaacatcacatttatttatttaaatcaagtcatatgggacttgggcatggttggaggttcttaccccaccccacttcccaaggccagtgcacaggcagggccttgaggtcacccttagccgatgcttgggtctaggtgctcagacccaagcccctgtggtcccatcatgtgggcactggcatctttgctgaggctgagaatttcaaagccaggatccagcccatttaggtaaacccaaagtcactctcccaggtggcccagtcatcttcttgagaacaagagccatgagcctcagttccctgcctcaagagcctctgttcaaccctaggcttgtagacaactctgccccttcttctctcccttcagtgtcacggtcccctgtcccatccctctctgggacaggtaccacaacctccccaccatacacagggaaagggtcagccctcaggtttttggcctggcatcttgaatctcctcccaggcaacaaaccacagagggcctggcattctcctgtgaaaagcagggcggaaaggaaacacagagaacaaacccacagacaacaaacccacagaaaacaaacccacagaaaacaaacccgcagagaacaaacccacagacaacaaacccacatcaacaaacccacaacaacaaatctacaacaacaaacccacagagagcaagcccacagggaaccagccaaattatgtctgctgtgcatctcggcagacgatgctgccaccgtctgtgtatgagcatgtgtgtgtcagactttcccatcgtctccaaacttgttttcagaataatgcttccagtgaaatgagtcggccacatgaggtcacaaagcccctactctgttcagcacctggggtaagtaataatattttggagcacttagtgtggggagtagccctgacccctttacatgtcatgtcttagttcattcttgttgccatccttggaattgaggccaacatcatctgcccatttgccagacaagctgctcaggaggagagggccacagccccttatctcctcgccaaacaagagaagatccccagttgctttttttttctgtggaagagattcttttaaaaacatttttttcatggagaagaaaatctgaaaaaaaagaatgaaaccgaaccaatagtcccatagacagttagttgttgttgttgttgttttgtttgtttgtttgtttttgatgaatacagaaattgacccttctggtcttaaagcttgaaaattaaatttgttttatctgagttgcttcctcaggaaaggagcccaagtcctctccaaaagtatcagagaactgaaactcaccagatcatcttgtctagacaatgagacgtcaggccctccattcatcatgactgcttccttacccctcccgagttcctgttacatttcttccctgctatataaacccctaattttagtgggtccagaagatggatttgagactgagctccatctcctgggcagcagcacccaattaaagccttcttccctggcaatactgattgtctcaatgattgccttccttccttcctttctttttgagatagagtctcactctgtcacccaagctggagcacagtcgctctatcttggctcactgcaacctctgcctcccaggttcaagcacttctcctgcctcagcctcctgagtagctgggattataggtacccgctactacagctggctaatttttgtatttttttttttttatagagatggggtttcactatgttggccaggctggtctcaaactcctgacctcaggtgatccacctgcctcggcctcccaaagttctgggatgagaggtgtgagccatcacgcccagctgagtatgtgtgtgtgtgtgtatgcttatggggatgtgcaaatgtgtgtgtgaatgtgtgcacgtgtccttgtgaattgtgaatacccaggacttgagcacactcagttcctgatgcacttcctgttttctcagcagctgagctcaggcctggaactgagtgacagcacacccgggcacctgtctccctgggcacccctcccacgcctgcttcccacggcattcccagctcccaccactgggaaggagctggaatcatgagtcgggataatcaccgaattctcttcgaccttcctcagctcctggtttgttaaggcaaacccccatctctggcttctcctggaacctcacctgggaaagaaagaggcagccccggagctggaagctgcttcagggctcacccggaacagcagactcaacctggacccatccaggcatctcctgggagtttcacccaattgcttctgcctggcaccagctcagaggttctgacagaattggcctggggtgagacctggcatccatgggatttttacaagcttccaggtgattctacagggaagccaaggtgagaacccctgtcctagaaccaggtctgatcaggggccggtggggaactgtgggtggagaacattagtgcttccagagcctcagggttggttttgaaaggaacgtaacacatttttttttctcacaaagacatatagagagagactttttaaaatagacatatatatagagagatgttttaataaagagaggtttgggtttatataagtaaaaaagatgatagaaaatagagaatgagattaggcttccccttgctctcaaaaaatggtttgagagtcttggaactggttccactctatgatagccatgagtactgttcgcccaacttttggttctcagccttccaggccccggtgggatgagacttccctgcccccacggttgagaggagccatgggcttattctagccaatgaatggtggatggacgtgactcgtgtctcttccaggctggagcatttaattgtccaggtgagatactcagggactcgtccctccagagctgagaatggccatgtttccagagggtctgcaggagcaacctgagtctcagagcacagccaccagcagacctgctgcacgcatgtggcgggggaaagaaagaaagccagctgtctgcagccactgagagtttggggtggttgtttctcatgacaaaaccagctcaccctgacttatacaaagtctttgagttatatagatggagaatgaggctcttgggtccctctattctcacaaagcaatagcctagctaaatccatctaactagggagcagaaaaggggatgtgctggcttgcacaccctagacagttgttcaagaagtcaggacaccaggcctggagtgatacttcagccatccttctaggtgagggtcttgaggccacacagacagaagtggcagagatgggacacacattcgtcttctcactcacagtctggcactgagctgtgggctgctgggacaccatgcccccatacgagtagccttcccactccttaccttgaaggaaaagtgttttttggacaaatacctgatggaaacattacatgggccttggaatctgttagatctagcttcctgaaactcttgctagctgtgtgacaatatacaagtttcttaacctctctgagcctcagtgctctaattacactcccctcatagagtttctaagagcatcctggggctggcacgtgtcaacgcacccagtatttgatagagtttgttaaacgttggttatcctctctccctatcgcacctcaaatggtaagggctgcctgccagcttccatatccccagcagtgccctgagttgttcagatgttcatccatctcccacagaactcaggttcctttggaggaagccacatcaagtcctgctccaagcttaagccagtcagcacattccatgctctgccccattgccagggttcaggagtatgctcgtgatctaagcacccccactcccagatacagctcaagattcttgcttggacttctgggcactcaggctcctccgagagggaatcaaacttacttctctccatgtcctttcctctaggagatgcgtctttcataaaactctcatcaacactgttcagacatgtcccaccccagcaggggacagcctgggctcaagctgggatccctactttatttattttctgctaattaaacttcctaatatactccacactaagtgtgcttgcaaggcagggggtgtgggataagcggccctgcctggctgggagagggggcagctccctgctgtactatgtattaataaagagacacatgcatggcagggcttgtctgggccttggtggcagcttaggacagaaggcacgtgacagtcaggggttcaaacaacccagggagaacactgcttcagggaagacagctcagcatcttcctggcaaagataatgacattgataatactctccaaagaatttcaggattttgagcaatcagaaaagcaacacagaaattcatgtcatcaaaacgatatggctctattggacacttaagacatttattggaggctcaacaacataatcctgctggttggttttacttcattgattttccgttgtgtctgattacattgctaatgctgatggtggatgagctacggctcttttcctgcctgtcctgaggtttatccaccaatgtttcagttctgttttaagatattgtcctaagcccccagcatcgcatgcatgctgtttttttgttttgttttgttttgttttttacaaagagttcatagcccgtggaagactctcctccatcacacacttaggttccctccacaccaggcctggaaggagtctagcttctggggactgtacatatgctgtggaccatgcagaacctggagaggcggtgaccccttctagaagtgatctgcctgaatccttccctctggaggaggcatttattaaatgccaggttcctgaaaggctctgagatgggcactccctctcctgagtcgtcccttccattactgctttcctatttctggccagggttccctggcccctcctccctgctcccatgggaccccagttcatccccatctttgctcaattgccctgcactgtagtaatccattggcactcttgtcttctccaggagaaatagttggaggagaagtttatagggtttcctgggccagggctggtctacagtcactggacagcaggaaacgacccttcggggcctaggagggccaaggctggtgggcaggtacagggggagccagcactgctgtccaccactgtgcagcctggaggctgtttcccatgaccctgctgatgggacccaaggcaccccaggccacccactcccctgcccccagcagggtgtcagctccccggcttccctgcatgcctgcctgacatggacagtgcaccttcgggccacacttgccctgctagcgagcctccagtgaactgggaattccacagagtgtagaggactcgccccagcactgtgctgagaggcttcaccaaactgtagcctggcttccacctgcactaagctgcatccccagaggcgaccccagccctggctgagtcttggctcaagactttgcaatgcagccaaatcacaaaatgcacctcgtccagcccaccccgctaaaccattttcagtagttctcccctcaccgttctggaactttccatttccacgtggcccccacgttctgttttcatttctccttcagtccctttttgttccctttctgttctctctttgaagacctcagtcaccgttttctgagttggggttgagcttggtcggtactggaatctctttccgctgctgcaggagtctgaaggaatcagtcttgccgcctgtaacacatgtccagcgctgctttttctctgatgagtgtcttagtcagtttgggctgttaaaacaaatggcttaggcaacacacgtttctgtctcacagttctggaagctggaagtctgagatcaaggtgttggcagattcggtacccggtgaggacctgcttcctggttcgcgggtagaacacttcttgctgtgtcctcacaaggtgcagagagagaggggggtctggtgtctcttcctgtaagggcactgatcccatcatgggggccttacaatcgcgacctcatctaaacctcccgaaacctcatctaaatctcacctccatactatcacattggggattaaggctttaacatgtggatttcaggggacaaaggacaaaagcattcagtccatggcacatgctcagtgcctcgactcttgcaagtgccacaccacagcctctctggggctgtgtcctggaggcgtgtgccatgggccctgtgtgccatgggcaaggcgcacagcatcctcccggccaccccaccagcgagtgagctcctggcaccctggctctctctggtcacccatctaccagtcttggtgctcctgtgcactagaggaccagctgcctggggactgtgggccaactgtggccccggccacccacgaacttcccctccggccagtggctgcaatcacatcttctctaaagacgtctgaagcccagccttggggagccgagttggtccttccctgggtactgagccctagggaacccttgagagttctctttgtatctttgtagtttcttcctcaccacttaatcatttccttacaggaaacttcctgtgttcaagtgactgtatggtttctgcctccagcttcatttgtgggtgccataagagaggcaagcatggagcactaggcgcaggggatgggcaactggcaagcggggagatgcatgcagcgcacttagtgcctggtacataccaagtccttttagtctggatttcattatttttaaatgggtattgctatttttaaaagaatagttacaaatatttattgtgtgttttgaaataagtgggtcaagatcaataagatattgttgatcaattgatcaataagatatcttttattcttaaaaatcatattcttctggttcagtggggaagagactgccgacctgtatttacagcattatgtgataagtgttctccttttcaggtatgtattagtctgttctcatgctgtcaataaagacatacctgagactgggtaatttataaaggaaagatgcttaattgactcacaattcctcatggctgaggaggcctcaggaaacttacaatcatggcagaaaaggaagcaagcatatccttcttcgcatgatggcaggaaggagaaatacagagcaaagtggggaaagccccttataaaaccatcagatcttgtgagaacgcaatcactatcaagagaacagcatggaggtaattacccaccggctccctcccatgacgcatggggattatgggaactatagttcaagatgagatttgagtggggacacaggcaaaccatatcaaggtgactcctgcaagcacctacctccacccctccttcatccttgccctcattctacaatgatttggtgaaatctggtccctgcctcagttttacagcctccccatgactctggttacttcctgattagcttaaacgaaacctaactaggttgccctaggaaagcatttctgttcctgacaccccccatctgcctgctgcttccgttccacctgtatgtgtctgggcacatccctgcatccctttgctggcttctagcctactcacttcaagcatttatcccatgagtttcataaaatcgtagaagaaaagggcttgaggcagtggtggggaaatgataggaaagtcatttctggatgcattctgccatcctgcagatccctaaaccacctctccctctccattccctccctccagagaacagcttctccttgtctcctgtggaatagttccgcccacattcatgggcccttcctgtaccaaaactgtacaggtctctcttgcttaccaaacacttggcaaacaaatgtgccgtccttggaaaaattctgttgaataaaattttctctctttgatccatccaaatgttttacaaagtgctacagaagccatggaggaacaagcaattctgccttagggatcaaggtttcacacagggggtgatatctgagcaacagtgcttttttggtttgtttgttttgttttgagatggagtctcgatctgttgcccaggctggagtgtggtggcacaatctcggctcactgcaacctccgcctcccaggtttaagtgattctcctgcttcagcctcctgagtagttgggattacaggtgcccgccaccatacccagctaatttttgtatttttagtagagacggggtttcaccatgttggccaggctggtctcgaactcctgacctcaagtgatctgcccacctcggcctcccaaagtgctaggattataggcatgagccacagtgcccagccaacagtgcttttaattggcattttcttcaaagactttgatgtcctataggagggggcctatgactcagcctcagccaatcagagcgctccattccctgggtcacctgcacacctgctcttccctgatccactgcagtgccctcaccctgagatctgaaacttgagcagaggcactaaaaggcagacatgggagctgagctgtcttttgggagaatcctagtgagaaggttctccaactggggccgccaagtaagggcctcatggcagactaacccctctccttcctaaggctgggaggagctgctgtccttttgattctgtgagctacctcagttaccttcctcaaaatcacacacacgcgcacacacacacacacacacacacacacacacacacacatttgcatgcgctaggtagagctgttttccataattgccaacagaagactaactgtatttgaagaatgagctggcattcttctgctccggtagaagtcaaggcaatcagttatgagaatcagagcccacctgtgactccagaaagaggtgcataaataccaagaatttagtctctaaagtctttctttaagtccttttttaaaaaatgtgatgagtacatcacccaggaaaatcaaattgtaatgcaaccgagtcgatgcaagttttatttaggagatgggttacaatcacctggggaggctctagttaccttgatttggtctggtacaaaccctagcaccatcatccacagatccccagaggaagtcattcctggatgacttcctcatcgattttaaataatttccatttcagaggaaggcctttatctgacctgatcccctaaatattgggggaaacctacatagggacaaagacagcaggtgtctgcaatgttgagaatcagtgtgttctgtcactgtctctatcagggctggtggcacatgcaaatctctttcccactctccagttgaacactaacgccatggtgcccacaccttccttattagtccatgtacatggggtttgtcaagacagtggttcatggctctgaccctgagcatgtcagatttcaggggctttatgcaaaatatccataccagttggggtcatttcccatcagtattgctcacaatggagcctacaaacccctagttcccatccaacacatctccaaggcagactctcagaccagctcccagaaatgaggtgagtttagatcaggcagcagagaggtggcctaggaaggagtccttggagctcatgcacctgtgtctgggcaccaacaggaagatggtggcttttgctctttgggagatatctttggagccagtctctgaccacatgtccaacaggacaggcatccttggggtttccatggcagtctactgacagtcaggggtgaggattaaatggtacagagtctcactgagtgctctttgagaggtcaagcaatgagaagtcctgcaaatgattattgagctgaagtaagaagtgtaccgaatctgtttttcccctataaatataaaagcctataaatataaaaatcttggtgaaaaaaaatgatcccagcctcccacacagcacatcacacatcttctcttttcaaatttgactccaaggcccacttccttcgggaaatcatttatccagtggtatcatttaggatatttttggttgtgaggaacaaaagcctagctccaaaagacttaataaaaggatctcattggttcacaaactgaaaaactccagtggtaaatgaaggctctgggtacagttggtacaggctctggtctctgtaatttcctagttcttctcccttctagatgctgggtttttgccttcaagttggctttcttcatggtggcaaaatggatccagcaattctgtcagaggttttcgaagcagagtgactccatcttgattaaaggctgtgtaaaatgaggatgagacttgctggactgcattccaggagggtaggcattcttagtcacagggtgagacaggaggccagcaggattgatatcacaagacacaggtcacaaagaccctgctgataaaacaagatgcaataaagaagccagccaaaacccaccaaaaccaagatggtgattaatgtgacctctggtcttcctcactgctcattatatggtaattgtaatgcattagtgtggtaaaagacacttctactaactccatgacagcttacaaatgccatggcaatgtccagaagttaccctatatggtctaaaaggagaacctatatagtctaaaagaactgagggttctgagaaatccctgaccctttcctggaaaatttatgaataatccactccttgtttagcatacaatcaagaaataaccatagtgtactcagtcaagcagtccctgctgctgctctgcctatggagtagccattcttttgttctttactttcttaataaacttgctttcattttactttatggacttgccctcaattctttcttgtgcaagattcaagaaccctcccttggggtctggatcaggatcccattcccgtaacaatttcaggctcagatcggcttttaacaccatccagagcaagagaaagcttttttgttccagaattccccattaaagttctcctggtcactcttattgggttgtttcgcttagggtcaggtgtccatcctggtcccaagcaatgaggccaggagatgggatgcaacgactggatcaatctaggcctcttattcccactttttaaaacacttattattattattttttaaaaattatttttcattcagctttttcatttgaaacttattccaattcttgaactgggggtagtttcaactttcctagagctgtatgggtcctcaaatgaaaattcggggcagctggattagagaagggggaaatgcatgctgcaggggcaaccaacaaggggagattgtgccaattcactcttcctatcctcagattcacctaagttctgaccattcagccccatttgaatgcattctgtattcctatgactgtggattacatttttgtctacctttgtgtcttctgttttgtcctgccctataagcatctcaaacatatgcataaagcctatataaactttataaataaactaacacttctgttttcaacctgtaggatgatgacaatgatgatgacgacaatgatgatggtaatgatgtggaaaatgtgaaaagagaaagaaatacttggaaatatatctcaccctccataaacaaagctcggggtttaattctgacctgtatgagttcatggggtgaactgcagaccgctgtctgtggacaggaaaacgatatttcatctctagccccagggacatctccaaaagctgagctagatgaactttatataaattggtacaaaatataattttctctttgcctgctgaaagccatttctagaaattctgttaatcagaatctccctaagttaatcagtcatctagacagatcttatttcttttttagacaaagaaaagtatataagtaacaggtattggtaaaccacttgagtgaagcatatgatatctaatgtaaggaaatctaaaagtgtccacaggcaaaatctcatggattcaattgatagcacaggtcatcaactgacatgcagacggaattctcttgtggaacaagacaatacagccattgcttagagactaattgtcaaggaattagtcatttcctgtttcagaatagcatcatcaccaccaccattaatgccaacatcaaccaccaccacctacgccaccaccgttagcatcataaccaccaccaataacatcaccaacagcaacactgccatcaacataaaccatcaccaccaccaaaaccattagcatcacctagaaccaccagtcaccaccatcaccacttaccacaacaaggcttatatttacatacttattttacttttcgaaatacattcacatgcatggtttcattagatcttatctacttggtaaggttggcagatctgacatcattagcctcattttatctgtatggaaactaagttctagagaagcgaagtgatgtgtgaaaggacaccagagtgattgataatcaaatccagactagagtttggttcttctgactccaaaattaatacatttttcttaaaagaaaaaaattttttttgagacagggtctcactctgtcacccaagcttgagtgcagtggcatgatcacagcttactgcagcctcgacttcccaagctcaagcaatcctcccacctcagcctctcaagtacctgggaccataggcacatgcctggctaatgtgttttaaacattttttggctgggcacggtggctcatgcttgtaatcccagcactttggtaggccaaggcaggcggaccacaaggtcaggatatcgagaccagactggccaaaatggtgaaacctcatctctactaaaaatacaaaaaaattagccaggcgtggtggcacatgcctgtagtcccagctactcaggaggctgaggtaggagaattgcttgaacccaggaggcagaggttgcagtgagctgagattgtgacattgcactccagcctgggcgacaagagcaaactccgtctcaaaacaaaacaaaacaaaacaaaacaaaacaaaacaaaacaaaacaaaacttttttttttttttttttgtagaaacggggtctccctaggttgcccaggctggactcaatcttctgggctcaagtgatcctactgcctcagggtctctaaatgctgggattcaggcatgagccaccacacccagctccaatgctttttttgtcgtacctaattctttcaatgaaaatgaagaatttccaacttctgatattaacaactttggtcctatattcaagctagagtctttcaaataaaatagacttttaaaaccatctgtctccaaaccctaaatgtctcaggtgagcaactaagctgctcagtttatgtgactccccagaagttgaattttaacccagaactgactccaagttcattcttctttccacgacaaggagtcacctccttgtatgcccccaggagtctcccggattcctccgagaacagtggaatagtgctcctccccagagcacaggttttgccagtgaagattgaatttggctagaaaccgctgccctgctctctcttctcgaagcacctggaagtctgagaaggaactgggtggctggctctggtcacaaactagcagccagaagcaccccttgtcagtgatgcacccccagtccccctcaagggctccaagtaaacccaaagctgctcccctccaagaagtctggggccaccctagggaaggcctcctggccttgactctcagggggtctctggggttgcggtttggggcccgctgcttccgccctttgcccccaggtgggcctggcagggctgcagcacagctctgttgctgatagacagggtggagcacttggcgaccttgccctgcagccctgtcattttgagttcagaggtcagatttgagtaataaacatcttctaaggacttgtcattctttctgaggatgttgctggccagccggaagacgaaaatcaccgcgtagatgccgatgatggtgagtatataccaggcagcgctggttccgtctggcaccttcagagccctggtggagttggtgtcattcctcccctctgtgtggtcacccagcaggagccccaggagggtgctggcctgggtctggttggaggcttcatggggagtccacttggcccctgagaaacagagaggtccggatgagatccagcgtcctgggctgagggctgcctggccacaccaaggagaatggagccctcatatccgtgaaaacgtgtcgctgctcaaagaggccttctctgaggcatgagcaggagtgtaacaacaggtatgtcaatatatttttaaaaatcaaaagagtccaaaacactattttgttgttgttttgttttgttttttgttttgtttgttttagagagacagagtctctgtcacccaggctggagtgcagtggcatgatcataacttactacagcctcaacctcctgggctcaattgatcctcctgcctcagcctcacaaatagacatgcagcaccatgccgggctaatttttttcttttttctctctctttttttttttgtagagatagggtcttgccatgttgaccaggctggttttgaattcctggtctcaagagctcctctcaccttagcctcccaagccctgggattacaggcaggagccactgtgcccagaaaaacactaagttcttgaataggagacacaacatcataaagatgtcagttatccctcaaataatttatacaacaaacataattgcaataaaaacagcaataggatttctttgtgaaatcaataaactattcatttagaaaaatcaactgttggccgggcatggtgtctcatgcctgtaatcccagcactttgggaggctaaggtgggaagattgcttgagcccaggaggttgagaccagcctggccaacatgacaagaccctgtctctacaagaaataaaaaaactagccaggtgtggtgtgcaagcctatggtcctaactactcaggaggctgaggccggaggatcacttgagcccaggaggttgaggctgcagtgagctgtgttcacaccactgcattccagcatgggaccctatttaaaaaaaacaaaaaaagaaagaaagaaaaagaaaaagaaaaatcaactgtcaagactaattagaaaaaaaaatctgaataaaaagaatgactaatgaattagcctagccacaaattttaaatcagccagctataaaaactaatttacatttttttcaatgaatgaaagctttatatgcacaaagcccagctgggacttgctgggctttgcagagtgtgtgggctgggggttcttcagaaccaggtacaactctccctataaaactacaacagtgctgggcatggtggctcacacctgtaatcccagcactttgggaggctgaggcaggtggatcacctgaggtcaggagttcgagaccagccctgccaaaatggagaaaccccgtctttactaaaaatacaaaaattaaccaggcgtggtggcacacacctgtagttccagctactagggaggctgaggcaggagaatcgcttgagtccaggaggtggaggttgcagtgagccaagtgatgcctgtagttccagcaagacagagcaagactctatcttaaaaagtaaaaaaataaaaaataaaactacaacagctaaaatagtgtgatgcctgtagttccagctactagggaggccgaggcaggagaatcgcttgagtccaggaggtggaggttgcagtgagccaagatcgggccactgcactccagcctgggtgacagagcaagactctgtcttaaaaaataaaaaaaataaaaaataaaactacaacagctaaaatagtgtggtgctgaaaacacaggcaagcagaccaatgaaacagagtaaaaacagcatcaatagttagcaattagaatttgatagctagctaataaaggagcatttctgatcggtgggaaaagatgaattattcaatatgtagcattgggggaaatagcattagatccacatctctccaccatatgaccagataaatcggtccagattaaaaaaaaaacagcccagataaatcaaatattttaacataaaaagtgaaataatttatagtactagagtacagcatggcagattttttctttatcatctcagagtggaatattcttttaagcataacaaaaattcagaagaaacaagaaatagaaatcaaattcaactacataaaaaaaattaagctatttcataccataaaaccaacaggcagatgacaaagtgcaatttatatcactgattttctaaatagccttcggttctgtaagaaaaagtttaaaactgcagtagaaaaatgtgcaaaagatatggacaaatagttcacagggaaaaaatgaacattcaacataagaagagcttctcaatatcactcatataagaaaaatgcaaattaagataataactagataccattttgttacctattggacttgcaaattcatgatgtttcagaataaactaacaaaaaaatggcttttttttgttcttttgtccagcttagaagaaaggtgtctaaattgggagcaaaggtggcaatgacgtggacttgacaccaaaaaaaaatttttttaaagaaaagaaacaagtgcctctgcatttcaggggtttaggattggcatttttaaaatgtcaacaaataaatgttcatatccacacttgacattttttccaaggagaattttaattgtataattgctggtaaattcatgcagccaacatggagggcacacggacaagatctatgagcattacaagtgcacttacctttgacccagcaattctatctctaggaatctatcctaaagatgctccagaacatctagagacaacatatgctgaaggttagtcattgcagtcctccttgtgatgacgaatgcctgggaacagcctgaatagcaccaactgagggatggtgaaatacattttggaacctccatgcagtggagtactacacagtcataaaaagcaatgagttttttatggtactgaatgttaataagtgaaaaaataagctaatggtgacatgctctgcaatgccacttgtaaagaagggggaagttatatgttatttgcttgtactttttttatgtatagaacatctctggaagaatgaataagaaattagtatctgcaattgcctctggggaagaaacctgggggaagaagatatattttttactgtttgcccttttgtacacttagtaccgtgtatacttatttttgaaaagcaagagtgtaccagttggtacttttctggtctccctggtgaggtgcccctgggtaaagccgttgtatgcccttgtaagaccagaagattaagatctcaattgctgttcaattcaaaactgttttctctgcttggagagctggtggagaaaatgaaacaatgaaaaccagagctgtagagtgcaatcctgtgagacatttcccagtggggccttactggctcaaacccccatttcttgctctaatgtgaacacagatgtatttaaaaacacatcataggatcaatcttgcagcctgctgtgcagaacaaaggtgctccaaaatgcttcccatttgatcgttgtttgttgctaattcattttgcgaacgcaagactcagagaggccagtattttttattatagttagttgccagaatgtgtgaatgagcttattacttttagatgaaggaagaaactatttaaaaattacttttcaaactacatgtgacaaagcccaggacaaatgaacagatttaattacataaaattagtcactcgcaagaaacaacaccacaagcataaatttacaccattgtttggtagaatggtttgagacattaaagtaaggaaggtgaaaaattcccgtaattattgcaacaaacaaacagacagcaaatcaacccaacaagaacacaatatccttatattagggcaagagaacttattgaaactcagaacacatgtataaactcatagaactttctagaaattgtcatagaatgatgcaacacattcaaatacaaataaaatatccccaactaagagctacacacagaacattaaattatttaaaaaccagtccattttctacacgaaagaaactcactatattaattactgcaatacattacattttacctttcttacaaaggtaaaagtaagttaggttgtatcttaatggacaaacatatcctgtagaagagagaaactttttcctctgtgctattttgtacttgtaatttaatgacgtgaaatatgtaaaatctcaacctgcccatccttgcattgtagctgagtactcacattccatggggtggtcttgtccttgactcttggaggggcaagttcaagcggctaccatgcacagaaggggaagatgatgaaaggagaactccgtctcctagggaagaatcagtcctactgcagttgagctgcactgagtttccagagtggggagtaatatgatcttccaacaatcttagggcagcaccaaacagaaacttagtaagtggatgactttgctttcatgcaattaatcagaggatccgatttgctgtgtcttctgttgcatcagaacagaaagcacttcccagctttgacttgttaagaagttctcaatcaaaacaaatttttaaaacgtgctggtattaaggaatctccatctctcaggtcccatcatgaactgaggtggccagaagctccccctgaggctggctctccgcttagagcttggatggctattgaattcccctgtgttctgcacctgttgcaggtgtggcagatggccaggtgtggcagagatctgtcatcatagggccaggaaactccatggtcaagagtcaccagcttcctctggacagtctcccagatgaggaaacccagacaggaagggagtgacaccccaagggtgacacacctgaggggacttgggctttccctgaggggtcagtgggcagtggactcctgtgccaggtggtgagaaatggctcttctctttcccagagtcacagaccccattggagttgaggtaggcttaattggaaagtgttagagtaagtgtctgcgggtaaagtttccccaggagcagggagggaaaagttggaagactggcaagttaaatcatccagccattgtttccagttccatttcttcctaatcctcactctaggactctaacttgccacgtttgtgatggttgctggtttttaagatacaatttgatgaaatttccatcaatggggtactgggtaagtaagttataaaataagccatatgatccagcaattctactcctgggtatcttcccaggagaaataaaaatgtaagtttacacaaaaacttgaacacacatgttcaaagcagcattatctgtaatagcaaaaaatggaaacaacccaaatatccaacaactgactaatgaataaataaaatgtggtttatccatacaatggaatgttattcagcaataaacaggaatgaagtactgatatatgccataacacggatgaaacttgcaaacattgtgctaaataaaagaagtcagtcacaaaggactacatattgtaggatttcatttatatgaaatgccaagaataggcaaatctacaaagatagaaaatagattagtggttcactagcgggagggattgggggtgataactaagggtatatagcatttttggaggggtaataaaacttctaaaattgtggtgctcactgtacacaatctgtgaatatacaaaaaaattgaatgcatactttaaatggatgaattttatggtatatgaattatatttcaataaaactgttaaaaattataatatacaagctgggtgcagtggctcacacctgtaatcccagcactttgggaggccgaggtgggtggatcccctgaggttgggagttcgagaccagcctgaccaacatggagaaaccctgtctctactaaaagtacaaaaaattagccgggcatagtggagcatgcctgtaatcccagttacttgggaggctgaggcaggagaattgcttgaacccaggaggcggaggttgcagtgagcagaggttgtgccattgcactccagcctgggcaataagagtgaaactccatctcaaaaaaaaaattataatatacatatacaatggagtattacacagctgtgaaaaagaacgaggaagctatttatgtactgatgtataaagctctctaaggtgtgctgttatgaaaaaggtaaagaagagagcatgttaacatgtatccaaaaattgagaggaagcatatatatatatatctgattttgccactgtaagcatttaaaacaccagtggaatatccaagaaattaagaagaggggttacctattggaggagagaaccaggtagatatatggcaggtgtgggagggagagctctcactaaatatttttatgctttaaatatttttaaccgtatgtgtattacctattcaataataaatgcacccatttgttagatatctttgttgaagattcatttggctcctgctgtctcttgctatgggatggaccatggcatcccccctctgccacacagacaagggatttggacactgccagtgggacgtgggaggggagagcacctgacccgtgataataaggggctcgtggcagtgataagggctgggagtcagggctctggccccagccacatccttgctgcatgaccctgggccagccccctcatctttgtgagcctcagtttcctcatctgtgaggtgaaggtggtgaaggaggtgaaggatgagcaggatcttatgtccttggtcctgagaaggcaggagagaagcctggggctctgtgtgggaagagccgctctctggggaggtatctgaatagatgagggagagcacaccgggcagccaatgtgccagaggtggaggctttggagagtgtttcatttgtgaagtcaacagatttaacattcagatcaggaggacgttggcatgagatgtggggaatcataagctccaaaacaatcgtgagacagaaggaaagatggccttttgttgagcagccattctcctccacggagagtcctgtctagtctgcctgttgaaggggcactgatgttagggaatagatctgtgtcaaatgcttcccacctcccagaatcctgtgaggcaggagtattatccccatttaaagagaggacactcaggctcagggaagtgactggcccaatgtcccatagctcataggtgccagaggtgggtcatccacaccaaagtcattctccttccataccctgaatgtcaccttcacgctggacccaggatcctgtgtggtgaactgtctcgatcacttccctaaaggttaaatcataaactcttactgccaaggcatatccacgaccttaaactctccctgttgggcaaaaacaatctctgatgttaaaaggcaggatagtggatacttttcagggaagggtaaatgacaagggcatgaggggaactctgggtgccggtcatattctgttttacaggtttgttcaatttgagacacttcatagagctgtagccttgtgcacaggcacttttttgcatgcatcgtctgcttcaatataaacctcttcctgttgtcttgtttttgtttttgtttttgttttctcttgttttcttgtcctgctctgtcacccaggctggagctcagtggtgtgatctcagctcactgcagcccctgcctcccaggttcaagcgattcttctgctcggcctcctgagtagctgggattacagaggtgtgctaccacacctggcttccctgttgtttctttaatgtagaaagccctgatagatggtgggaaaacaaagtttaaggtattcatagaaaaatacaaatactatttttaaggattctatatctggccacatggtgccatctcacgaagagtgtccccgtcccttgagggggagtggtcgggatcatggtcagtgtggggccctgcagctgcctgcttccctatgctgtgtggatgacgcccgcctccggtcattcccctgtgcttacataacagtgaaatggaacaacctgtatcagcacgagggccaagaattttcttctgacttgtggatacctccttccttaggcctctgatcagtctggacaaatattgccctgaacgcaaccaagcaaagccactcacctggtaaatatttgtatgagctacagttctggaagaacaaattccaatatcctgcagtccccttgacatcaaagacccaactctcccagagggcaatggcttttttgtccactgagaagccagtcagcttcgaagaaaggtgtctaaattgggagcaaaggtggcaatgatgtggacttgactccaaaagaaattttaaaaagaaaagaagtgcctttgcatttcagggggtcagtattggcatttttaaaatgtcaacaaataaatgttcatatccacacttgacattctttccaaggagaattttctagaggagacagacctcatcggtcagctctgatgccctgcagtgcaaaaagacattaaaaatgacggtaaaggacccctgcagagaacaactgagtctcttccttgccctgcgtctccagataaaggatgccctgcatccatcccctcctggctaagagcacagactccagaggctttttcctctcctggaggttaaagaggcatcacatatgtttaaaatctttaatttatatgtcacctttgtccttccttttaacttcatttttctcttatccagcatttagggactcatctttagggaggttcaaaggaaagctcatggcctttagaactggaagaaccatgttccagttgggacttgatcatttactaattgtgggattacagccaagtcacttcatccctctgctgtaaaaaaaaaaaaaaaaacaaaaaaaaacatatgatgacatttgtggaatggctccccaagccaaagagggcaaatattgtcacagctcatttcttctctcagttaattacttgcgtcctcggctgcctggctggcaggacaacctatattcgcctccctcttaaagcctcctgggttggccaggactccaagcggctttgtccagaatgagtagggtggttggcctggcctcctcagccaatcagagaggactagcatctgaacactcctctgtgctattgcttctagctgccacatggggacgctgttgaaacaccggcctggtgcagttggccatatgatgcttcagggtcttctgagacttcaagaatgtgctcacagggaaggtattagctctaaacacttgcctctgctagtttacatcacagaacagacagacaagactgttttgctccctcagctctctccttttcctagcttcagtcctggggagctcagaagctacagtttgttttttgttttttgtttttgtttttttcttgagggagtcttgctctgttgcccaatctggagttcagtggtgtgatcttggttcactgcaacctccgtctcccaggttcaagcaattctcctgcctcagcctcccgagtagctgggactacaggtgcctgccaccatgccaatctaatttctgcatttttagtagagtcaggatttcaccatgttggccaggctggtcttgaattcctgacctctggtgatcacccacctcagcctcccaaagttctgagattataggcgtaagccaccgcacccggccagaagccacagtttacaaatctgggggatttggggcatgggaacagaaacagaagagtcccaatgaaaggaagataccagctgagctgcccactctcccagctgcagttctcctgcccacagcaggccctagctgggacagggaggagccccagccttaaatcaaattcagaattttgtttatgacataagactgcacatcttaattactgaattaagactatattttccaacctatcatgactataggtgcagggcaagatcaaactccagtgtatgtggggcccgcagaagagatttaaagaaacagtgggggcagaaataaagctgtgtggttatcagatcccatgagtcttgtctgtaaggatgatggttacagtcgggatgctccagagtgcaaagccacatctcaaccagagttagtaacaagggagagtttactggttcatgtgaggaagagagaggaaggggagggctagccaaggggctggatgcaggaaggagggtccccagggttctctgtccccctcctgtcttccatctctgcctctctcagcaggttggcctaatttcctccgactgcagagaagcacacaagctgtggcacctggtgctcagactcacactgcaacacttccaccagtagatggcagagaggtactttcctgcctgttcagccacgaaaatcccaggggatggctctgactagcctaagtcaggaacctgctgtgggcaatcactgtagcattaagatgggggccagtgatggagccggtctgcagcacatgctcagcaaaagacaaaacccgcctgttttagatcactccggctgcatcacagagtgtggattgaacaggcacagaactggaggcagagaaacaagttaggcagctgcaggcataatccaggcaggagatgacagtatttgaaagaaggagtgggagcaagtctggagagaagtcgatggatccaagagatttttagaaggtagaatgtgcagaacttaattagttggtgcagtgggttgaatggtgtctccctaaaagatatgttcacctggaacctcagcatgtgaccttatttggaataagggctcttgcagaagtaagtaaggtgagaatcttgaggtgagatcgtcctggattacagtggaccttgtatccaatggcaaatgtccttataagagacagaaaaggaaaagaaagagacacagggaagaagatgtgaagatggaggcagggattggagtgatgcagcctcaagccgcagaatgcctggagccaccagaggttgggagaggcaagaaaaggtcctcccctagagccttcacagggagtagcgtcctgccaacgctttgattttgagctggtctccagaactaagagagaatagatatctgtttttctaatccaccaagtttgtggttattttgatgcagggcaggcaagcccccaaattgggttgtagcctgagagggttcttgggttcattcaggaaggaattcaagggcaagctggtggtattagacagcaacttctgttgaagcagcagtggacagcagcagcagaggtcctgctctttgcagagcagggctaccccataggcagtgtgcccagagtagcagctcgaaggcagttctgtagtcctatttacacccacttttaattatatgcaaattaaggggcagattatgcagaaaattttagaaaaagagtgctaatttccaggttgtcgggttgttgccatggaaaggggccgcaacttccggtgaactccatagtatgtggcacacactggtgggcgtgtcccatggaaaggtgcttccgccctgtacctgttttagctagtccttaatatggtccagtatccgcgccctgcctttggagtcaagttcaacttcctacctcaattgatgatagcagtttctgaaaactaacacatgtagatataaatataagtccttaagtctatcattattatgcatatcctataggggagtcatcgcgaatgaaactgaacttattgtggttcattcattcagatatttatttaaaaatatttattaaagcttactgtctgccagtccgatactgcactaggtaagtgctggggttacaaacagaacaagatagacagattagttgcccgcatggaacttatatctagtgggaagagaagcaaaaaaaaagtaagcaagcaataaacagtaaaaaaaaaaatactgggatttgagccataaaaaaagaaataagatgcagaaatcagcaataaggaggttggggagaagatccttctttagaaagaattgccagagaaggtggttgggataggcagaaaaaatagtaatattcctcttttatcttcacctatattagatgatcaatagatatttcctgagaaatgaaggactgagtatattataagaaggtatgattaaaaacaatcaccagaatgaatggctaacaagcacatgaaaagatgctcagaatcattagtaatgaaagaaacacaaattaaaccacaatgagataccacttcacacataaaaaggaattaacacttgctggtgaggatgtggggaaatgtcatatttccccacagcagccatagtacactgctggtgggaatataatatgatgcatctgctatggaagagaatatagtggctcttcaaaacgttaatcctagaaagcctgggcatggtggctcccgcctataattccagcacttcgagaggccaaggtgagaggactgtttgagcccaggagtttgagagcagccttggtaacatagcaagaccctgtctctataaaaatcaaataaaaaataaatagaggaaaagcacattaatcatagaactgccatatccaccacttccactccttggtatataccccaaagaactgaaaacagctattcaaagaaatacttgcacatgagtgttcagattattaacggaaaccaaaaggtggaaataacccacatgtctaccaatggatgaatcaataaacaacacatggtctatccatacagtagaatattgttgagccataaaaaggagtgaagtgctggtacattgccagaacatcaaagacccttgaaaacattatgctaagtgaaataagccagatgcgaaaggacatgaattatatgatttcattgatataaaatgtccagaaaaggtaaaaaatatccattgagaccaaaagcagattgtggttgccccggactaaagaaagagtaattacttaattttcctgggggtttcctcttggcatgatgttctgtatacaggacatacaaaaagcctttattttttattcttagcaaatacttaattagtactcaccatgagctgggcatgttctaagtcactttccaattactaacaaatcacttaattatattgacacaaaaagaatgggcataatgcataaagcaaatacgaacataaaaaagaaatctccctattaatatcatttatgttgaattcaatgcagggagcatttaaataagataaagggagatacttcataatccacactggtcagctaacatcatgactatctatgcagaagataaaccagcatcaaaactcataaagaaaaatttatagagagtaagaaaaaaatgaagaaacagtttagaggtaggtaatttgaatttactgttcggtgcataaaagaacaaataggccagacgtggtggctcaggcctgtggtcccagcacttcgggaggccgaggcaggcagatctcgaggtcaggagttcgcgatcagcctgaccaacatggtgaaacctgtctctactaaaaatacaaaaaattagctgagtgtggtggcgtgcactgtaatcccagctactcaggaggctgaggcaggagaatcgcttgaacctgggaggcaggctgggcgcagtgactcacgtccgtaatcccagcactttgggaggccgaggcgggtggatcatgaggtcaggagatcgagaccatcctggctaacacggtgaaacctcgtttctactaaaaaaatacaaaaaaattaaccaggcatggtggtgggcacctgtagtcccagctactcgggaggctgaggcaggagaatggcgtgaacccgggaggaagagcttgcagtgagccgagattgcgccactgaactccagcctgggtgacagagcaagactctgtctcaaaaaaaaaaaaaaaaaaaaagaaagaaaatacaggccacacagatggggagatgataattgcaagttatatatttgataaaggactttcattcagaatatatgaaatagtcttacaatttaataaaagaggacaaacaacccagtaaaatgtaggaaaaatatttgaacagatgtttcaccaaggaaaaaatacaaatggctaatcagcacatgaaaagatgctcaacatcatttagtcattaaggaaatacgaactaaaaccaccataatatatcactacacacctgccagaatggctataattttaaaaaaatggacaatactgagtgctggtaaggatgtggaaaaacagaaactctcataccttgccagtggcaatgttaaatgatacagctattctggaaaacagtttggcattttcttaaaaatttaaacttattatatgacccaacaattccactcctaggtatctacccaagaaaaataaaaatacatgtccacacaaggggacttgtgcataatgttcatatcagccctatttgtaataacaccaaattggaaggaatccaaatgtccattaactatgaatggaaaaccaacattcttacaaataattcaacaataaaccttcatgaaccttagaaacattattctgagtgaaagaaaccagacacagaagaccacaaggtgtaggactgtatttatttgacatttctagagaaagcaaaactgtagagacagcagatcagtgactgccaggggctagagacggaggcaagggttgatacaagcaggcaggaggttgctttctgggctgatggaaatgttcttatgctggattgtggtaatggttcacaactgtataaattaacaaaaaattatcagactatacccttacaatggtatgtacatttcatccaagtaacgctgctttaaaatttgaaattaagcacctaatgatattaagaaatgaataacaaaataaacccaaagaaagcaggggggaaaaaaagcaattggaaaagatgagagcaaaaataatgaaaaaaaaaacatctataatacatctagcggttggttccttgaagaaaaagaaagaaagaaatgaaaaaatcattaactatcctaataaagaaacaaaggagaaagaacaaatatacaaaataagaattgtgaatgaaataattgtagacacagaggatatcaaatgagtgactcctcaatccctctgcaaatagattcaaaatcttgaccaaatggatgattttctaggaaaatataaattaccaaaactgaccaccaaagagattttaaaaatcagaaaatatcgtttatcacagagatggtaaaaaccttgataaaaagtcatttacccagagaagcatctggttccaacagctttgcaagtgcatcctattaaaactttattgattggcaaacgctaattttttttaatttttatttttaattatactttaagttctagggtacatgtgtacaacgtgcagttttgttacatatgtatacgtgtgccatgttggtgtactgcacccattaactcgtcatttacattaggtatatctcctaatgctatcccttccccctcccctctccccacgacaggccccagtgtgtgatgttccccactctgtgttcaagtgttctcattgttcaattcccacctatgagtgagaacatgcggtgtttggtcttctgtcctttcaatagtttgctcagaatgatggtttccagctgcatccatatccctacaaaggacatgaactcatccttttttatggctgcttagtattccacggtgtatatgtgccacattttcttaatccagtctatcattgctggacatttgggttggttccaagtctttgctattgttaatagtgccgcaataaacatacatgtgcatgtgtctttgtaacagcatgatttataatcctttgggtatataccctgtaatgggacggctgggtcaaatggtatttctagttctagatccttgaggaattgccacactgtcttccacaatggttgaactactttacagtcccaccaacagtgtaaaagtgttcctatttctccacatcctctccaacatctgttgtttcctgacttttaatgatcgcccttctaactggtgtgaaatggtatctcattgtggttttgatttgcatttctctgatggccattgatgatgagcgttttttcatgtgtctgttggctgcaaaaatgtcttcttttgaaaagtgtctgttcatatcctttgcccactttttgatggggttgtttgatttttttcttgtaaatttgtttaagttctttgtagattctggatattagccctttgtcaggtgggtagattgcaaaaattttcacccattctgtaggttgcctgttcactctgatggtagtttcttttgctgtgcagaagctctttagtttaattagatcccatttgtcaattttggcttttgctgccattgcttttggtgttttagacgtgaagtccttgcccatgcctatgtcctgaatggtattgcctaggttttcttctaggttttaggtcggacatttaagtctttaatccgtcttgaattaatttttgtataaggtgtaaagaagggatccaatttcagctttttacatatggctagccagttttcccaacaccatttattaaatagggaatcctttccccatttcttgtttttgtcaggtttgtcaaagatcaggtggttgtagatgtgtggtattacttccaagggctctgttctgttccattggttctgttctgtctctgttttcgtaccagtaccatgctgttttggttactgtagccttgtagtatagtttgaagtcaggtagcatgatgcctccagctttgttcttttggcttagaattgtcttggcaatgcgggctcttttttggttccatatggacgttaaagtagttttttccaattctgtgaagaaagtcattggtagcttgatggggatgccactgaatctataaattaccttgggcagtatggccattggcaaacactaatgtttttaaactgttctagagagcatggagaaaggagaaaaccttccaaattattcctgtgaagcttgcatgtcaatgattccataacaataactatagaatcaaataaccacaataaaagaaaaacacagaccaactccacttatggatatagatgtaaatattctaaatacaatattagctgatagatctaacactgcattaaaagatttgtggaaggagttgttcaatattaggaaatccactctgtgattatctcaagttagcaattagatgtatattcaatgctgaaataacagaagcaccccagtttagtcagaaataagacccaattacccattatcaccaccaccatttagtattgcactggggaattaccaattcagttagacaagagtggggaagaggtacaaaaactagaaagaaggtggcaaaaacaatcattgactgtatgattggaaaaaataagagaatcaattgcaaaaccattagaaagagcaggataattcaggaagctcagggggcacaaaataaatgtttttacaaaacaatatccaagaatctatattaacaacaatatctttgagatataattgaatagaagattccatttacaataggaaaccccaaagatagaacacccaagagttgcacaaaatttacacaaagaaaatctaaacaacagagggacaaaacggaagatttgactacatgcaagtatatttcctagtcttgggtagaaagactcatctgcataaagatgacaatccttcctgaattaatctataaatttagtataattccaatggaaatttcccttgttttgttgttgttgtgctgtttttgttttgttttccagactacactgaatgccaaatattccatttagtgattttcttcttcccttttcctttctaatgacatattttgtgcttttcagacctgcctttctttctctcggcaccaatgaataaagttccagctttaaggcttgaaaaatcacagcaaagttgcagcaaaattaaaaggaaaaaaatgttctttttttttcctgcagctgcagagagtggcagatagcatcctgcgtgataaacgcctattcttggctaggcgcagtggctcacgtctgtaatctcagcaacttgggaggccaaggcaggcaggtcacctgaggtcaggagttcgaggccagcctggccaacaaggtgaaaccccgtctctactaaaaatacaaaaattagttgggtggtggcgcacacctgtaatcccacctacttgggaggctgaggcaggagaattgcttgaacctgggacgtggaggttgcagtgagctgagatagtgccactgcactccagcctgggtgaaaagagtgagactctatctcaaaacaaacaaacaaacaaacacctatccttgcctatgtcattttaacaaaggaggaagtaaatcccctggatttcagaggctgatgctctgcccaagaaaagcaaccctaacttccccaaaggctaaaattcagactgattggctctggcagagatatttaaattgatacctctgtttcctcaaaggtataagcctttgcgaactttctttggtttctctcttctctcacaggaggcaggggataaacaaatatgttagatttcttatttaaacaaagagcttgagggttttgcctcatcgaaattaacagagacaagttgatgctaatatttttatggaaaatcgaatatgcaaaaatagccaaggaaattccagggaaaaagtaatgaaagaaaatatcaccaaaagatgttaaaacattttggaaagccacagaaattaaaagtgtttgatcctagcatataaacaagcagacaaggggctgggcatggtgactcatgcctgtaatcccagcactttgtgaggccgaggctggtggatcacccgaggtcaggagttcgagaccagcctggccaacatggtgaaacctcgtctgtactaaaaatacaaaaattagccaggcatggtggcacgcacctgtagtcccagctacttggcaggccgaggcaggagaattgctggaccctgggaagcagaggttgcagtaagccgagattgcaccactgcactccatcctgggcgacagagcaagactctatctcaaaattaaaataaacaaacaaacaaataaataaataaacaggcagatagatcagtggaacagaataaaatccagaaatagactgaaaacattcaggaaaacagtataaaataaaggggacatttcaaatcaatggagaaaagattagttatctcagaaatgaatgggacgattgagtagactgggaaagagtaaaactggagctctacacacaccaaaatacattccagatggggctaagattttatatatctatatatgtttaaataaagccatgaaagaactagagcaaacatgagagatttatttttataatcccagacggtggcaatctttccaagtgtggcacaaaagtcagaaatcattaaaaaaagactgataaatccaactacacaaagttagacatttctttatggcaaaaaatgctatcaaaaagtcaagagatcaatgataatgggggaaacatttgtaacacatacaataagctgtccaattttttaatagtcaaagactttaacattaagaaacagaccagctggctgggcatggtggctcgaggctgggggatcacttgaggtcaggagttcaagatcagtctggccaacatggcaaaaccccgtctctaccaaaaatacaaaaattagctgggcatggtggggcatggtggtgcatgccagtaatcccagctactcaggaggttcttctggcttctcagcttgcagacagcctattgtgggaccttatgattgtgtgagttaatacttaataaactcctgtttatattatgtgtgtgtgtgtgtatatatatgtgtgtgtgtgtgtgtgtgtatacacacatatactggaatatatgtatatacatatatacatatatacacatacatatatatacacatatacatatatacacatatatatacacatatacatatatacatatatacacgtatacatatatacatatatacacatatacataaatacatatatacacatatatatacatatactatatatatacatatacatatattcattccattagttctgtccctctagagaaccctgatgaatacagtgggctacacacctattggaatggccaaaacccagaacactgacaacaccaaatgctggtaaggatgtggcgttttttatccgcattcattgctgatggtaatgcaaaatagtgcagccagtttggaacacagtttggcagctctttacaaaacggcgtgtactcttaccatacgatccagaaactgtattcctaggtatctacccaaaggagttgaaaacttgtaaccacacaaaaacttgcacacagatgctcatagcaagctttatttattattgcccaaacttggaagcaaacaagatgtccatcagtaggtgaatggataaataaactgtggtgtatccacacagtagaatattattcagtgctaaaaagaaatgagctatcaagacatgaaaagacatggaggaaactgaaatgcatatgactgagtgaaagaagcccttatgaaaagctacatactgtatgactctaactatgtgacattctgaaaaaggcaaaactatggtgaaaacatcagtggttgccagcagttgagacgggtggggggaagataaccaggtagagcatagaggactttaagggcagcgaaaatgctctgtatattactacgatggtggatacatgtcattatacagcaggtccttggatgacactatctcattcaacatcattttgctataaagttgatgagaaaaaaaagtcaattcctagccaggccactgtctctgtggagggtgtgcgttctccccatgtctgtgtgggtttcctctgggtcctccagtttcctcccacatcccaaagctatgcacggtaggtgaactggcatgtctacatggtcccagtgtgagcgagtgtggaagtgggtgagtgtgccctatgatggaagaggaccctgtccagggttggtgtctgccttgaccctgtgcctctgggatgggctctgccatccacagctctgaagtggaataagccagtcaataatattctcgcttgttttttgttgttgttgttgtttgtttgtttttgtgacagagtctcactctgttgcccaggctaaagtgcagtggcactaactcggctcactgcaacctccacctccagggttcaagtgattcctgtgtctcagcctactgagtagctgggactacaggcatgcgccaccatgcccagctaatttttgtatttttagtagaatcaggattttgccacgttggccaggctggtcttgaactcttgacctcaggtgatctgcctgcctcagcctcccaaagtgctgggattacaggcgtgagccaccgtgctcagctttcacttgtttgtattaatctttcctaaatgtatgtatggctcacatttatttcaatgtttagtattagaagtgtttgaggtctttgtaagtttggtgatgttttgtgaccagaaacaggccataggaacttaactcttgtttatattaattagcttatggtaaaattggataaatgttttataagagacatgaaagggcatacagacacacaggagagaaggccacgtgaagatggaggtggaggagacagtgatgcagccacaagccaagggatgcaagcggccacctgcagttgagagaggcaggaaggatcctcagaaggcatggagcctacgaggaagcctggccctgctggtaccttaattttggacttccagcctccagaaccatgagagattacatttctgttgtttgaagccactgatttttgtggtcattggttatggcagccacaggaaataagataatcacccacttaattttcctagaaaagctgtgttttgaaagtcctcttgaagcctgggttcctctctctgcatctcccagttttccctcaaagcttgtggattctccattcctcacattaactcaggcctttcattgccaagtgaccccgagtcctgccttcgcgggtgctgggggagccttcctgacccactggaagtggacctgcccatctccttgctgtgaaactgcatgagggggcttgtgtctgaggattgtctggcgtgaggggagagacaccacgtggggacagaggagtggatgagcaggccggggcatgacggggccgtgacagggacctggccttccattctgtggaagcctgagacaagcagcaacttctctcattcctcctctctatgacaagacaggaactgggacactcaccttactaccctaattcgctgagcctcggaagaaaagcagcttagatttttaatcccatccaagatggaggccctcctgctcctgctgccttgttctcaccccctttcgtgatgtgcgaggccatcggaaggtgtggaatttctccactgattcctctcattgtccctttctccctactcctggggaggctgcaatggtgacctcatccaccttcagaggcaggtgctggaggaggaaaggatgtgggagttcaagccggctgcagaggcccaagagcccagatggtgtccttccagcaaactggagaggcactcctcctaccaggcagccactgccccactccagggcccctggctcagctagggaagtggggctgggtttcaccccctgctcatcccctaaggcccagtgctggactcagtgcagcacctgcccagccatctctagcagcggcataaagcataaaatcaaggccaatgttacgtgctgccttgacatgtggtaaaatgtgaagggcctcaagtggcctaaatgcaagctcctgtcccacctctgctcccataaatagggtctcccagctgggcaacccttctcatcccagggaccaggtaccacccctgtttgttgccaagtagcaggcttcagttccctgccagtctgcggaattatttaacaacctcatgaagaaaccaggggccactccaccctctgtattagcctgttctcaggcagctaataaagatacccaagactgggtaatttataaagaaaagaagtttaattgactcacagttccacatggcttgggaggcctcagaaaacctacaatcatggtagaaggggaggcaaacatgtcctccttcacgtggcagcaggaaggagaagtgctgagcaaaagggggaaaagtcccttataaatccatcagatctcatgagaattcactcactgtcatgagaacagcatggaggtaacccccacatgattcaatcacctcccactgggtccctcccacgacatgtggggattatgggaattacaattcaagatgagatgtggaaggggtcacggccaaaccatatcactcttgttactaccaaacctgctgtccaacaaccctgctgttcactctgctcttgagcaccacctcatgtggccctgcatagcctgcagtggcccttcccctgggctacgagtatatgtgactagaaaattgccgtgggtctcacctatccagtgttgggtgttgtgtgtccagccctagagtgggactccttccctcacgaatggggtgaatagaaggtgataaaaagatctgagtctagggatacctaggaggtggaatctcttctccatgcatagcatgagtgatcacaggcctgaaaccaaaagggacttaggtctgggggagagattattttccaggtgctgaatattcctgggataggggagggagctaaacaggttcctgcccaaaggaagtgagaagggggtcctagcaacttctcagggatttagagctgtgactccagggcctttgttcagaggagctaccttgcaaggaacttctagaagaatgcttctctttctcagcatccatcctcccatttcatagtcgtgcccacgatgggccccgtctccctgaacttgatggctgaatagaagtgtagcctcccaggggcatctaaaggcactcagagccccttacccagccccagcaggcacctgcctggctgcccggtcctcagggttccctgtgcattgagcaatatcctcaaagtgaccaccagggggcagcagcacccagactgccttccactgcacctgcagatcaacaaattccagtattttgggggaatatctgtgataacttggctactgctttactgacctcaggtaaatagacagaccaatgtgcttgaggagccaattgctttaaatctcctgactcattttttgtattaagatttgttttatttatgcaattattctgtttactcaaagactttaccagaagctgggtgcagtggctcatgcctgtaaccccagcactttgggatgccaaggtgagaggatcgttggagcccagacattggagaccagcctgggcaacatagtgagaccccatctctacaaaaaatttaaaaattagctgggcgccactcatggtggctcaggcctgtcatcccagaactttgggaggccaaggcaggtggatcacctgaggacaggagttcgagaccagcctggtcagcatggtgaaaccccgtttctactaaaaatacaaaaattagctgggtgtggcggtgggcacctgtaatcccagctactcgggaggctgagacagcagaattggttgaatctgggaggcagaggttgcagcgagccgagattacaccactgcactccagcctgggcaacagagtgaaactcagccctccatcccgaccccagaaaaaattacctgggcatggtggtttgagtctatagtcccagctactcaggaggccaaggtgggaggatagcttgagtctgggagggtgggagtctggcttgagtctgggagggcgaggctgcagtgagctatgattgcaccactgtactccagcctgggtgagagagccagaccctatctcaaaaaaaaaaaaaaaaagtaccagcccctatctacccattcatagctttatgtccatttcttttgtcttcaagcactggtatcctttacttatctctcctcacctgatctagtgtttacatctcatttgcgcccatagagaagtcatacactgatgtggattttagatagggcacgctctcaagacagccacatgtattattctgtgctcacacagcctggcctggagatgcaaagattatggaatccagaatctaaatgagaggatcagattaatgggatgttctcacagtgtcaggtgaggacagcctgatgcagcctttcatcatgaggctgggacctctgggtcccttggccccaggaccacactcgaggacatgcctgttcctgccaacatggctgggcagagttcctcttttctttccttttcttttcttttctcttctcttctcttcttttcttttttctttcttttcctcttttccttccttccttccttcctttcttctttcttctttctttcttttctttctttttctttttccttctttctatttttttttgaaatggagtcttgctctgttgcccaggctggagtgcagtggcacactcttggctcactgcaacctccacctcccgggttcaagcgattctcccacttcaggctcccaagtggctgggattacaggcacccaccaccacacccagctaatttttgtacttttagtagaaatggggtttcgccatgttggccaggctggtctcaaactcctgacctcaggtgatccacccgcctaggcctcccaaagtgttgggattacaggcgtgagccaccacaccctagccctgagtctgtttatgcttctgtcaggtgtggcatgggcctgcctgggagctattctttttctgtaaagcacaggcagttaatcagtggtctctgggaagaatccagctcagggttatatttcgtttgacccactcaagttttaaaaagtaaattagttgccaatgtgcaaacattagaagagttcacagcttctccaacaatacctagaagttcatccgatggtgcccgcattccctgctctgtctagatggtgcccacattccctgctctgtctagatggtgccgacataccctgatctgtccagacagtgcctacattccctgctccatctggatggtgcccacattccctgctctgtccagacggtgcccacattccctgctctgtctggacggtgcccacattccctgatctgtccggacagtgcccacattccctcctccgtccggaaggtgcccacattccctgctgtgtctggacggtgcccacatttcctgctccgtccagacagtgcccacattccctgctgtgtctagatggtgcccacattccctgctccgtccagacagtgcccacattccctgctctgtctagatggtgcccacattccctgctccgtccggacggtgcccacattccctgctccatccggacggtgcccacattccctgctccgtccggacggtgcccacattccctgctccgtccggacggtgcccacattccctgctctgtccagatggtgcccacattccctgctccatctggacggtgcccacattccctcctctgtctagacagtgcccacattccctgctccgtccggacggtgcccacattccctgctctgtctggacggtgctcacattccctgctctgtctagacagtgcccacattccctgctccatccagacggtgcccatactccctgctctgtctagatggtgtccacattccctgctccgtctagactgtgcccatattcgctgctggctgcaaatgcgaggagttgacagcagcctcccctttacaaggcaggaggtgccactgttcgccattgtctccacctagggcttcacttgctttctatctgcagacatcagagggacccacatctctctgttctgacacgctgtgtgttgatggcagagtttaattatccacatgcaatcttactttccttattcccaagtccgtggggctgcctcatcaaagcattgtaagaactgataaccatcttctagaagtatcatagtgatattaagaacacacatcacagatcatagtaaatggctttaattttttagcgaaatctcactactgcaaatgcattgttgtcctagctaatgaatgcatagagtattgcctgcaaaataataattgagattctatttttaagaagcttagaacagtacatggtgcatagcaaagactctgtgtatgtgaagccagattttaaaatatggtaacaagtgtctgaaaatatgtggctcaatttgtctcccggttacttttccctctccccctttaaaatgtagaggaaggagaagaagagataagaggtttgtgagtgaagacaagggccctttaaggcctgggaagactaacgccatagggatctccctctgccttaaaaggcacaggaatcttagtggggaaaaagaagtggtgataaatagccagtccgtgtgcctggaatatcaaagtcagtgcgtgccagggatcacactgcgggtcacgtgcactctgggtctctctctgcaaacctgccctgcctcagtctgggaatatgcaactgcctaagaagggtctggcttacacaggggccatgagacgtggcaggcatagctgggctgctactggtcatgaatcctggacacggcaggcaaggtgtggtgtccatatgcattattcgggtggggcaaagatcacagctctcactagactttcagaggactttgtaacccaaagaaccactcatctcaaggactgtggtaactcaggggctgagccatgccagtgtttattatgtgaaacaaggactggaacctcacaagaccaagtctgtccatttgaggatggcccaagatgcacacgggctgcttttatcttatgcgcaggttttaaaaaaatatgtttcatttaaatattccatactcttcaggaatgcccaggcagctgagctttcaggatgtcgcattgcagaggactccaatgctacatatggcagctggagaccctttcaaggcaggtggcagaacggaggccctctctatctgctggggcagccctccgggtgccccgctggaaggcagagcagctccatctctgggtgggtgagaggtgctgcatgggctcactatagtatcccaatactgtatggcagtaggctgccagagtatcctaagctgggtggcttcaacaacaggtactgactcacagttctggaggccaaaagtttgaattcaagcagggctgtgcttcctctgaaacctgtgggagaggagccttcctggcttcttcccgacttcttgggatggggatgcgcatccatcctcggccttccttggtttgtggctgtgtcactgcaccctctgcctctgtcacggcatggtgtcctccctatgcatctgtgtctgaatttccctcttccgataaggactccagtcatattgggtgagggcccaccccaatgacctcatctcaactagatcatctgcaaagactctatttcccaattaggtcacattgaaggtacctgtctttttgggggatacaattcatctcacaaaaccggcccatcacctcaaaaggacctgccaccccagtgctatgtgtccctctctgcccagagccactccttcccctggctctcggggagtgggggcacctttccctgctcccacagtgaccgagcaccttccccttggtatgcattctgaagggggcatttttttctcctccatctcagccctgtacaaagcaagttctttctagattgaggtgtgtatgtgtgtctatgtatatgagtgtatgtgcctgtgtgttttcagggagatgtgtgcaggatgggtgcaagggaggagtggaaggcggaagggcaggaggaggatagagccacaagagtgagcacagaagtgacaagggcagaatcagtgtgtgcttgtgacaagtatggaaatgtcatgcctttaggttcagtcctataaggtaggtgtatcagtaagggcattgattctgcgaccttaacagagatcagaataacagtggcttaaggaagagtggagtggatttctctctcctgtaaatctggcctggtgggtggtaaggaggatccacgtcgcccaggcccagatgtgtgtggctctttgagtgccccgttctttcccagtctcataactgctctccacctcctccacatccagccactgggaagcaggacaaagttagttaagggcacgttcttttctttccaaggattacttggacattacagtcttcacttccatgcctactggccagggcttagtcacacaaccttgctagctgcaagggagtctgggaaatgcagctgctattctcagaggccatgtcctcagggattctgctaaatttagcaaggcagggacagatatgggggaaccactgacagtctatcacaaaagaacgtgattttagagaaacagtgaaacagtgtcattaatccacccctcaccccttacaacagccaaaaagaaatccagtggtatccatcacaataaagagtatgagaggaatgtgattagaaaatcaggttgccaggcaggatgtgtccagttttagccagtggtggggttcatggggaaggcttcgtctcaggaaggtgtgtgttgggttgttctatggccagatggttttcaacgacatagcacgacctgtagctctccaggacccgggcctggacataggccttgtccttctcttgccaggcatcggactcgatgtagacgttgaatgggtcgttcgagtgctccagcttcttggagcggatgtagctcagcatgatgcccagggtgaagaagccgaagaatcccagtaccatgaggacgtagagggcctccagcttgccgtcactgctgcggggggacctgcgggccaggcccgacatgttgccaccctgctgaactgtctcctgccacagcttggtcagaaagggcgtcaccgctgtggtgttagacaggatcatcctgggcattaaggttccactgctgcagctcaaacttcccaggcacacctcttaaaggaaaaatgcaaccccaaatcaaaaagtacgtattggccaaaacccacacgtacgcacacacacgtatacaattttaaaatctcaggtgagaggggtgagctgacactccacaggccatggcatgtgccatcttgggtctgtgcaatgccttctcttgatagatgaatggatacattgattcccttctatttccatccaccactccaatctccacccctataggtgaccatcagaatgagcttctttaatatgcattgctgcatattgtggcgtatggggttgcatgtgtgcattttggtttacctaaatggtatcaggtcatccaactcattcagtctccttctttttcctcatgactgtgcttttgtggctcacttgcggtgctgggtgtgtgcattcccttgcttccaatgtttgtatacaacccatggtgaacacccacttcttttctctgccttctcccccagagatggacactgctgtggctgctgactccataaacaagggggagacaaatatccccatccttgacctcctatggacctaaaaaaaatcacgcatctcatacaactagttcctggcagcttatgcaagactagtcagactggttgcccttggaagcaacgtgcaattggtggtctgtttcaccacagagcatctctctatgaacagttacattcatctgaatgaaaaatctatgctgtcacgtggtggacttcagaatgtctagggagatttcacagagagctcccctcgaagaggccagtgttgtagcttgtgctatgtttttccctccctgcccacacacaggcacacacacgtacacaatcttaaaacctcaggtgagaggggtgagctcacatgctccctagtccatggtatgtgccgtcttgggtctacacaatgccctctcttgattgagcaatggtacatggattgccttttatttccattcactactctctggctatgcagaaagtgacattttccctatcgtttaatcttgatatcactgtccctgtatactcagagtgggcctgggaattggaaaaattgtctccaagtagctgtaagattctgtcaggggtttggtttgctgtggaaaccccatctaggtgaccttgagatcattggtaagctgaaaaaaaacaggtcttgtttttatttatttatttatttatttatttaggtttgagcaaatgccagcctctacccccagttcctgctgggaaacaaaagctccgaggccaagttgttgatgtcacattccaaactcaagccagagggggccactgggagcttatcacacgtaagtgctcccactcagttctttctttttctgttttattgagacagggtctcactcttgtcactcaggctggagtacagtggcacaatcttggctcactgcagcctcaacctcctggactcaggtgatcctcctaccctaccctccagagtagatgggactataggtatgcaccaccatgactggctaattttcgtattttttgcagaggtgaggttgccctatgttgccgaggctggtcttgaactcctgggcttacaggatccgcccacctcggccccccaaattgctgggattacaggcatgagccaccctgcttgtcccctgctcacttcttaggagcttaaagtagctgagtagaacatggcctggagtagaacatggcctcggggggactgttgtaactacaggtgaaggatgtatttgggaagacagtttatggccagaatcgctatggaaagacaaattccaacacttgccgggacggcgctgtctttcccagccaggatggggactgtgacattgcacatcatcttgtgtaggacaaataacctcagaaacctagctcctctccagcttagacccagagctatttcttcattgaattggtttaattgtaaaacataccctgaacccagcaccagctgaagacatctggcacctttccgaggcccctcttcctctacccatctctgaactctggctgtgtctcagagttctgttacctgctctcttctcttcctactctcctctctcccagggtgactgcacctgctccaggctcacctgcatggccatgacccagggctctctttaagctccagagccatgcgtccagtgacctgctaaggagatggttccctttggccatccccaggctccttaaagttaacactccaccctgtcctgtcagagactggcccctgcctcattgagctgagtggcaacaccactcactccaaaatctgcatcactctcactgatgactgcaatctcatcatggcagttcccatcttgaaggccttccttggctcctccctgccttcagggtgaaactcctggtcttctgcatggatacaggccctaaatttgagagtctatgcatccctctccaattccactgttgccactgtgcccagaccctatgctccatggtccctgctggccccggagcctctacacattgtgcgtctccagctcagactgcgccttcttcttggcccatgaaacttctcagcaatgcctactcatgcttaaaattcagcccagctctcacctccttcccgaagcctgctctgatacatggggctggatcagcactgtgcacaccatgacccctgctaacctcatcatggtcaggatctccaggccccttatcccatccctgccctgccatccagcctggtgctgggcacgcaaccacacaggagctgcccatgaatgtttatcgaatagatgccaccagaacttaataccttttgaccagtggggcttgactctttataacctgcttactccaatgaacagatgccaatgagctgtctccgaagctctaactgactcccttttccagaagggcagtcatctcccaccctgaaccacagtctcagaaggcaggagtgaggagcagaaagagctcagattttgggattccactgccgccacaggtttggattctagctttgctacttcctggccacatgatcctggacagtttccttagaattgttcagtcaagttttttttttttttctttccaaagtagcgagaaacaccactgacatttgcgggctgttgaatcactgagcaggtgtgtagagtggctgacagcatgtggcacatggcaggtgcacactcagtggtcctgggtaggagtttattggtttttctacctcattaagaaattgctgcccaaggatttggggctttgggggtttaggcttggctttcctgtggctgaccatggcagctgtcttctctacgttgtggagagatcagacatgaatgagaatcaaagattgtttgtggcctttcctggtttctaggcttttgagtctgtgcagagatctgtcaggggttaagctgcctgggctcaagagattcaggtccttgttcttgtacaaaactagcatttagccccattctaaccatcggggtaggcaggaattgtttggtaacagatccaaactcaacgctcaaccatttctttttaaatgacccgaaaccacttatgaatgcataaaaccctgccccagaaaacagacagacctggacctgatactatgatgtaatttccaaaaacccagaatgatcacaattggcaaataattctgccaccaatcactgttagagagtctttccaacttcatgaccatgtgaaggtagaattatggcaggcgacatttgaagatccacaagttaattggtttaaaactgataaatccatacagcaaattaagagttacatctgcaattaattcataatagtgagttcactgagaaggcttgttacttagatccagatggactttcttatgtccaaagaagcaaccaaaaacatctgctttgaaaacctcccaagcccaaaccatcctcagccttgttctttagaatgctttagaatgaccttgttaaaatgcagattgctcctgtaatcccagcactttgggaggccaaagcaggtggatcacttgatgtcaggagtttgacaccagcctggccaacatactgaaaccccgtctctactaaaaatacaaaaataagacaggcgtggtggcgggcacctgtattcccagctatttgggaggctgaggcaggagaatcacttgaacccaggaggtggaggttgcagtgagccaagattgtgccattgcactcctgcctgggtgacacagcgagactctgtctcaaaaaaaaatgcagattgctgggctctattttcagagtttctgatttggtagaactggagcgggcctgggaatctgcattcctaacacattcccacgtggtgctaatactgctggtctggaggccctgcttggtgatctattggaatcaccgggggagcttttagaaaataatggttcctggatctcacccctagagattttaatgtctttggtctgggttcctgcctgactcagagactttttagaaacctcccaaatgatcctaatttgtagccaagattgagaaccactgggctgtggtgtgggaccctaggaaaatgaccaatggccttttgtgctgcagggtacctggaagaattttgcaaaaatatagaaatatgatctcactgactgtttttcaaatcttgtttgttttttacattttcttttttggccttgtttgcctctgatacagtctgaaaagaaattgcagaaagaaactctccagtcttcagtgtaacctcagctgtccccagtctcacacacgctggtgccttcaattacaattctcctgtcagagcttaagtccagctaattaactgcctttcaaatgacaaccctatatttttaaagaatttttttaaaacttcacatgtaatttattgcattgcttttgctaaatgtcctccacacccccaatgcctgctaggctgggtcgccatggtatttttgtgtaacgagtctcaaaatgagtttggcaatgtctccgtaatagtcagcatggtgtaaatgacagtctggatctgcatgtcatttgggattttatatcagattctctaggttcatttctatgatacgtgatgccaaagcacccacatgccccgtggctgcactttcagacagttggactcaaacagagtgggagagcaactgatccaacaatctgaattttcagaaaacggggctccttagagatgagatggcttgccaaaagtaatctctcctatcagaagtacatatcctcagcaaactaacgcaggagcagaaaaccaaacaccgcatattctcacttataagtgggagctgaacagtgagaacacatggacacagggaggggaacaacacacactggggcttgtcggggaaaggtgggtggggaagagcattagggaaaagagctaatgcacgctgggcttaacacctagatgatgggttgacaggtgccgcaaaccatcatggcacatatttatgtctgtaacaaacctgcacatcctgcacatgttccctggaacttaaaaaaaaaaaaaaagaaacaaaaacaaccaaccaaaaatatatctaaaatgtcatctgttagcaattgactcacatattattagtatagaaaagagcaattcccaggaccttgtacagaggaagcaggctcaaaacagctgaggaataggccacttttatcagatagcattggatccatgcacatgggggttggcttcttacctaaatatgccatcagaaatcatccttgttcctgtcccctcagcttttgtagcttgcacagtgagtaaagggatggtggaggcagaaatggtaggagccagagatgttcaaaatccatctgatgcttggcctgtgctgaacgttctcaaactgtggccttgtcaggcccagaaagtggggtggattcctggccgtttctgcttctgcctgggtgtgagatgtgaatgctgcccctactgaagggtagtgcaattttttttttttcttaaaagcttagacccagagctgctaatctactggaaatcactcaggacacagggctctgggcagctgcgctgagcgagacacctgcaaatggagaccaacggggcctccagcaccctggagttccgtaaggcccccagctgaacccaggggagaagagggcagtgggtggcgcctcgctgctctgggcacacaccacctcttctgacttctcccacgtgctccggctgtgtcgcctatcagcactgataacagcctggaagctttcagaacagaagctttcccagcatgggaaactctcattctttcttttttttaattttccaaagcctttatttctaagaccaactgtggcctacccgtgcataaactgggcaggctgtagacagcaggcttgccaagtaaatactacagccttcctcccaatattgagccctgtcccattgatcctgcaggggagatgtgtagggcatttgttcacggggaggccacaagtttgggcctctcatcactgtgacctcacacccctgttgagtgtgtcgtaaacagaggagccgttcttcaagccccctgccctgagtgcacccctctcattcttttgttattattagcaaattcccccagttcctgatcattctttcttaagcctttagtgactgggtaaggttcttgtggctgactccaagctttcttctaaaaggaatagattctaggggtgagtagaggagacaggaatgtcggagtcagagctcaggaatctgggttccagccccaggtcaatcctagataaactagagggctccttaacttacttccctaggttgactctgggtttttttatcacgctcgacaggactccttatgcatttcttcgaaagagcatccagtcttaacatcatcatttggcctcatttggtttaagaagcagaattagggtaaaagcaatgatggataagcctcattttggtgaatatgatcttattgagacaagaattctgagtcaattgtccttggaaccaactgtctattgttttcatctttatcacaacactatgtccaaattttggaaacatgtttccctcctagcagaaaagaagccacccagggccaccagatgcttcccgacacttggctggctttgtctggtcttctatccttcccctatccccagagtcagtgggtactaaggtggccagggtcgctcaaggaatcagaatgcaaccgtccagaggcccagaatcagactgtcctctctgattaggaaagtgtttcctgccacctcccagggggatgtggggtgtggcttgagtctggtgcttttaccaggagcttcccagacctctctatgggtgatggagagagagtctggggtgtgaaggatggaaatataaacactgaatactcagagagtcaggccagcgagctggtgaggagactccatcaaactcaatatgaaaacatgggtgggcgtttggggatggataatgaatgacagctgaagtcacacatcaggagggaaggaaggactctcatcttcagcaaatgacataaatctggggagcctcagtttcctcacctgaacagtgagaatgatggaatctaccctgagtatatatcccaggtctttgtgcagcatggaatcagcccccagccttcctagctcttgctttattttattttattttagagaccaagcctcgctctgtcactcaggctggagtgcagtggcgcgatcttgacttactacaacctcctccgcctcctgggttcaagtgattctcctgcctcagcctcctaagtagttgggtttacaggtgcatgccactatgcctggttaatttttgtatttttagtagagacggggtttcaccatgttgcccaggctcaacatgcctgggctgaagcgatccacctgccttggcctcccaaagtgctaggattacaggcgtgagccaccgtgcctggcctcttgttttatgtttgctgttctcgctgtaggtgaggcattcccaccatattcccttgttaaaatcctactggcccttcaaatgccagctcatatccacactgtgtctgaccctgatccagaatcctctccccttctttgaccccttgtatatatctttcctacagtgctggtcatattctaacttctaatcatttgtagacttatctgggcattttctcccaggaggtcccaaaggacagagaccatcttgctcacctttgtccccactccagcacccagaatgtttcaataaggttttcctgaattaagtgggaagcaaagtattagattcaataccactacaatgggaaagtgagtgaataattgataaagtatactatgctctggaaatagattattgagtaatcagaggaaaacctcttgcaatgtagtatgtttttatatattaaattttatattatttatttatatttatattattaacatatttttgttattcattttgtttattaagatttactctatatatatatgtgtgtgtgtgtgcgcgtgtgtgtgtgtgtaacccaccttatttctaaaaaaaaattttggcagtcagtgactattaatagtagagcacaaactcttaacctttttttcctcccgctttcccagagtccttatcaccttctaatataataaagttttcttaactgtgatgttgattgttcaacgtttgtctttcccaccaaaatgtaagctattgcgggcagggatctttgcatgggttgttcttcattgcatcccaggccactagaacagtgtgtggcatatggcaggagtgcaataaacattcagcgaatgcaggcaggcatgcgtgaacgcatgaataataagtgttcctttcaactaccagaaagagcgaaaccacacaggagctgtctcaccctcagagatttaagacgacagcaatgagtccctctgatgtcttctaagtgagctttattttcaaaacaaatcattttccaataacctatcaaaagcaggtcctacctgaatatccttggctatttcctctggatacagataatgccttcctccaatggatcctcggctcacctgatgattgtgtgaaactggccagagaaagcagagggatttttcctggtgattggaaatcagagtcacggctgaatttaagcaactgtgtgaactcagcaaagtccccaccacctggaccatacatgacagctatggttattgacaaggtccttccttaatggagctggaacctccttcttaatctaaatgtggactcaaatgaactgtcaattcacatagagcaatgtgacaaatccggggggcaaagcattatgcaatagattgggcacatgcgcacgtctctgctacttagtcaactttcttctaaagtttcccacttttcccattaccacaatcaagatcagatatcagtaatattctccgcgtcccatgccctttcctcagggagatgccaaggcccaagagctggtccccccaaaaagctgaaggtctttgaaaaaaagtggggactcaggtcccgtgagtggttttgattttcttttcttttgaaagttgcatgtaagtgttttccaaatactatacaagaatgtctataacttaataatggaggaatgttttcgttgttctgtgttggtgagatgctttcctatgcgtttgttgtataagtaagtgagaatggcagaaggaaaggaggggagaggctgatcatcctatcccgtctcccactctgggggtctcggcgcttccagcctgaagcgcgcccgctgcgcgtccggagacgcaggttccaggagccccccggggttgcccgactaggccacttgcgccccggaagagggccgcggaggtgggagaccctaacttacccgggtctgagatgccgagagagccgggtgtggagctgagtgcgcgcctgccgagcgctgaggccacagacagccccgccccggggcggcaccttctaagggcctgagcgctgcacagggatgggggcgggcggggcctcccagagccgccaggcgtcccgccccactccgcccacacgcacggcccagcccagggttccccgggaccaccccagaccagccccggcccccccgggtcctccacactctgcaccccagaccaacaccaacgcgcgtagggaagcgttttagatcctgtcggagaggctcaaggccggcagaaggtttgcattaggatcgagaaagccgaccaacggacagatctacctaccttcctgcgggagtttgaggttgccaggggggaagccactgcagccaggagaaaggccgtctgggaaccaccccaccctcggacgtgcgggccttcaaatatctctgaacataatcctccaaagaccgctcaacctccgctcccgacgactctttctagcctcgtcccgcaccccagctgtggccacacacctattaggcaaacatttatgaagcacccacttactgggtgtgcagccctgagctgggggtgcagctgtggaccagacaggagggggcccggagcggcagacagtcgctggaggcacccgagccttggcgagcacaccctaacgtccttggggcctttcagcccgagccgtccttgtccagagagcaaaccatgcaatgatgcaggtgacttcccagcaaatttcatagcgttgctcaccagcttggcaggcaagaggagaaggggcagtccccaaaagacaatcccatgaaccttctagggaatgacgtccaggctccaggctcctgctctgcaggcgggtcgcagaggcaggttcctgacctaggactagaagacattctctagggtcactgcctccatggtcttccttggcaggtcacttcttcctgggcttcgacctcggtgttctcatggggacgagggtgattggaggccctccaaggggtgcaccgatgtgtcctgtgcaccaggcagaaccagcattgccctacagtgtgggtgcaaaatgaacccacatggccacgttggaaagtcctgaaatgttcatagcctatgacatgaaattgcactgtgtgaaatctatttattctttttttttttttcttttttttctgagacggagtctcaccctgtcgccccggctggagtgcaatggcacgatctcggctcactgcaacctccacctccctggttcaagcgattctcctgcctcagcctcccgagttgctgggattacaggcaccggccaccataccctgctgattttttgttatttttagtagagacggagtttcgccatgttggccaggctggtctcgaactcctgacttcaggtgatccacccgcctcggcctcccaaagtgctgggattacaggtgtgagtcagcgtgcccagactgaaatctatttattctatggaaaggatcagagctgtagaaaaatccttatgcatgtaaaagttctttgtgtttttacttgcaataactagaatctaaacatccaaaaatagaaaatataggtaattaaattgtagtacatatgatattattctacatagtagaatattatgtggagtccttaaaatgtttacaaataatttataacaacatggggccgggcacagtggcttacacctgtaattccagcactttgggaggccaaggtgggtgggtcacctgaggttaggagttcaagaccagcctggccaacatggtgaaacctgtctctactaaaaacacaaaaatttagctgggcgtggtggtgggcacctgtaatcccagctacttgggagtctgaggcaagagattcacttgaacccaggaggcggaggttgcagtgagccaaggtcacgccactgcactccagcctgggcgacaagagtgaagctctgactcaaaacaaacaaacaaacaaaaacccaacaggggagatctttatattatcatgttacgtgaaaaatacaaaaacagaaaacaaaacaaaaaccccacaaaactcaaggccttaaattgtaaatatgagatgccaggcattattcccaaaatctataaggaagcacaccaaccaccatgttaacattgtctattagtggtaggcctattggagatttattatcttatttatgctacttcatgttttccttccttttttgttttgcaacaactctgtattagtctgttctcacactgttataaagaactgcctgaaactgggtaatttataaaggaaaaagtcttaattgattcacagttcagcatggctgaggcctcaggaacttacaatcatggccgaaggggaagcaaacatgtccttctttacatggcagcaggagagagaagtgcagagcaaagtggaaggaaaagccccgtataaaaccatcagatctcgtgagaactcactcattatcatgaaaacagcatggaagaaccgcctccatgatccaatcacttcccacaaagtccctcctgcaacatgtggggattacaatttggattacaattcaagatgagatttgggtggggacacagccaaaccatatcaaactctatgtactttaatatttaaggaaaattacataaactttatctgaaaattccctggattcttctcctcaaggtcatgctgtacatatgcaggatcctcctgcctacatctccaaatggataatggattgaagcaaaatgtttgtcccaccaaacattgatgtgtaacccttttagtaacaattcatagaccaggtaaacatgtgtggagcagcccagatcagctggagggagcattattctcatcagagtgtggaacaatgcctacccatctgtcagctggttatggtgaagacagtataaatggaagcactgtcagaaactaaaggcttatgtaagtgatgctgttcttatgtctcctcttcctctctatcccacatatgatgaattattttattatgatgatgctgtagttgttattttctatttgaaatgaaaagacatgattacagatgagaaagagtgtattttgttatccttgatgcacttgaaatggtttcctcttttttttttcctgttttcttcttcttccttctctcttcactctttcattcttgcctctctccattttataggtatgattgatcttgaaataatgatgataacagaatgatagcccatatagtccttgggtcacttctgtctctatttctctttcttccctcctctttttttcttggtccttttgctagatgggttgcgagcatggctccctgcccctctcagtgggtttatcttcattttccatgagctcccacctcactgcatgtgacatcaaagccaaagctccagccacttctcatcctttcttaggaaaatctcaagtcttaacttgaaagttgaatgtcctgcttttgttcctaactctgctcacaggatagtggaagaagaaaggctcagcctgttaccagaaagaacagatctgcagtgtaacccatctaggatgaagggtcatgtggacagttcttcactcttccctctggctgcccttcagttggtgtccagatgcccctgtgttagcctgggggcattccttgccctttggcccctcttttgttgcctttttttttcttagacaaggtctaactccgctgcccaggctggagtgcagtggtgcgaccattgctcattgcaacctcgaactcctggctcaagacattctccccgctcagcctcgtgagtagctggaactacagttgtacaccaccatgccttggctaatttaaaaagtctttttgtagggatgggagtctcaggctggtcttgaacttctggcctccagtgatcctcctgccttggcctcccaaagttttgggattacaggcgtgagccattgtgccctgccctttgttgctttttcactcctttaccctgtggtttctactatcctcagggacaactctcattgccctggggcttctggatcttcagcaagacacacccctaaaggcaaacaatctttctttagcagcagcagagcacaaacggagtgttgctgtatgctaaaagcatactatttcccccccatgagaaagtctaaggggtccaggggtcctgaagtccctatcctgcccccgccagtgacaggtgatggggaacagaatgctgaaagagaccccacccaaccatctggtgtgtcaacacccgcccctgccggtgctgtccacaggttgatccccatcccacatgggtggcttaacacattcatgtcttctgagagccatttgttgctccccagtctcttctgtcctccttctttgaccaacatttccctgtctgatttgcttggtcacaggtagcctgtgtcagtttcctcccaggactctctttgcctgggaataagtgctatacatgagaagcatccctcccaagcgctgtgtgtggcctcctggcattctcctaagacttcctagaggattgctctggttccaaaaagaaaccagccacaacctgtttggagccctgcaggatgttccagctcaccgacttcattccccactacttgcccctctaacagagcttctagaccagcagccaaggtgcctccattcactgtacatgactcacctttcccccttctagacacttcttcacgggtctagcctctgtttccagctttacacagcacaggctgatgcctgccactgaacgggatccaggactttttcaaatctcagatccccagcaaaggtatgacaatgggcaccctgaccagctctgagcctctaagcatagggtctatttgtttaattttgaattcacttttaagatttaaaaattgagagctctcatcttcaacaaaattcaggggatctagaaacatcatgcccacttgtggcagagtagccaactggcagaaactgatgctgatgctgtctgtttcctacacaaaatgcaaaccagagtttaccacaggccccgctactccacattctcctccacaaggacactcagcaggctcctgtgctgaacggcttgcctggttctgtaggtgtagggtttgttactctgttccaggacctattttccgcaaagccccttacctggctgtttcttcccttggcatacgtgagaactcctttcctcactgaattgctttgaccttgctaattagttgtgttgcaagtgctttttggctgtgtcagacatgtatgttctgtttatcattttagagcaatggttattaatttttttcagattcttcttgggaattagagaaaaacttatggctctcctccaccatcctgcccccacctaccccaatgcacacatgtgcaacatttgttctacaattcaagagctctctagacccctgatatgctaggcaattcgtggctgccacattaggaacccctgatttaggggaaggactgttttctccattagacaaactcaactgtgtagcatcaatttcatgggtctactgtgtactgtgcccaaaaaatgccactgaatgctgcctgactggtggatagcaagactgtccattaatgtggtcatttaggttgcctctgcccaggtcttgaggtcattggcactaattcacaacaccctcaagtcacccagggaagatgagacacagttggctgtagacccacagtttgggcattacagctgcccttgaagttgacaaataaccacaaccttcaaattgttatgaaaagagcacaaatccaattaagaaagcttttccaaaagaaataacagtgttcctaccccctctgtcactctccaccccctttttgtcccagaataatgttgtgctgataggaacatggataaattaattacagtctggaatgttattcatgggtaggaaagaacactaaatctactcgcacaatgtttgatatttaaagataaacattgcctttatgttttttttttaaacctcagtcagcctagtttacgaagacataggtataatccttttaaatgctgtggattttttaatcgcaaaggtaacaatatgctgggtgttttacccagccagagaaccaggagatgcaggaatgagattagcatctctttagttccttgcatatttgatattattttggtgtacctccaattcctgataacatagaagaactcttgtggttgaagtccctgaaatggaaggatattggtaaccctgaatttaaaacaagcacaggcagcctttgtgggaatgtgtgtgaaggtcaccttctagaaacaggactgtccatagccattgccatggtttctgtgtcatttcaaccagaaccttaggcctggaagtctggatggatgtgggttggcatggtcctctatgggcattaaatgaataaatggatatagcagagggagtatccagcatgactcaaagaaggatgagaggaaacatattcaaataaaatctttagaaaagcaaatttcaaaaaaaaatgcttaagtataaaatattttgatgacaaccatgattttcaaattgaattcttattctaagtaatggtctaatctgaacttagacctctttccttaatttttttctcaataagcctttggtgtctagtcagttcaattcagtatttactgagtctctatacagacagggtataaggcattaatcaaatgtatgtccaaaattgcccaccatgcagggcagagctaaaatgcctaacacccctcctctcaccaacacatcccccacccacatctccaaagacttcctggcagaggtgatctctgcctgctgggacagatgtataggctccaacagcagcagggtggcccctctgaccaccaccttgggacccacattgctcttagaactattcctcttttttcatccttgaagcccccagcaaagctcagcctgaatcaactttttctaggaatctgacaagtttccaggctgatttcctgacccagtcagatcctcttccatctttctttgggtgttcaattttctac

(19) United States
(12) Patent Application Publication
(10) Pub. No.: US 2004/0053232 A1
(43) Pub. Date: Mar. 18, 2004

(54) Haplotype structures of chromosome 21

(75) Inventors: David R. Cox, Belmont, CA (US); Deana A. Arnold, Los Gatos, CA (US)

Correspondence Address: PERLEGEN SCIENCES, INC., LEGAL DEPARTMENT, 2021 STIERLIN COURT, MOUNTAIN VIEW, CA, 94043, US

(73) Assignee: Perlegen Sciences, Inc., Mountain View, CA (US)

(21) Appl. No.: 10/227,152

(22) Filed: Aug. 22, 2002

Related U.S. Application Data
(60) Provisional application No. 60/327,006, filed on Oct. 5, 2001.
(60) Provisional application No. 60/332,550, filed on Nov. 26, 2001.

(51) Int. Cl. C12Q 1/68; G06F 19/00; G01N 33/48; G01N 33/50

(52) U.S. Cl. 435/6; 702/20

(57) ABSTRACT

The present invention includes the use of any of the polymorphisms, SNP haplotype blocks or SNP haplotype patterns. In one embodiment, susceptibility to a phenotype resulting from an allele or marker in linkage disequilibrium with such polymorphic forms is evaluated. Novel therapeutic and diagnostic compounds and methods are also disclosed.

### CROSS REFERENCE TO RELATED APPLICATIONS

The present application is related to United States patent application U.S. Ser. No. unassigned, filed Aug. 22, 2002 (Attorney docket number 1030U-1); U.S. provisional patent application 60/323,059 filed Sep. 18, 2001 entitled “Human Genetic Polymorphisms”; U.S. provisional patent applications 60/280,530 filed Mar. 30, 2001, 60/313,264 filed Aug. 17, 2001, and 60/327,006 filed Oct. 5, 2001 entitled “Identifying Human SNP Haplotypes, Informative SNPs and Uses Thereof”; U.S. provisional patent application 60/332,550 filed Nov. 26, 2001 entitled “Methods for Genomic Analysis”; and the U.S. patent application Ser. No. 10/106,097 filed Mar. 26, 2002 entitled “Methods for Genomic Analysis”, which are incorporated by reference in their entirety for all purposes.

### BRIEF DESCRIPTION OF THE SEQUENCE LISTING

The Sequence Listing, which is incorporated herein by reference in its entirety, provides two sequences, each of which is identified by a sequence identification number (SEQ ID NO). SEQ ID NO: 2 is a DNA sequence that extends from position 21301951 to position 21415555 of the genomic DNA sequence identified by the GenBank accession number NT_{—}002836. SEQ ID NO: 1 is based on SEQ ID NO: 2, but further comprises nucleotide positions, each designated by an “n” in the sequence listing, that may contain an alternate base.

### BACKGROUND OF THE INVENTION

Variations or mutations in DNA are directly related to almost all human phenotypic traits and diseases, including infectious disease, cancer, inherited disorders, and autoimmune disorders. The most common type of DNA variation is a single nucleotide polymorphism (SNP), which is a base pair substitution at a single position in the genome. It has been estimated that SNPs account for the bulk of the DNA sequence difference between humans (Patil, N. et al., Science, 294:1719 (2001)). Blocks of such SNPs in close physical proximity in the genome are often genetically linked, resulting in reduced genetic variability and defining a limited number of “SNP haplotypes”, each of which reflects descent from a single, ancient chromosome (Fullerton, S. M., et al., Am. J. Hum. Genet. 67: 881 (2000)).

Patterns of human DNA sequence variation (haplotypes) defined by SNPs have important implications for identifying associations between phenotypic traits and genetic loci. However, the complexity of local haplotype structure in the human genome and the distance over which individual haplotype blocks extend is poorly defined, with some haplotype blocks extending for only a few kilobases and others extending for more than 100 kilobases (Patil, N. et al., Science, 294:1719 (2001)). These findings suggest that any comprehensive description of the haplotype structure of the human genome, defined by common SNPs, will require empirical analysis of a dense set of SNPs in many independent copies of the human genome. As a first step toward achieving this goal, high-density oligonucleotide arrays were used to identify a large fraction of all human chromosome 21 SNPs and to analyze the haplotype structure they define (Patil, N. et al., Science, 294:1719 (2001).

The haplotype structure of the human genome is of great value for various applications. For example, specific regions of interest may be further analyzed to associate SNPs in haplotype blocks with phenotypic traits—for example, disease susceptibility or resistance, a predisposition to a genetic disorder, or drug response—and this information may be invaluable in understanding the biological basis for the trait as well as identifying candidate genes useful in the development of therapeutics and diagnostics. The haplotype structure may also be used to identify individuals from biological samples, for example, in paternity testing or criminal investigations.

One such region of interest is found on the long arm of chromosome 21. This region contains two genes, KCNE1 and KCNE2, both of which code for proteins that are subunits of cardiac potassium channels, key components of the electrical system of the heart. Malfunction of these channels can cause abnormalities in the repolarization of the heart resulting in less efficient pumping of oxygenated blood through the body. Long QT Syndrome (LQTS), a familial and potentially fatal disorder of the electrical system of the heart, is also caused by malfunction of the cardiac potassium ion channels, which can lead to cardiac arrhythmia that may degenerate into ventricular tachycardia and even result in death. Currently, there is no quick and reliable method of identifying individuals with malfunctions of these potassium ion channels or a predisposition to LQTS.

### SUMMARY OF THE INVENTION

The present invention provides an isolated nucleic acid molecule comprising SEQ ID NO: 1 and fragments thereof. The present invention also provides sequences that are complementary to SEQ ID NO: 1, as well as isolated nucleic acid molecules that hybridize to SEQ ID NO: 1 under stringent conditions. The present invention also provides a database, which is on a computer-readable medium, comprising at least one SNP allele of SEQ ID NO: 1 that was derived from the analysis of at least one genome. In a preferred embodiment, the SNP allele of SEQ ID NO: 1 is associated with a phenotypic trait.

In some embodiments of the present invention, a method for identifying a genetic locus associated with a phenotypic trait of interest is provided. The method includes the following steps: obtaining a biological sample from a control population that does not possess the phenotypic trait of interest and a biological sample from a clinical population that possesses the phenotypic trait of interest, determining an allelic frequency for at least one single nucleotide polymorphism listed in FIG. 1 or FIG. 2 in the control population and the clinical population, and comparing the allelic frequencies from the two populations to identify those that indicate the presence of a genetic locus associated with the phenotypic trait of interest.

In some aspects, the present invention provides a method of screening an individual for a predisposition, susceptibility, or resistance to a phenotypic trait of interest. The method includes the following steps: obtaining a biological sample from an individual, analyzing the biological sample for the presence of a nucleic acid molecule that comprises at least 10 nucleotides of SEQ ID NO: 1 and at least one alternative base as listed in FIG. 1 or FIG. 2, or a complementary sequence thereto, and determining the predisposition, susceptibility, or resistance of the individual to the phenotypic trait of interest based on the presence or absence of the nucleic acid molecule. In preferred embodiments, the presence or absence of the nucleic acid molecule indicates a predisposition, susceptibility, or resistance to a cardiovascular disorder, a response to a drug, a hearing disability, or a potassium ion channel disorder.

In further embodiments, the present invention provides a method for selecting a therapeutic for an individual that has or is predisposed to a phenotypic trait of interest that is associated with an isolated nucleic acid molecule that comprises at least 10 nucleotides of SEQ ID NO: 1 and at least one alternative base as listed in FIG. 1 or FIG. 2, or a complementary sequence thereto. The method includes the following steps: detecting whether the individual possesses the isolated nucleic acid molecule, and selecting a therapeutic that compensates for a causative functional mutation that is in linkage disequilibrium with the isolated nucleic acid molecule.

The present invention further provides a kit for diagnosing a disease, disease susceptibility, or therapy response associated with an isolated nucleic acid molecule that comprises at least 10 nucleotides of SEQ ID NO: 1 and at least one alternative base as listed in FIG. 1 or FIG. 2, or a complementary sequence thereto. The kit includes a means for detecting a presence or absence of the isolated nucleic acid molecule in a DNA sample from a patient, as well as a data set of associations of the nucleic acid molecule with the disease, disease susceptibility, or therapy response. In preferred embodiments, the data set of associations is on a computer-readable medium.

### BRIEF DESCRIPTION OF THE FIGURES

The following figures and drawings form part of the present specification and are included to further demonstrate certain aspects of the patent invention. The invention may be better understood by reference to one or more of these drawings in combination with the detailed description of the specific embodiments presented herein. All publications mentioned herein are cited for the purpose of describing and disclosing reagents, methodologies and concepts that may be used in connection with the present invention. Nothing herein is to be construed as an admission that these references are prior art in relation to the inventions described.

FIG. 1 shows common SNPs in the region of interest.

FIG. 2 shows rare SNPs in the region of interest.

FIG. 3 shows haplotype blocks B137313, B137314, and B137315.

### DETAILED DESCRIPTION OF PREFERRED EMBODIMENTS

I. Glossary

II. General

III. Polymorphisms, Haplotype Blocks and Haplotype Patterns

IV. Detection of Haplotype Structure of the Invention in Target DNA

V. Methods of Use

A. Identification of Genetic Loci Associated With Phenotypic Traits

B. Production and Use of Peptides

C. Diagnostics

D. Pharmacogenomics

E. Therapeutics

F. Other Uses and Aspects of the Invention

VI. Conclusion

I. Glossary

As used in the specification, “a” or “an” means one or more. As used in the claim(s), when used in conjunction with the word “comprising”, the words “a” or “an” mean one or more. As used herein, “another” means at least a second or more.

“Gene” is intended to mean the ORF (open reading frame) encoding an RNA or polypeptide, intronic regions, and the adjacent 5′ and 3′ non-coding nucleotide sequences, which may extend up to about 10 kb beyond the coding region, but possibly further in either direction. The adjacent and intronic sequences may be involved in the regulation of expression of the encoded RNA or polypeptide.

“Haplotype structure” refers to the combination of polymorphisms, haplotype patterns and haplotype blocks in a nucleic acid sequence of interest.

“Hybridization probes” or “probes” are oligonucleotides capable of binding in a base-specific manner to a partially or completely complementary strand of nucleic acid. Such probes include peptide nucleic acids, as described in Nielsen et al., Science 254: 1497-1500 (1991), as well as all other kinds of oligonucleotides, as described supra.

Hybridizations are usually performed under stringent conditions. Stringent conditions are sequence-dependent and are different in different circumstances. Generally, stringent conditions are selected to be about 5° C. lower than the thermal melting point (Tm) for the specific sequence at a defined ionic strength and pH. The Tm is the temperature (under defined ionic strength, pH, and nucleic acid concentration) at which 50% of the probes complementary to the target sequence hybridize to the target sequence at equilibrium. As the target sequences are generally present in excess, at Tm, 50% of the probes are occupied at equilibrium. Typically, stringent conditions include a salt concentration of at least about 0.01 to 1.0 M Na ion concentration (or other salts) at pH 7.0 to 8.3 and the temperature is at least about 25° C. for short probes (e.g., 10 to 50 nucleotides). Stringent conditions can also be achieved with the addition of destabilizing agents such as formamide. For example, conditions of 5×SSPE (750 mM NaCl, 50 mM NaPhosphate, 5 mM EDTA, pH 7.4) and a temperature of 25-30° C. are suitable for allele-specific probe hybridizations.

“Informative SNP” refers to a SNP (or plurality of SNPs) which has been selected from the set of all SNPs in a SNP haplotype pattern and that tends to distinguish one SNP haplotype pattern from other SNP haplotype patterns within a SNP haplotype block. Thus, once SNP haplotype patterns for a particular SNP haplotype block are known, one can select one or more informative SNPs from each SNP haplotype pattern to 1) identify the genotype of all other SNPs in that SNP haplotype pattern, and 2) distinguish the SNP haplotype pattern from other SNP haplotype patterns that belong to a particular SNP haplotype block.

An “isolated nucleic acid” means an object species invention that is the predominant species present (e.g., on a molar basis it is more abundant than any other individual species in the composition). Preferably, an isolated nucleic acid comprises at least about 50, 80, or 90 percent (on a molar basis) of all macromolecular species present. Most preferably, the object species is purified to essential homogeneity (contaminant species cannot be detected in the composition by conventional detection methods).

“Linkage” or “linked” describes or relates to the tendency of genes, alleles, loci or genetic markers to be inherited together from generation to generation as a result of the proximity of their locations on the same chromosome; e.g., genetic loci that are inherited non-randomly.

“Linkage disequilibrium” or “allelic association” means the preferential association of a particular allele or genetic marker with a specific allele or genetic marker at a nearby chromosomal location more frequently than expected by chance for any particular allele frequency in the population. For example, if locus X has alleles a and b, which occur equally frequently, and linked locus Y has alleles c and d, which occur equally frequently, one would expect the combination ac to occur with a frequency of 0.25. If ac occurs more frequently, then alleles a and c are in linkage disequilibrium. Linkage disequilibrium may result from natural selection of certain combination of alleles or because an allele has been introduced into a population too recently to have reached equilibrium with linked alleles. A marker in linkage disequilibrium can be particularly useful in detecting susceptibility to disease (or other phenotype) notwithstanding that the marker does not cause the disease. For example, a marker (X) that is not itself a causative element of a disease, but which is in linkage disequilibrium with a gene (including regulatory sequences) (Y) that is a causative element of a phenotype, can be used detected to indicate susceptibility to the disease in circumstances in which the gene Y may not have been identified or may not be readily detectable.

“Nucleic acids” include but are not limited to DNA, RNA, single- or double-stranded, genomic, cloned, naturally occurring or synthetic molecules and may be polynucleotides, amplicons, RNA transcripts, protein nucleic acids, nucleic acid mimetics, and the like.

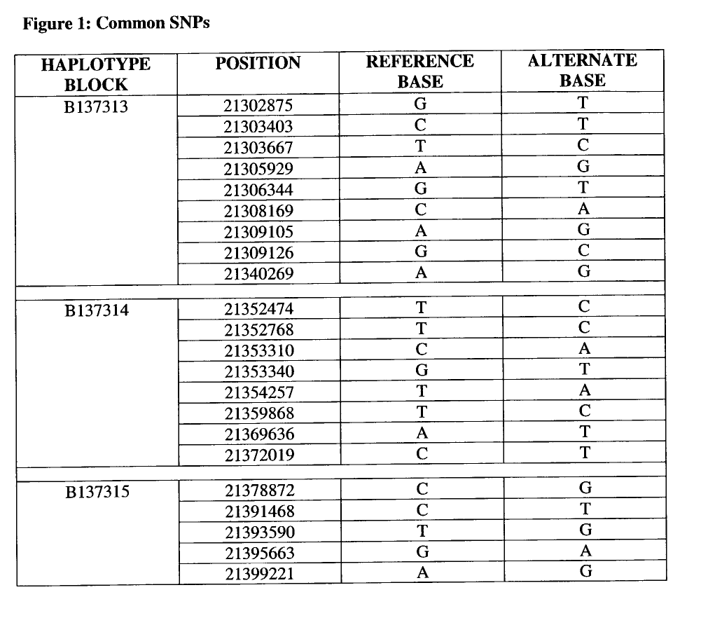

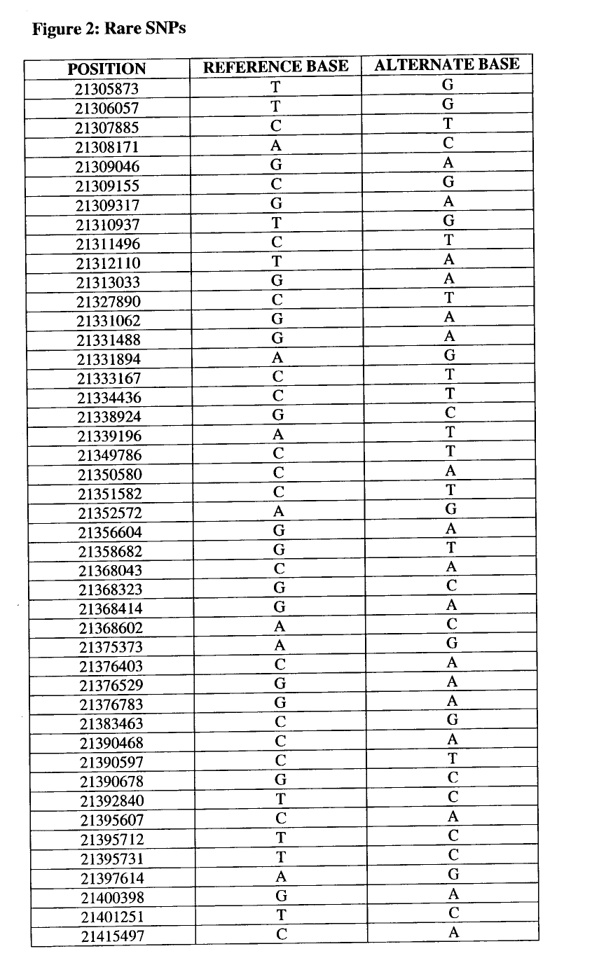

“Oligonucleotides” are nucleic acids that are usually between 5 and 100 contiguous bases, and often between 5-10, 5-20, 10-20, 10-50, 15-50, 15-100, 20-50, or 20-100 contiguous bases. An oligonucleotide that is longer than about 20 contiguous bases may be referred to as a polynucleotide. A polymorphic site (polymorphism) can occur at any position within an oligonucleotide. An oligonucleotide may include any of the allelic forms of the polymorphic sites (polymorphisms) shown in FIG. 1 or FIG. 2.

A “polymorphic site” refers the position in a nucleic acid sequence at which a polymorphism occurs. A polymorphic site may be as small as one base pair. A “SNP location” or “SNP locus” is a polymorphic site at which a SNP occurs.

“Polymorphism” refers to a genetic variation, or the occurrence of two or more genetically determined alternative sequences or alleles at a single genetic locus in a population. Preferred polymorphisms have two alleles, with the minor allele occurring at a frequency of greater than 1%, and more preferably greater than 10% or 20% of a selected population. The allelic form occurring most frequently in a selected population is sometimes referenced as the “wildtype” form. Diploid organisms may be homozygous or heterozygous for allelic forms. A biallelic polymorphism has two forms. A triallelic polymorphism has three forms. Examples of polymorphisms include restriction fragment length polymorphisms (RFLPs), variable number of tandem repeats (VNTRs), single nucleotide polymorphisms (SNPs), dinucleotide repeats, trinucleotide repeats, tetranucleotide repeats, simple sequence repeats, and insertion elements such as Alu.

A “SNP” or “single nucleotide polymorphism” is a polymorphism that occurs at a polymorphic site occupied by a single nucleotide. The site of the SNP is usually preceded by and followed by highly conserved sequences (e.g., sequences that vary in less that 1/100 or 1/1000 members of a population). As used herein, “SNPs” is the plural of SNP. SNPs are most frequently diallelic. A most common allele of a SNP is called a “major allele” and an alternative allele of said SNP is called a “minor allele”. A SNP usually arises due to substitution of one nucleotide for another at the polymorphic site. A transition is the replacement of one purine by another purine or one pyrimidine by another pyrimidine. A transversion is the replacement of a purine by a pyrimidine or vice versa. SNPs can also arise from a deletion of a nucleotide or an insertion of a nucleotide relative to a reference allele.

A “SNP haplotype block” or “haplotype block” is a nucleic acid sequence containing a group of SNPs or polymorphisms that do not appear to recombine independently but are passed from generation to generation in variable-length blocks.

A “SNP haplotype pattern” or “haplotype pattern” refers to the set of genotypes for SNPs or other polymorphisms in a haplotype block in a single strand of nucleic acid, preferably a single strand of genomic DNA.

II. General

Throughout the disclosure various patents, patent applications and publications are referenced. Unless otherwise indicated, each is incorporated by reference in its entirety for all purposes.

It readily should be apparent to one skilled in the art that various embodiments and modifications may be made to the invention disclosed in this application without departing from the scope and spirit of the invention.

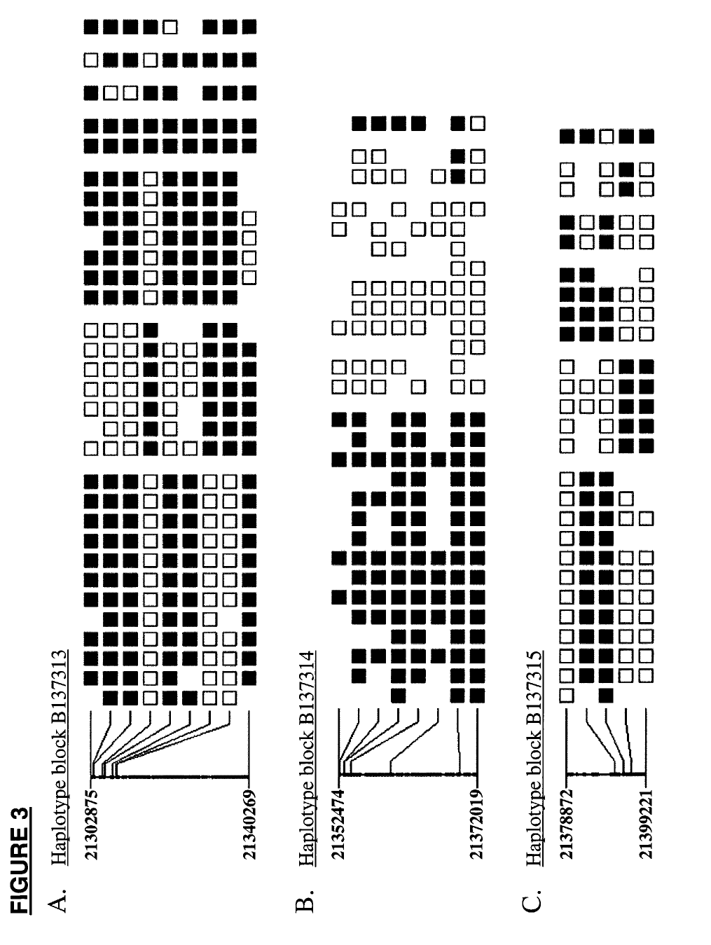

The present invention includes the use of any of the polymorphisms, SNP haplotype blocks or SNP haplotype patterns shown in FIG. 1, FIG. 2 and FIG. 3, as well as polymorphisms, alleles, or markers in linkage disequilibrium with them, as a means to study a phenotype for a variety of purposes including drug target identification, diagnostics, and therapeutics. In the present invention, the DNA composition of a plurality of biological samples was analyzed to reveal novel polymorphisms (e.g., SNPs) and SNP haplotype patterns. In one embodiment they, or polymorphisms in linkage disequilibrium with them, may be predictive of or used to study cardiovascular disorders (e.g., LQTS or ventricular fibrillation), drug response (e.g., clarithromycin-induced arrhythmia) and other phenotypes related to cardiovascular disorders, drug response, or the LQTS1 and LQTS2 genes. The approach of the present invention has tremendous advantages in conducting genetic association studies over other whole genome or genotyping methods known in the art. Instead of reading all bases of each individual's DNA, or even reading the common SNPs that may be found, only informative SNPs from the sample population need to be determined and scanned.

Polymorphisms of the present invention are shown in FIGS. 1 and 2 and were identified by, e.g., the methods described in the earlier patent applications U.S. Ser. No. 10/106,097, filed Mar. 26, 2002, and U.S. Ser. No. 10/134,510, filed Apr. 29, 2002, both entitled “Methods for Genomic Analysis” and incorporated herein in their entirety by reference. These polymorphisms occur in a region of chromosome 21 that contains genes that code for subunits of potassium ion channels known to be involved in several disorders including Long QT Syndrome (LQTS), ventricular fibrillation, clarithromycin-induced arrhythmia, and deafness. Polymorphisms of the present invention also include those in haplotype blocks with one or more of the polymorphisms shown in FIG. 1 or 2.

LQTS is a familial and potentially fatal disorder of the electrical system of the heart characterized by an abnormally prolonged “QT interval” and is one phenotype measure of the time it takes for the heart to undergo ventricular depolarization (contraction) and repolarization (recharging/rest) between each heart beat. When this interval is prolonged, patients may develop an extremely rapid, abnormal heart rhythm (arrhythmia) that can degenerate into a severe ventricular tachycardia known as “torsade de pointes”. When this occurs, the heart can no longer effectively pump blood through the body and the resulting decrease of bloodflow to the brain can cause loss of consciousness (syncope). If treatment is not immediate, this tachycardia can lead to ventricular fibrillation and, eventually, cardiac arrest and sudden death.

The symptoms of LQTS are caused by abnormalities of protein structures, called “ion channels”, which regulate the flow of ions, such as potassium, in and out of heart cells, thereby controlling the electrical activity of the heart. When the ion channels are dysfunctional, as in the case of LQTS, the depolarization and repolarization of the heart takes longer, and the result is a prolonged QT interval. These abnormalities can be passed on from parent to child when a mutation is present in a gene that codes for one of the ion channel proteins. Since a variety of mutations can cause the disorder, several forms of LQTS exist. To date, mutations causing LQTS have been identified in genes encoding both potassium channels and sodium channels (Splawski, et al., Circulation 102: 1178 (2000)). At least two of these genes, KCNE1 and KCNE2, are located on chromosome 21 in the 21q22.1-22.2 region. These genes code for proteins in potassium (K⁺) channels: the “slowly activating delayed rectifier K⁺ (Iₖₛ) channel” and the “rapidly activating delayed rectifier K⁺ (Iₖᵣ) channel”, respectively. In addition to the cardiac phenotype, complete loss of the Iₖₛ channel also causes hearing disability (partial hearing loss to complete deafness) in the LQTS subtype known as Jervell and Lange-Nielsen syndrome (Schulze-Bahr, et al., Nature Genetics 17:267 (1997)).

LQTS-related deaths are largely preventable with treatment, but unfortunately, individuals with LQTS often remain undiagnosed until it is too late. Many carriers are asymptomatic until under some kind of physical or emotional stress, and even at that time the severity of the symptoms varies widely depending on the length of time the arrhythmia persists. For a short episode, the individual may experience only a few seconds of extreme dizziness or syncope, which may not prompt them to seek medical attention. As such, a diagnosis is often not made until after a serious cardiac incident, such as ventricular tachycardia or cardiac arrest, or after the LQTS-related death of a family member.

Currently, individuals suspected of having LQTS are tested by electrocardiogram (ECG), which measures and records the electrical activity of the heart and can thereby detect a clearly prolonged QT interval. However, this symptom is not apparent in all affected persons; some patients have normal or borderline-prolonged QT intervals based on their resting ECG. In fact, in a study of nine families with “sporadic” cases of LQTS, 33% of family members who were considered on clinical grounds to be non-affected were found to be carriers (Priori, et al., Circulation 99:518 (1999)). This, along with the day to day variability of an individual's QT intervals, makes ECG diagnosis of LQTS unreliable. As a result, some LQTS carriers are not identified as such, and others are misclassified as having the disorder when they do not; both of these misdiagnoses lead to inappropriate treatment of the individual. Since this is a hereditary disease, proper diagnosis of both symptomatic and asymptomatic individuals is needed to allow informed decisions regarding the risk of LQTS to their offspring. Therefore, improved diagnosis of LQTS is desperately needed to properly identify and treat those individuals at risk to prevent the potentially lethal LQTS-related syncope and ventricular tachycardia, as well as to predict the risk of LQTS to their offspring.

Another aspect of LQTS is that affected individuals are sensitive to certain drugs and can experience ventricular tachycardia if these drugs are administered to them (Priori, et al., Circulation, 99:518 (1999)). Ironically, many of these drugs are antiarrhythmia drugs, but they also include certain antidepressants, antihistamines, and the antibiotic erythromycin. Clearly, if an individual is not properly diagnosed with LQTS or is an otherwise asymptomatic carrier, they may unnecessarily be put at risk by being prescribed these medications, especially since their symptoms in the absence of a clearly prolonged QT interval may suggest a need for antiarrhythmia drugs, so improved diagnosis of LQTS would protect LQTS patients from drugs dangerous to their condition.

The most common treatment for LQTS patients is beta-blocker drug therapy, which blunts the surges of adrenaline that trigger episodes of ventricular tachycardia. However, current research suggests that while beta-blockers can be quite effective for individuals carrying certain LQTS genotypes, patients with other forms of LQTS seem to respond better to the administration of potassium or a sodium channel blocker, and still others require the implantation of an artificial pacemaker or an implantable cardioverter defibrillator (ICD) (Moss, et al., Circulation, 101:616-623 (2000); Priori, et al., Circulation, 99:518 (1999)). So although several treatments are available, their efficacy is dependent on the genotype of the LQTS patient. As such, the most effective treatment could be more quickly and correctly determined if tailored to the specific LQTS subtype carried by the affected individual. specific LQTS subtype carried by the affected individual.

In addition, potassium channels not only control repolarization, but also affect other aspects of normal heart function, such as resting membrane potential. As such, it is likely that identifying the genotypes involved in the function of ion channels would not only facilitate the understanding and treatment of LQTS, but also other more general disorders that involve ion channel function. For example, individuals with inefficient ion channels may have a greater risk of developing heart disease. Another example is the involvement of these ion channels in normal hearing as evidenced by the loss of hearing or complete deafness that affects some LQTS patients.

III. Polymorphisms, Haplotype Blocks and Haplotype Patterns

SEQ ID NO: 1 extends from position 21301951 to position 21415555 of the genomic DNA sequence identified by the GenBank accession number NT_{—}002836. More specifically, this region contains the KCNE1 and KCNE2 genes, both of which are known to be involved in LQTS. This region may also contain additional genes as evidenced by a RefSeq gene prediction, C21orf51, several GenScan and Acembly gene predictions, and multiple sites that align with human mRNAs and other ESTs in GenBank. The present invention provides nucleic acids containing polymorphisms, haplotype blocks and haplotype patterns based on SEQ ID NO: 2, including SEQ ID NO: 1 or fragments thereof with at least one single nucleotide polymorphism listed in FIG. 1 or 2, as well as nucleic acid derivatives of these SEQ ID NO: 1 variants or fragments thereof, such as but not limited to RNA, cDNA and nucleic acid mimetics, provided that the sequence is not a fragment of SEQ ID NO: 2. These nucleic acids may further comprise genic or nongenic regions. Genic regions further comprise coding regions (exons) and intronic regions. In addition, genic regions also comprise regulatory regions that may be found hundreds, and possibly thousands of kilobases upstream from the transcriptional start site or downstream of the most distal base pair transcribed. These nucleic acids may be studied substantially free of other nucleic acid sequences, and may be amplified prior to evaluation, as discussed infra.

Polymorphisms of the present invention were identified within SEQ ID NO: 1 by scanning the genomes of a plurality of individuals from a diverse population spanning multiple ethnic and geographic backgrounds. In a preferred embodiment, the polymorphisms identified were SNPs, or “single nucleotide polymorphisms”. The location of these polymorphisms was mapped onto the human genome and analyzed to determine the haplotype structure of this genomic region. The analysis involves the determination of each allele (e.g., A, C, T or G) of a polymorphism. The allele that is present in the reference sequence (SEQ ID NO: 2) is referred to as the “reference base”, and the alternate allele is referred to as the “alternate base”.

The analysis also involves the determination of the frequency of each allele for each polymorphism. “Common SNPs” are those SNPs whose less common form (minor allele) is present at or above a certain minimum frequency in a given population. For example, common SNPs are those SNPs that are found in at least about 2% to 25% of the population. Preferably, common SNPs are those SNPs that are found in at least about 5% to 15% of the population. More preferably, common SNPs are those that are found in at least about 10% of the population. Common SNPs are listed in FIG. 1 in order of their location (nucleotide position) (column 2) relative to the genomic DNA sequence identified by the GenBank accession number NT_{—}002836; also included are the reference (column 3) and alternate (column 4) bases for each SNP, as well as a haplotype block (column 1) to which each SNP may be assigned according to one embodiment of the invention (discussed infra).

Common SNPs likely result from mutations that occurred early in the evolution of a species. Focusing on common SNPs decreases the false positives that result from recent population anomalies; i.e., allele or variant differences between control and experimental populations that appear as disease or drug-response associated, yet are result of migratory history or mating practices. Moreover, common SNPs are relevant to a larger proportion of the human population, making the present methods more broadly applicable to disease and drug response studies. However, the present invention also includes “rare SNPs” (FIG. 2) since certain analyses may be performed including some or all rare SNPs, particularly when looking at individuals in a population, specific subpopulations, the migratory history of populations, the environmental effect on the genetic makeup of a population, investigation of rare phenotypic traits and the like. Rare SNPs are listed in FIG. 2 in order of their location (nucleotide position) (column 1) relative to the genomic DNA sequence identified by the GenBank accession number NT_{—}002836; also included are the reference (column 2) and alternate (column 3) bases for each SNP.

Sequences from different origins were compared, SNPs were scored, and a SNP map was constructed. Once the individual SNPs were identified and mapped to the genome, the SNP haplotype blocks and SNP haplotype patterns within the SNP haplotype blocks were defined. SNP haplotype blocks are sequences containing a set of one or more SNPs that do not recombine independently but are passed from generation to generation in variable-length blocks. The set of genotypes for all the SNPs in a SNP haplotype block on a single chromosome of an individual is a SNP haplotype pattern. It is important to note that blocks are defined based on their genetic information content and not on knowledge of how this information originated or why it exists. As such, blocks do not have absolute boundaries, and may be defined in different ways, depending on the specific application. The algorithm in this embodiment provides only one of many possible approaches. Those with skill in the art recognize a variety of algorithms can be used to define a set of haplotype blocks for a given region, including but not limited to greedy algorithms and shortest path algorithms. Further, parameters within an algorithm may be adjusted so to attain more or less stringent criteria for grouping SNPs into a haplotype block. For more detailed methods useful for defining the boundaries of haplotype blocks, see the U.S. patent application Ser. No. 10/134,510 filed Apr. 29, 2002 entitled “Methods for Genomic Analysis”, incorporated herein in its entirety for all purposes.

According to one embodiment of the invention, SNP haplotype blocks and SNP haplotype patterns within each SNP haplotype block were constructed using common SNPs and are shown in FIG. 3. Three haplotype blocks, B137313, B137314, and B137315, were constructed for the region comprising SEQ ID NO: 1. Each row of boxes represents a single common SNP within the haplotype block. As in FIG. 1, these SNPs are ordered based on their position within SEQ ID NO: 1; the position numbers are shown for only the most proximal and most distal common SNP in each haplotype block. For example, for haplotype block B137313 containing nine common SNPs, the common SNP at position 21302875 is shown in the top row, the common SNP at position 21303403 is shown in the second row, and so forth. Each column of boxes in a haplotype block represents a haplotype pattern. For example, for haplotype block B137313 containing seven haplotype patterns, the first twelve columns represent twelve individual chromosomes, each containing the same haplotype pattern. For each haplotype block, the dark boxes represent the reference base and the light boxes represent the alternate base, both of which are listed in FIG. 1 for each common SNP position. In summary, FIG. 3 illustrates that SNPs occur in haplotype blocks in a genome, and that more than one haplotype pattern can occur within each haplotype block.

The boundaries between haplotype blocks may be defined in several different ways, including, but not limited to the following examples. One method of defining the boundaries of haplotype blocks is to extend them only to the most distal SNP in each block as shown in FIG. 3. In this case, there would most often be gaps between adjacent blocks. Another method of defining the boundaries of haplotype blocks is to extend them up to (but not including) the most proximal SNP of the adjacent block. In this case, the blocks would overlap. Yet another way is to extend the blocks to the nucleotide position that is halfway in between the most distal SNP in the block and the most proximal SNP in the next block. In this case there would be no gaps nor overlap between adjacent blocks. As mentioned above, the boundaries of the haplotype blocks shown in FIG. 3 are the outermost common SNPs in each block. It is expected that the boundaries between haplotype blocks will be adjusted accordingly if additional common SNPs are identified in this genomic region.

An informative SNP is a SNP, which has been selected from the set of all SNPs in a haplotype pattern, that, either alone or in combination with other informative haplotype block. Thus, once haplotype patterns for a particular haplotype block are known, one can select one or more informative SNPs from each haplotype pattern to 1) identify the genotype of all other SNPs in that haplotype pattern, and 2) distinguish the haplotype pattern from other haplotype patterns that belong to a particular haplotype block. Informative SNPs are selected so that the genotype of an informative SNP predicts the genotype of other, preferably all remaining, SNPs in that haplotype pattern. Knowing the informative SNPs for all patterns in all haplotype blocks allows for the design of less expensive genotyping assays that retain most of the power of an assay constructed using all SNPs.

The number of informative SNPs required for each block is the number of SNPs necessary to distinguish between the common SNP haplotype patterns in each SNP haplotype block. The number of informative SNPs required for haplotype blocks B137313, B137314 and B137315 is 2, 1 and 2, respectively. However, more than one SNP in a haplotype pattern may serve as an informative SNP. For example, if there exist only two haplotype patterns in a haplotype block, then any SNP that has a different genotype in one versus the other may be used to distinguish between them. If there are three or four haplotype patterns, then at least two SNPs are required. Given a sufficient number of informative SNPs to distinguish between all haplotype patterns, the existence of a particular haplotype pattern in an unknown sample may be inferred with accuracy. For example, for haplotype block B137314 any of the seven SNPs in the block can distinguish greater than 89% of the haplotype patterns, and two SNPs can distinguish greater than 96% of the haplotype patterns. In one embodiment, an algorithm was used to identify informative SNPs for each haplotype block.

IV. Detection of Haplotype Structure of the Invention in Target DNA

Detecting polymorphisms involves comparing DNA sequences in different individuals to identify points of variation, i.e., polymorphic sites or polymorphisms. By analyzing groups of individuals, haplotype structure comprising the frequencies of variation at each SNP locus (allelic frequency) and haplotype patterns in a population can be determined. Once a baseline of allelic or haplotype pattern frequencies is determined for a population, allelic or haplotype pattern frequencies can be determined for subpopulations characterized by many different criteria including, but not limited to geography, race, gender, disease susceptibility or resistance, and response to therapeutics.

The polymorphisms, haplotype patterns, and haplotype blocks of the invention may be detected in sample nucleic acids (“target DNA”) from an individual being screened, and this target DNA may be obtained from virtually any biological sample (other than pure red blood cells). For example, convenient tissue samples include whole blood, semen, saliva, tears, fecal matter, urine, sweat, buccal, skin and hair. For assays of cDNA or mRNA, the tissue should be obtained from an organ in which the target nucleic acid is expressed. For example, if the target nucleic acid is KCNE1 or KCNE2 mRNA, the heart is a suitable source.

Sample nucleic acids may be prepared for analysis using any technique known to those skilled in the art. Preferably, such techniques result in the production of a nucleic acid molecule sufficiently pure to determine the presence or absence of one or more polymorphisms at one or more locations in the nucleic acid molecule. Such techniques may be found, for example, in Sambrook, et al., Molecular Cloning: A Laboratory Manual (Cold Spring Harbor Laboratory, New York) (2001), incorporated herein by reference. In addition, the methods disclosed in pending US patent application U.S. Ser. No. 10/134,510, filed Apr. 29, 2002 entitled “Methods for Genomic Analysis” are particularly suitable for preparing nucleic acids for use in the methods of the present invention and are incorporated herein in their entirety.

It may be desirable to amplify and/or label one or more nucleic acids of interest before determining the presence or absence of one or more polymorphisms in the nucleic acid. Any amplification technique known to those of skill in the art may be used in conjunction with the present invention including, but not limited to, polymerase chain reaction (PCR) techniques. PCR may be carried out using materials and methods known to those of skill in the art. See generally PCR Technology: Principals and Applications for DNA Amplification (ed. H. A. Erlich, Freeman Press, NY, N.Y., 1992); PCR Protocols: A Guide to Methods and Applications (eds. Innis, et al., Academic Press, San Diego, Calif., 1990); Matilla et al., Nucleic Acids Res. 19: 4967 (1991); Eckert et al., PCR Methods and Applications 1: 17 (1991); PCR (eds. McPherson et al., IRL Press, Oxford); and U.S. Pat. No. 4,683,202 (each of which is incorporated by reference for all purposes). Other suitable amplification methods include the ligase chain reaction (LCR) (see Wu and Wallace, Genomics 4: 560 (1989) and Landegren et al., Science 241: 1077 (1988)), transcription amplification (Kwoh et al., Proc. Natl. Acad. Sci. USA 86: 1173 (1989)), self-sustained sequence replication (Guatelli et al., Proc. Nat. Acad. Sci. USA, 87: 1874 (1990)) and nucleic acid-based sequence amplification (NASBA). Further, the methods disclosed in pending U.S. patent applications U.S. Ser. No. 10/134,510, filed Apr. 29, 2002 entitled “Methods for Genomic Analysis”; U.S. Ser. No. 10/042,492, filed Jan. 9, 2002 entitled “Methods for Amplification of Nucleic Acids”; and U.S. Ser. No. [unassigned], attorney docket number 1027U-1, filed Jun. 17, 2002 entitled “Methods for Storage of Reaction Cocktails” particularly suitable for amplifying, labeling, or further manipulating (i.e. fragmentation) nucleic acids for use in the methods of the present invention (incorporated by reference in their entirety for all purposes).

Determination of the presence or absence of one or more polymorphisms in a nucleic acid may be made using any technique known to those of skill in the art. Any technique that permits the accurate determination of a variation can be used. Preferred techniques permit rapid, accurate determination of multiple variations with a minimum of sample handling. Some examples of suitable techniques involve but are not limited to direct DNA sequencing, capillary electrophoresis, hybridization, allele-specific probes or primers, single-strand conformation polymorphism analysis, nucleic acid arrays and other techniques well known in the art. Several methods for DNA sequencing are well known and generally available in the art and may be used to determine the location of SNPs in a genome. See, for example, Sambrook, et al., Molecular Cloning: A Laboratory Manual (Cold Spring Harbor Laboratory, New York) (2001), and Ausubel, et al., Current Protocols in Molecular Biology (John Wiley and Sons, New York) (1997), incorporated herein by reference. Descriptions of the use of these methodologies are also detailed in provisional patent application serial No. 60/323,059, filed Sep. 18, 2001, entitled “Human Genomic Polymorphisms”, incorporated by reference in its entirety for all purposes. Some examples of these are described by WO 95/11995 (incorporated by reference in its entirety for all purposes). WO 95/11995 also describes subarrays that are optimized for detection of different allelic forms of precharacterized polymorphisms, such as those of the present invention. For details on the use of nucleic acid arrays (DNA chips) for the detection of, for example, SNPs, see U.S. Pat. No. 6,300,063 issued to Lipshultz, et al., and U.S. Pat. No. 5,837,832 to Chee, et al., HuSNP Mapping Assay, reagent kit and user manual, Affymetrix Part No. 90094 (Affymetrix, Santa Clara, Calif.), all incorporated by reference herein.

V. Methods of Use

The invention has utility for identifying polymorphisms, haplotype patterns, and haplotype blocks in biological samples. This information may then be used in any number of ways including, but not limited to association studies, forensics, paternity testing, genetic mapping of phenotypic traits (e.g., disease resistance or susceptibility, drug response, etc.), diagnostics, identification of candidate drug targets, drug (or other treatment) efficacy trials, development of protein, small molecule, antisense, antibody, or other therapeutics, and to reveal the biological basis for a phenotypic trait. More details of these various utilities are provided infra.

The nucleic acids of the invention may be used in Southern or Northern analysis, dot blot, or other membrane based technologies, in PCR technologies, in dipstick assays, and in microarrays utilizing fluids or tissue extracts from patients. The polynucleotide sequences of the present invention, and longer or shorter sequences derived therefrom, also may be used as targets in a microarray or other genotyping system. These systems can be used to detect the presence or absence of a large number of particular allelic SNP forms or to monitor the expression of a large number of gene products simultaneously.

In a preferred embodiment, it is possible to use allele-specific probes to determine the genotype of the polymorphisms (e.g., the haplotype structure) in a target DNA molecule. The design and use of allele-specific probes for analyzing polymorphisms is described by e.g., U.S. Pat. No. 6,361,947 issued to Dong, et al. Allele-specific probes can be designed that hybridize to a segment of target DNA from one individual but do not hybridize to the corresponding segment from another individual due to the presence of different polymorphic forms (alleles) in the respective segments from the two individuals. Hybridization conditions should be sufficiently stringent such that there is a significant difference in hybridization intensity between alleles, and preferably an essentially binary response, whereby a probe hybridizes to only one of the alleles. Some probes are designed to hybridize to a segment of target DNA such that the polymorphic site aligns with a central position (e.g., in a 15-mer at the 7^{th} position; in a 25-mer at the 13^{th} position) of the probe. This design of probe achieves good discrimination in hybridization between different allelic forms. In a preferred embodiment, a nucleic acid of the invention is specifically hybridized to a target nucleic acid as a means of detecting a polymorphism in the target nucleic acid. These allele-specific probes can also be immobilized on a nucleic acid array, some examples of which are described by WO 95/11995 (incorporated by reference in its entirety for all purposes). An example of hybridization to a nucleic acid array involves the use of DNA chips (oligonucleotide arrays), for example, those available from Affymetrix, Inc. Santa Clara, Calif. In a preferred embodiment, nucleic acid arrays are used to detect the polymorphisms of the invention in a target DNA sample.

A. Identification of Genetic Loci Associated With Phenotypic Traits

The polymorphisms, haplotype patterns, and haplotype blocks are useful for the identification of genetic components associated with phenotypic traits, whether causative or predictive, whether at one specific locus in the genome or at multiple loci on the same or different chromosomes. Association (or “correlation”) studies may be performed for this purpose by determining the genotype of a set of at least one polymorphism for two populations of individuals, one of which exhibits a particular phenotypic trait, and one of which lacks the trait. In another embodiment, the genotypes of more than two populations may be compared, for example, by age, ethnicity, or geographic location. The characteristics of the set of polymorphisms that are compared between the populations include, but are not limited to, the frequency of each genotype of each polymorphism, haplotype patterns that include at least one of the polymorphisms, and haplotype blocks that include at least one haplotype pattern. For example, sets of polymorphisms that occur at a higher or lower frequency in one population than in another indicate areas in the genome where phenotypic trait-related loci may be located. In preferred embodiments, an analysis may be performed by comparing the haplotype structure of a region of interest present in two populations to identify those polymorphisms or haplotype patterns that associate (or “correlate”) with a phenotypic trait of interest. For example, the haplotype structure of the genomic region corresponding to SEQ ID NO: 1 may be used to identify polymorphisms or haplotype patterns that associate with such phenotypic traits as LQTS susceptibility, LQTS-related drug sensitivity, Iₖₛ channel-related hearing loss, or other phenotypic traits that are in linkage disequilibrium with the polymorphisms or haplotype patterns of the invention, such as those that may be related to the gene predictions discussed supra.

An association between a polymorphism or haplotype pattern and a phenotypic trait can be determined by standard statistical methods and statistically significant associations between the haplotype structure and the phenotypic trait are then noted. For example, it may be found that a G at position 21393590 (haplotype block B137315) correlates with hearing impairment. As a further example, it might be found that the combined presence of a G at position 21393590 (haplotype block B137315) and a G at position 21340269 (haplotype block B137313) correlates with increased risk for heart disease. In some aspects, polymorphisms used in an association study constitute at least one SNP haplotype block and its constituent haplotype patterns. In yet another aspect, only informative SNPs are screened for association with a phenotypic trait of interest.

The haplotype blocks and haplotype patterns of the present invention also are useful for identifying a genetic locus, preferably a gene, within SEQ ID NO: 1 associated with a phenotypic trait of interest that is not associated with LQTS. See Lander et al., Proc. Natl. Acad. Sci. USA 84: 2363-2367 (1987) (incorporated by reference in its entirety for all purposes). Prime candidates for such a genetic locus include the gene predictions discussed supra. This can be accomplished as long as the polymorphisms, haplotype blocks or haplotype patterns of the present invention co-segregate with the genetic locus responsible for the trait; they need not be causally related to the trait. In some embodiments, a polymorphism of the invention is directly responsible for a phenotypic trait by changing the expression, function, or activity of a gene encoded by SEQ ID NO: 1. Several putative genes have already been identified in this genomic region as described supra. Such analysis is useful not only for defining associations, but also for elucidating the function of a new gene or regulatory locus, or for defining new functions of known genes, such as KCNE1 and KCNE2. Genes localized in an association study can be cloned by a process known as directional cloning and can be used to study the biological basis of the trait of interest. Further, if the trait of interest is a disease or disorder, this information could be used to develop preventative treatments or to find potential drug targets. See Collins, Nature Genetics 1: 3-6 (1992) (incorporated by reference in its entirety for all purposes).

Associations also may identify a genetic locus that could reveal information about the normal expression and function of biological molecules and complexes (e.g., the I_{Ks} and I_{Kr} ion channels), as well as the biological basis of their related disorders (e.g., LQTS). For example, heart disease is a multifactorial trait caused by both environmental and genetic factors, many of which remain unknown. By identifying the genetic factors, an individual's risk of developing heart disease could be much more accurately calculated. However, this is no small task as many of these genetic factors have very small effects on the overall phenotype. For example, a small change in the activity or function of the KCNE1 or KCNE2 proteins may not appear to have a phenotypic effect unless combined with changes in the activity or function of other proteins in a related biological pathway, such as other components of ion channels. So, even though the combination of these factors may be predictive of a susceptibility to general cardiovascular disease, their small contributions are difficult to detect when these loci are examined individually. However, by identifying these loci through the methods of the invention, their biological basis can be studied and potentially used for the development of, for example, diagnostics to identify, or therapeutics to treat, individuals at a high risk of developing heart disease. Scanning multiple regions of a genome is a powerful tool for identifying loci involved in complex phenotypic traits, especially those that result from the action of many loci that have only a small or weak individual effect. In preferred embodiments, the polymorphisms of the invention are scanned in combination with polymorphisms elsewhere in the genome to identify additional loci associated with a phenotypic trait, such as risk of LQTS-related sudden death. In more preferred embodiments, loci from all chromosomes are scanned (whole genome scanning). For example, whole genome scanning that utilizes the haplotype structure of the invention may be used in a broad screen to examine factors involved in cardiovascular disease, other disorders related to ion channel dysfunction, or other phenotypic traits in linkage disequilibrium with the polymorphisms and haplotype patterns provided herein.

B. Production and Use of Peptides

The nucleic acids of the invention may be employed for producing all or portions of an encoded RNA or polypeptide, for example, a KCNE1 or KCNE2 variant protein or the product of a gene identified in an association study as described supra. The nucleic acids of the invention may also alter the expression of a protein, which may be encoded within SEQ ID NO: 1 or in other regions of the genome, and so may be used to study the biological effect of the altered expression as well as the structure-function and regulatory characteristics of the protein. To express an RNA or protein product, an expression cassette incorporating the corresponding nucleic acid may be employed. The expression cassette or vector generally provides a transcriptional and translational initiation region, which may be inducible or constitutive, where the coding region is operably linked under the transcriptional control of the transcriptional initiation region, and a transcriptional and translational termination region. These control regions may be native to the identified gene, or may be derived from exogenous sources.

The peptide may be expressed in prokaryotes or eukaryotes in accordance with conventional methods, depending on the purpose for expression. For large scale production of a protein, such as a KCNE1 or KCNE2 variant, a unicellular organism, such as E. coli, B. subtilis, S. cerevisiae, insect cells in combination with baculovirus vectors, or cells of a higher organism such as vertebrates, particularly mammals, e.g. COS 7 cells, may be used as the expression host cells. In many situations, it may be desirable to express a gene, such as a KCNE1 or KCNE2 variant, or the gene predictions discussed supra, in eukaryotic cells where the gene will benefit from native folding and post-translational modifications. Peptides also may be synthesized in the laboratory.

The modified cells or animals are useful in the study of protein function and regulation. For example, a polymorphism that correlates with the expression of a dysfunctional protein or altered expression of a normal protein would provide insight into the biological basis for the normal function and expression of that protein. In addition, mutations may be made in one or more haplotype blocks in various ways known in the art to generate targeted changes in expression level, or changes in the sequence of the encoded RNA or protein, etc. to determine the biological role of different regions of the haplotype block and to study the expression and function of encoded genes, such as KCNE1 and KCNE2. The mutations may be substitutions, insertions, translocations or deletions. Deletions may include large changes, such as deletions of an entire domain or exon. Techniques for in vitro mutagenesis of cloned genes are known. Examples of protocols for site specific mutagenesis may be found in Sambrook, et al., Molecular Cloning: A Laboratory Manual (Cold Spring Harbor Laboratory, New York) (2001). Specific constructs of interest include, but are not limited to, antisense constructs to block gene expression, polymorphisms that reduce or prevent transcription, and polymorphisms that cause over-expression of the encoded gene. For example, a polymorphism may associate with increased expression of KCNE1. Further investigation of the biological basis of this correlation could reveal ways to produce large amounts of KCNE1 protein for further study. One may also provide for expression of a gene or variants thereof in cells or tissues where it is not normally expressed or at abnormal times of development. For example, one of the polymorphisms or a targeted mutation of a haplotype block may correlate with aberrant expression of KCNE2 in skeletal muscle. The phenotype associated with this expression pattern may provide insight into the normal function of the protein.

Variant proteins encoded by the nucleic acids of the present invention are also provided. With the availability of the protein or fragments thereof in large amounts, the protein may be isolated and purified in accordance with conventional ways. A lysate may be prepared of the expression host and the RNA or protein purified using HPLC, exclusion chromatography, gel electrophoresis, affinity chromatography, or other purification techniques. An expressed protein variant may be used for the production of antibodies, where short fragments induce the expression of antibodies specific for the particular polypeptide (monoclonal antibodies), and larger fragments or the entire protein allow for the production of antibodies over the length of the polypeptide (polyclonal antibodies). Antibodies are prepared in accordance with conventional ways, where the expressed polypeptide or protein is used as an immunogen, by itself or conjugated to known immunogenic carriers, e.g. KLH, pre-S HBsAg, other viral or eukaryotic proteins, or the like. Various adjuvants may be employed, with a series of injections, as appropriate. For monoclonal antibodies, after one or more booster injections, the spleen is isolated, the lymphocytes immortalized are by cell fusion and screened for high affinity antibody binding. The immortalized cells, i.e., hybridomas, producing the desired antibodies may then be expanded. For further description, see Monoclonal Antibodies: A Laboratory Manual, Harlow and Lane, eds. (Cold Spring Harbor Laboratories, Cold Spring Harbor, N.Y.) (1988). If desired, the mRNA encoding the heavy and light chains may be isolated and mutagenized by cloning in E. coli, and the heavy and light chains mixed to further enhance the affinity of the antibody. Alternatives to in vivo immunization as a method of raising antibodies include binding to phage “display” libraries, usually in conjunction with in vitro affinity maturation. The antibodies may be used with or without modification, and may be labeled by covalent or non-covalent attachment of a reporter molecule.

C. Diagnostics

Preventative measures are very successful in preventing LQTS-related ventricular fibrillation and sudden death, but many individuals with this disorder remain unidentified due to the variable phenotype and unreliable testing methods. Associations may be utilized to assess risk or susceptibility to a disease or other condition (diagnostics). For example, detection of the polymorphisms, haplotype patterns, and haplotype blocks of the invention in a target DNA sample may be used to determine whether an individual has an increased risk of LQTS or LQTS-related drug sensitivity, or other phenotypic trait in linkage disequilibrium with the polymorphisms, haplotype patterns, and haplotype blocks of the invention. In the case of an association between a set of one or more polymorphisms and an increased risk of LQTS, detection of the set of polymorphisms in an individual may justify the institution of preventative measures (e.g., avoidance of extreme physical exertion) or immediate administration of a treatment regimen (e.g., beta-blocker drug therapy). Alternatively, they may also be used to identify individuals who are resistant to a disease, infection, or other condition. For example, some individuals who display a lengthened QT interval never experience ventricular tachycardia and so are at a very low risk of sudden death. This knowledge could preclude more drastic treatments, such as the use of an implantable cardioverter defibrillator (ICD) in these individuals. Associations may also be used to identify individuals with increased risk of adverse, non-disease conditions and to motivate life-style changes to prevent onset of the condition. For example, an association between a haplotype pattern and obesity could provide strong incentive to exercise and eat a healthy diet. Further, an association between a haplotype pattern and an LQTS-related drug sensitivity would disallow administration of that drug to an individual.

An association may or may not be due to direct effects of the polymorphisms on the phenotypic trait of interest. For example, a polymorphism that is found to associate with a high risk of LQTS-related sudden death may affect the expression or function of the KCNE1 or KCNE2 protein directly, or may be in linkage disequilibrium with (and so predictive of) another locus that affects the expression or function of one or both of these proteins. As such, a polymorphism within a nucleic acid may be used for diagnosis of a disorder that is associated with a genetic locus that is linked to the polymorphism, but not necessarily within the nucleic acid. Examples of direct effects to the expression or function of a protein include, but are not limited to, a polymorphism that alters the polypeptide sequence of the protein, and a polymorphism that occurs in a regulatory region (i.e., promoter, enhancer, etc.) resulting in the increased or decreased expression of the protein. However, the polymorphisms themselves need not be directly involved in the manifestation of the phenotypic trait of interest in order to serve as a means to identify genomic regions that are involved; they need only be correlated with that trait and genetically linked to the genomic region. In preferred embodiments, the set of polymorphisms used in the association studies would be chosen based on the genomic haplotype structure of an organism. In more preferred embodiments, the polymorphisms would be SNPs in identifiable haplotype patterns. In more preferred embodiments, at least one of the polymorphisms would be an informative SNP.

The nucleic acids and haplotype structure of the invention may also be used to detect or quantify expression of an encoded gene, such as KCNE1 or KCNE2, or other genes in linkage disequilibrium with the nucleic acids and haplotype structure in a biological specimen for use as a diagnostic marker, e.g., to predict a phenotypic characteristic such as disease susceptibility or drug responsiveness by using nucleic acids of the invention as probes to determine whether a particular polymorphism or a set of polymorphisms is present in the genome of an organism being tested. For example, the nucleic acids may be used as oligonucleotide probes to monitor RNA or mRNA levels within the organism to be tested or a part thereof, such as a specific tissue or organ, so as to determine the expression level of the gene encoding the RNA or mRNA, where the expression level can be correlated to a particular phenotypic characteristic of the organism. Likewise, the expression of the gene may be assayed at the protein level using any customary technique such as immunological methods (e.g., Western blots, radioimmune precipitation and the like) or activity based assays measuring an activity associated with the gene product. The manner in which cells are probed for the presence of particular nucleotide or polypeptide sequences is well established in the literature and does not require further elaboration here, however, see, e.g., Sambrook, et al., Molecular Cloning: A Laboratory Manual (Cold Spring Harbor Laboratory, New York) (2001).

Antibodies which bind specifically to the gene products of the present invention (i.e., KCNE1 or KCNE2 variants) also may be used for the diagnosis of disorders characterized by their expression, or in assays to monitor patients being treated with the gene products or with agonists, antagonists or inhibitors of the gene products. Diagnostic assays for the gene products of the present invention include methods which utilize an antibody and a label to detect the gene product in human body fluids or in extract of cells or tissues, such as heart muscle.

D. Pharmacogenomics

Associations may be used for pharmacogenomic studies and drug development. For example, since the response of individuals with LQTS to different treatments varies, identifying sets of polymorphisms that associate with positive (or negative) response or side-effects to an administered drug or other treatment would be useful for stratifying patient populations and individualizing treatment regimens. In addition, associations may be used to develop clinical trials for new treatments for LQTS and other disorders or diseases by allowing stratification of the patient population. For example, if an antiarrythmia drug were to be tested for efficacy and safety, it would be valuable to identify and remove individuals with LQTS from the population to be tested, since these individuals are at a higher risk of ventricular fibrillation when these types of drugs are administered. Further, if a new drug for treatment of potassium channel-related LQTS were being tested, then a population of individuals with LQTS could be stratified based on the type of LQTS that they possess. For example, individuals with a sodium channel-related LQTS would likely be nonresponders and could be excluded while individuals with a potassium channel-related LQTS would be more likely to be responders and could be included in the study. Even a population of individuals with potassium channel-related LQTS may be further stratified based on polymorphisms that associate with responses to different classes of drugs and thereby distinguish probable responders from nonresponders from individuals likely to have toxic side effects.

E. Therapeutics

The nucleic acids, or the encoded protein variant or fragments thereof may be useful in gene therapy to treat potassium ion channel-related disorders, such as LQTS, and other disorders found to be in linkage disequilibrium with the polymorphisms and haplotype structure of the invention. For example, expression vectors may be used to introduce an identified gene (e.g., a beneficial variant of KCNE1) into a cell. Such vectors generally have convenient restriction sites located near the promoter sequence to provide for the insertion of nucleic acid sequences in a recipient genome. Transcription cassettes may be prepared comprising a transcription initiation region, the target gene or fragment thereof, and a transcriptional termination region. The transcription cassettes may be introduced into a variety of vectors, e.g. plasmid; retrovirus, e.g. lentivirus; adenovirus; and the like, where the vectors are able to be transiently or stably maintained in the cells. The gene or protein product may be introduced directly into tissues or host cells by any number of routes, including viral infection, microinjection, or fusion of vesicles. Jet injection may also be used for intramuscular administration, as described by Furth, et al., Anal. Biochem, 205: 365-68 (1992). Alternatively, the DNA may be coated onto gold microparticles, and delivered intradermally by a particle bombardment device or “gene gun” as described in the literature (see, for example, Tang, et al., Nature, 356: 152-54 (1992)).

Antibodies which bind specifically to the gene products of the present invention (i.e., KCNE1 or KCNE2 variants) may be used as therapeutics. For example, such antibodies may be administered to a patient as a means to inhibit the activity of a detrimental variant of KCNE1, KCNE2, or another variant protein encoded by SEQ ID NO: 1.

Antisense molecules may be used to down-regulate expression of an identified gene (e.g., a detrimental variant of KCNE2) in cells. An antisense molecule forms a duplex with the mRNA of a gene whose expression is to be down-regulated, blocking translation of the corresponding protein. For example, if a KCNE2 variant is found to be correlated with an increased risk of LQTS in a patient who is heterozygous for the wildtype (normal) version of KCNE2, then an antisense reagent may be developed based on the sequence of the mRNA of the KCNE2 variant. This antisense agent may then be administered to the patient to decrease the expression of the detrimental KCNE2 variant, allowing the expression of the wildtype KCNE2 to predominate. The antisense reagent may be antisense oligonucleotides, particularly synthetic antisense oligonucleotides having chemical modifications, or nucleic acid constructs that express such antisense molecules as RNA. A combination of antisense molecules may be administered, where a combination may comprise multiple different sequences.

As an alternative to antisense inhibitors, catalytic nucleic acid compounds, e.g., ribozymes, anti-sense conjugates, etc., may be used to inhibit expression of detrimental gene variants. Ribozymes may be synthesized in vitro and administered to the patient, or may be encoded on an expression vector, from which the ribozyme is synthesized in the targeted cell (for example, see International patent application WO 9523225, and Beigelman, et al., Nucl. Acids Res. 23: 4434-42 (1995)). Examples of oligonucleotides with catalytic activity are described in WO 9506764. Conjugates of antisense oligonucleotides with a metal complex, e.g. terpyridylCu(II), capable of mediating mRNA hydrolysis are described in Bashkin, et al., Appl. Biochem. Biotechnol. 54: 43-56 (1995).

An expressed protein encoded by a nucleic acid of the invention also may be used in drug screening assays to identify ligands or substrates that bind to, modulate or mimic the action of that protein product, and thereby identify therapeutic agents to provide, for example, a replacement or enhancement for protein function in affected cells, or an agent that modulates or negates protein function. A wide variety of assays may be used for this purpose, including labeled in vitro protein-protein binding assays, protein-DNA binding assays, electrophoretic mobility shift assays, immunoassays for protein binding, and the like. The term “agent” as used herein describes any molecule, e.g., a protein or small molecule, with the capability of altering, mimicking or masking, either directly or indirectly, the physiological function of an identified gene or gene product. Generally pluralities of assays are run in parallel with different concentrations of the agent to obtain a differential response to the various concentrations. Typically, one of these concentrations serves as a negative control, e.g., at zero concentration or below the level of detection. Also, all or a fragment of a purified protein variant may be used for determination of three-dimensional crystal structure, which can be used for determining the biological function of the protein or a part thereof, modeling intermolecular interactions, membrane fusion, etc.

Candidate agents encompass numerous chemical classes, though typically they are organic molecules or complexes, preferably small organic compounds, having a molecular weight of more than 50 and less than about 2,500 daltons. Candidate agents comprise functional groups necessary for structural interaction with proteins, particularly hydrogen bonding, and typically include at least an amine, carbonyl, hydroxyl or carboxyl group, and frequently at least two of the functional chemical groups. The candidate agents often comprise cyclical carbon or heterocyclic structures and/or aromatic or polyaromatic structures substituted with one or more of the above functional groups. Candidate agents are also found among biomolecules including, but not limited to: peptides, saccharides, fatty acids, steroids, purines, pyrimidines, derivatives, structural analogs or combinations thereof.

Candidate agents are obtained from a wide variety of sources including libraries of synthetic or natural compounds. For example, numerous means are available for random and directed synthesis of a wide variety of organic compounds and biomolecules, including expression of randomized oligonucleotides and oligopeptides. Alternatively, libraries of natural compounds in the form of bacterial, fungal, plant and animal extracts are available or readily produced. Additionally, natural or synthetically produced libraries and compounds are readily modified through conventional chemical, physical and biochemical means, and may be used to produce combinatorial libraries. Known pharmacological agents may be subjected to directed or random chemical modifications, such as acylation, alkylation, esterification, amidification, etc., to produce structural analogs.

Where the screening assay is a binding assay, one or more of the molecules may be coupled to a label, where the label can directly or indirectly provide a detectable signal. Various labels include radioisotopes, fluorescers, chemiluminescers, enzymes, specific binding molecules, particles, e.g., magnetic particles, and the like. Specific binding molecules include pairs, such as biotin and streptavidin, digoxin and antidigoxin, etc. For the specific binding members, the complementary member would normally be labeled with a molecule that provides for detection, in accordance with known procedures. A variety of other reagents may be included in the screening assay. These include reagents like salts, neutral proteins, e.g., albumin, detergents, etc that are used to facilitate optimal protein-protein binding and/or reduce non-specific or background interactions. Reagents that improve the efficiency of the assay, such as protease inhibitors, nuclease inhibitors, anti-microbial agents, etc., may be used.

Agents may be combined with a pharmaceutically acceptable carrier or diluent, including any and all solvents, dispersion media, coatings, anti-oxidant, isotonic and absorption delaying agents and the like. The agent may be combined with conventional additives, such as lactose, mannitol, corn starch or potato starch; with binders, such as crystalline cellulose, cellulose derivatives, acacia, corn starch or gelatins; with disintegrators, such as corn starch, potato starch or sodium carboxymethylcellulose; with lubricants, such as talc or magnesium stearate; and if desired, with buffering agents, moistening agents, preservatives and flavoring agents. The use of such media and agents for pharmaceutically active substances is well known in the art and are readily available to the public. Moreover, pharmaceutically acceptable auxiliary substances, such as pH adjusting and buffering agents, tonicity adjusting agents, stabilizers, wetting agents and the like, are readily available to the public. Except insofar as any conventional media or agent is incompatible with the active ingredient, its use in the therapeutic compositions and methods described herein is contemplated. Supplementary active ingredients can also be incorporated into the compositions.

The following methods and excipients are merely exemplary and are in no way limiting. Identified agents of the invention can be incorporated into a variety of formulations for therapeutic administration. More particularly, the complexes can be formulated into pharmaceutical compositions by combination with appropriate, pharmaceutically acceptable carriers or diluents as discussed supra, and may be formulated into preparations in solid, semi-solid, liquid or gaseous forms, such as tablets, capsules, powders, granules, ointments, solutions, gels, microspheres, and aerosols. Additionally, agents may be formulated into preparations for injections by dissolving, suspending or emulsifying them in an aqueous or nonaqueous solvent, such as vegetable or other similar oils, synthetic aliphatic acid glycerides, esters of higher aliphatic acids or propylene glycol; and if desired, with conventional additives such as solubilizers, isotonic agents, suspending agents, emulsifying agents, stabilizers and preservatives. Further, agents may be utilized in aerosol formulation to be administered via inhalation. The agents identified by the present invention can be formulated into pressurized acceptable propellants such as dichlorodifluoromethane, propane, nitrogen and the like. Alternatively, agents may be made into suppositories for rectal administration by mixing with a variety of bases such as emulsifying bases or water-soluble bases and can include vehicles such as cocoa butter, carbowaxes and polyethylene glycols, which melt at body temperature, yet are solid at room temperature.

Implants for sustained release formulations are well known in the art. Implants are formulated as microspheres, slabs, etc. with biodegradable or non-biodegradable polymers. For example, polymers of lactic acid and/or glycolic acid form an erodible polymer that is well-tolerated by the host. The implant containing identified agents of the present invention may be placed in proximity to the site of action, so that the local concentration of active agent is increased relative to the rest of the body. Unit dosage forms for oral or rectal administration such as syrups, elixirs, and suspensions may be provided wherein each dosage unit, for example, teaspoonful, tablespoonful, gel capsule, tablet or suppository, contains a predetermined amount of the compositions of the present invention. Similarly, unit dosage forms for injection or intravenous administration may comprise the compound of the present invention in a composition as a solution in sterile water, normal saline or another pharmaceutically acceptable carrier. The specifications for the novel unit dosage forms of the present invention depend on the particular compound employed and the effect to be achieved, and the pharmacodynamics associated with each active agent in the host.

Administration of the agents can be achieved in various ways. The formulation may be given orally, by inhalation, or may be injected, e.g. intravascular, intratumor, subcutaneous, intraperitoneal, intramuscular, etc. Agents may be topical, systemic, or may be localized by the use of an implant that acts to retain the active dose at the site of implantation. The dosage of the therapeutic formulation will vary, depending on the specific agent and formulation utilized, the nature of the disease, the frequency of administration, the manner of administration, the clearance of the agent from the host, and the like, such that it is sufficient to address the disease or symptoms thereof, while minimizing side effects. In some cases, oral administration will require a different dose than if administered intravenously. The compounds will be administered at an effective dosage such that over a suitable period of time the disease progression may be substantially arrested. The initial dose may be larger, followed by smaller maintenance doses. The dose may be administered as infrequently as once, weekly or biweekly, or fractionated into smaller doses and administered daily, semi-weekly, etc., to maintain an effective dosage level. Treatment may be for short periods of time, e.g., after ventricular fibrillation, or for extended periods of time, e.g., in the prevention of further episodes of ventricular fibrillation. It is contemplated that the composition will be obtained and used under the guidance of a physician for in vivo use.

F. Other Uses and Aspects of the Invention

It should be apparent that the methods of the present invention can be used on organisms aside from humans. For example, when the organism is an animal, the methods of the invention may be used to identify loci associated, e.g., with disease resistance or susceptibility, environmental tolerance, drug response or the like, and when the organism is a plant, the method of the invention may be used to identify loci associated with disease resistance or susceptibility, environmental tolerance and or herbicide resistance. The nucleic acids of the invention may be used to generate genetically modified non-human animals to create animal models of LQTS or other ion channel-related disorders, or to generate site-specific gene modifications in cell lines for the study of protein function or regulation. Transgenic animals may be made through homologous recombination, where the endogenous gene locus is altered, replaced or otherwise disrupted. Alternatively, a nucleic acid construct may be randomly integrated into the genome. Vectors for stable integration include plasmids, retroviruses and other animal viruses, YACs, and the like. Of interest are transgenic mammals including, but not limited to: cows, pigs, goats, horses, etc., and, particularly, rodents, e.g., rats, mice, etc. Investigation of genetic function may also utilize non-mammalian models, particularly using those organisms that are biologically and genetically well-characterized, such as C. elegans, D. melanogaster and S. cerevisiae. The nucleic acid construct may be used to knock-out corresponding gene function or to complement defined genetic lesions in order to determine the physiological and biochemical pathways involved in protein function. Drug screening may be performed in combination with complementation or knock-out studies, e.g., to study LQTS-related phenotypic traits, to test therapies, or for drug discovery. test therapies, or for drug discovery.

The invention further provides kits comprising at least one nucleic acid of the invention, preferably an oligonucleotide, more preferably an oligonucleotide primer or probe that may be used to detect a polymorphism or haplotype pattern of the invention. Often, the kits contain one or more pairs of oligonucleotide primers that hybridize to a target nucleic acid to allow amplification of one or more regions of the target that contain or are a portion of one or more haplotype blocks of the invention. In preferred embodiments, the amplification product could be analyzed to determine the genotype of the polymorphisms and/or haplotype patterns contained within the target nucleic acid. In some kits, oligonucleotide probes are provided immobilized to a substrate. In preferred embodiments, an oligonucleotide probe immobilized to a substrate hybridizes to a specific allele of a given polymorphism of the invention. For example, the same substrate can comprise oligonucleotide probes for detecting multiple or all of the polymorphisms listed in FIGS. 1 and 2. Optional additional components of the kit include, for example, restriction enzymes, reverse-transcriptase or polymerase, the substrate nucleoside triphosphates, means used to label (for example, an avidin-enzyme conjugate and enzyme substrate and chromogen if the label is biotin), and the appropriate buffers for reverse transcription, PCR, or hybridization reactions. Usually, the kit also contains instructions for carrying out the methods. These kits may facilitate both identifying those at risk of LQTS, those sensitive to the drugs that exacerbate LQTS symptoms, individuals with other phenotypic traits in linkage disequilibrium with the polymorphisms and haplotype patterns of the invention, and could also be useful for genetic counseling.

In addition, the polymorphisms, haplotype patterns and haplotype blocks of biological matter. Rare SNPs may be particularly useful for this application. This biological matter may be collected at a crime scene or from the victim of a crime, and could be used to construct a genetic profile of the perpetrator of the crime. This technology could provide a genetic profile to match a given sample to a specific individual, and may both provide stronger evidence for convicting the guilty and definitive evidence to clear many who have been wrongly convicted, some of whom may be awaiting a death sentence. Further, associations also may be used to help couples make informed reproductive decisions based on the genetic makeup and haplotype structure of their own genomes.

A database is also provided for use in recording and cataloging the polymorphisms, haplotype blocks, and haplotype patterns of the invention. The database may also contain data obtained from association studies, drug screening studies, and other utilities of the invention. The database may also contain information on LQTS or other disorders in linkage disequilibrium with the polymorphisms of the invention including, but not limited to, environmental factors, genetic factors from genomic regions outside of SEQ ID NO: 1, biochemical or genetic markers, behaviors, other polymorphisms such as insertions, deletions, inversions, translocations, RFLPs, and the like. The database may be stored on a computer-readable medium.

VI. Conclusion

The present inventions provide nucleic acids comprising polymorphisms, haplotype patterns and haplotype blocks, as well as greatly improved methods for developing diagnostics and therapeutics, and discovering the biological basis underlying a plethora of phenotypic traits. It is to be understood that the above description is intended to be illustrative and not restrictive, and that the invention is not limited to the particular methodology, protocols, cell lines, animal species or genera, and reagents described, as such may vary. It is also to be understood that the terminology used herein is for the purpose of describing particular embodiments only, and is not intended to limit the scope of the present invention. One skilled in the art will readily recognize that the polymorphisms, haplotype blocks, haplotype patterns, and nucleic acids of the invention may be used in many different applications in addition to the examples described herein. The scope of the invention should, therefore, be determined not with reference to the above description, but should instead be determined with reference to the appended claims, along with the full scope of equivalents to which such claims are entitled.

What is claimed is:

1. A method identifying a genetic locus involved in the biological basis of a phenotypic trait of interest, said method comprising:
a) obtaining a biological sample containing nucleic acids from a control population that does not possess said phenotypic trait;
b) obtaining a biological sample containing nucleic acids from a clinical population that does possess said phenotypic trait;
c) determining allelic frequency for at least one single nucleotide polymorphism listed in FIG. 1 or FIG. 2 in said control population;
d) determining the allelic frequency for the same one or more single nucleotide polymorphisms as used in step c) in said clinical population; and
e) comparing said allelic frequency from step c) to said allelic frequency from step d), wherein differences in said allelic frequencies indicate the presence of genetic loci involved in the biological basis of said phenotypic trait of interest.

2. The method of claim 1, wherein said isolated nucleic acid molecule comprises at least one haplotype pattern of FIG. 3.

3. The method of claim 1, further comprising the step of selecting said genetic loci as drug discovery targets or diagnostic markers for said phenotypic trait of interest.

4. The method of claim 1, wherein said frequencies are determined in more than 10 individuals.

5. The method of claim 1, wherein said frequencies are determined in more than 100 individuals.

6. The method of claim 1, wherein said frequencies are determined in more than 1000 individuals.

7. The method of claim 1, wherein said phenotypic trait of interest is a cardiovascular disorder, a response to a drug, a hearing disability, or a potassium ion channel disorder.

8. A method of screening an individual for a predisposition, susceptibility, or resistance to a phenotypic trait of interest, said method comprising use of an isolated nucleic acid molecule comprising at least 10 nucleotides of SEQ ID NO: 1 wherein said isolated nucleic acid molecule includes at least one alternative base as listed in FIG. 1 or FIG. 2, or a complementary sequence thereto and wherein the presence or absence of said isolated nucleic acid molecule indicates a predisposition, susceptibility, or resistance to cardiovascular disorder, response to a drug, a hearing disability, or a potassium ion channel disorder, comprising the steps of:
a) obtaining a biological sample from an individual;
b) analyzing the biological sample for the presence of said isolated nucleic acid molecule; and
c) determining the predisposition, susceptibility, or resistance of the individual to said phenotypic trait of interest based on the presence or absence of said isolated nucleic acid molecule in said individual.

9. The method of claim 8, wherein at least 10 individuals are screened for a predisposition, susceptibility, or resistance to said phenotypic trait of interest.

10. The method of claim 9, wherein at least 100 individuals are screened for a predisposition, susceptibility, or resistance to said phenotypic trait of interest.

11. The method of claim 9, wherein at least 1000 individuals are screened for a predisposition, susceptibility, or resistance to said phenotypic trait of interest.

12. A method for selecting an appropriate therapeutic for an individual that has or is predisposed to a phenotypic trait of interest, wherein said phenotypic trait of interest is associated with an isolated nucleic acid molecule comprising at least 10 nucleotides of SEQ ID NO: 1 and wherein said isolated nucleic acid molecule includes at least one alternative base as listed in FIG. 1 or FIG. 2, or a complementary sequence thereto, comprising the steps of:
a) detecting whether said individual possesses said isolated nucleic acid molecule; and
b) selecting a therapeutic that compensates for a causative functional mutation that is in linkage disequilibrium with said isolated nucleic acid molecule.

13. The method of claim 12, wherein said therapeutic is a protein, peptide, peptidomimetic, carbohydrate, small molecule, or nucleic acid, or derivative thereof.

14. The method of claim 12, wherein said method is used to stratify a clinical population.

15. A kit for diagnosing a disease, disease susceptibility, or therapy response associated with an isolated nucleic acid molecule comprising at least 10 nucleotides of SEQ ID NO: 1 wherein said isolated nucleic acid molecule includes at least one alternative base as listed in FIG. 1 or FIG. 2, or a complementary sequence thereto, comprising means for detecting a presence or absence of said isolated nucleic acid molecule in a sample of DNA from a patient and a data set of associations of said isolated nucleic acid molecule with said disease, disease susceptibility, or therapy response on a computer-readable medium.

16. The kit of claim 15, wherein said data set of associations further comprises haplotype patterns or informative SNPs within said isolated nucleic acid molecule that are associated with said disease, disease susceptibility, or therapy response.
